(19) United States
(12) Patent Application Publication
(10) Pub. No.: US 2025/0166821 A1
(43) Pub. Date: May 22, 2025

(54) METHODS FOR PREDICTION OF TREATMENT RESPONSE IN NON-ALCOHOLIC FATTY LIVER DISEASE

(71) Applicant: Sagimet Biosciences Inc., San Mateo, CA (US)

(72) Inventor: Anne-Marie O'Farrell, San Mateo, CA (US)

(21) Appl. No.: 18/709,271

(22) PCT Filed: Nov. 10, 2022
(86) PCT No.: PCT/US2022/049566

§ 371 Date: May 10, 2024

Related U.S. Application Data
(60) Provisional application No. 63/417,970, filed on Oct. 20, 2022.
(60) Provisional application No. 63/278,298, filed on Nov. 11, 2021.

(51) Int. Cl. G16H 50/20; A61K 31/4155; A61K 31/4196; A61P 1/16; G16H 10/40; G16H 50/70

(52) U.S. Cl. CPC G16H 50/20; A61K 31/4155; A61K 31/4196; A61P 1/16; G16H 10/40; G16H 50/70

(57) ABSTRACT

The present disclosure relates to a method for determining a treatment regimen to administer to a subject having or suspected of having non-alcoholic steatohepatitis (NASH) or non-alcoholic fatty liver disease (NAFLD), comprising receiving, by processing circuitry, input data associated with the subject, the input data including biochemical data characterizing a state of the subject, applying, by the processing circuitry, a machine learning model to the input data to generate an output, and comparing, by the processing circuitry, the generated output from the machine learning model to a treatment threshold. In embodiments, the present disclosure further relates to administering the treatment regimen when the generated output satisfies the treatment threshold. In embodiments, the treatment regimen may be an ATP-citrate lyase inhibitor, an acetyl-CoA carboxylase inhibitor, a fatty acid synthase inhibitor, and/or a stearoyl-CoA desaturase inhibitor.

### RELATED APPLICATIONS

This application claims the benefit of and priority to U.S. provisional application Nos. 63/278,298, filed Nov. 11, 2021, and 63/417,970, filed Oct. 20, 2022, the entire contents of which are incorporated herein by reference in its entirety.

### BACKGROUND

Non-alcoholic fatty liver disease (NAFLD), a condition in which the liver contains more than 5% fat by weight and is not caused by alcohol consumption, is a disease which currently affects ˜20-30% of the US and general western world population and is associated with a significantly increased risk of morbidity extending beyond the liver to cardiovascular disease (i.e., carotid atherosclerotic plaques and endothelial dysfunction), chronic kidney disease, and malignancy. Obesity and metabolic syndrome are two key risk factors for NAFLD which are characterized as imbalance in energy utilization and storage. This imbalance leads to dysregulated metabolic pathways and inflammatory responses that drive further changes leading to liver damage and comorbid conditions. Along with the progression of metabolic syndrome, NAFLD leads to more advanced liver disease starting with non-alcoholic steatohepatitis (NASH) which can then progress to significant liver disease including cirrhosis and hepatocellular carcinoma.

The synthesis of fatty acids in the liver, a pathway termed hepatic de novo lipogenesis (DNL), is increased in subjects with metabolic syndrome and NAFLD (Donnelly, K. L., et al., “Sources of Fatty Acids Stored in Liver and Secreted Via Lipoproteins in Patients with Nonalcoholic Fatty Liver Disease,” J. Clin. Invest. 115 (5): 2005, 1343-51; Lambert, J. E., et Al., “Increased De Novo Lipogenesis Is a Distinct Characteristic of Individuals with Nonalcoholic Fatty Liver Disease,” Ygast 146 (3): 2014, 726-35). The DNL pathway not only produces fatty acids that contribute to elevated liver stores of triglycerides, but the fatty acids that are produced are saturated fatty acid species, primarily palmitate, which contribute to signaling events that increase liver inflammation (Wei, Y., “Saturated Fatty Acids Induce Endoplasmic Reticulum Stress and Apoptosis Independently of Ceramide in Liver Cells,” Am. J. Physio. Endocrinol. Metab. 291 (2): 2006, E275-81; Kakazu, E., et al., “Hepatocytes Release Ceramide-rich Proinflammatory Extracellular Vesicles in an IRE1alpha dependent manner,” Abstract 58. AASLD—The Liver Meeting, San Francisco, CA, USA, 13-17, November 2015).

Among other enzymes, one of the key enzymes in the DNL pathway is a fatty acid synthase (FASN) which is solely responsible for synthesizing palmitate. Targeting this and other enzymes of the DNL pathway (e.g., ATP-citrate lyase, acetyl-CoA carboxylases 1, and/or stearoyl-CoA desaturase-1) may result in disruption of the DNL pathway and serve as an important strategy for therapeutic intervention to reduce the consequences associated with metabolic syndrome and NAFLD.

In particular, inhibition of FASN has the potential to be a treatment for a wide range of diseases including cancer, acne, viral disease, metabolic disease, NAFLD, NASH, and inflammatory disease (e.g., rheumatoid arthritis, gout, pulmonary fibrosis, COPD, IBD, and transplant rejection). Additionally, FASN inhibition may provide therapeutic benefits in cardiovascular disease, type II diabetes, and metabolic syndrome. Successful treatment of these diseases is still a highly unmet need. While there are treatments available for diabetes and cardiovascular disease, there are currently no drugs approved to treat metabolic syndrome, NAFLD, or NASH.

Further to the above, and appreciating that few, if any, treatments may be effective, there currently exists no way to determine, in advance, whether a patient will be responsive to one treatment or another. As a result, patients and physicians may dedicate time to futile treatments. Thus, there is a need for a method of determining a treatment regimen for a patient by predicting responsiveness of the patient to a particular treatment strategy.

### SUMMARY

According to some embodiments, the present disclosure relates to a method for determining a treatment regimen to administer to a subject having or suspected of having non-alcoholic steatohepatitis (NASH) or non-alcoholic fatty liver disease (NAFLD), comprising (i) receiving, by processing circuitry, input data associated with the subject, the input data including biochemical data characterizing a state of the subject, (ii) applying, by the processing circuitry, a machine learning model to the input data to generate an output, and (iii) comparing, by the processing circuitry, the generated output from the machine learning model to a treatment threshold.

In some embodiments, the method further comprises administering the treatment regimen when the generated output satisfies the treatment threshold.

In some embodiments, the treatment regimen includes an ATP-citrate lyase (ACLY) inhibitor, an acetyl-CoA carboxylases 1 (ACC1) inhibitor, a fatty acid synthase (FASN) inhibitor, and/or a stearoyl-CoA desaturase-1 (SCD1) inhibitor.

In some embodiments, the treatment regimen is a FASN inhibitor and the generated output is a predicted percent change in liver fat following administration of the FASN inhibitor.

In some embodiments, the machine learning model is trained on a database of training data that includes biochemical data characterizing a state of a reference subject before administration of the treatment regimen and a corresponding percent change in liver fat following administration of the treatment regimen.

According to some embodiments, the present disclosure relates to a method of treating a subject having or suspected of having non-alcoholic steatohepatitis (NASH) or non-alcoholic fatty liver disease (NAFLD), comprising obtaining biochemical data characterizing a state of the subject, determining a result via processing circuitry configured to generate the result by applying a machine learning model to the obtained biochemical data, and administering a treatment to the subject when the determined result satisfies a treatment threshold.

According to some embodiments, the present disclosure relates to a method of treating a subject having or suspected of having non-alcoholic steatohepatitis (NASH) or non-alcoholic fatty liver disease (NAFLD) with a fatty acid synthase (FASN) inhibitor, comprising (i) receiving, by processing circuitry, input data associated with the subject, the input data including biochemical data characterizing a state of the subject, (ii) applying, by the processing circuitry, a machine learning model to the input data to generate an output value predicting a percent change in liver fat following administration of the FASN inhibitor, (iii) comparing, by the processing circuitry, the output value of the machine learning model to a treatment threshold value, and (iv) administering an effective amount of the FASN inhibitor to the subject if the comparing indicates the predicted percent change in liver fat exceeds the treatment threshold value.

In some embodiments, the biochemical data characterizing the state of the subject includes at least one bile acid, at least one secondary bile acid, at least one bile acid derivative, at least one amino acid, at least one amino acid derivative, and/or at least one glycerophospholipid.

In some embodiments, the biochemical data characterizing the state of the subject include ursodeoxycholic acid, hyodeoxycholic acid, DL-2-aminocaprylic acid, D(−)-2-aminobutyric acid, sarcosine, glycoursodeoxycholic acid, and/or PC(O-18:0/22:4).

In some embodiments, the biochemical data characterizing the state of the subject include ursodeoxycholic acid, hyodeoxycholic acid, DL-2-aminocaprylic acid, sarcosine, glycoursodeoxycholic acid, and/or PC(O-18:0/22:4).

In some embodiments, the biochemical data characterizing the state of the subject include ursodeoxycholic acid, DL-2-aminocaprylic acid, sarcosine, glycoursodeoxycholic acid, D(−)-2-aminobutyric acid, and/or PC(O-18:0/22:4).

In some embodiments, the machine learning model is a random decision forest.

In some embodiments, the biochemical data included in the random decision forest is based on mean decrease in impurity.

In some embodiments, the machine learning model is a support-vector machine.

In some embodiments, the biochemical data included in the support-vector machine is based on a permutation importance algorithm.

In some aspects, the present disclosure relates to an apparatus for treating a subject having or suspected of having non-alcoholic steatohepatitis (NASH) or non-alcoholic fatty liver disease (NAFLD) with a fatty acid synthase (FASN), comprising processing circuitry configured to: (i) receive input data associated with the subject, the input data including biochemical data characterizing a state of the subject, (ii) apply a machine learning model to the input data to generate an output value predicting a percent change in liver fat following administration of the FASN inhibitor, and (iii) compare the output value of the machine learning model to a treatment threshold value.

According to some embodiments, the present disclosure relates to a non-transitory computer-readable storage medium storing computer-readable instructions that, when executed by a computer, cause the computer to perform a method for determining a regimen of a fatty acid synthase (FASN) inhibitor to administer to a subject having or suspected of having non-alcoholic steatohepatitis (NASH) or non-alcoholic fatty liver disease (NAFLD), comprising: (i) receiving input data associated with the subject, the input data including biochemical data characterizing a state of the subject, (ii) applying a machine learning model to the input data to generate an output value predicting a percent change in liver fat following administration of the FASN inhibitor, and (iii) compare the output value of the machine learning model to a treatment threshold value.

In some embodiments of the non-transitory computer-readable storage medium storing computer-readable instructions, the method for determining a regimen of a fatty acid synthase (FASN) inhibitor to administer to a subject having or suspected of having non-alcoholic steatohepatitis (NASH) or non-alcoholic fatty liver disease (NAFLD) further comprises administering an effective amount of the FASN inhibitor to the subject if the comparing indicates the predicted percent change in liver fat exceeds the treatment threshold value.

According to some embodiments, the present disclosure further relates to a method for administering a fatty acid synthase (FASN) inhibitor to a subject having non-alcoholic steatohepatitis (NASH), comprising determining a predicted responsiveness of the subject having NASH to the FASN inhibitor by calculating a prognostic value based on results of a biomarker assay performed on a biological sample obtained from the subject having NASH, and administering the FASN inhibitor to the subject having NASH when the calculated prognostic value indicates a high predicted responsiveness of the subject having NASH to the FASN inhibitor.

In some embodiments, the biological sample obtained from the subject having NASH includes, as biomarkers, ursodeoxycholic acid, hyodeoxycholic acid, DL-2-aminocaprylic acid, glycoursodeoxycholic acid, sarcosine, and/or PC(0-18:0/22:4).

In some embodiments, the prognostic value is obtained by applying a machine learning model to the results of the biomarker assay, the machine learning model being trained on a database of training data, the database including biomarker data characterizing a state of a reference subject before administration of the FASN inhibitor and a corresponding percent change in liver fat following administration of the FASN inhibitor.

According to some embodiments, the present disclosure relates to a method for administration of a treatment to a subject with non-alcoholic steatohepatitis (NASH), comprising: (i) determining a predicted responsiveness of the subject with NASH to the treatment by calculating a prognostic value based on results of a biomarker assay performed on a biological sample obtained from the subject with NASH, and (ii) administering the treatment to the subject with NASH when the calculated prognostic value indicates a high predicted responsiveness of the subject with NASH to the treatment.

According to some embodiments, the FASN inhibitor of the method, apparatus, or non-transitory computer-readable storage medium of any one of the above aspects or embodiments is a compound of
(i) Formula (IX-1):

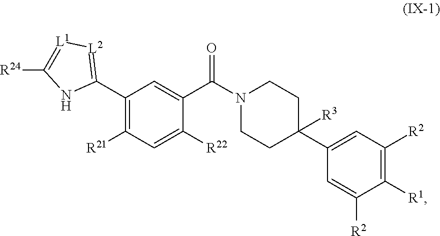

or a pharmaceutically acceptable salt thereof, wherein: R¹ is H, —CN, halogen, C₁-C₄ straight or branched alkyl, —O—(C₃-C₅ cycloalkyl), —O-(4- to 6-membered heterocycle), or —O—(C₁-C₄ straight or branched alkyl), wherein when R¹ is not H, —CN or halogen, it is optionally substituted with one or more halogens; each R² is independently H, halogen or C₁-C₄ straight or branched alkyl; R³ is H, —OH, or halogen; R²¹ is H, halogen, C₁-C₄ straight or branched alkyl, C₃-C₅ cycloalkyl wherein the C₃-C₅ cycloalkyl optionally includes an oxygen or nitrogen heteroatom; R²² is H, halogen, or C₁-C₂ alkyl; R²⁴ is H, C₁-C₄ straight or branched alkyl, —(C₁-C₄ alkyl)ₜ-OH, —(C₁-C₄ alkyl)ₜ-Oₜ—(C₃-C₅ cycloalkyl), or —(C₁-C₄ alkyl)ₜ-O—(C₁-C₄ straight or branched alkyl) wherein: t is 0 or 1; the C₃-C₅ cycloalkyl optionally includes an oxygen or nitrogen heteroatom; L¹ is N; and L² is N;
(ii) Formula (XII-1):

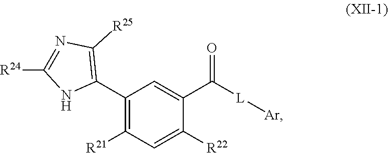

or pharmaceutically acceptable salts thereof, wherein:
L-Ar is

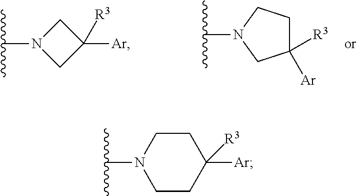

Ar is

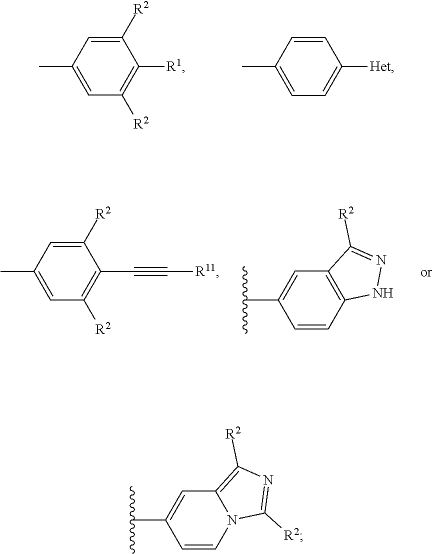

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently H, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; R²⁴ is H, —CN, —(C₁-C₄ alkyl)-CN, C₁-C₄ alkyl, C₁-C₄ haloalkyl, —(C₁-C₄ alkyl)-OH, —(C₁-C₄ alkyl)-N(R²⁴¹)₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl), wherein: t is 0 or 1; u is 0 or 1; each R²⁴¹ is independently H or C₁-C₂ alkyl; and R²⁵ is halogen, —CN, —(C₁-C₄ alkyl)-CN, C₁-C₂ alkyl, C₁-C₄ haloalkyl, —(C₁-C₄ alkyl)-O—(C₁-C₄ alkyl), or cyclopropyl;
(iii) Formula (XIII-1):

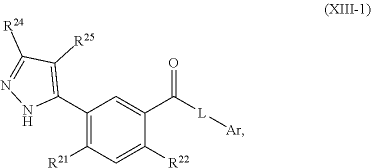

or pharmaceutically acceptable salts thereof, wherein:
L-Ar is

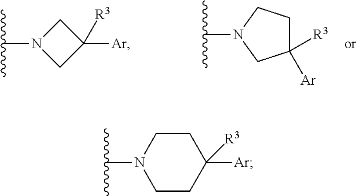

Ar is

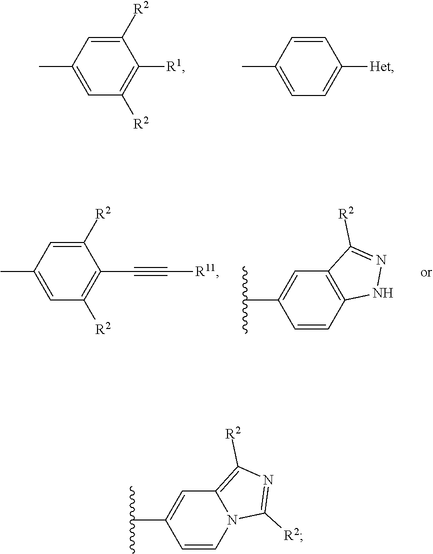

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and each R²⁴ and R²⁵ is independently H, halogen, —CN, —(C₁-C₄ alkyl)-CN, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₅ cycloalkyl), —(C₁-C₄ alkyl)ₜ-Oᵤ—(4- to 6-membered heterocycle) or —(C₁-C₄ alkyl)ₜ-O—(C₁-C₄ alkyl), wherein: each t is independently 0 or 1; each u is independently 0 or 1; and each R²⁴¹ is independently H or C₁-C₂ alkyl; or
(iv) Formula (XX-1):

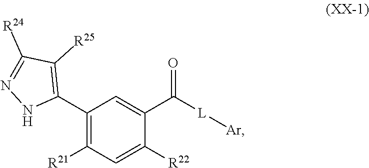

or a pharmaceutically acceptable salt thereof, wherein:
L-Ar is

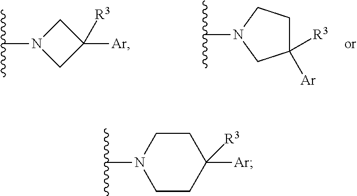

Ar is

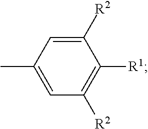

R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogen; each R² is independently hydrogen, halogen or C₁-C₄ alkyl; R³ is H or F; R²¹ is H, halogen, C₁-C₄ alkyl, C₃-C₅ cycloalkyl or 4- to 6-membered heterocycle; R²² is H, halogen or C₁-C₂ alkyl; R²⁴ is —O—(C₁-C₄ alkyl), —O—(C₁-C₄ alkyl)-O—(C₁-C₄ alkyl), —O—(C₃-C₅ cycloalkyl), or —O-(4- to 6-membered heterocycle), wherein R²⁴ is optionally substituted with one or more hydroxyl or halogen; and R²⁵ is H, halogen, C₁-C₄ alkyl or C₃-C₅ cycloalkyl, wherein R²⁵ is optionally substituted with one or more halogen.

In some embodiments, the FASN inhibitor of the method, apparatus, or non-transitory computer-readable storage medium of the above is a compound of Formula (IX-1), and wherein R¹ is H, —CN, halogen, or C₁-C₄ straight or branched alkyl; each R² is independently H; R³ is H or halogen; R²¹ is C₁-C₄ straight or branched alkyl or C₃-C₅ cycloalkyl; R²² is H or C₁-C₂ alkyl; and R²⁴ is H or C₁-C₄ straight or branched alkyl.

In some embodiments, the FASN inhibitor of the method, apparatus, or non-transitory computer-readable storage medium of the above is a compound of Formula (IX-1), and wherein R¹ is —CN; each R² is independently H; R³ is H; R²¹ is C₃-C₅ cycloalkyl; R²² is H or C₁-C₂ alkyl; and R²⁴ is C₁-C₄ straight or branched alkyl.

In some embodiments, the FASN inhibitor of the method, apparatus, or non-transitory computer-readable storage medium of the above is a compound of Formula (XII-1), and wherein L-Ar is

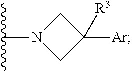

### Ar is

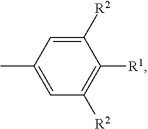

R¹ is H, —CN, halogen, or C₁-C₄ alkyl; each R² is independently H; R³ is H or F; R²¹ is H, halogen, or C₁-C₄ alkyl; R²² is H, halogen, or C₁-C₂ alkyl; R²⁴ is C₁-C₄ alkyl, C₁-C₄ haloalkyl, —(C₁-C₄ alkyl)ₜ-Oᵤ-(4- to 6-membered heterocycle), or —(C₁-C₄ alkyl)-O—(C₁-C₄ alkyl); R²⁵ is C₁-C₂ alkyl, C₁-C₄ haloalkyl, or —(C₁-C₄ alkyl)-O—(C₁-C₄ alkyl).

In some embodiments, the FASN inhibitor of the method, apparatus, or non-transitory computer-readable storage medium of the above is a compound of Formula (XII-1), and wherein L-Ar is

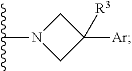

### Ar is

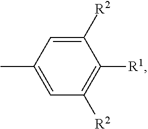

R¹ is —CN; each R² is independently H; R³ is H; R²¹ is C₁-C₄ alkyl; R²² is H or C₁-C₂ alkyl; R²⁴ is C₁-C₄ haloalkyl, or —(C₁-C₄ alkyl)ₜ-Oᵤ-(4- to 6-membered heterocycle), and R²⁵ is —(C₁-C₄ alkyl)-O—(C₁-C₄ alkyl).

In some embodiments, the FASN inhibitor of the method, apparatus, or non-transitory computer-readable storage medium of the above is a compound of Formula (XIII-1), wherein L-Ar is

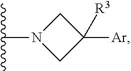

### Ar is

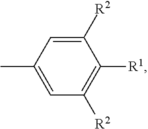

R¹ is H, —CN, halogen, or C₁-C₄ alkyl; each R² is independently H; R³ is H or F; R²¹ is H, halogen, or C₁-C₄ alkyl; R²² is H, halogen, or C₁-C₂ alkyl; and each R²⁴ and R²⁵ is independently halogen, C₁-C₄ alkyl, or —(C₁-C₄ alkyl)ₜ-O—(C₁-C₄ alkyl).

In some embodiments, the FASN inhibitor of the method, apparatus, or non-transitory computer-readable storage medium of the above is a compound of Formula (XIII-1), and wherein L-Ar is

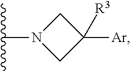

### Ar is

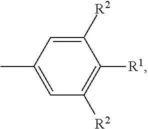

R¹ is —CN; each R² is independently H; R³ is H; R²¹ is C₁-C₄ alkyl; R²² is H or C₁-C₂ alkyl; and each R²⁴ and R²⁵ is independently halogen, C₁-C₄ alkyl, or —(C₁-C₄ alkyl)ₜ-O—(C₁-C₄ alkyl).

In some embodiments, the FASN inhibitor of the method, apparatus, or non-transitory computer-readable storage medium of the above is a compound of Formula (XX-1), and wherein L-Ar is

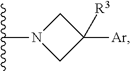

### Ar is

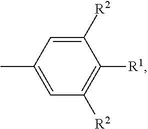

R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl), —O—(C₁-C₄ alkyl)-O—(C₁-C₄ alkyl), or —O-(4- to 6-membered heterocycle), wherein R²⁴ is optionally substituted with one or more hydroxyl or halogen; and R²⁵ is H, halogen, or C₁-C₄ alkyl.

In some embodiments, the FASN inhibitor of the method, apparatus, or non-transitory computer-readable storage medium of the above is a compound of Formula (XX-1), and wherein L-Ar is

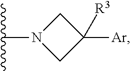

### Ar is

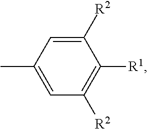

R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl) substituted with one or more hydroxyl or halogen; and R²⁵ is C₁-C₄ alkyl.

In some embodiments, the FASN inhibitor of the method, apparatus, or non-transitory computer-readable storage medium of the above is a compound selected from:

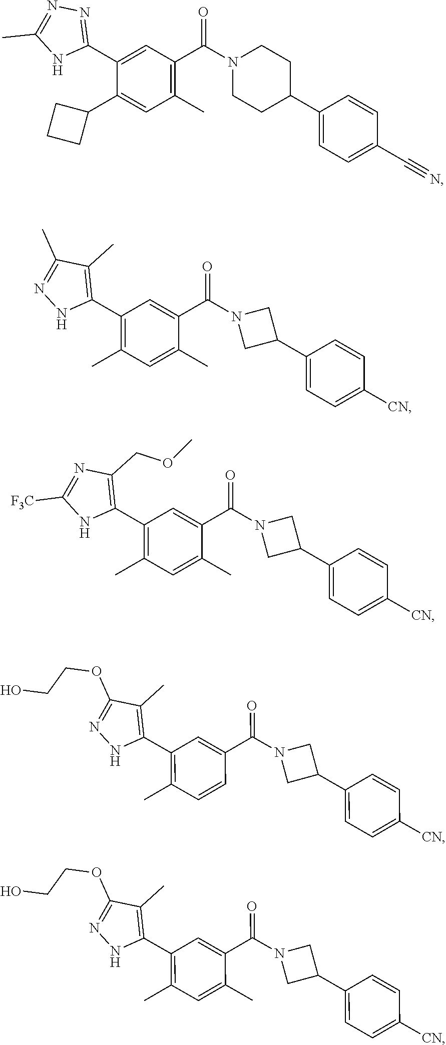 [SMILES: Cc1nnc(-c2cc(C(=O)N3CCC(c4ccc(C#N)cc4)CC3)c(C)cc2C2CCC2)[nH]1 ; Cc1cc(C)c(-c2[nH]nc(C)c2C)cc1C(=O)N1CC(c2ccc(C#N)cc2)C1 ; COCc1nc(C(F)(F)F)[nH]c1-c1cc(C(=O)N2CC(c3ccc(C#N)cc3)C2)c(C)cc1C ; Cc1ccc(C(=O)N2CC(c3ccc(C#N)cc3)C2)cc1-c1[nH]nc(OCCO)c1C ; Cc1cc(C)c(-c2[nH]nc(OCCO)c2C)cc1C(=O)N1CC(c2ccc(C#N)cc2)C1]

and pharmaceutically acceptable salts thereof.

In some embodiments, the FASN inhibitor of the method, apparatus, or non-transitory computer-readable storage medium of the above is a compound selected from:

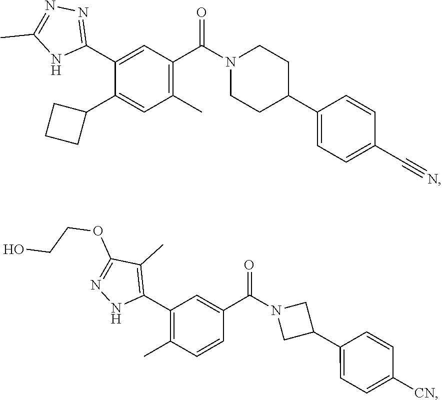 [SMILES: Cc1nnc(-c2cc(C(=O)N3CCC(c4ccc(C#N)cc4)CC3)c(C)cc2C2CCC2)[nH]1 ; Cc1ccc(C(=O)N2CC(c3ccc(C#N)cc3)C2)cc1-c1[nH]nc(OCCO)c1C]

and pharmaceutically acceptable salts thereof.

In some embodiments, the FASN inhibitor of the method, apparatus, or non-transitory computer-readable storage medium of the above is a compound selected from Tables C-1, C-2, and C-3.

In some embodiments, the fatty acid synthase inhibitor is a compound of Formula (IX-1), (XII-1), (XIII-1), or (XX-1), or a pharmaceutically acceptable salt thereof.

### BRIEF DESCRIPTION OF THE SEVERAL VIEWS OF THE DRAWINGS

FIG. 1 is a flow diagram of a method for prediction of treatment response in non-alcoholic fatty liver disease, according to an exemplary embodiment of the present disclosure.

FIG. 2A is a flow diagram of a method for prediction of treatment response in non-alcoholic fatty liver disease, according to an exemplary embodiment of the present disclosure.

FIG. 2B is a flow diagram of a method for prediction of treatment response in non-alcoholic fatty liver disease, according to an exemplary embodiment of the present disclosure.

FIG. 3 is an illustration of feature importance as determined via random forest algorithm.

FIG. 4 is an illustration, for a random forest algorithm, of average root mean square error in a training cohort and a validation cohort depending on the percentage of samples included in each cohort.

FIG. 5A is an illustration, for a random forest algorithm, of average root mean square error in a training cohort based on number of variables considered.

FIG. 5B is an illustration, for a random forest algorithm, of average root mean square error in a validation cohort based on number of variables considered.

FIG. 6A is an illustration, for a random forest algorithm, of median root mean square error in a training cohort based on number of variables considered.

FIG. 6B is an illustration, for a random forest algorithm, of median root mean square error in a validation cohort based on number of variables considered.

FIG. 7A is an illustration, for a random forest algorithm, of a distribution of root mean square error in a training cohort based on number of variables considered.

FIG. 7B is an illustration, for a random forest algorithm, of a distribution of root mean square error in a validation cohort based on number of variables considered.

FIG. 8 is a hardware schematic of a computing system configured to perform methods of determining a treatment regimen to administer to a subject having or suspected of having non-alcoholic steatohepatitis (NASH) or non-alcoholic fatty liver disease (NAFLD).

FIG. 9A is an illustration, for a random forest algorithm, of a correlation between observed % liver fat and predicted % liver fat for a training cohort.

FIG. 9B is an illustration, for a random forest algorithm, of a correlation between observed % liver fat and predicted % liver fat for a validation cohort.

FIG. 10 is a diagram of an exemplary random forest algorithm, including trees and decision structure.

FIG. 11A is an illustration, for a support vector machine algorithm, of average root mean square error in a training cohort based on number of variables and kernel applied.

FIG. 11B is an illustration, for a support vector machine algorithm, of average root mean square error in a validation cohort based on number of variables and kernel applied.

FIG. 12A is an illustration, for a support vector machine algorithm, of median root mean square error in a training cohort based on number of variables and kernel applied.

FIG. 12B is an illustration, for a support vector machine algorithm, of median root mean square error in a validation cohort based on number of variables and kernel applied.

FIG. 13 is an illustration of a relationship between root mean square error in a training cohort and a validation cohort. Each point represents results obtained in one model built with N variables.

FIG. 14A is an illustration, for a support vector machine algorithm, of a correlation between observed % liver fat and predicted % liver fat for a training cohort.

FIG. 14B is an illustration, for a support vector machine algorithm, of a correlation between observed % liver fat and predicted % liver fat for a validation cohort.

FIG. 15A is an illustration, for a random forest algorithm and a support vector machine algorithm, of a correlation between observed % liver fat and predicted % liver fat for a training cohort. Each observation is plotted twice, one from the random forest algorithm (blue dot) and one from the support vector machine algorithm (orange dot).

FIG. 15B is an illustration, for a random forest algorithm and a support vector machine algorithm, of a correlation between observed % liver fat and predicted % liver fat for a validation cohort. Each observation is plotted twice, one from the random forest algorithm (blue dot) and one from the support vector machine algorithm (orange dot).

FIG. 15C is an illustration, for a random forest algorithm, of a correlation between observed % liver fit and predicted % liver fat for a validation cohort.

FIG. 15D is an illustration of selected features and predicted outputs via random forest algorithm and a support vector machine algorithm.

FIG. 16A is an illustration of a receiver operator characteristic analysis for a random forest algorithm. The analysis is divided into a training cohort (solid line) and a testing cohort (dashed line).

FIG. 16B is an illustration of a receiver operator characteristic analysis for a support vector machine algorithm. The analysis is divided into a training cohort (solid line) and a testing cohort (dashed line).

FIG. 17A is an illustration of a results comparison of % liver fat predicted by a random forest algorithm and a probability generated by a previous, logistic regression model. The previous, logistic regression model was a categorical response based on a 30% reduction in % liver fat. Each point is an observation and has been colored according to the response to treatment (responders are green, non-responders are red).

FIG. 17B is an illustration of a results comparison of % liver fat predicted by a support vector machine algorithm and a probability generated by a previous, logistic regression model. The previous, logistic regression model was a categorical response based on a 30% reduction in % liver fat. Each point is an observation and has been colored according to the response to treatment (responders are green, non-responders are red).

FIG. 17C is an illustration of predicted outputs via binary classification tool and via random forest algorithm and a support vector machine algorithm.

FIG. 18 is a panel of diagrams of the de novo lipogenesis (DNL) pathway, fatty acid synthesis (FASN) inhibition targets (left panels), and the development of compound 001-152 in nonalcoholic steatohepatitis (NASH) (right panel).

FIG. 19 is a diagram of the study design.

FIG. 20 is a list of biomarkers included in the model.

FIG. 21A is an illustration, for a SVM algorithm, of a correlation between observed % liver fat change and predicted % liver fat change for participants receiving 50 mg compound 001-152 (“active”) for 12 weeks (blue) and 50 mg compound 001-152 (“active”) for 16 weeks (orange). % liver fat change ranges between −0.8 and 0.1 for most of the samples. Arrows indicate % liver fat change range for which models are undertrained.

FIG. 21B is an illustration, for a random forest algorithm, of a correlation between observed % liver fat change and predicted % liver fat change for participants receiving 50 mg compound 001-152 (“active”) for 12 weeks (blue) and 50 mg compound 001-152 (“active”) for 16 weeks (orange). % liver fat change ranges between −0.8 and 0.1 for most of the samples. Arrows indicate % liver fat change range for which models are undertrained.

FIG. 22A is an illustration, for a SVM algorithm, of a correlation between observed % liver fat change and predicted % liver fat change for participants receiving placebo (green), 25 mg compound 001-152 (“active”) for 12 weeks (red), 50 mg compound 001-152 (“active”) for 12 weeks (blue), and 50 mg compound 001-152 (“active”) for 16 weeks (orange). Arrows indicate % liver fat change range for which models are undertrained.

FIG. 22B is an illustration, for a random forest algorithm, of a correlation between observed % liver fat change and predicted % liver fat change for participants receiving placebo (green), 25 mg compound 001-152 (“active”) for 12 weeks (red), 50 mg compound 001-152 (“active”) for 12 weeks (blue), and 50 mg compound 001-152 (“active”) for 16 weeks (orange). Arrows indicate % liver fat change range for which models are undertrained.

FIG. 23 is a diagram of the de novo lipogenesis (DNL) pathway (left panel) and a graph showing % change in tripalmitin over the course of 12 weeks of the study for participants receiving placebo (grey) or 50 mg compound 001-152 for 12 weeks (green).

FIG. 24A is an illustration, for a SVM algorithm, of observed Root Mean Square Error (RMSE) by % Tripalmitin change cutoff for participants receiving placebo (green), 25 mg compound 001-152 (“active”) for 12 weeks (red), 50 mg compound 001-152 (“active”) for 12 weeks (blue), 75 mg compound 001-152 (“active”) for 12 weeks (purple), and 50 mg compound 001-152 (“active”) for 16 weeks (orange). Vertical red lines show the RMSE for each cohort removing the tripalmitin responders (<−50% and <−30%).

FIG. 24B is an illustration, for a random forest algorithm, of observed Root Mean Square Error (RMSE) by % Tripalmitin change cutoff for participants receiving placebo (green), 25 mg compound 001-152 (“active”) for 12 weeks (red), 50 mg compound 001-152 (“active”) for 12 weeks (blue), 75 mg compound 001-152 (“active”) for 12 weeks (purple), and 50 mg compound 001-152 (“active”) for 16 weeks (orange). Vertical red lines show the RMSE for each cohort removing the tripalmitin responders (<−50% and <−30%).

FIG. 25A is an illustration, for a SVM algorithm, of a correlation between observed % liver fat change and predicted % liver fat change for participants receiving placebo (green), 25 mg compound 001-152 (“active”) for 12 weeks (red), 50 mg compound 001-152 (“active”) for 12 weeks (blue), 75 mg compound 001-152 (“active”) for 12 weeks (purple), and 50 mg compound 001-152 (“active”) for 16 weeks (orange). All patients below % tripalmitin change cutoff (0.3) are shown.

FIG. 25B is an illustration, for a random forest algorithm, of a correlation between observed % liver fat change and predicted % liver fat change for participants receiving placebo (green), 25 mg compound 001-152 (“active”) for 12 weeks (red), 50 mg compound 001-152 (“active”) for 12 weeks (blue), 75 mg compound 001-152 (“active”) for 12 weeks (purple), and 50 mg compound 001-152 (“active”) for 16 weeks (orange). All patients below % tripalmitin change cutoff (0.3) are shown.

FIG. 26A is an illustration, for a random forest algorithm, of a correlation between observed % liver fat change measured by MRI-PDFF and a value derived by random forest for participants receiving 50 mg compound 001-152 (“active”) for 12 weeks (blue) and 50 mg compound 001-152 (“active”) for 16 weeks (orange).

FIG. 26B is an illustration, for a random forest algorithm, of a correlation between observed % liver fat change measured by Magnetic Resonance Imaging Proton Density Fat Fraction (MRI-PDFF) and a value derived by random forest for participants receiving placebo (green), 25 mg compound 001-152 for 12 weeks (red), 50 mg compound 001-152 for 12 weeks (blue), and 50 mg compound 001-152 for 16 weeks (orange).

FIG. 26C is an illustration, for a random forest algorithm, of a correlation between observed % liver fat change measured by MRI-PDFF and a value derived by random forest wherein the data has been filtered on pharmacodynamics of tripalmitin with a decrease >40%.

FIG. 27A is an illustration of Mass Spectrometry analytic platforms. Platform 1 using MeOH extraction, platform 2 using CHCl₃/MeOPH extraction, and platform 3 using Derivatized MeOH extraction and the corresponding ultra-high performance liquid chromatography-mass spectrometry (UHPLC-MS) analysis for participants receiving placebo (n=30; green) or 50 mg compound 001-152 (n=33).

FIG. 27B is an illustration of lipidomic analysis of 476 analytes for participants receiving placebo (green) or 50 mg compound 001-152 at Week 4 of the study described in Example 2 compared to Baseline.

FIG. 28A is a graph showing % change in total diacylglycerols (DG) (top panel) and total saturated diacylglycerols (DG) over the course of 12 weeks of the study described in Example 2 for participants receiving placebo (grey) or 50 mg compound 001-152 for 12 weeks (green).

FIG. 28B is a plot showing the number of double bonds vs. the number of carbons depicting long chain polyunsaturated fatty acid (DG) content for participants receiving placebo or 50 mg compound 001-152 at Week 12 of the study described in Example 2 compared to Baseline.

FIG. 29A is a graph showing % change in total triacylglycerols (TG) (top panel) and total saturated triacylglycerols (TG) over the course of 12 weeks of the study described in Example 2 for participants receiving placebo (grey) or 50 mg compound 001-152 for 12 weeks (green).

FIG. 29B is a plot showing the number of double bonds vs. the number of carbons depicting long chain polyunsaturated fatty acid (TG) content for participants receiving placebo or 50 mg compound 001-152 at Week 12 of the study described in Example 2 compared to Baseline.

FIG. 30A is a graph showing change in LDL-cholesterol in mg/dL over the course of 12 weeks of the study described in Example 2 for participants receiving placebo (grey) or 50 mg compound 001-152 for 12 weeks (green).

FIG. 30B is a graph showing % change in C16-ceramide over the course of 12 weeks of the study described in Example 2 for participants receiving placebo (grey) or 50 mg compound 001-152 for 12 weeks (green).

FIG. 30C is a graph showing % change in C22-ceramide over the course of 12 weeks of the study described in Example 2 for participants receiving placebo (grey) or 50 mg compound 001-152 for 12 weeks (green).

### DETAILED DESCRIPTION

### Definitions

Chemical moieties referred to as univalent chemical moieties (e.g., alkyl, aryl, etc.) also encompass structurally permissible multivalent moieties, as understood by those skilled in the art. For example, while an “alkyl” moiety generally refers to a monovalent radical (e.g., CH₃CH₂—), in appropriate circumstances an “alkyl” moiety can also refer to a divalent radical (e.g., —CH₂CH₂—, which is equivalent to an “alkylene” group). Similarly, under circumstances where a divalent moiety is required, those skilled in the art will understand that the term “aryl” refers to the corresponding divalent arylene group.

All atoms are understood to have their normal number of valences for bond formation (e.g., 4 for carbon, 3 for N, 2 for O, and 2, 4, or 6 for S, depending on the atom's oxidation state). On occasion a moiety can be defined, for example, as (A)ₐB, wherein a is 0 or 1. In such instances, when a is 0 the moiety is B and when a is 1 the moiety is AB.

Where a substituent can vary in the number of atoms or groups of the same kind (e.g., alkyl groups can be C₁, C₂, C₃, etc.), the number of repeated atoms or groups can be represented by a range (e.g., C₁-C₆ alkyl) which includes each and every number in the range and any and all sub ranges. For example, C₁-C₃ alkyl includes C₁, C₂, C₃, C₁₋₂, C₁₋₃, and C₂₋₃ alkyl.

“Alkanoyl” refers to a carbonyl group with a lower alkyl group as a substituent.

“Alkylamino” refers to an amino group substituted by an alkyl group.

“Alkoxy” refers to an O-atom substituted by an alkyl group as defined herein, for example, methoxy [—OCH₃, a C₁alkoxy]. The term “C₁₋₆ alkoxy” encompasses C₁ alkoxy, C₂ alkoxy, C₃ alkoxy, C₄ alkoxy, C₅ alkoxy, C₆ alkoxy, and any sub-range thereof.

“Alkoxycarbonyl” refers to a carbonyl group with an alkoxy group as a substituent.

“Alkyl,” “alkenyl,” and “alkynyl,” refer to optionally substituted, straight and branched chain aliphatic groups having from 1 to 30 carbon atoms, or preferably from 1 to 15 carbon atoms, or more preferably from 1 to 6 carbon atoms. Examples of alkyl groups include, without limitation, methyl, ethyl, propyl, isopropyl, butyl, tert-butyl, isobutyl, pentyl, hexyl, vinyl, allyl, isobutenyl, ethynyl, and propynyl. The term “heteroalkyl” as used herein contemplates an alkyl with one or more heteroatoms.

“Alkylene” refers to an optionally substituted divalent radical which is a branched or unbranched hydrocarbon fragment containing the specified number of carbon atoms, and having two points of attachment. An example is propylene [—CH₂CH₂CH₂—, a C₃alkylene].

“Amino” refers to the group —NH₂.

“Aryl” refers to optionally substituted aromatic groups which have at least one ring having a conjugated pi electron system and includes carbocyclic aryl, and biaryl groups, all of which can be optionally substituted. Phenyl and naphthyl groups are preferred carbocyclic aryl groups.

“Aralkyl” or “arylalkyl” refer to alkyl-substituted aryl groups. Examples of aralkyl groups include butylphenyl, propylphenyl, ethylphenyl, methylphenyl, 3,5-dimethylphenyl, tert-butylphenyl.

“Carbamoyl” as used herein contemplates a group of the Formula

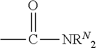

where in R^{N} is selected from the group consisting of hydrogen, —OH, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, sulfonyl, sulfonate and sulfonamide.

“Carbonyl” refers to a group of the Formula

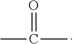

“Cycloalkyl” refers to an optionally substituted ring, which can be saturated or unsaturated and monocyclic, bicyclic, or tricyclic formed entirely from carbon atoms. An example of a cycloalkyl group is the cyclopentenyl group (C₅H₇—), which is a five carbon (C₅) unsaturated cycloalkyl group.

“Heterocycle” refers to an optionally substituted 5- to 7-membered cycloalkyl ring system containing 1, 2 or 3 heteroatoms, which can be the same or different, selected from N, O or S, and optionally containing one double bond.

“Halogen” refers to a chloro, bromo, fluoro or iodo atom radical. The term “halogen” also contemplates terms “halo” or “halide.”

“Heteroatom” refers to a non-carbon atom, where boron, nitrogen, oxygen, sulfur and phosphorus are preferred heteroatoms, with nitrogen, oxygen and sulfur being particularly preferred heteroatoms in the compounds of the present disclosure.

“Heteroaryl” refers to optionally substituted aryl groups having from 1 to 9 carbon atoms and the remainder of the atoms are heteroatoms, and includes those heterocyclic systems described in “Handbook of Chemistry and Physics,” 49th edition, 1968, R. C. Weast, editor; The Chemical Rubber Co., Cleveland, Ohio. See particularly Section C, Rules for Naming Organic Compounds, B. Fundamental Heterocyclic Systems. Suitable heteroaryls include thienyl, pyrrolyl, furyl, pyridyl, pyrimidyl, pyrazinyl, pyrazolyl, oxazolyl, isoxazolyl, imidazolyl, thiazolyl, pyranyl, tetrazolyl, pyrrolyl, pyrrolinyl, pyridazinyl, triazolyl, indolyl, isoindolyl, indolizinyl, benzimidazolyl, quinolyl, isoquinolyl, indazolyl, benzotriazolyl, tetrazolopyridazinyl, oxadiazolyl, benzoxazolyl, benzoxadiazolyl, thiadiazolyl, benzothiazolyl, benzothiadiazolyl, and the like.

An “optionally substituted” moiety can be substituted with from one to four, or preferably from one to three, or more preferably one or two non-hydrogen substituents. Unless otherwise specified, when the substituent is on a carbon, it is selected from the group consisting of —OH, —CN, —NO₂, halogen, C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, substituted sulfonyl, sulfonate, sulfonamide and amino, none of which are further substituted. Unless otherwise specified, when the substituent is on a nitrogen, it is selected from the group consisting of C₁ to C₁₂ alkyl, C₁ to C₁₂ heteroalkyl, cycloalkyl, heterocycle, aryl, heteroaryl, aralkyl, alkoxy, alkoxycarbonyl, alkanoyl, carbamoyl, sulfonyl, sulfonate and sulfonamide none of which are further substituted.

The term “sulfonamide” as used herein contemplates a group having the Formula

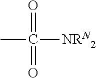

wherein R^{N} is selected from the group consisting of hydrogen, —OH, C₁ to C₁₂ alkyl, C₁ to C₁₂ heteroalkyl, alkenyl, alkynyl, cycloalkyl, heterocycle, aryl, heteroaryl, aralkyl, alkoxy, alkoxycarbonyl, alkanoyl, carbamoyl, substituted sulfonyl, sulfonate and sulfonamide.

The term “sulfonate” as used herein contemplates a group having the Formula

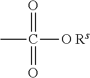

wherein R^{s} is selected from the group consisting of hydrogen, C₁-C₁₀ alkyl, C₂-C₁₀ alkenyl, C₂-C₁₀ alkynyl, C₁-C₁₀ alkanoyl, or C₁-C₁₀ alkoxycarbonyl.

“Sulfonyl” as used herein alone or as part of another group, refers to an SO₂ group. The SO₂ moiety is optionally substituted.

Compounds of the present disclosure can exist as stereoisomers, wherein asymmetric or chiral centers are present. Stereoisomers are designated (R) or (S) depending on the configuration of substituents around the chiral carbon atom. The terms (R) and (S) used herein are configurations as defined in IUPAC 1974 Recommendations for Section E, Fundamental Stereochemistry, Pure Appl. Chem., (1976), 45: 13-30, hereby incorporated by reference. The present disclosure contemplates various stereoisomers and mixtures thereof and are specifically included within the scope of the present disclosure. Stereoisomers include enantiomers, diastereomers, and mixtures of enantiomers or diastereomers. Individual stereoisomers of compounds of the present disclosure can be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Also, moieties disclosed herein which exist in multiple tautomeric forms include all such forms encompassed by a given tautomeric Formula.

Individual atoms in the disclosed compounds may be any isotope of that element. For example hydrogen may be in the form of deuterium.

“Pharmaceutically acceptable” means approved or approvable by a regulatory agency of the Federal or state government or listed in the U.S. Pharmacopoeia or other generally recognized pharmacopoeia for use in animals, and more particularly in humans. It can be material which is not biologically or otherwise undesirable, i.e., the material can be administered to an individual without causing any undesirable biological effects or interacting in a deleterious manner with any of the components of the composition in which it is contained.

The term “pharmaceutically acceptable salt” of a compound means a salt that is pharmaceutically acceptable and that possesses the desired pharmacological activity of the parent compound. Such salts include, for example, acid addition salts and base addition salts.

“Acid addition salts” according to the present disclosure, are formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

“Base addition salts” according to the present disclosure are formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature can cause a single crystal form to dominate.

The term “treating” and/or “treatment” includes the administration of the compounds or agents of the present invention to a subject to prevent or delay, to alleviate, or to arrest or inhibit development of the symptoms or conditions associated with fatty acid synthase-associated disorders, e.g., tumor growth associated with cancer. A skilled medical practitioner will know how to use standard methods to determine whether a patient is suffering from a disease associated with activity of fatty acid synthase, e.g., by examining the patient and determining whether the patient is suffering from a disease known to be associated with fatty acid synthase activity or by assaying for fatty acid synthase levels in blood plasma or tissue of the individual suspected of suffering from fatty acid synthase associated disease and comparing fatty acid synthase levels in the blood plasma or tissue of the individual suspected of suffering from a fatty acid synthase associated disease with fatty acid synthase levels in the blood plasma or tissue of a healthy individual. Increased securing levels are indicative of disease. Accordingly, the present invention provides, inter alia, methods of administering a compound of the present invention to a subject and determining fatty acid synthase activity in the subject. Fatty acid synthase activity in the subject can be determined before and/or after administration of the compound.

The term “inhibitor”, as used herein, should be understood to describe any substance which slows down or prevents a particular process or which reduces the activity of a particular reactant, catalyst, or enzyme. A skilled artisan will appreciate that the inhibitor, as it relates to the present disclosure, may be any substance that slows down, prevents, or reduces the activity of a particular process or a particular enzyme within the de novo lipogenesis pathway. The inhibitor may be competitive, noncompetitive, uncompetitive, and/or mixed inhibitor.

A “therapeutically effective amount” or “pharmaceutically effective amount” means the amount that, when administered to a subject, produces effects for which it is administered. For example, a “therapeutically effective amount,” when administered to a subject to inhibit fatty acid synthase activity, is sufficient to inhibit fatty acid synthase activity. A “therapeutically effective amount,” when administered to a subject for treating a disease, is sufficient to effect treatment for that disease.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary aspect of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that are associated with the targeted or suspected disease or condition. These and other routine methods allow the clinician to select patients in need of therapy using the methods and formulations of the present invention.

The term “a” or “an” refers to one or more of that entity, i.e. can refer to plural referents. As such, the terms “a,” “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

In some embodiments, quantitative data related to biomarkers can be obtained by, without limitation, refractive index spectroscopy (RI), UltraViolet spectroscopy (UV), fluorescent analysis, radiochemical analysis, Infrared spectroscopy (IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, enzyme-linked immunosorbent assay (ELISA), ADVIA Centaur XP Immunoassay System, liquid chromatography combined with mass spectroscopy, supercritical fluid chromatography combined with mass spectroscopy, MALDI combined with mass spectroscopy, ion spray spectroscopy combined with mass spectroscopy, capillary electrophoresis combined with mass spectrometry, NMR combined with mass spectrometry, and IR combined with mass spectrometry.

In a particular embodiment, as it relates to biochemical data, a determination of a level of the biochemical data is carried out by mass spectrometry. As used herein, “mass spectrometry” (MS analysis) refers to an analytical technique to identify unknown compounds including: (1) ionizing the compounds and potentially fractionating the compounds parent ion formed into daughter ions; and (2) detecting the charged compounds and calculating a mass-to-charge ratio (m/z). The compounds may be ionized and detected by any suitable means. A “mass spectrometer” includes means for ionizing compounds and for detecting charged compounds.

Preferably, mass spectrometry is used in particular gas chromatography coupled to mass spectrometry (GCMS), liquid chromatography coupled to mass spectrometry (LCMS), direct infusion mass spectrometry or Fourier transform ion-cyclotron resonance mass spectrometry, capillary electrophoresis coupled to mass spectrometry, high-performance liquid chromatography coupled to mass spectrometry, ultra high-performance liquid chromatography coupled to mass spectrometry, supercritical fluid chromatography coupled to mass spectroscopy, flow injection analysis with mass spectrometry, including quadrupole mass spectrometry, any sequentially coupled mass spectrometry, such as MS-MS or MS-MS-MS, inductively coupled plasma mass spectrometry, pyrolysis mass spectrometry, ion mobility mass spectrometry or time-of flight mass spectrometry, electrospray ionization mass spectrometry, matrix-assisted laser desorption ionization time-of-flight mass spectrometry, surface-enhanced laser desorption/ionization time-of-flight mass spectrometry, desorption/ionization on silicon, secondary ion mass spectrometry, quadrupole time-of-flight, atmospheric pressure chemical ionization mass spectrometry, atmospheric pressure photoionization mass spectrometry, quadrupole mass spectrometry, Fourier transform mass spectrometry, and ion trap mass spectrometry, where n is an integer greater than zero. In some embodiments, LCMS and/or MS can be used, as described in detail below. Said techniques are disclosed in, e.g., Nissen, Journal of Chromatography A, 703, 1995: 37-57, U.S. Pat. No. 4,540,884, or U.S. Pat. No. 5,397,894, incorporated herein by reference.

The above-mentioned ionization methods generally produce an ion resulting from the addition of one or more atoms or by cleavage of the molecule. These ions can then be used as surrogate markers for the biochemical markers used in methods described herein. The term “surrogate marker” as used herein means a biological or clinical parameter that is measured in place of the biologically definitive or clinically most meaningful parameter.

The term “non-alcoholic fatty liver disease”, “NAFLD”, as used herein, refers to a group of conditions having in common the accumulation of fat in the hepatocytes, NAFLD ranges from simply fatty liver (steatosis), to nonalcoholic steatohepatitis (NASH), to cirrhosis (irreversible, advanced scarring of the liver). The term “NAFLD” includes any stage or degree of progression of the disease, including NAFLD without fibrosis, NAFLD with fibrosis stage 1-2 and NAFLD with fibrosis stage 3-4. In a particular embodiment, the NAFLD is selected from the group consisting of NAFLD without fibrosis, NAFLD with fibrosis stage 1-2 and NAFLD with fibrosis stage 3-4.

The terms “NAFLD without fibrosis” or “NAFLD with no fibrosis”, as used herein, refer to a NAFLD with fibrosis stage 0.

The terms “NAFLD with fibrosis stage 1-2” or “NAFLD with early stage fibrosis”, as used herein, refer to a NAFLD with fibrosis stage 1, 1A, 1B, 1C, or 2.

The terms “NAFLD with fibrosis stage 3-4” or “NAFLD with advanced fibrosis”, as used herein, refer to a NAFLD with fibrosis stage 3 or 4.

The fibrosis stages are defined by Kleiner et al. (Hepatology 2005, 41(6): 1313-21) and are the following: Stage 0→no fibrosis; Stage 1→perisinusoidal or periportal fibrosis, Stage 1A→mild, zone 3, perisinusoidal fibrosis, Stage 1B→moderate, zone 3, perisinusoidal fibrosis, Stage 1C→portal/periportal fibrosis; Stage 2→perisinusoidal and portal/periportal fibrosis; Stage 3→bridging fibrosis; Stage 4→cirrhosis.

Non-alcoholic fatty liver disease (NAFLD) encompasses a wide range of conditions characterized by the build-up of fat in the liver cells in absence of alcohol abuse. At one end of the scale is the relatively harmless simple fatty liver, or steatosis, that does not cause significant liver damage. If left unattended, this condition may progress to more advanced conditions, some of which may be life threatening. Nonalcoholic steatohepatitis (NASH) is a significant development in NAFLD, corresponding to an aggressive condition characterized by swelling and tenderness in the liver. With intense, on-going inflammation a buildup of scar tissue (fibrosis) may form, eventually leading to cirrhosis where irregular bumps, known as nodules, replace the smooth liver tissue and the liver becomes harder. The effect of this, together with continued scarring from fibrosis, means that the liver will run out of healthy cells to support normal functions. This can lead to complete liver failure. For this reason, diagnosis and treatment of NAFLD is critical.

Pharmaceutical interventions for NAFLD, however, much like initially prescribed lifestyle changes, require a patient to undergo treatment for a sustained length of time before any determination of efficacy can be made. Accordingly, there exists a need for a method to predict whether a patient is likely to respond positively to a particular treatment.

The de novo lipogenesis (DNL) pathway is elevated in patients with fatty liver disease, and converts dietary sugars topalmitate, the building block for lipid synthesis. Without wishing to be bound by theory, activation of the DNL pathway not only causes liver fat accumulation, but activates pro-inflammatory cells and is required in stellate cells for activation and fibrogenesis. Fatty acid synthase (FASN) is the last committed step in DNL and therefore provides an approach to target three hallmarks of NASH; steatosis, inflammation and fibrosis. The FASN inhibitor compound 001-152 is in Phase 2b development for NASH. A Phase 2a study showed that compound 001-152 significantly reduced liver fat in NASH, and decreased disease biomarkers including ALT, CK-18, PRO—C3 and lipotoxins.

Compound 001-152 is a first in class potent FASN inhibitor. A Phase 2a study showed significant liver fat (LF) reduction at 50 mg compound 001-152, and decreased biomarkers including ALT, PRO—C3, PIIINP, CK-18 and lipotoxins. Without wishing to be bound by theory, enrichment of patients most likely to respond to a specific therapy is a part of NASH drug development and has potential to improve response rates and direct patients to the most appropriate treatment. This work explored baseline metabolomic biomarkers to predict response to compound 001-152. Example 2 demonstrates the effect of baseline lipidomic markers that predict liver fat response to compound 001-152 and the effect of compound 001-152 on the circulating lipidome.

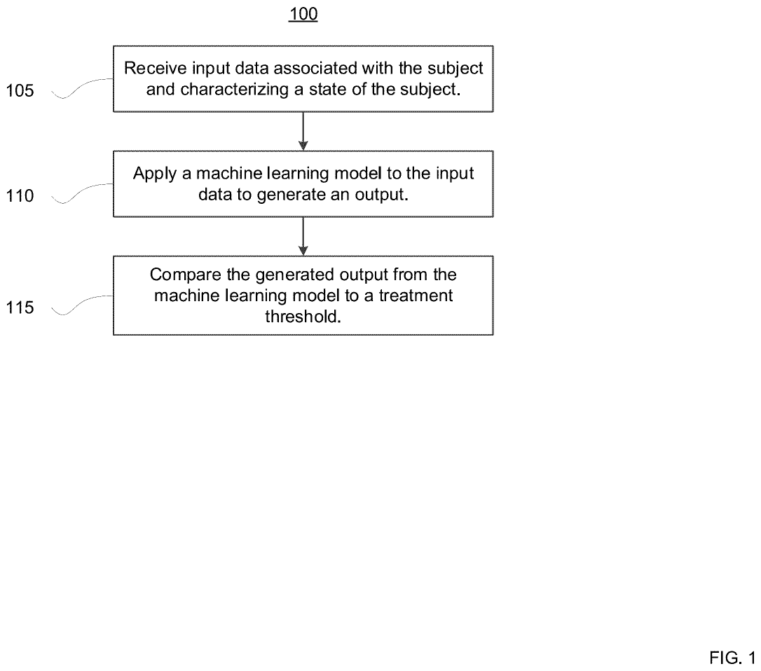

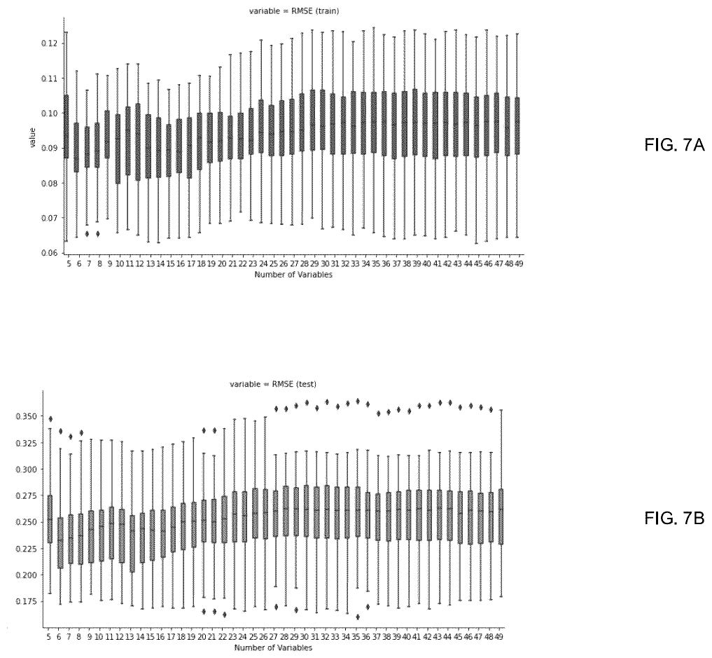

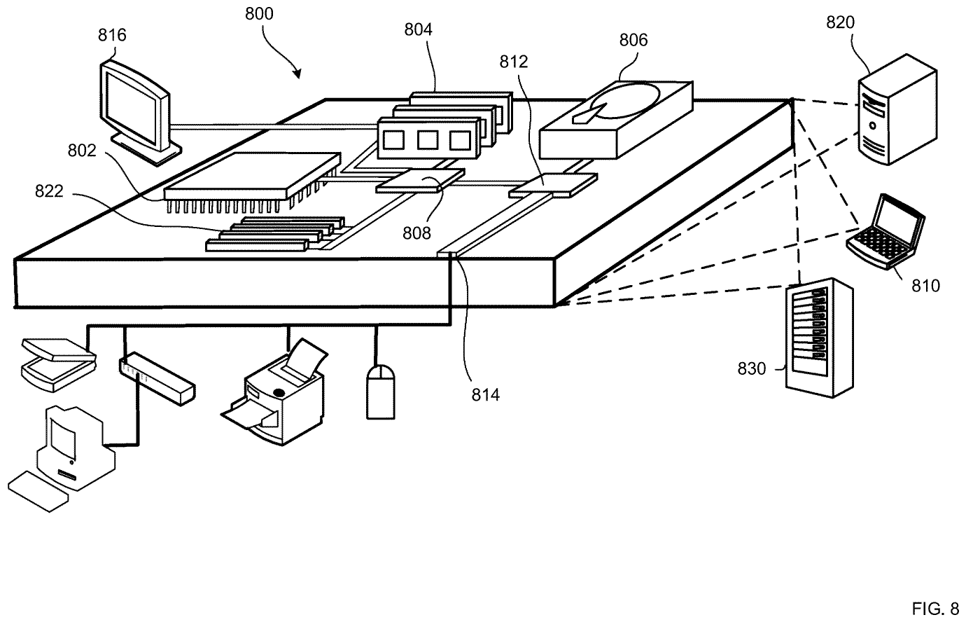

Referring now to the Drawings, FIG. 1 is a flow diagram of a method for predicting responsiveness of a patient, or subject, to a particular treatment regimen. Method 100 of FIG. 1 can be performed by a computing device having processing circuitry configured to control, instruct, and/or perform the method, such as one or more of the computing devices of described with reference to FIG. 8.

At a high-level, the methods described herein seek to compare characteristics of a current patient with characteristics of reference patients to determine if the current patient will respond to a particular treatment regimen in the same way that the reference patients did. For example, assuming the reference patients responded to the particular treatment regimen by experiencing, e.g., a 50% reduction in liver fat percentage, the question is whether the current patient is expected to experience the same level of benefit. This comparison can be performed on the basis of one or more characteristics (or features) describing those patients and on a machine learning model developed in view of the one or more characteristics and a treatment metric (e.g., percentage of liver fat). If the machine learning model generates an output that satisfies a treatment threshold (i.e., it determines that the current patient will experience a benefit, relative to the treatment metric, from the particular treatment regimen), then the treatment regimen will be administered to the current patient.

At a low-level, and beginning at step 105 of method 100, input data associated with a subject can be received. At step 110 of method 100, a machine learning model can be applied to the received input data characterizing the state of the subject. Understanding that the trained machine learning model is based on at least one feature of the plurality of possible features, it can be appreciated that the same subset of features would be implemented as the received input data of step 105 of method 100. In other words, in developing the machine learning model, the features that generate the most predictive model will be the features received as input data during implementation of method 100.

In some embodiments of the methods, apparatus, or non-transitory computer-readable storage medium disclosed herein (e.g., method 100), the subject is an individual with NAFLD (e.g., NASH) and the like, or derivatives thereof. In some embodiments of the methods, apparatus, or non-transitory computer-readable storage medium disclosed herein (e.g., method 100), the input data associated with the subject characterizes a state of the subject at a ‘baseline’ condition, or reference condition. The reference condition may be one that reflects a medical condition of the subject before the initiation of a treatment regimen.

In some embodiments of the methods, apparatus, or non-transitory computer-readable storage medium disclosed herein (e.g., method 100), the input data is one or more of biochemical data, histological data, biometric data, medical imaging data, and the like. In some embodiments, the biochemical data includes, as will be described detail below, proteomic data, metabolomic data, transcriptomics data, genomic data, and the like. In some embodiments, the biochemical data includes fibrosis markers such as pro-peptide of type III collagen (PRO—C3), tissue inhibitor of metalloproteinases 1 (TIMP-1), and amino-terminal pro-peptide of type III procollagen (PIIINP) and/or liver biomarkers such as alanine aminotransferase (ALT). In some embodiments, the biochemical data includes an enhanced liver fibrosis (ELF) score based on TIMP-1, PIIINP, and hyaluronic acid. In some embodiments, the genomic data includes inter alia data indicating the presence or absence of variant single nucleotide polymorphisms (SNPs) in certain genes. In some embodiments, the genomic data includes, among others, body weight, height, gender, race, and ethnicity, as well as biomechanical alignment, gait, and the like. In some embodiments, the medical imaging data includes, for instance, images acquired by, among others, magnetic resonance imaging (MRI), computed tomography, ultrasound, and derivatives thereof. In some embodiments, the medical imaging data is image data (e.g. DICOM) and/or processed image data, where the processed image data includes quantified metrics describing the image data. In some embodiments, t the processed image data is a percentage of liver fat and/or a hepatic fat fraction measured via MRI proton density fat fraction (MRI-PDFF).

In some embodiments of the methods, apparatus, or non-transitory computer-readable storage medium disclosed herein (e.g., method 100), the machine learning model comprises one or more machine learning models trained to generate an output corresponding to a treatment metric. In some embodiments, the trained machine learning model is based on a subset of features obtained from a plurality of possible features, where the plurality of possible features describes the totality of possible input data. In other words, in some embodiments, the plurality of possible features includes every feature associated with the biochemical data, every feature associated with the biometric data, every feature associated with the medical imaging data, and every feature associated with the histological data. In some embodiments, the subset of features, which may be any portion of the plurality of possible features (or every one of the plurality of possible features), includes features determined to be highly important to or predictive of, either individually or in combination, treatment responsiveness. In some embodiments, the trained machine learning model is based on one feature, is based on two features, is based on three features, is based on four features, is based on five features, is based on six features, is based on seven features, is based on eight features, is based on nine features, or is based on ten or more features. In the event the trained machine learning model is based on two or more features, it can be appreciated that the two or more features may be synergistic and/or independent of one another.

In some embodiments of the methods, apparatus, or non-transitory computer-readable storage medium disclosed herein (e.g., method 100), the treatment metric includes, among other things, percentage of liver fat, histology, clinical outcomes, fibrosis stage/status, MRI-PDFF, NASH resolution (e.g., absence of fatty liver disease or isolated or simple steatosis without steatohepatitis and a NAFLD Activity Score (NAS) score of 0-1 for inflammation, 0 for ballooning, and any value for steatosis), and the like. For instance, in some embodiments, when the treatment metric is percentage of liver fat (i.e., a measure for the amount of the liver that is occupied by fat), the one or more machine learning models are configured to generate, based on the input data, a value representing a percentage of the liver that is occupied by fat.

In some embodiments of the methods, apparatus, or non-transitory computer-readable storage medium disclosed herein (e.g., method 100), the machine learning model is a regression-based model or a classification-based model. In some embodiments, the output generated by the machine learning model is a continuous variable or a discrete variable. In some embodiments of the methods, apparatus, or non-transitory computer-readable storage medium disclosed herein (e.g., method 100), the machine learning model is selected from a decision tree, a linear classifier, a neural network, random forests, k-nearest neighbors, support vector machines, and the like. In some embodiments, the neural network is selected from a multi-layer perceptron, a convolutional neural network, a recurrent neural network, and the like. In some embodiments of the methods, apparatus, or non-transitory computer-readable storage medium disclosed herein (e.g., method 100), the convolutional neural network is selected from LeNet, AlexNet, VGGNet 16, GoogleNet/Inception, ResNets, and the like. The type of machine learning model used depends, inherently, on the type of input data used and the type of generated output that is desired. In some embodiments of the methods, apparatus, or non-transitory computer-readable storage medium disclosed herein (e.g., method 100), when the input data is biochemical data, the machine learning model is at least one of a decision tree, a linear classifier, random forests, and a support vector machine. In some embodiments of the methods, apparatus, or non-transitory computer-readable storage medium disclosed herein (e.g., method 100), where the input data is medical imaging data, the machine learning model is a convolutional neural network.

In some embodiments of the methods, apparatus, or non-transitory computer-readable storage medium disclosed herein (e.g., method 100), the machine learning model is trained on reference data corresponding to a type of the input data and type of the treatment metric. In some embodiments, the reference data includes input data characterizing a state of reference subjects at a reference condition and at, following administration of a treatment regimen, a treated condition. In some embodiments, the treatment regimen administered to each reference subject is the same being considered for the subject that a method or apparatus of the disclosure (e.g., method 100) is being applied on behalf of. In this way, in some embodiments, the output generated by the machine learning model is based on features of the current subject in view of features of reference subjects and their responsiveness to the treatment regimen.

In some embodiments of the methods, apparatus, or non-transitory computer-readable storage medium disclosed herein (e.g., method 100, e.g. at step 115), the output generated (e.g., at step 110 of method 100) is compared to a treatment threshold corresponding to the treatment metric. In some embodiments, if the treatment metric is a measure of fibrosis stage/status, the treatment threshold is an improvement in fibrosis greater than or equal to one stage. In some embodiments, if the treatment metric is percentage of liver fat, the treatment threshold is a reduction in liver fat content by greater than or equal to 30%. In some embodiments, if the comparison of the generated output from the machine learning model to a treatment threshold (e.g., at step 115 of method 100) indicates the generated output of the machine learning model satisfies the treatment threshold, then the treatment regimen being considered is administered to the patient.

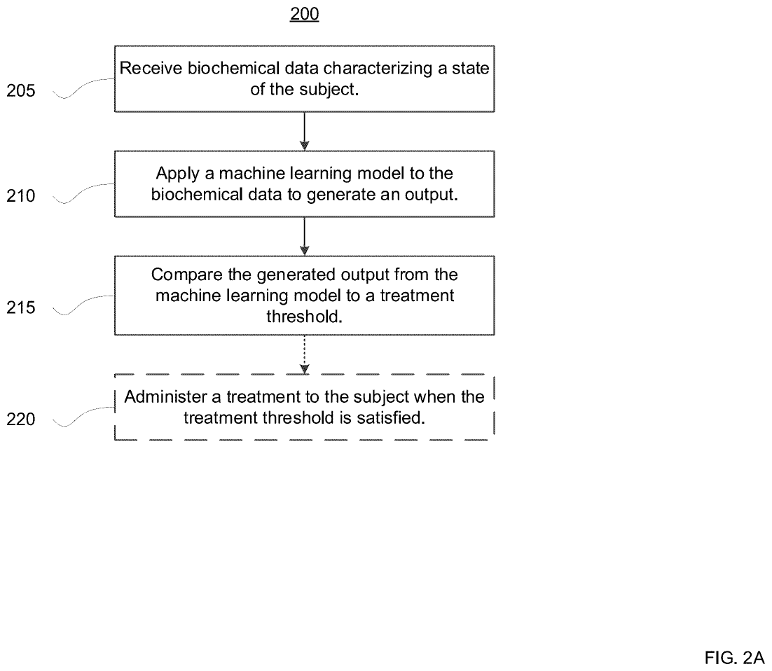

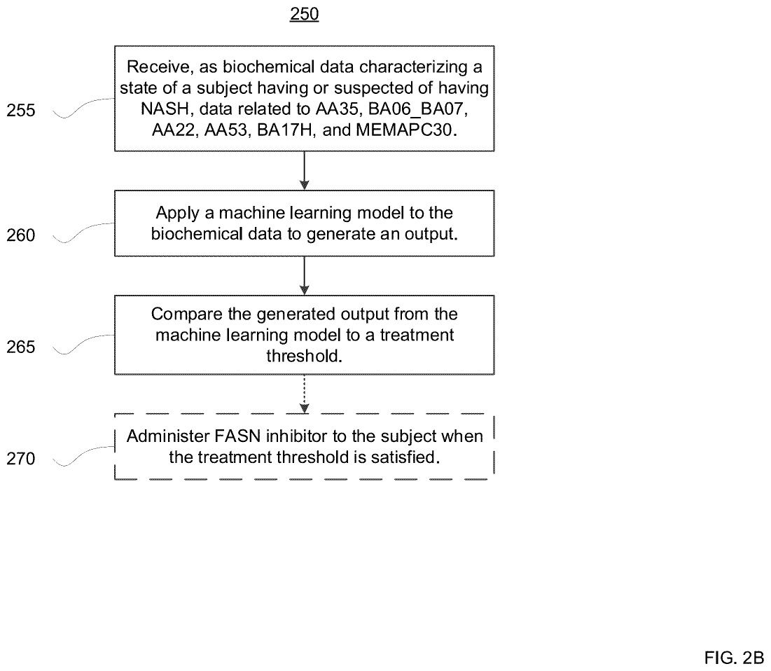

To this end, FIG. 2A and FIG. 2B are flow diagrams describing methods for predicting responsiveness of a patient, or subject, to a particular treatment regimen and, when it is determined the subject may be responsive, administering the treatment regimen to the subject. As with method 100 of FIG. 1, method 200 of FIG. 2A and method 250 FIG. 2B can be performed by a computing device having processing circuitry configured to control, instruct, and/or perform the methods, such as one or more of the computing devices of described with reference to FIG. 8.

For example, in method 200 of FIG. 2A, wherein the input data is biochemical data, biochemical data characterizing a state of a subject can be received at step 205 of method 200. At step 210 of method 200, a machine learning model can be applied to the received biochemical data characterizing the state of the subject. In some embodiments, the trained machine learning model may be one that is based on a set of features received at step 205 of method 200 as the biochemical data.

In some embodiments of the methods, apparatus, or non-transitory computer-readable storage medium disclosed herein, the subject may be an individual with NAFLD and the like, or derivatives thereof. In some embodiments, the biochemical data characterizing the state of the subject describes a ‘baseline’ condition, or reference condition. In some embodiments, the reference condition is one that reflects a medical condition of the subject before the initiation of a treatment regimen.

In some embodiments of the methods, apparatus, or non-transitory computer-readable storage medium disclosed herein, the biochemical data includes, as will be described detail below, proteomic data, metabolomic data, transcriptomics data, genomic data, and the like. Exemplary biochemical data can be found in Table 1. In some embodiments, the biochemical data includes a biochemical data variable selected from the data in Table 1. In some embodiments, the biochemical data includes fibrosis markers such as PRO—C3, TIMP-1, and PIIINP and/or liver biomarkers such as ALT. In some embodiments, the biochemical data includes an ELF score based on TIMP-1, PIIINP, and hyaluronic acid. In some embodiments, the biochemical data includes biometric data such as body weight and/or medical imaging data such as processed image data quantifying percentage of liver fat.

TABLE 1
Biochemical data variables.

| | | | Individual |
|---|---|---|---|
| | | | Notation/Composition |
| Variable ID | Class | Subclass | or Probable ID |
| BA06_BA07 | Bile acids | | Ursodeoxycholic + |
| | | | Hyodeoxycholic acid |
| AA35 | Amino Acids | Alpha Amino Acids and | DL-2-Aminocaprylic |
| | and Derivatives | Derivatives | acid |
| AA22 | Amino Acids | Alpha Amino Acids and | Sarcosine |
| | and Derivatives | Derivatives | |
| BA17H | Bile acids | Glycine-conjugated bile | Glycoursodeoxycholic |
| | | acids | acid |
| AA53 | AA derivative | Alpha Amino Acids and | D(−)-2-Aminobutyric |
| | | Derivatives | acid |
| MEMAPC30 | Glycerophospholipids | 1-ether, 2- | PC(O-18:0/22:4) |
| | | acylglycerophosphocholine | |
| TIMP-1 (ng/ml) | | | tissue inhibitor of |
| | | | metalloproteinases 1 |
| DAPC32 | Glycerophospholipids | Diacylglycerophosphocholine | PC(18:1/22:6) |
| AA05 | Amino acids | non-essential | Serine |
| BA11 | Bile acids | Taurine-conjugated bile | Taurochenodeoxycholic |
| | | acids | acid |
| PIIINP (ng/ml) | | | type III procollagen |
| | | | pro-peptide |
| AC07 | Fatty esters | Acylcarnitines | AC(8:1n-x) |
| SphLip_16 | Sphingolipids | Sphingomyelin | SM(38:1) |
| ST09 | Sterols | Steroid sulfates | isomer androsterone |
| | | | sulfate |
| IP10 (ng/ml) | | | interferon gamma- |
| | | | induced protein 10 |
| FFA21 | Fatty acids | Polyunsaturated fatty | 20:3n-x |
| | | acids | |
| AA15 | Amino acids | essential | Lysine |
| AA03 | Amino acids | non-essential | Glycine |
| FFAox37 | Oxidized fatty | Trihydroxy- | 9,12,13-TriHOME |
| | acids | octadecadenoic acids | |
| FFA25 | Fatty acids | Polyunsaturated fatty | 20:4n-3 |
| | | acids | |
| FFA04 | Fatty acids | Monounsaturated fatty | 16:1n-7 |
| | | acids | |
| BA15 | Bile acids | Glycine-conjugated bile | Glycocholic acid |
| | | acids | |
| MEMAPE15 | Glycerophospholipids | 1-ether, 2- | PE(P-20:0/20:4) |
| | | acylglycerophosphoethanolamine | |
| FFA19 | Fatty acids | Polyunsaturated fatty | 20:2n-6 |
| | | acids | |
| BA04 | Bile acids | Secondary bile acids | Deoxycholic acid |
| BA05 | Bile acids | Primary bile acids | Chenodeoxycholic acid |
| MAPC49 | Glycerophospholipids | 1- | PC(22:6/0:0) |
| | | Monoacylglycerophosphocholine | |
| ST01 | Sterols | Steroids | Cortisol |
| | | | (hydrocortisone) |
| FFA14 | Fatty acids | Polyunsaturated fatty | 18:3n-3 |
| | | acids | |
| Cer_13 | Sphingolipids | Ceramides | Cer(d18:1/18:0) |
| MEMAPC21 | Glycerophospholipids | 1-ether, 2- | PC(O-16:0/22:4) |
| | | acylglycerophosphocholine | |
| FFA20 | Fatty acids | Polyunsaturated fatty | 20:3n-3 |
| | | acids | |
| SphLip_13 | Sphingolipids | Sphingomyelin | SM(d18:1/18:0) |
| BA09 | Bile acids | Taurine-conjugated bile | Taurocholic acid |
| | | acids | |
| FFA41 | Fatty acids | Polyunsaturated fatty | 18:4n-3 |
| | | acids | |
| MEMAPC37 | Glycerophospholipids | 1-ether, 2- | PC(O-22:0/20:4) |
| | | acylglycerophosphocholine | |
| MEMAPC06 | Glycerophospholipids | 1-ether, 2- | PC(O-34:1) |
| | | acylglycerophosphocholine | |
| FFA30 | Fatty acids | Polyunsaturated fatty | 22:6n-3 |
| | | acids | |
| SphLip_05 | Sphingolipids | Sphingomyelin | SM(d18:0/18:0) |
| FFA28 | Fatty acids | Polyunsaturated fatty | 22:5n-3 |
| | | acids | |
| MAPC48 | Glycerophospholipids | 2- | PC(0:0/22:6) |
| | | Monoacylglycerophosphocholine | |
| AA20 | Amino acids | conditionally essential | Tyrosine |
| | | (phenylalanine) | |
| FFA47 | Fatty acids | Polyunsaturated fatty | 16:3n-x |
| | | acids | |
| MEMAPC26 | Glycerophospholipids | 1-ether, 2- | PC(P-18:0/20:4) |
| | | acylglycerophosphocholine | |
| FFA27 | Fatty acids | Polyunsaturated fatty | 22:4n-6 |
| | | acids | |
| DG11 | Glycerolipids | Diacylglycerols | DG(36:4) |
| FFA46 | Fatty acids | Polyunsaturated fatty | 22:3n-x |
| | | acids | |
| FFA22 | Fatty acids | Polyunsaturated fatty | 20:3n-9 |
| | | acids | |
| AA14 | Amino acids | non-essential | Glutamine |
| FFA17 | Fatty acids | Monounsaturated fatty | 20:1n-6 |
| | | acids | |
| FFA33 | Fatty acids | Saturated fatty acids | 15:0 |
| TG49 | Glycerolipids | Triacylglycerols | TG(54:2) |
| ChoE_07 | Sterols | Cholesteryl Ester | ChoE(18:1) |
| FFA05 | Fatty acids | Monounsaturated fatty | 16:1n-9 |
| | | acids | |
| FFA12 | Fatty acids | Polyunsaturated fatty | 18:2n-x |
| | | acids | |
| TG121 | Glycerolipids | Triacylglycerols | TG(40:0) |
| FFA23 | Fatty acids | Polyunsaturated fatty | 20:4n-6 |
| | | acids | |
| AA08 | Amino acids | essential | Threonine |
| FFA29 | Fatty acids | Polyunsaturated fatty | 22:5n-6 |
| | | acids | |
| FFA40 | Fatty acids | Monounsaturated fatty | 14:1n-5 trans |
| | | acids | |
| SphLip_10 | Sphingolipids | Sphingomyelin | SM(d18:1/16:0) |
| ST04 | Sterols | Steroid sulfates | isomer |
| | | | Dehydroepiandrosterone |
| | | | sulfate (DHEAS) |
| FFAox15 | Oxidized fatty | Hydroxy- | x-HODE |
| | acids | octadecadienoic acids | |
| TG56 | Glycerolipids | Triacylglycerols | TG(54:7) |
| DAPC26 | Glycerophospholipids | Diacylglycerophosphocholine | PC(18:0/22:4) |
| MEMAPC03 | Glycerophospholipids | 1-ether, 2- | PC(O-16:0/16:0) |
| | | acylglycerophosphocholine | |
| MEMAPC11 | Glycerophospholipids | 1-ether, 2- | PC(O-18:1/18:1) |
| | | acylglycerophosphocholine | |
| FFA08 | Fatty acids | Monounsaturated fatty | 18:1n-9 |
| | | acids | |
| TG50 | Glycerolipids | Triacylglycerols | TG(54:3) |
| TG74 | Glycerolipids | Triacylglycerols | TG(58:7) |
| FFA45 | Fatty acids | Polyunsaturated fatty | 22:3n-x |
| | | acids | |
| AA04 | Amino acids | non-essential | Alanine |
| MEMAPC38 | Glycerophospholipids | 1-ether, 2- | PC(O-24:1/20:4) |
| | | acylglycerophosphocholine | |
| DAPC18 | Glycerophospholipids | Diacylglycerophosphocholine | PC(18:0/20:3) |
| SphLip_20 | Sphingolipids | Sphingomyelin | SM(d18:1/22:0) |
| FFA44 | Fatty acids | Monounsaturated fatty | 17:1n-x |
| | | acids | |
| AA19 | Amino acids | non-essential | Arginine |
| MAPC53 | Glycerophospholipids | Monoacylglycerophosphocholine | LPC(22:0) |
| FFA35 | Fatty acids | Monounsaturated fatty | 19:1n-x |
| | | acids | |
| DAPC30 | Glycerophospholipids | Diacylglycerophosphocholine | PC(18:0/22:6) |
| AC02 | Fatty esters | Acylcarnitines | AC(8:0) |
| AA02 | Amino acids | non-essential | Glutamic Acid |
| MAPC42 | Glycerophospholipids | 1- | PC(22:4/0:0) |
| | | Monoacylglycerophosphocholine | |
| DAPE12 | Glycerophospholipids | Diacylglycerophosphocholine | PC(16:0/19:1) |
| FFA24 | Fatty acids | Polyunsaturated fatty | 20:4n-x |
| | | acids | |
| FSB01 | Sphingolipids | Free Sphingoid base | Sphingosine-1- |
| | | | phosphate |
| MEMAPC22 | Glycerophospholipids | 1-ether, 2- | PC(O-18:0/20:4) |
| | | acylglycerophosphocholine | |
| FFA39 | Fatty acids | Monounsaturated fatty | 14:1n-5 |
| | | acids | |
| MEMAPC18 | Glycerophospholipids | 1-ether, 2- | PC(P-16:0/20:3) |
| | | acylglycerophosphocholine | |
| FFA03 | Fatty acids | Saturated fatty acids | 16:0 |
| MAPC25 | Glycerophospholipids | 2- | PC(0:0/20:0) |
| | | Monoacylglycerophosphocholine | |
| SphLip_04 | Sphingolipids | Sphingomyelin | SM(d18:0/16:0) |
| AA13 | Amino acids | non-essential | Aspartic Acid |
| FFA02 | Fatty acids | Saturated fatty acids | 14:0 |
| AA12 | Amino acids | non-essential | Asparagine |
| ST03 | Sterols | Steroid sulfates | Pregnenolone sulfate |
| FFA11 | Fatty acids | Polyunsaturated fatty | 18:2n-6 |
| | | acids | |
| TG64 | Glycerolipids | Triacylglycerols | TG(56:6) |
| MAPC46 | Glycerophospholipids | 2- | PC(0:0/22:5) |
| | | Monoacylglycerophosphocholine | |
| SphLip_21 | Sphingolipids | Sphingomyelin | SM(d16:1/24:1) |
| NAE_04 | Fatty amides | N-acyl ethanolamines | NAE(18:0) |
| SphLip_07 | Sphingolipids | Sphingomyelin | SM(d18:1/12:0) |
| AA32 | Peptides | Dipeptide | Gly-Gly |
| ALT (U/L) | | | alanine |
| | | | aminotransferase |
| MEMAPE11 | Glycerophospholipids | 1-ether, 2- | PE(P-16:0/22:6) |
| | | acylglycerophosphoethanolamine | |
| SphLip_12 | Sphingolipids | Sphingomyelin | SM(d18:1/17:0) |
| MEMAPC05 | Glycerophospholipids | 1-ether, 2- | PC(O-34:0) |
| | | acylglycerophosphocholine | |
| TG63 | Glycerolipids | Triacylglycerols | TG(56:5) |
| MEMAPE13 | Glycerophospholipids | 1-ether, 2- | PC(P-17:0/20:4) |
| | | acylglycerophosphocholine | |
| MAPC47 | Glycerophospholipids | 1- | PC(22:5/0:0) |
| | | Monoacylglycerophosphocholine | |
| TG163 | Glycerolipids | Triacylglycerols | TG(52:4) |
| MEMAPC02 | Glycerophospholipids | 1-ether, 2- | PC(P-16:0/14:0) |
| | | acylglycerophosphocholine | |
| BA03 | Bile acids | Primary bile acids | Cholic acid |
| DAPE30 | Glycerophospholipids | Diacylglycerophosphocholine | PC(15:0/22:6) |
| MEMAPC04 | Glycerophospholipids | 1-ether, 2- | PC(P-16:0/16:0) |
| | | acylglycerophosphocholine | |
| AA09 | Amino acids | non-essential | Taurine |
| AA34 | Amino Acids | Alpha Amino Acids and | L-Citrulline |
| | and Derivatives | Derivatives | |
| MEMAPC32 | Glycerophospholipids | 1-ether, 2- | PC(O-18:1/22:4) |
| | | acylglycerophosphocholine | |
| MAPC45 | Glycerophospholipids | 1- | PC(22:5/0:0) |
| | | Monoacylglycerophosphocholine | |
| MEMAPC17 | Glycerophospholipids | 1-ether, 2- | PC(O-16:0/20:4) |
| | | acylglycerophosphocholine | |
| FFA49 | Fatty acids | Polyunsaturated fatty | 20:2n-x |
| | | acids | |
| MAPE36 | Glycerophospholipids | Monoacylglycerophosphoethanolamine | PE(22:5/0:0) |
| Adiponectin (ug/ml) | | | Adiponectin (ug/ml) |
| CMH_02 | Sphingolipids | Monohexosylceramides | CMH(d18:1/22:0) |
| MEMAPC35 | Glycerophospholipids | 1-ether, 2- | PC(O-22:1/20:4) |
| | | acylglycerophosphocholine | |
| TG73 | Glycerolipids | Triacylglycerols | TG(58:6) |
| CMH_01 | Sphingolipids | Monohexosylceramides | CMH(d18:1/16:0) |
| AA29 | Amino Acids | Alpha Amino Acids | Kynurenine |
| | and Derivatives | and Derivatives | |
| MEPE09 | Glycerophospholipids | 1- | PE(P-16:1/0:0) |
| | | Monoetherglycerophosphoethanolamine | |
| AA28 | Amino Acids | Alpha Amino Acids | DL-2-Aminoadipic |
| | and Derivatives | and Derivatives | acid |
| DAPE05 | Glycerophospholipids | Diacylglycerophosphocholine | PC(33:1) |
| DAPC28 | Glycerophospholipids | Diacylglycerophosphocholine | PC(40:5) |
| DG09 | Glycerolipids | Diacylglycerols | DG(36:3) |
| DAPC24 | Glycerophospholipids | Diacylglycerophosphocholine | PC(16:0/22:6) |
| MAPE22 | Glycerophospholipids | Monoacylglycerophosphoethanolamine | PE(18:3/0:0) |
| MEMAPC01 | Glycerophospholipids | 1-ether, 2- | PC(O-16:0/14:0) |
| | | acylglycerophosphocholine | |
| ST08 | Sterols | Steroid sulfates | androsterone sulfate + |
| | | | etiocholanolone sulfate |
| FFA38 | Fatty acids | Monounsaturated fatty | 24:1n-9 |
| | | acids | |
| MAPC09 | Glycerophospholipids | 1- | PC(16:1/0:0) |
| | | Monoacylglycerophosphocholine | |
| MEPE19 | Glycerophospholipids | 1- | PE(P-20:1/0:0) |
| | | Monoetherglycerophosphoethanolamine | |
| SphLip_19 | Sphingolipids | Sphingomyelin | SM(39:1) |
| TG66 | Glycerolipids | Triacylglycerols | TG(56:7) |
| DAPE25 | Glycerophospholipids | Diacylglycerophosphocholine | PC(17:0/20:3) |
| MEMAPE29 | Glycerophospholipids | 1-ether, 2- | PE(18:1e/22:6) |
| | | acylglycerophosphoethanolamine | |
| AC06 | Fatty esters | Acylcarnitines | AC(14:1n-x) |
| SphLip_02 | Sphingolipids | Sphingomyelin | SM(d18:0/14:0) |
| ProC3 D1 | | | ProC3 D1 collagen |
| (ng/ml) | | | neo-epitope |
| SphLip_29 | Sphingolipids | Sphingomyelin | SM(d18:2/14:0) |
| DAPE10 | Glycerophospholipids | Diacylglycerophosphoethanolamine | PE(20:5/16:0) |
| TG54 | Glycerolipids | Triacylglycerols | TG(54:6) |
| TG80 | Glycerolipids | Triacylglycerols | TG(60:2) |
| ST02 | Sterols | Steroid sulfates | Dehydroepiandrosterone |
| | | | sulfate (DHEAS) |
| MAPC50 | Glycerophospholipids | 1- | PC(17:0/0:0) |
| | | Monoacylglycerophosphocholine | |
| BA18 | Bile acids | Glycine-conjugated bile | Glycodeoxycholic acid |
| | | acids | |
| TG71 | Glycerolipids | Triacylglycerols | TG(58:3) |
| SphLip_06 | Sphingolipids | Sphingomyelin | SM(d18:0/22:0) |
| MEMAPC31 | Glycerophospholipids | 1-ether, 2- | PC(O-20:0/20:4) |
| | | acylglycerophosphocholine | |
| Cer_07 | Sphingolipids | Ceramides | Cer(d18:1/23:0) |
| SphLip_30 | Sphingolipids | Sphingomyelin | SM(42:1) |
| MAPE19 | Glycerophospholipids | 1- | PE(18:2/0:0) |
| | | Monoacylglycerophosphoethanolamine | |
| MAPE39 | Glycerophospholipids | Monoacylglycerophosphoethanolamine | PE(22:5/0:0) |
| MAPE18 | Glycerophospholipids | 2- | PE(0:0/18:2) |
| | | Monoacylglycerophosphoethanolamine | |
| TG45 | Glycerolipids | Triacylglycerols | TG(53:2) |
| ChoE_08 | Sterols | Cholesteryl Ester | ChoE(18:2) |
| ChoE_09 | Sterols | Cholesteryl Ester | ChoE(18:3) |
| TG68 | Glycerolipids | Triacylglycerols | TG(56:8) |
| MAPC26 | Glycerophospholipids | 1- | PC(20:0/0:0) |
| | | Monoacylglycerophosphocholine | |
| FFA07 | Fatty acids | Saturated fatty acids | 18:0 |
| DAPE17 | Glycerophospholipids | Diacylglycerophosphocholine | PC(15:0/20:4) |
| DAPC27 | Glycerophospholipids | Diacylglycerophosphocholine | PC(20:0/20:4) |
| MEMAPC24_MEMAPC25 | Glycerophospholipids | 1-ether, 2- | PC(O-38:5) |
| | | acylglycerophosphocholine | |
| MAPC39 | Glycerophospholipids | 2- | PC(0:0/20:5) |
| | | Monoacylglycerophosphocholine | |
| CMH_09 | Sphingolipids | Monohexosylceramides | CMH(d18:1/23:0) |
| DAPE21 | Glycerophospholipids | Diacylglycerophosphoethanolamine | PE(16:0/22:6) |
| MEMAPE06 | Glycerophospholipids | 1-ether, 2- | PE(P-16:0/20:4) |
| | | acylglycerophosphoethanolamine | |
| FFA34 | Fatty acids | Saturated fatty acids | 17:0 |
| DAPC29 | Glycerophospholipids | Diacylglycerophosphocholine | PC(18:0/22:5) |
| DAPC04 | Glycerophospholipids | Diacylglycerophosphocholine | PC(32:1) |
| SphLip_35 | Sphingolipids | Sphingomyelin | SM(31:1) |
| ST10 | Sterols | Steroid sulfates | isomer pregn-5-ene- |
| | | | 3,20-diol sulfate |
| MAPE29 | Glycerophospholipids | 1- | PE(20:3/0:0) |
| | | Monoacylglycerophosphoethanolamine | |
| SphLip_11 | Sphingolipids | Sphingomyelin | SM(d18:2/16:0) |
| AA16 | Amino acids | essential | Methionine |
| SphLip_22 | Sphingolipids | Sphingomyelin | SM(d18:2/22:0) |
| DG12 | Glycerolipids | Diacylglycerols | DG(36:4) |
| FFA26 | Fatty acids | Polyunsaturated fatty | 20:5n-3 |
| | | acids | |
| BA16H | Bile acids | Glycine-conjugated bile | Glycochenodeoxycholic |
| | | acids | acid |
| MEMAPC33_MEMAPC34 | Glycerophospholipids | 1-ether, 2- | PC(O-40:5) |
| | | acylglycerophosphocholine | |
| DAPC45 | Glycerophospholipids | Diacylglycerophosphocholine | PC(31:0) |
| MEMAPC29 | Glycerophospholipids | 1-ether, 2- | PC(P-16:0/22:6) |
| | | acylglycerophosphocholine | |
| AC18 | Fatty esters | Acylcarnitines | AC(18:0) |
| FFA13 | Fatty acids | Polyunsaturated fatty | 18:2n-6 trans |
| | | acids | |
| DAPC47 | Glycerophospholipids | Diacylglycerophosphocholine | PC(30:0) |
| TG39 | Glycerolipids | Triacylglycerols | TG(52:2) |
| DAPC46 | Glycerophospholipids | Diacylglycerophosphocholine | PC(14:0/14:0) |
| DAPI06 | Glycerophospholipids | Diacylglycerophosphoinositol | PI(18:0/20:4) |
| SphLip_34 | Sphingolipids | Sphingomyelin | SM(38:0) |
| MAPC40 | Glycerophospholipids | 1- | PC(20:5/0:0) |
| | | Monoacylglycerophosphocholine | |
| FFA15 | Fatty acids | Polyunsaturated fatty | 18:3n-6 |
| | | acids | |
| TG40 | Glycerolipids | Triacylglycerols | TG(52:3) |
| TG60 | Glycerolipids | Triacylglycerols | TG(56:2) |
| MEMAPE30 | Glycerophospholipids | 1-ether, 2- | PE(18:2e/22:6) |
| | | acylglycerophosphoethanolamine | |
| MAPC57 | Glycerophospholipids | Monoacylglycerophosphocholine | LPC(22:1) |
| AA44 | AA derivative | essential | Cystine-double charge |
| SphLip_17 | Sphingolipids | Sphingomyelin | SM(d18:2/20:0) |
| MAPI11 | Glycerophospholipids | Monoacylglycerophosphoinositol | LPI(20:4) |
| SphLip_09 | Sphingolipids | Sphingomyelin | SM(33:1) |
| AC05 | Fatty esters | Acylcarnitines | AC(12:0) |
| MEMAPC13 | Glycerophospholipids | 1-ether, 2- | PC(O-18:1/18:2) |
| | | acylglycerophosphocholine | |
| MEPE12 | Glycerophospholipids | 1- | PE(P-18:0/0:0) |
| | | Monoetherglycerophosphoethanolamine | |
| Cer_02 | Sphingolipids | Ceramides | Cer(d18:1/16:0) |
| MAPC19 | Glycerophospholipids | 1- | PC(18:2/0:0) |
| | | Monoacylglycerophosphocholine | |
| DAPE28 | Glycerophospholipids | Diacylglycerophosphocholine | PC(37:5) |
| MAPC06 | Glycerophospholipids | 1- | PC(16:0/0:0) |
| | | Monoacylglycerophosphocholine | |
| ChoE_11 | Sterols | Cholesteryl Ester | ChoE(20:3) |
| MEMAPC09 | Glycerophospholipids | 1-ether, 2- | PC(P-16:0/18:2) |
| | | acylglycerophosphocholine | |
| MEMAPC15_MEMAPC16 | Glycerophospholipids | 1-ether, 2- | PC(P-36:2) |
| | | acylglycerophosphocholine | |
| MEPE08 | Glycerophospholipids | 1- | PE(O-16:0/0:0) |
| | | Monoetherglycerophosphoethanolamine | |
| MAPE41 | Glycerophospholipids | 1- | PE(22:6/0:0) |
| | | Monoacylglycerophosphoethanolamine | |
| DAPE06 | Glycerophospholipids | Diacylglycerophosphocholine | PC(15:0/18:2) |
| MAPE32 | Glycerophospholipids | Monoacylglycerophosphoethanolamine | LPE(20:5) |
| MAPC44 | Glycerophospholipids | 2- | PC(0:0/22:5) |
| | | Monoacylglycerophosphocholine | |
| Cer_03 | Sphingolipids | Ceramides | Cer(d18:1/20:0) |
| ST05 | Sterols | Steroid sulfates | isomer androsterone |
| | | | sulfate |
| AC10 | Fatty esters | Acylcarnitines | AC(18:2n-6) |
| DAPE08 | Glycerophospholipids | Diacylglycerophosphoethanolamine | PE(16:0/20:4) |
| DAPC44 | Glycerophospholipids | Diacylglycerophosphocholine | PC(16:1/20:4) |
| MAPE17 | Glycerophospholipids | 1- | PE(18:1/0:0) |
| | | Monoacylglycerophosphoethanolamine | |
| DAPE03 | Glycerophospholipids | Diacylglycerophosphoethanolamine | PE(16:0/18:2) |
| SphLip_03 | Sphingolipids | Sphingomyelin | SM(d18:0/15:0) |
| MAPC33 | Glycerophospholipids | 2- | PC(0:0/20:3) |
| | | Monoacylglycerophosphocholine | |
| DAPC20 | Glycerophospholipids | Diacylglycerophosphocholine | PC(38:5) |
| DG57 | Glycerolipids | Diacylglycerols | DG(36:1) |
| AA10 | Amino acids | essential | Isoleucine |
| MEPC02 | Glycerophospholipids | 1- | PC(O-16:0/0:0) |
| | | Monoetherglycerophosphocholine | |
| MEPE18 | Glycerophospholipids | 1- | PE(P-20:0/0:0) |
| | | Monoetherglycerophosphoethanolamine | |
| MEMAPC20 | Glycerophospholipids | 1-ether, 2- | PC(O-38:4) |
| | | acylglycerophosphocholine | |
| MAPE40 | Glycerophospholipids | 2- | PE(0:0/22:6) |
| | | Monoacylglycerophosphoethanolamine | |
| MAPC05 | Glycerophospholipids | 2- | PC(0:0/15:0) |
| | | Monoacylglycerophosphocholine | |
| TG51 | Glycerolipids | Triacylglycerols | TG(54:4) |
| DAPC08 | Glycerophospholipids | Diacylglycerophosphocholine | PC(16:0/18:2) |
| SphLip_08 | Sphingolipids | Sphingomyelin | SM(32:1) |
| MAPI13 | Glycerophospholipids | Monoacylglycerophosphoinositol | LPI(22:6) |
| DG07 | Glycerolipids | Diacylglycerols | DG(34:2) |
| MEMAPE14 | Glycerophospholipids | 1-ether, 2- | PC(P-17:0/20:4) |
| | | acylglycerophosphocholine | |
| AA18 | Amino acids | essential | Phenylalanine |
| DAPC11 | Glycerophospholipids | Diacylglycerophosphocholine | PC(18:0/18:1) |
| TG59 | Glycerolipids | Triacylglycerols | TG(56:1) |
| MEMAPC08 | Glycerophospholipids | 1-ether, 2- | PC(P-16:0/18:1) |
| | | acylglycerophosphocholine | |
| TG82 | Glycerolipids | Triacylglycerols | TG(42:1) |
| DAPC12 | Glycerophospholipids | Diacylglycerophosphocholine | PC(18:0/18:2) |
| DAPE18 | Glycerophospholipids | Diacylglycerophosphoethanolamine | PE(18:0/20:4) |
| DAPE14 | Glycerophospholipids | Diacylglycerophosphocholine | PC(17:0/18:2) |
| ChoE_03 | Sterols | Cholesteryl Ester | ChoE(16:0) |
| MAPC54 | Glycerophospholipids | 1- | PC(17:1/0:0) |
| | | Monoacylglycerophosphocholine | |
| MAPE15 | Glycerophospholipids | 2- | PE(0:0/18:1) |
| | | Monoacylglycerophosphoethanolamine | |
| TG20 | Glycerolipids | Triacylglycerols | TG(48:4) |
| MAPI08 | Glycerophospholipids | Monoacylglycerophosphoinositol | LPI(18:2) |
| MEMAPC36 | Glycerophospholipids | 1-ether, 2- | PC(O-42:6) |
| | | acylglycerophosphocholine | |
| DAPE11 | Glycerophospholipids | Diacylglycerophosphocholine | PC(17:0/18:1) |
| Cer_04 | Sphingolipids | Ceramides | Cer(d18:1/21:0) |
| TG52 | Glycerolipids | Triacylglycerols | TG(54:5) |
| DAPC05 | Glycerophospholipids | Diacylglycerophosphocholine | PC(14:0/18:2) |
| MAPC37 | Glycerophospholipids | 2- | PC(0:0/20:4) |
| | | Monoacylglycerophosphocholine | |
| DAPE04 | Glycerophospholipids | Diacylglycerophosphocholine | PC(16:0/17:0) |
| FFAox12 | Oxidized fatty | Hydroxy- | 13-HODE |
| | acids | octadecadienoic acids | |
| DAPC15 | Glycerophospholipids | Diacylglycerophosphocholine | PC(16:0/20:5) |
| MAPC41 | Glycerophospholipids | 2- | PC(0:0/22:4) |
| | | Monoacylglycerophosphocholine | |
| MEMAPC14 | Glycerophospholipids | 1-ether, 2- | PC(O-16:0/20:3) |
| | | acylglycerophosphocholine | |
| TG70 | Glycerolipids | Triacylglycerols | TG(58:2) |
| MAPI09 | Glycerophospholipids | Monoacylglycerophosphoinositol | LPI(18:2) |
| TG46 | Glycerolipids | Triacylglycerols | TG(53:3) |
| AA06 | Amino acids | non-essential | Proline |
| DAPE23 | Glycerophospholipids | Diacylglycerophosphocholine | PC(37:2) |
| TG61 | Glycerolipids | Triacylglycerols | TG(56:3) |
| MAPC12 | Glycerophospholipids | 1- | PC(16:1/0:0) |
| | | Monoacylglycerophosphocholine | |
| DG10 | Glycerolipids | Diacylglycerols | DG(36:4) |
| DAPC37 | Glycerophospholipids | Diacylglycerophosphocholine | PC(22:4/20:4) |
| SphLip_14 | Sphingolipids | Sphingomyelin | SM(36:2) |
| TG06 | Glycerolipids | Triacylglycerols | TG(44:2) |
| DAPC38 | Glycerophospholipids | Diacylglycerophosphocholine | PC(16:1/18:2) |
| DAPC09 | Glycerophospholipids | Diacylglycerophosphocholine | PC(14:0/20:4) |
| DG33 | Glycerolipids | Diacylglycerols | DG(30:0) |
| MAPE37 | Glycerophospholipids | Monoacylglycerophosphoethanolamine | PE(22:5/0:0) |
| MEMAPC07 | Glycerophospholipids | 1-ether, 2- | PC(O-16:0/18:2) |
| | | acylglycerophosphocholine | |
| ChoE_17 | Sterols | Cholesteryl Ester | ChoE(22:6) |
| SphLip_25 | Sphingolipids | Sphingomyelin | SM(d18:2/23:0) |
| CMH_03 | Sphingolipids | Monohexosylceramides | CMH(d18:1/24:0) |
| MAPI06 | Glycerophospholipids | Monoacylglycerophosphoinositol | LPI(18:1) |
| MEMAPE01 | Glycerophospholipids | 1-ether, 2- | PE(O-16:0/18:1) |
| | | acylglycerophosphoethanolamine | |
| MAPE16 | Glycerophospholipids | 1- | PE(18:1/0:0) |
| | | Monoacylglycerophosphoethanolamine | |
| SphLip_26_SphLip_27 | Sphingolipids | Sphingomyelin | SM(d18:1/24:1) + |
| | | | SM(d18:2/24:0) |
| MEPC09 | Glycerophospholipids | 1- | PC(P-18:1/0:0) |
| | | Monoetherglycerophosphocholine | |
| MAPC04 | Glycerophospholipids | 1- | PC(15:0/0:0) |
| | | Monoacylglycerophosphocholine | |
| MEMAPE16 | Glycerophospholipids | 1-ether, 2- | PE(P-18:0/22:5) + |
| | | acylglycerophosphoethanolamine | PE(P-20:1/20:4) |
| DAPC16 | Glycerophospholipids | Diacylglycerophosphocholine | PC(18:3/18:3) |
| TG27 | Glycerolipids | Triacylglycerols | TG(50:1) |
| FFAox14 | Oxidized fatty | Hydroxy- | x-HODE |
| | acids | octadecadienoic acids | |
| DG06 | Glycerolipids | Diacylglycerols | DG(34:1) |
| MAPE21 | Glycerophospholipids | Monoacylglycerophosphoethanolamine | PE(18:3/0:0) |
| DG04 | Glycerolipids | Diacylglycerols | DG(32:2) |
| MAPC03 | Glycerophospholipids | 2- | PC(0:0/14:0) |
| | | Monoacylglycerophosphocholine | |
| MAPC55 | Glycerophospholipids | 2- | PC(0:0/17:1) |
| | | Monoacylglycerophosphocholine | |
| AC11 | Fatty esters | Acylcarnitines | AC(18:1n-9) |
| DAPC39_DAPC40 | Glycerophospholipids | Diacylglycerophosphocholine | PC(34:3) |
| MAPC20 | Glycerophospholipids | 1- | PC(18:2/0:0) |
| | | Monoacylglycerophosphocholine | |
| MAPE20 | Glycerophospholipids | 1- | PE(18:2/0:0) |
| | | Monoacylglycerophosphoethanolamine | |
| FFA09 | Fatty acids | Monounsaturated fatty | 18:1n-x |
| | | acids | |
| MAPC35 | Glycerophospholipids | 1- | PC(20:3/0:0) |
| | | Monoacylglycerophosphocholine | |
| TG12 | Glycerolipids | Triacylglycerols | TG(46:3) |
| TG44 | Glycerolipids | Triacylglycerols | TG(53:1) |
| Cer_15 | Sphingolipids | Ceramides | Cer(d18:1/25:0) |
| MEMAPE02 | Glycerophospholipids | 1-ether, 2- | PE(P-16:0/18:2) |
| | | acylglycerophosphoethanolamine | |
| MEMAPE05 | Glycerophospholipids | 1-ether, 2- | PE(P-18:0/18:1) |
| | | acylglycerophosphoethanolamine | |
| HA (ng/ml) | | | hyaluronic acid (ng/ml) |
| MEMAPE09 | Glycerophospholipids | 1-ether, 2- | PE(P-18:1/20:4) |
| | | acylglycerophosphoethanolamine | |
| ChoE_15 | Sterols | Cholesteryl Ester | ChoE(22:5) |
| SphLip_33 | Sphingolipids | Sphingomyelin | SM(43:2) |
| SphLip_32 | Sphingolipids | Sphingomyelin | SM(43:1) |
| TG169 | Glycerolipids | Triacylglycerols | TG(53:5) |
| DG08 | Glycerolipids | Diacylglycerols | DG(36:2) |
| DG03 | Glycerolipids | Diacylglycerols | DG(32:1) |
| DAPE15 | Glycerophospholipids | Diacylglycerophosphocholine | PC(17:1/18:2) |
| MEPE15 | Glycerophospholipids | 1- | PE(P-18:2/0:0) |
| | | Monoetherglycerophosphoethanolamine | |
| MAPE28 | Glycerophospholipids | Monoacylglycerophosphoethanolamine | PE(0:0/20:3) |
| DAPC41_DAPC42 | Glycerophospholipids | Diacylglycerophosphocholine | PC(36:3) |
| MAPE06 | Glycerophospholipids | 1- | PE(14:0/0:0) |
| | | Monoacylglycerophosphoethanolamine | |
| MAPC36 | Glycerophospholipids | 1- | PC(20:3/0:0) |
| | | Monoacylglycerophosphocholine | |
| DG66 | Glycerolipids | Diacylglycerols | DG(38:5) |
| MEMAPC28 | Glycerophospholipids | 1-ether, 2- | PC(O-18:2/20:4) |
| | | acylglycerophosphocholine | |
| MAPC13 | Glycerophospholipids | 2- | PC(0:0/18:0) |
| | | Monoacylglycerophosphocholine | |
| AC04 | Fatty esters | Acylcarnitines | AC(10:0) |
| MAPC51 | Glycerophospholipids | 2- | PC(0:0/17:0) |
| | | Monoacylglycerophosphocholine | |
| TG174 | Glycerolipids | Triacylglycerols | TG(55:3) |
| TG69 | Glycerolipids | Triacylglycerols | TG(58:1) |
| TG11 | Glycerolipids | Triacylglycerols | TG(46:2) |
| TG19 | Glycerolipids | Triacylglycerols | TG(48:3) |
| AC15 | Fatty esters | Acylcarnitines | AC(14:2n-x) |
| MEMAPE08 | Glycerophospholipids | 1-ether, 2- | PE(P-18:0/20:4) |
| | | acylglycerophosphoethanolamine | |
| MEMAPC23 | Glycerophospholipids | 1-ether, 2- | PC(18:1e/20:3) |
| | | acylglycerophosphocholine | |
| Cer_12 | Sphingolipids | Ceramides | Cer(42:3) |
| MAPI10 | Glycerophospholipids | Monoacylglycerophosphoinositol | LPI(20:3) |
| AA21 | Amino acids | essential | Tryptophan |
| Cer_09 | Sphingolipids | Ceramides | Cer(d18:1/24:0) |
| MAPI05 | Glycerophospholipids | Monoacylglycerophosphoinositol | LPI(18:1) |
| TG173 | Glycerolipids | Triacylglycerols | TG(55:2) |
| MAPC07 | Glycerophospholipids | 2- | PC(0:0/16:0) |
| | | Monoacylglycerophosphocholine | |
| TG83 | Glycerolipids | Triacylglycerols | TG(53:4) |
| TG33 | Glycerolipids | Triacylglycerols | TG(51:1) |
| AA26 | Sulfinic Acids | Sulfinic Acids | Hypotaurine |
| | and Derivatives | | |
| MAPC21 | Glycerophospholipids | 2- | PC(0:0/18:3) |
| | | Monoacylglycerophosphocholine | |
| DAPC06 | Glycerophospholipids | Diacylglycerophosphocholine | PC(16:0/18:0) |
| DAPC17 | Glycerophospholipids | Diacylglycerophosphocholine | PC(20:0/18:2) |
| DAPC21 | Glycerophospholipids | Diacylglycerophosphocholine | PC(38:5) |
| TG03 | Glycerolipids | Triacylglycerols | TG(42:0) |
| DG02 | Glycerolipids | Diacylglycerols | DG(32:0) |
| TG48 | Glycerolipids | Triacylglycerols | TG(54:1) |
| SphLip_23 | Sphingolipids | Sphingomyelin | SM(d18:1/23:0) |
| TG77 | Glycerolipids | Triacylglycerols | TG(58:10) |
| MAPC56 | Glycerophospholipids | Monoacylglycerophosphocholine | LPC(19:1) |
| DAPC13 | Glycerophospholipids | Diacylglycerophosphocholine | PC(18:2/18:2) |
| TG10 | Glycerolipids | Triacylglycerols | TG(46:1) |
| SphLip_28 | Sphingolipids | Sphingomyelin | SM(42:3) |
| ChoE_12 | Sterols | Cholesteryl Ester | ChoE(20:4) |
| MAPE09 | Glycerophospholipids | 2- | PE(0:0/16:1) |
| | | Monoacylglycerophosphoethanolamine | |
| TG13_TG14 | Glycerolipids | Triacylglycerols | TG(47:0) |
| TG35 | Glycerolipids | Triacylglycerols | TG(51:3) |
| MAPC28 | Glycerophospholipids | 1- | PC(20:1/0:0) |
| | | Monoacylglycerophosphocholine | |
| TG17 | Glycerolipids | Triacylglycerols | TG(48:1) |
| MAPE33 | Glycerophospholipids | Monoacylglycerophosphoethanolamine | PE(20:5/0:0) |
| MAPC32 | Glycerophospholipids | 1- | PC(20:2/0:0) |
| | | Monoacylglycerophosphocholine | |
| MEPE13 | Glycerophospholipids | 1- | PE(P-18:1/0:0) |
| | | Monoetherglycerophosphoethanolamine | |
| MAPC08 | Glycerophospholipids | 2- | PC(0:0/16:1) |
| | | Monoacylglycerophosphocholine | |
| MEMAPC19 | Glycerophospholipids | 1-ether, 2- | PC(P-16:0/20:4) |
| | | acylglycerophosphocholine | |
| TG04 | Glycerolipids | Triacylglycerols | TG(44:0) |
| MAPC52 | Glycerophospholipids | 1- | PC(19:0/0:0) |
| | | Monoacylglycerophosphocholine | |
| TG145 | Glycerolipids | Triacylglycerols | TG(46:3) |
| MAPC14 | Glycerophospholipids | 1- | PC(18:0/0:0) |
| | | Monoacylglycerophosphocholine | |
| MAPC27 | Glycerophospholipids | 2- | PC(0:0/20:1) |
| | | Monoacylglycerophosphocholine | |
| MEPC21 | Glycerophospholipids | 1- | PC(O-24:2/0:0) |
| | | Monoetherglycerophosphocholine | |
| TG65 | Glycerolipids | Triacylglycerols | TG(56:7) |
| TG67 | Glycerolipids | Triacylglycerols | TG(56:8) |
| FGF21 (pg/ml) | | | fibroblast growth factor |
| | | | 21 |
| AC16 | Fatty esters | Acylcarnitines | AC(16:0) |
| TG09 | Glycerolipids | Triacylglycerols | TG(46:0) |
| DAPC03 | Glycerophospholipids | Diacylglycerophosphocholine | PC(16:0/16:0) |
| MEPC08 | Glycerophospholipids | 1- | PC(P-18:1/0:0) |
| | | Monoetherglycerophosphocholine | |
| TG53 | Glycerolipids | Triacylglycerols | TG(54:5) |
| TG55 | Glycerolipids | Triacylglycerols | TG(54:6) |
| TG62 | Glycerolipids | Triacylglycerols | TG(56:5) |
| AA11 | Amino acids | essential | Leucine |
| DAPE42 | Glycerophospholipids | Diacylglycerophosphoethanolamine | PE(18:0/18:1) |
| SphLip_24 | Sphingolipids | Sphingomyelin | SM(d18:1/23:1) |
| MAPC15 | Glycerophospholipids | 2- | PC(0:0/18:1) |
| | | Monoacylglycerophosphocholine | |
| TG16 | Glycerolipids | Triacylglycerols | TG(48:0) |
| TG166 | Glycerolipids | Triacylglycerols | TG(52:6) |
| MAPC18 | Glycerophospholipids | 2- | PC(0:0/18:2) |
| | | Monoacylglycerophosphocholine | |
| MAPC38 | Glycerophospholipids | 1- | PC(20:4/0:0) |
| | | Monoacylglycerophosphocholine | |
| MEPC20 | Glycerophospholipids | 1- | PC(O-24:1/0:0) |
| | | Monoetherglycerophosphocholine | |
| MEMAPC27 | Glycerophospholipids | 1-ether, 2- | PC(P-18:0/20:4) |
| | | acylglycerophosphocholine | |
| MAPI12 | Glycerophospholipids | Monoacylglycerophosphoinositol | LPI(20:4) |
| TG175 | Glycerolipids | Triacylglycerols | TG(55:4) |
| MAPE38 | Glycerophospholipids | Monoacylglycerophosphoethanolamine | PE(22:5/0:0) |
| MAPC43 | Glycerophospholipids | 1- | PC(22:4/0:0) |
| | | Monoacylglycerophosphocholine | |
| MEPC06 | Glycerophospholipids | 1- | PC(O-18:1/0:0) |
| | | Monoetherglycerophosphocholine | |
| MEMAPC12 | Glycerophospholipids | 1-ether, 2- | PC(O-18:0/18:2) |
| | | acylglycerophosphocholine | |
| AA07 | Amino acids | essential | Valine |
| TG84 | Glycerolipids | Triacylglycerols | TG(45:1) |
| FFAox16 | Oxidized fatty | Hydroxy- | X-HODE |
| | acids | octadecadienoic acids | |
| DAPC19 | Glycerophospholipids | Diacylglycerophosphocholine | PC(18:0/20:4) |
| MEMAPE17 | Glycerophospholipids | 1-ether, 2- | PE(P-18:0/18:2) |
| | | acylglycerophosphoethanolamine | |
| MAPC22 | Glycerophospholipids | 1- | PC(18:3/0:0) |
| | | Monoacylglycerophosphocholine | |
| MAPC17 | Glycerophospholipids | 1- | PC(18:1/0:0) |
| | | Monoacylglycerophosphocholine | |
| TG07_TG08 | Glycerolipids | Triacylglycerols | TG(45:0) |
| Cer_10_Cer_11 | Sphingolipids | Ceramides | Cer(d18:1/24:1) + |
| | | | Cer(d18:2/24:0) |
| DAPC07 | Glycerophospholipids | Diacylglycerophosphocholine | PC(16:0/18:1) |
| ChoE_13 | Sterols | Cholesteryl Ester | ChoE(20:5) |
| TG76 | Glycerolipids | Triacylglycerols | TG(58:9) |
| TG05 | Glycerolipids | Triacylglycerols | TG(44:1) |
| MEPC22 | Glycerophospholipids | Monoetherglycerophosphocholine | LPC(O-22:2) |
| MEPC10 | Glycerophospholipids | 1- | PC(O-20:0/0:0) |
| | | Monoetherglycerophosphocholine | |
| Cer_14 | Sphingolipids | Ceramides | Cer(43:1) |
| Cer_05 | Sphingolipids | Ceramides | Cer(d18:1/22:0) |
| DAPE13 | Glycerophospholipids | Diacylglycerophosphocholine | PC(17:1/18:1) |
| AA17 | Amino acids | essential | Histidine |
| MEPC14 | Glycerophospholipids | 1- | PC(P-20:1/0:0) |
| | | Monoetherglycerophosphocholine | |
| DAPE16 | Glycerophospholipids | Diacylglycerophosphocholine | PC(15:0/20:3) |
| Cer_06 | Sphingolipids | Ceramides | Cer(40:2) |
| MAPC16 | Glycerophospholipids | 1- | PC(18:1/0:0) |
| | | Monoacylglycerophosphocholine | |
| MEPC13 | Glycerophospholipids | 1- | PC(O-20:2/0:0) |
| | | Monoetherglycerophosphocholine | |
| DAPE24 | Glycerophospholipids | Diacylglycerophosphocholine | PC(19:1/18:2) |
| MAPC30 | Glycerophospholipids | 2- | PC(0:0/20:2) |
| | | Monoacylglycerophosphocholine | |
| MAPC29 | Glycerophospholipids | 1- | PC(20:1/0:0) |
| | | Monoacylglycerophosphocholine | |
| MAPI03 | Glycerophospholipids | Monoacylglycerophosphoinositol | LPI(18:0) |
| MAPI02 | Glycerophospholipids | Monoacylglycerophosphoinositol | LPI(16:0) |
| TG15 | Glycerolipids | Triacylglycerols | TG(47:1) |
| MAPE31 | Glycerophospholipids | 1- | PE(20:4/0:0) |
| | | Monoacylglycerophosphoethanolamine | |
| MAPC23 | Glycerophospholipids | 1- | PC(18:3/0:0) |
| | | Monoacylglycerophosphocholine | |
| MEPC17 | Glycerophospholipids | 1- | PC(O-22:1/0:0) |
| | | Monoetherglycerophosphocholine | |
| TG22 | Glycerolipids | Triacylglycerols | TG(49:0) |
| MEPE14 | Glycerophospholipids | 1- | PE(P-18:1/0:0) |
| | | Monoetherglycerophosphoethanolamine | |
| TG37 | Glycerolipids | Triacylglycerols | TG(52:0) |
| TG23 | Glycerolipids | Triacylglycerols | TG(49:1) |
| DAPE26 | Glycerophospholipids | Diacylglycerophosphocholine | PC(17:0/20:4) |
| MEPC19 | Glycerophospholipids | 1- | PC(O-22:0/0:0) |
| | | Monoetherglycerophosphocholine | |
| MAPC02 | Glycerophospholipids | 1- | PC(14:0/0:0) |
| | | Monoacylglycerophosphocholine | |
| TG38 | Glycerolipids | Triacylglycerols | TG(52:1) |
| TG26 | Glycerolipids | Triacylglycerols | TG(50:0) |
| TG24 | Glycerolipids | Triacylglycerols | TG(49:2) |
| MAPI01 | Glycerophospholipids | Monoacylglycerophosphoinositol | LPI(16:0) |
| TG29 | Glycerolipids | Triacylglycerols | TG(50:3) |
| TG25 | Glycerolipids | Triacylglycerols | TG(49:3) |
| TG31 | Glycerolipids | Triacylglycerols | TG(50:5) |
| DAPC23 | Glycerophospholipids | Diacylglycerophosphocholine | PC(18:2/20:4) |
| TG18 | Glycerolipids | Triacylglycerols | TG(48:2) |
| TG28 | Glycerolipids | Triacylglycerols | TG(50:2) |
| TG47 | Glycerolipids | Triacylglycerols | TG(54:0) |
| TG41 | Glycerolipids | Triacylglycerols | TG(52:4) |
| AA47 | Amino Acids | Alpha Amino Acids | 1-Methyl-L-histidine |
| | and Derivatives | and Derivatives | |
| TG34 | Glycerolipids | Triacylglycerols | TG(51:2) |
| MAPI04 | Glycerophospholipids | Monoacylglycerophosphoinositol | LPI(18:0) |
| TG152 | Glycerolipids | Triacylglycerols | TG(47:2) |
| MAPC31 | Glycerophospholipids | 1- | PC(20:2/0:0) |
| | | Monoacylglycerophosphocholine | |
| FFA06 | Fatty acids | Monounsaturated fatty | 16:1n-x |
| | | acids | |
| SphLip_31 | Sphingolipids | Sphingomyelin | SM(d18:1/25:0) |
| MEPC03 | Glycerophospholipids | 1- | PC(P-16:0/0:0) |
| | | Monoetherglycerophosphocholine | |
| TG30 | Glycerolipids | Triacylglycerols | TG(50:4) |
| DAPE19 | Glycerophospholipids | Diacylglycerophosphoethanolamine | PE(38:5) |
| DAPC14 | Glycerophospholipids | Diacylglycerophosphocholine | PC(16:0/20:4) |
| MEPC11 | Glycerophospholipids | 1- | PC(O-20:1/0:0) |
| | | Monoetherglycerophosphocholine | |
| MAPE13 | Glycerophospholipids | 2- | PE(0:0/18:0) |
| | | Monoacylglycerophosphoethanolamine | |
| TG42 | Glycerolipids | Triacylglycerols | TG(52:5) |
| TG36 | Glycerolipids | Triacylglycerols | TG(51:4) |
| DAPC34 | Glycerophospholipids | Diacylglycerophosphocholine | PC(40:8) |
| MAPE35 | Glycerophospholipids | 1- | PE(22:4/0:0) |
| | | Monoacylglycerophosphoethanolamine | |
| MAPE14 | Glycerophospholipids | 1- | PE(18:0/0:0) |
| | | Monoacylglycerophosphoethanolamine | |
| MEPC05 | Glycerophospholipids | 1- | PC(O-18:1/0:0) |
| | | Monoetherglycerophosphocholine | |
| MAPE08 | Glycerophospholipids | 1- | PE(16:0/0:0) |
| | | Monoacylglycerophosphoethanolamine | |
| MAPE30 | Glycerophospholipids | 2- | PE(0:0/20:4) |
| | | Monoacylglycerophosphoethanolamine | |
| MEPC04 | Glycerophospholipids | 1- | PC(O-18:0/0:0) |
| | | Monoetherglycerophosphocholine | |
| MAPE07 | Glycerophospholipids | 2- | PE(0:0/16:0) |
| | | Monoacylglycerophosphoethanolamine | |
| MEPC07 | Glycerophospholipids | 1- | PC(P-18:0/0:0) |
| | | Monoetherglycerophosphocholine | |

In some embodiments of the methods, apparatus, or non-transitory computer-readable storage medium disclosed herein, the machine learning model comprises one or more machine learning models trained to generate an output corresponding to a treatment metric. In some embodiments, the treatment metric includes, among other things, percentage of liver fat, histology, clinical outcomes, fibrosis stage/status, MRI-PDFF, NASH resolution (e.g., absence of fatty liver disease or isolated or simple steatosis without steatohepatitis and a NAS score of 0-1 for inflammation, 0 for ballooning, and any value for steatosis), or the like. For instance, in some embodiments, when the treatment metric is percentage of liver fat (i.e., a measure for the amount of the liver that is occupied by fat), the one or more machine learning models are configured to generate, based on the features of the received biochemical data, a value representing a percentage of the liver that is occupied by fat.

In some embodiments of the methods, apparatus, or non-transitory computer-readable storage medium disclosed herein, the machine learning model can be a regression-based model or a classification-based model. In some embodiments, the output generated by the machine learning model is a continuous variable or a discrete variable. In some embodiments, the machine learning model is a decision tree, a linear classifier, a neural network, random forests, k-nearest neighbors, support vector machines, or the like. In some embodiments, the neural network is a multi-layer perceptron, a convolutional neural network, a recurrent neural network, or the like. In some embodiments, the convolutional neural network is LeNet, AlexNet, VGGNet 16, GoogleNet/Inception, ResNets, or the like.

In some embodiments of the methods, apparatus, or non-transitory computer-readable storage medium disclosed herein, the machine learning model can be trained on reference data corresponding to the revived biochemical data (e.g., the biochemical data received at step 210 of method 200) and the type of the treatment metric. In some embodiments, the reference data includes data characterizing a state of reference subjects at a reference condition and at, following administration of a treatment regimen, a treated condition. In some embodiments, the treatment regimen administered to each reference subject is the same as that being considered for the subject that a method disclosed herein (e.g., method 200) is being applied on behalf of. In this way, the output generated by the machine learning model is based on features of the current subject in view of features of reference subjects and responsiveness of a particular reference subject to a particular treatment regimen. This allows the output of the machine learning model to have predictive power.

In some embodiments of the methods, apparatus, or non-transitory computer-readable storage medium disclosed herein (e.g., in method 200 at step 215), the output generated (e.g., at step 210) is compared to a treatment threshold corresponding to the treatment metric. In some embodiments, if the treatment metric is a measure of fibrosis stage/status, the treatment threshold is an improvement in fibrosis greater than or equal to one stage. In some embodiments, if the treatment metric is percentage of liver fat, the treatment threshold is a reduction in liver fat content by greater than or equal to 30%. Whatever the treatment threshold may be, if the comparison of the generated output from the machine learning model to a treatment threshold (e.g., at step 215 of method 200) indicates the generated output of the machine learning model satisfies the treatment threshold, then the treatment regimen being considered should be administered to the patient (e.g., at step 220 of method 200).

In some embodiments of the methods, apparatus, or non-transitory computer-readable storage medium disclosed herein (e.g, method 200), the treatment regimen administered to the patient (e.g, at step 220 of method 200) depends on the condition of the patient. In some embodiments, when the patient has or is suspected of having NASH, the treatment regimen is treatment with an inhibitor of the de novo lipogenesis (DNL) pathway. In some embodiments, the inhibitor is any substance that slows, prevents, or reduces activity of a particular process or enzyme within the DNL pathway. Inhibitors of the DNL pathway include an ATP-citrate lyase (ACLY) inhibitor, an acetyl-CoA carboxylases 1 (ACC1) inhibitor, a fatty acid synthase (FASN) inhibitor, and/or a stearoyl-CoA desaturase-1 (SCD1) inhibitor. In some embodiments, the inhibitor is a small molecule ATP-citrate lyase (ACLY) inhibitor, an acetyl-CoA carboxylases 1 (ACC1) inhibitor, a fatty acid synthase (FASN) inhibitor, and/or a stearoyl-CoA desaturase-1 (SCD1) inhibitor. In some embodiments, the SCD1 inhibitor is a small molecule. In some embodiments, the FASN inhibitor is one of the compounds described herein. In some embodiments, the ACLY inhibitor is bempedoic acid, hydroxycitric acid tripotassium hydrate, (−)-hydroxycitric acid, 2-furoic acid, or (−)-hydroxycitric acid lactone. In some embodiments, ACC1 inhibitor is phenoxyisopropionic acid herbicide, graminicide, or quinolone or a quinolone derivative.

Referring second to method 250 of FIG. 2B, wherein the biochemical data is a subset of features of the biochemical data, the subset of features of the biochemical data characterizing a state of a subject can be received at step 255 of method 250. The subject may be an individual having or suspected of having NASH. In some embodiments, the biochemical data characterizing the state of the subject describes a ‘baseline’ condition, or reference condition. The reference condition may be one that reflects a medical condition of the subject before the initiation of a treatment regimen.

In some embodiments of the methods, apparatus, or non-transitory computer-readable storage medium disclosed herein, the biochemical data characterizing the state of the subject includes at least one bile acid, at least one secondary bile acid, at least one bile acid derivative, at least one amino acid, at least one amino acid derivative, and/or at least one glycerophospholipids.

In some embodiments of the methods, apparatus, or non-transitory computer-readable storage medium disclosed herein (e.g., method 250), the biochemical data characterizing the state of the subject include ursodeoxycholic and hyodeoxycholic acid (i.e., Variable ID AA35), DL-2-Aminocaprylic acid (i.e., Variable ID BA06_BA07), sarcosine (i.e., Variable ID AA22), glycoursodeoxycholic acid (i.e., Variable ID AA53), D(−)-2-Aminobutyric acid (i.e., Variable ID BA17H), and PC(0-18:0/22:4) (i.e., Variable ID MEMAPC30).

In some embodiments of the methods, apparatus, or non-transitory computer-readable storage medium disclosed herein (e.g., method 250, e.g., at step 260), the machine learning model applied to the biochemical data to generate an output is applied to the received biochemical data characterizing the state of the subject. In some embodiments, the trained machine learning model is one that is based on the set of features received as biochemical data (e.g., at step 255 of method 250).

In some embodiments of the methods, apparatus, or non-transitory computer-readable storage medium disclosed herein, the machine learning model is one or more machine learning models trained to generate an output corresponding to a treatment metric. In some embodiments, the treatment metric includes, among other things, percentage of liver fat, histology, clinical outcomes, fibrosis stage/status, MRI-PDFF, NASH resolution (e.g., absence of fatty liver disease or isolated or simple steatosis without steatohepatitis and a NAS score of 0-1 for inflammation, 0 for ballooning, and any value for steatosis), and the like. In some embodiments, when the treatment metric is percentage of liver fat (i.e., a measure for the amount of the liver that is occupied by fat), the one or more machine learning models are configured to generate, based on the features of the received biochemical data, a value representing a predicted percentage of the liver of the subject that is occupied by fat.

In some embodiments, the machine learning model is a regression-based model or a classification-based model. In some embodiments, the output generated by the machine learning model is a continuous variable or a discrete variable. In some embodiments, the machine learning model is a decision tree, a linear classifier, a neural network, random forests, k-nearest neighbors, support vector machines, and the like. The neural network may be a multi-layer perceptron, a convolutional neural network, a recurrent neural network, and the like. In some embodiments, the convolutional neural network may be LeNet, AlexNet, VGGNet 16, GoogleNet/Inception, ResNets, and the like.

In some embodiments, the machine learning model is trained on reference data corresponding to the biochemical data received from a subject having or suspected of having NASH (e.g., at step 260 of method 250) and the type of the treatment metric. In some embodiments, the reference data includes data characterizing a state of reference subjects at a reference condition and at, following administration of a treatment regimen, a treated condition. In some embodiments, the treatment regimen administered to each reference subject is the same as that being considered for the subject a method of the disclosure (e.g., method 200) is being applied on behalf of. In this way, in some embodiments, the output generated by the machine learning model is based on features of the current subject in view of features of reference subjects and responsiveness of a particular reference subject to a particular treatment regimen. Without wishing to be bound by theory, this allows the output of the machine learning model to have predictive power.

In some embodiments, (e.g., in method 250 of FIG. 2B), the machine learning model generates, via a random forest algorithm or a support vector machine algorithm, a continuous variable as an output. In some embodiments, the continuous variable is a predicted percentage of liver fat of the subject if the considered treatment regimen were administered. To this end, in some embodiments, the treatment regimen being considered for the subject is a FASN inhibitor regimen based on the FASN inhibitors described below.

In some embodiments of the methods, apparatus, or non-transitory computer-readable storage medium disclosed herein, (e.g., method 250 at step 265), the output generated by applying a machine learning model to the biochemical data (e.g., at step 260) is compared to a treatment threshold corresponding to the treatment metric. In some embodiments, where the treatment metric is percentage of liver fat, the treatment threshold is a reduction in liver fat content by greater than or equal to 30%. Thus, in some embodiments, if the comparison the output generated by applying a machine learning model to the biochemical data to a treatment threshold corresponding to the treatment metric (e.g., the comparison at step 265 of method 250) indicates that the generated output of the machine learning model satisfies the treatment threshold, then the treatment regimen being considered is administered to the patient (e.g., at step 270 of method 250).

In some embodiments of the methods, apparatus, or non-transitory computer-readable storage medium disclosed herein, the treatment regimen administered to the patient (e.g., at step 270 of method 250) is a FASN inhibitor (e.g., when the patient has or is suspected of having NASH). In some embodiments, the FASN inhibitor is a compound of Formula (I):

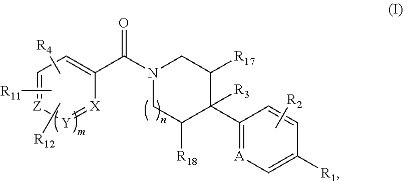

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; A is CH or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₅, R₆, R₇, R₈, R₉ R₁₀, R₁₃, and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, or alkylamino; R₁₇ and R₁₈ are each independently hydrogen or alkyl or can optionally join together to form a bond; n is 1 or 2; and m is 0 or 1.

In certain embodiments of Formula (I), R₃ is F.

In certain embodiments of Formula (I), A is CH.

In certain embodiments of Formula (I), A is N.

In certain embodiments of Formula (I), X, Y, and Z are NR′.

In certain embodiments of Formula (I), R₄ is heteroaryl, heterocyclyl, —C(═O)N(R₅R₆), —N(R₇)C(═O)R₈, —N(R₉R₁₀), C₁₋₆ alkyl, C₁₋₆ alkoxy, or R₄ and R₁₁ taken together with the atoms to which they are attached join together to form a heteroaryl.

In certain embodiments of Formula (I), R₅ is hydrogen and R₆ is aryl or heteroaryl.

In certain embodiments, the compounds of Formula (I) have one of the following Formulas (I-A) or (I-B):

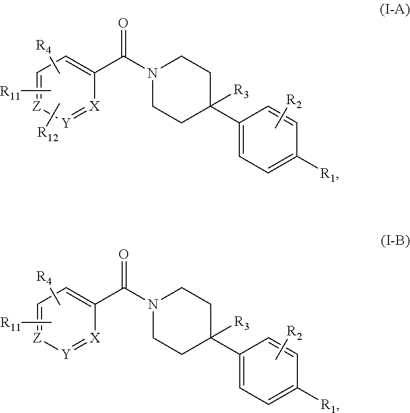

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, —S(═O)₂R₂₀, or R₁₁ and R₁₂ taken together with the atoms to which they are attached join together to form a heteroaryl; R₅, R₆, R₇, R₈, R₉ R₁₀, R₁₃, and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, or alkylamino; and R₁₇ and R₁₈ are each independently hydrogen or alkyl or can optionally join together to form a bond.

In certain embodiments, the compounds of Formula (I) have one of the following Formulas (I-C) or (I-D):

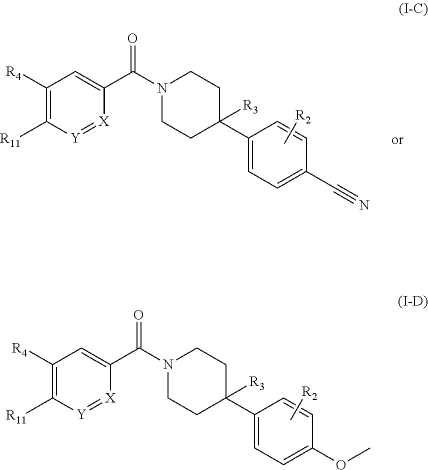

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, or C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —N(R₁₃)(R₁₄); R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the compounds of Formula (I) have one of the following Formulas (I-E), (I-F), (I-G), and (I-H):

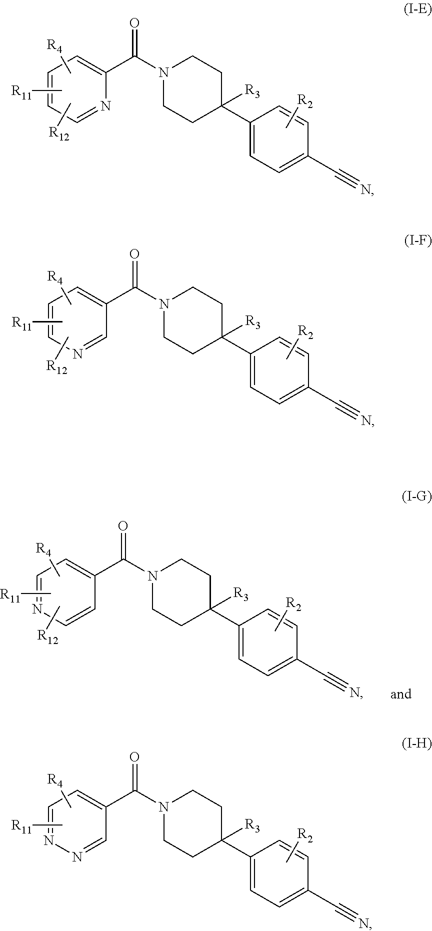

or a pharmaceutically acceptable salt thereof, wherein: R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, or C₁₋₆ alkoxy, or R₂ and R₃ taken together with the atoms to which they are attached form a 5-membered heterocyclyl; R⁴ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —N(R₁₃)(R₁₄); R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the compounds of Formula (I) have one of the following Formulas (I-I), (I-J), and (I-K):

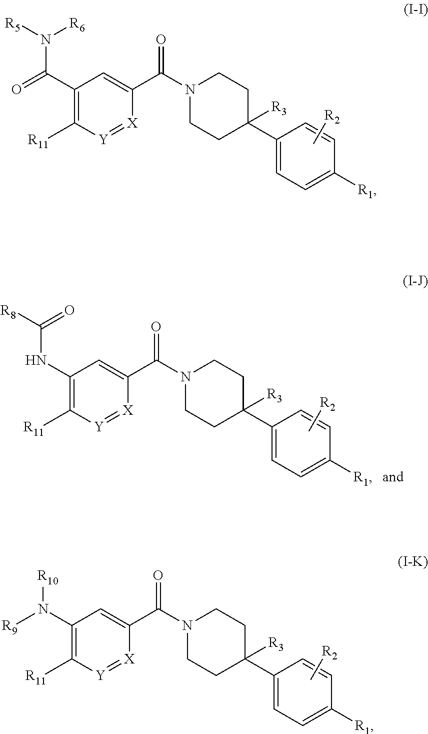

or a pharmaceutically acceptable salt thereof, wherein: X and Y are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₁ is hydrogen, halo, cyano, C₁₋₆ alkyl, C₁₋₆ alkoxy, —N(R₁₃R₁₄), CF₃, —OCF₃, or —S(═O)₂R₂₀; R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the compounds of Formula (I) have the following Formula (I-L) or (I-M):

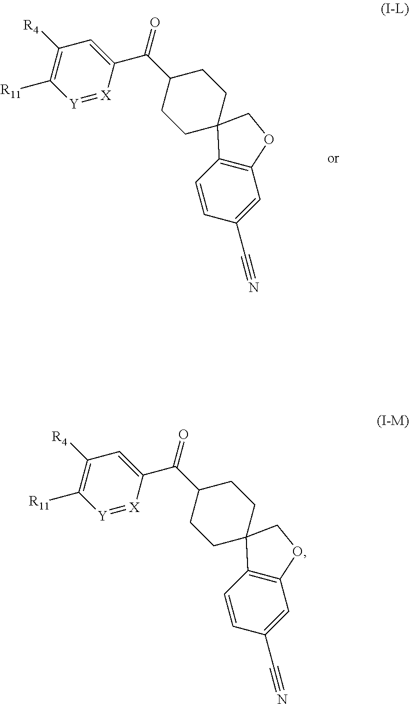

or a pharmaceutically acceptable salt thereof, wherein: X and Y are each independently CR or NR′, wherein R is H or C₁₋₆ alkyl and R′ is H, C₁₋₆ alkyl, or absent; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —N(R₁₃)(R₁₄); R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is Compound 001-346 or 001-495 of Table C-1, or a pharmaceutically acceptable salt thereof.

In certain embodiments, the compounds of Formula (I) have the following Formula (I-P):

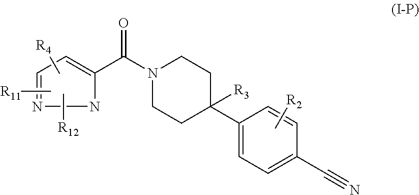

or a pharmaceutically acceptable salt thereof, wherein: R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, or C₁₋₆ alkoxy, or R₂ and R₃ taken together with the atoms to which they are attached form a 5-membered heterocyclyl; R⁴ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —N(R₁₃)(R₁₄); R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, —S(═O)₂R₂₀, or R₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is Compound 001-385, 001-387, or 001-496, of Table C-1, or a pharmaceutically acceptable salt thereof.

In certain embodiments, the compounds of Formula (I) have the following Formula (I-T):

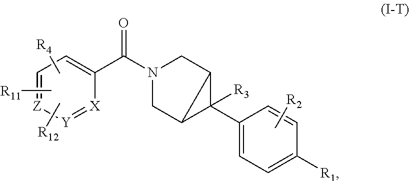

or a pharmaceutically acceptable salt thereof, wherein: X, Y, and Z are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is Compound 001-118 of Table C-1, or a pharmaceutically acceptable salt thereof.

In certain embodiments, the compounds of Formula (I) have the following Formula (I-V):

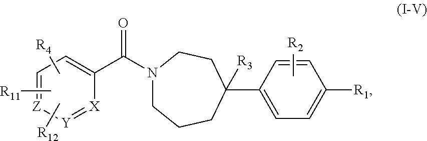

or a pharmaceutically acceptable salt thereof, wherein: X, Y, and Z are each independently CR or NR′, wherein R is H or C₁₋₆ alkyl and R′ is H, C₁₋₆ alkyl, or absent; R₁ is hydrogen, cyano, halo, C₁₋₆ alkyl, C₁₋₆ alkoxy, —C(═O)N(R¹³)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, —S(═O)₂R₂₀, or R₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is Compound 001-40 of Table C-1, or a pharmaceutically acceptable salt thereof.

In certain embodiments, the fatty acid synthase inhibitor is a compound selected from Compounds 001-500, 001-27, 001-73, 001-348, 001-349, 001-123, 001-497, 001-383, 001-280, 001-121, and 001-289 of Table C-1, or a pharmaceutically acceptable salt thereof.

In some embodiments, the fatty acid synthase inhibitor may be a compound of Formula (II):

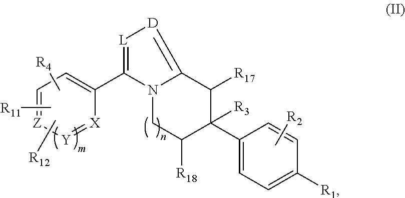

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; L and D are each independently C or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₅, R₆, R₇, R₈, R₉ R₁₀, R₁₃, and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, or alkylamino; R₁₇ and R₁₈ are each independently hydrogen or alkyl or can optionally join together to form a bond; n is 1 or 2; and m is 0 or 1.

In certain embodiments, the compounds of Formula (II) have the following Formula (II-A):

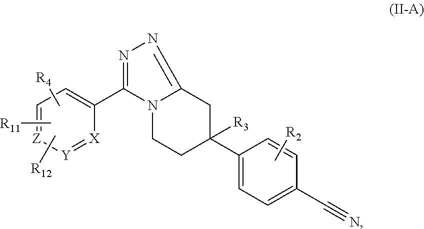

or a pharmaceutically acceptable salt thereof, wherein: X, Y, and Z are each independently CR or NR′, wherein R is H or C₁₋₆ alkyl and R′ is H, C₁₋₆ alkyl, or absent; R₂ is hydrogen, halo, C₁₋₆ alkoxy, C₁₋₆ alkyl, or R₂ and R₃ taken together with the atoms to which they are attached form a 5-membered heterocyclyl; R₃ is hydrogen, hydroxyl, halo, C₁₋₆ alkyl, C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁ taken together with the atoms to which they are attached join together to form a heteroaryl; R₂₀ is hydrogen or C₁₋₆ alkyl, C₁₋₆ alkoxy, or —N(R₁₃)(R₁₄); R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₅, R₆, R₇, R₈, R₉, R₁₀, R₁₃, and R₁₄ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the compounds of Formula (II) have the following Formula (II-B):

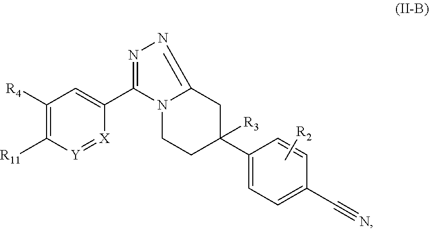

or a pharmaceutically acceptable salt thereof, wherein: X and Y are each independently CR or NR′, wherein R is H or C₁₋₆ alkyl and R′ is H, C₁₋₆ alkyl, or absent; R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, or C₁₋₆ alkoxy, or R₂ and R₃ taken together with the atoms to which they are attached form a 5-membered heterocyclyl; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₂₀ is hydrogen or C₁₋₆ alkyl, C₁₋₆ alkoxy, or —N(R₁₃)(R₁₄); R₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the compounds of Formula (II) have one of the following Formulas (II-C), (II-D), and (II-E):

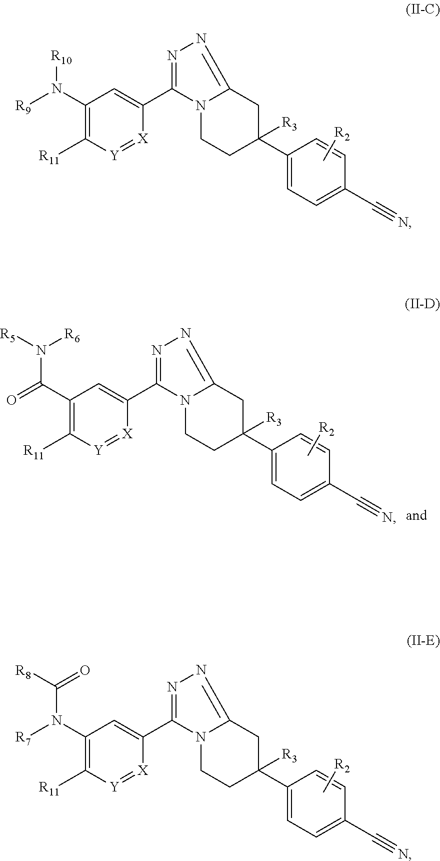

or a pharmaceutically acceptable salt thereof, wherein: X and Y are each independently CR or NR′, wherein R is H or C₁₋₆ alkyl and R′ is H, C₁₋₆ alkyl, or absent; R₂ is hydrogen, halo, C₁₋₆ alkoxy, or C₁₋₆ alkyl, or R₂ and R₃ taken together with the atoms to which they are attached form a 5-membered heterocyclyl; R₃ is hydrogen, hydroxyl, halo, C₁₋₆ alkyl, or C₁₋₆ alkoxy, or R₂ and R₃ taken together with the atoms to which they are attached form a 5-membered heterocyclyl; R₁₁ is hydrogen, halo, cyano, C₁₋₆ alkyl, C₁₋₆ alkoxy, —N(R₁₃R₁₄), CF₃, —OCF₃, or —S(═O)₂R₂₀; R₂₀ is hydrogen or C₁₋₆ alkyl, C₁₋₆ alkoxy, or —N(R₁₃)(R₁₄); R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is Compound 001-347 of Table C-1, or a pharmaceutically acceptable salt thereof.

In some embodiments, the fatty acid synthase inhibitor may be a compound of Formula (III):

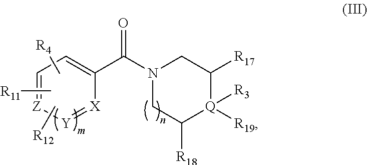

or a pharmaceutically acceptable salt thereof, wherein: X, Y, and Z are each independently CR or NR′, wherein R is hydrogen or C₁₋₆ alkyl and R′ is hydrogen, C₁₋₆ alkyl, or absent; Q is C or N; R₃ is hydrogen, hydroxyl, halo, C₁₋₆ alkyl, C₁₋₆ alkoxy, or if Q is N then R₃ is absent; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, —S(═O)₂R₂₀, or R₄ and R₁₁ taken together with the atoms to which they are attached join together to form a heteroaryl, or R₁₁ and R₁₂ taken together with the atoms to which they are attached join together to form a heteroaryl; R₁₂ is hydrogen, halo, cyano, C₁₋₆ alkyl, C₁₋₆ alkoxy, —N(R₁₃R₁₄), CF₃, —OCF₃, or —S(═O)₂R₂₀, or R₁₁ and R₁₂ taken together with the atoms to which they are attached join together to form a heteroaryl; R₂₀ is hydrogen or C₁₋₆ alkyl, C₁₋₆ alkoxy, or —N(R₁₃)(R₁₄); R₅, R₆, R₇, R₈, R₉ R₁₀, R₁₃, and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, or alkylamino; R₁₇ and R₁₈ are each independently hydrogen or alkyl or can optionally join together to form a bond; R₁₉ is aryl, heteroaryl, cycloalkyl, or heterocyclyl; n is 0, 1, or 2; and m is 0 or 1.

In certain embodiments, the compounds of Formula (III) have one of the following Formulas (III-A), (III-B), and (III-C):

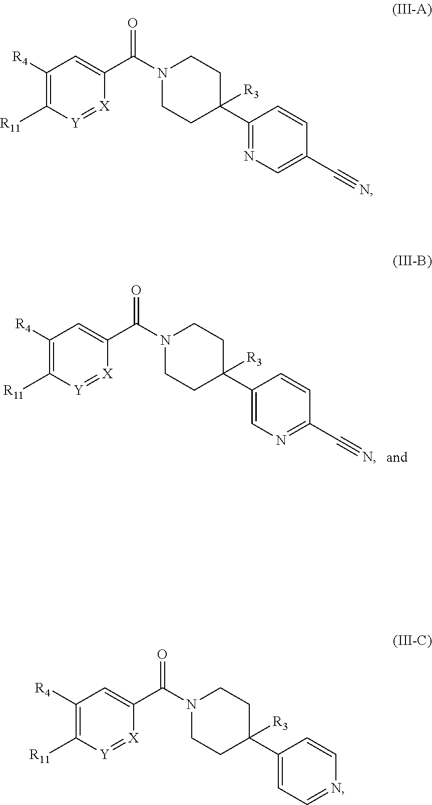

or a pharmaceutically acceptable salt thereof, wherein: X and Y are each independently CR or NR′, wherein R is H or C₁₋₆ alkyl and R′ is H, C₁₋₆ alkyl, or absent; R₃ is hydrogen, hydroxyl, halo, C₁₋₆ alkyl, or C₁₋₆ alkoxy; R₄ is hydrogen, heteroaryl, heterocyclyl, —C(═O)N(R₅R₆), —N(R₇)C(═O)R₈, —N(R₉R₁₀), C₁₋₆ alkyl, C₁₋₆ alkoxy, or —S(═O)₂R₂₀, or R₄ and R₁₁ taken together with the atoms to which they are attached join together to form a heteroaryl; R₁₁ is hydrogen, halo, cyano, C₁₋₆ alkyl, C₁₋₆ alkoxy, —N(R₁₃R₁₄), CF₃, —OCF₃, or —S(═O)₂R₂₀, or R₄ and R₁₁ taken together with the atoms to which they are attached join together to form a heteroaryl; R₅, R₆, R₇, R₈, R₉, and R₁₀ are each independently H, C₁₋₆ alkyl, cycloalkyl, aryl, heterocyclyl, heteroaryl, alkylamino, or —N(R₁₅R₁₆); and R₁₅ and R₁₆ are each independently H, C₁₋₆ alkyl, cycloalkyl, aryl, heterocyclyl, heteroaryl, or alkylamino.

In certain embodiments, the fatty acid synthase inhibitor is a compound selected from Compounds 001-50, 001-51, and 001-326 of Table C-1, or a pharmaceutically acceptable salt thereof.

In some embodiments, the fatty acid synthase inhibitor may be a compound of Formula (IV-A), (IV-B), or (IV-C):

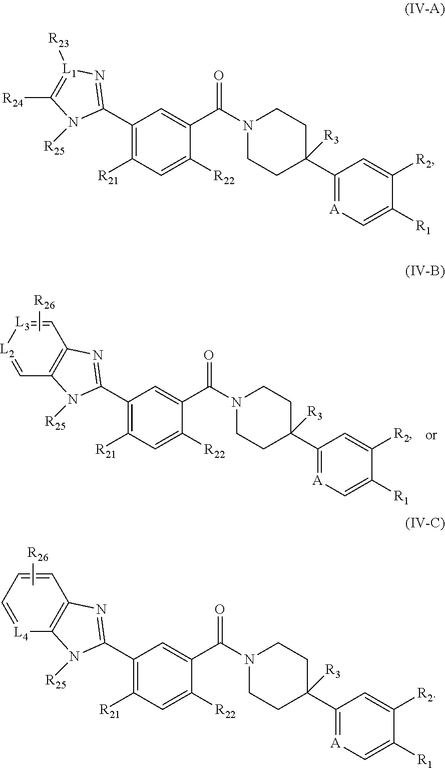

or a pharmaceutically acceptable salt thereof, wherein: L₁, L₂, L₃, L₄, and A are each independently CH or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₂₃ is hydrogen, —N(R₁₃)(R₁₄), C₁₋₆ alkyl, C₁₋₆ alkoxy, is absent if Li is N, or R₂₃ and R₂₄ taken together with the atoms to which they are attached join together to form a heterocyclyl, heteroaryl, or cycloalkyl; R₂₄ is hydrogen, —N(R₁₃)(R₁₄), C₁₋₆ alkyl, C₁₋₆ alkoxy, —(C₁₋₆ alkoxy)(heterocyclyl), heterocyclyl, or R₂₃ and R₂₄ taken together with the atoms to which they are attached join together to form a heterocyclyl, heteroaryl, or cycloalkyl; R₂₆ is hydrogen, heteroaryl, heterocyclyl, —N(R₁₃)(R₁₄), or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₂₅ is hydrogen, C₁₋₆ alkyl, or C₁₋₆ alkoxy; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In certain embodiments, the compounds of Formula (IV) have one of the following Formulas (IV-D) and (IV-E):

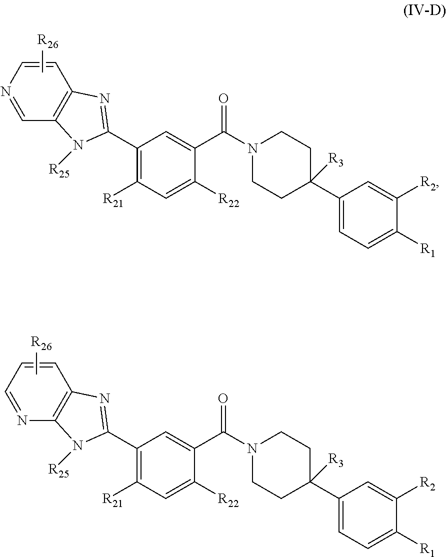

(IV-E), or a pharmaceutically acceptable salt thereof wherein: R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₂₆ is hydrogen, heteroaryl, heterocyclyl, —N(R₁₃)(R₁₄), or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₂₅ is hydrogen, C₁₋₆ alkyl, or C₁₋₆ alkoxy; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In certain embodiments, the compounds of Formula (IV) have one of the following Formulas (IV-F) and (IV-G):

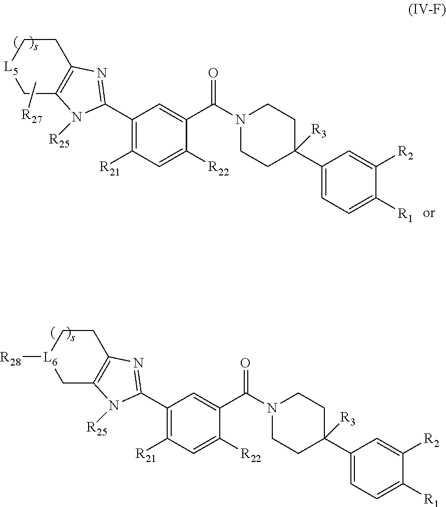

(IV-G), or a pharmaceutically acceptable salt thereof, wherein: R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₂₅ is hydrogen, C₁₋₆ alkyl, or C₁₋₆ alkoxy; R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino; s is 0, 1, or 2; L₅ is CH₂, NH, S, or O; L₆ is CH or N; R²⁷ is hydrogen, —C(═O)R″, —S(═O)₂R₂₀; R₂₈ is hydrogen, —C(═O)R″, —S(═O)₂R₂₀, or is absent if L₆ is O; and R″ is hydrogen, C₁₋₆ alkyl, C₁₋₆ alkoxy, —C(═O)N(R₁₃)(R₁₄), or —N(R₁₃)(R₁₄).

In certain embodiments of Formula (IV), R₁ is hydrogen, cyano, C₁₋₆ alkyl, C₁₋₆ alkoxy, or —C(═O)N(R₁₃)(R₁₄).

In certain embodiments of Formula (IV), R₁ is cyano.

In certain embodiments of Formula (IV), R₂ is hydrogen or halo; R₂ is hydrogen.

In certain embodiments of Formula (IV), R₃ is hydrogen.

In certain embodiments of Formula (IV), R₂₁ and R₂₂ are each independently hydrogen or C₁₋₆ alkyl.

In certain embodiments of Formula (IV), R₂₁ and R₂₂ are each independently C₁₋₆ alkyl.

In certain embodiments of Formula (IV), R₂₅ is hydrogen.

In certain embodiments of Formula (IV), L₂ is N.

In certain embodiments of Formula (IV), Li is CH.

In certain embodiments of Formula (IV), L₃ is CH.

In certain embodiments of Formula (IV), L₄ is CH.

In certain embodiments of Formula (IV), A is N.

In certain embodiments of Formula (IV), A is CH.

In certain embodiments of Formula (IV), R₂₆ is heterocyclyl.

In certain embodiments of Formula (IV), R₂₄ is —N(R₁₃)(R₁₄).

In certain embodiments of Formula (IV), L₅ and L₆ are each independently N. In certain embodiments of Formula (IV), s is 1.

In certain embodiments of Formula (IV), s is 0.

In certain embodiments, the fatty acid synthase inhibitor is a compound selected from Compounds 001-1, 001-3, 001-4, 001-14, 001-20, 001-27, 001-31, and 001-36 of Table C-1, or a pharmaceutically acceptable salt thereof.

In some embodiments, the fatty acid synthase inhibitor may be a compound of Formula (V):

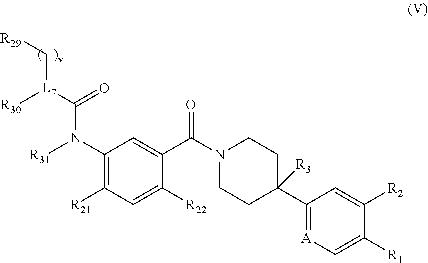

or a pharmaceutically acceptable salt thereof, wherein: L₇ is N or O, wherein R₃₀ is absent if L₇ is O; A is CH or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₂₉ and R₃₀ are each independently hydrogen, C₁₋₆ alkyl, C₁₋₆ alkoxy, hydroxyalkyl, heteroaryl, heterocyclyl, —N(R₁₅R₁₆), —C(═O)R₄₆, OR—R₄₈C(═O)R₄₇, or R₂₉ and R₃₀ taken together with the atoms to which they are attached join together to form a heteroaryl or heterocyclyl, wherein R₃₀ is absent if L₇ is O; R₄₆ and R₄₇ are each independently hydrogen, C₁₋₆ alkyl, cycloalkyl, aryl, heterocyclyl, heteroaryl, hydroxyalkyl, —N(R₁₅R₁₆), or —S(═O)₂R₂₀; R₄₈ is alkyl or is absent; R₃₁ is hydrogen, C₁₋₆ alkyl, or C₁₋₆ alkoxy; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, hydroxyalkyl, or alkylamino; and v is 0 or 1.

In certain embodiments, the compounds of Formula (V) have one of the following Formulas (V-A), (V-B), (V-C), and (V-D):

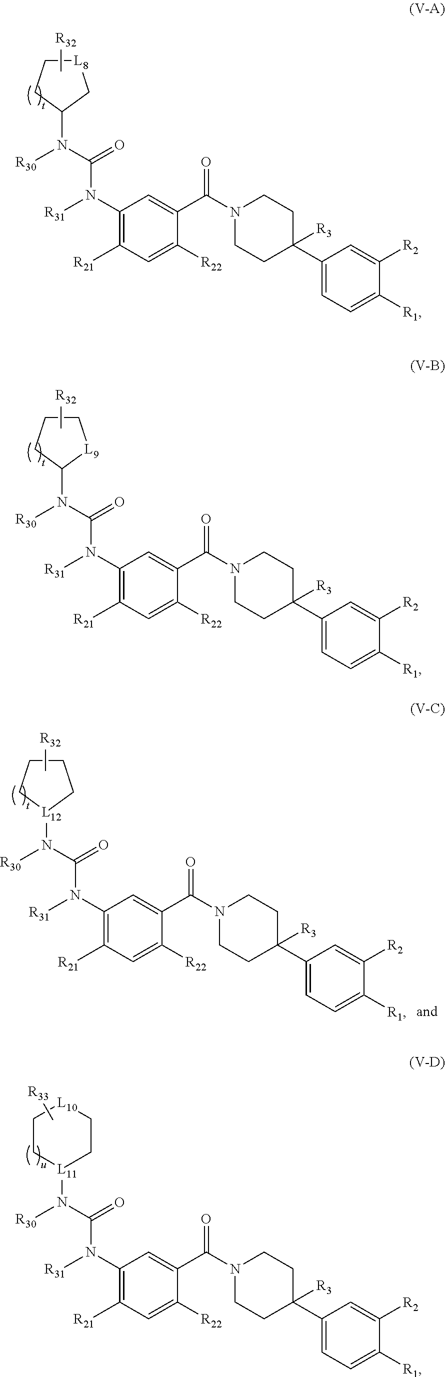

or a pharmaceutically acceptable salt thereof, wherein: R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₀ is hydrogen, C₁₋₆ alkyl, C₁₋₆ alkoxy, hydroxyalkyl, heteroaryl, heterocyclyl, —N(R₁₅R₁₆), —C(═O)R₄₆, or —R₄₈C(═O)R₄₇, wherein R₃₀ is absent if L₇ is O; R₄₆ and R₄₇ are each independently hydrogen, C₁₋₆ alkyl, cycloalkyl, aryl, heterocyclyl, heteroaryl, hydroxyalkyl, —N(R₁₅R₁₆), or —S(═O)₂R₂₀; R₄₈ is alkyl or is absent; R₃₁ is hydrogen, C₁₋₆ alkyl, or C₁₋₆ alkoxy; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, cycloalkyl, aryl, heterocyclyl, heteroaryl, hydroxyalkyl, or alkylamino; Ls, L₉, and L₁₀ are each independently CH₂, NH, or O; L₁₁ and L₁₂ are each independently CH or N; R₃₂ and R₃₃ are each independently hydrogen, C₁₋₆ alkyl, C₁₋₆ alkoxy, —S(═O)₂R₂₀, —C(═O)R₄₆, hydroxyalkyl, hydroxyl, or are absent; u is 0, 1, or 2; and t is 0, 1, or 2.

In certain embodiments of Formula (V), L₇ is N.

In certain embodiments of Formula (V), L₇ is O.

In certain embodiments of Formula (V), A is N.

In certain embodiments of Formula (V), A is CH.

In certain embodiments of Formula (V), R₁ is hydrogen, cyano, C₁₋₆ alkyl, C₁₋₆ alkoxy, or —C(═O)N(R₁₃)(R₁₄).

In certain embodiments of Formula (V), R₁ is cyano.

In certain embodiments of Formula (V), R₂ is hydrogen or halo.

In certain embodiments of Formula (V), R₂ is hydrogen.

In certain embodiments of Formula (V), R₃ is fluorine.

In certain embodiments of Formula (V), R₂₁ and R₂₂ are each independently hydrogen or C₁₋₆ alkyl.

In certain embodiments of Formula (V), R₂₁ and R₂₂ are each independently C₁₋₆ alkyl.

In certain embodiments of Formula (V), R₃₁ is hydrogen.

In certain embodiments of Formula (V), R₃₀ is hydrogen.

In certain embodiments of Formula (V), Ls is O.

In certain embodiments of Formula (V), L₉ is O.

In certain embodiments of Formula (V), L₁₀ is O and L₁₁ is N.

In certain embodiments of Formula (V), L₁₂ is N.

In certain embodiments of Formula (V), R₃₂ and R₃₃ are each independently hydrogen.

In certain embodiments, the fatty acid synthase inhibitor is a compound selected from Compounds 001-64, 001-65, 001-70, 001-78, 001-498, 001-336, and 001-338 of Table C-1, or a pharmaceutically acceptable salt thereof.

In some embodiments, the fatty acid synthase inhibitor may be a compound of Formula (VI-A) or (VI-B):

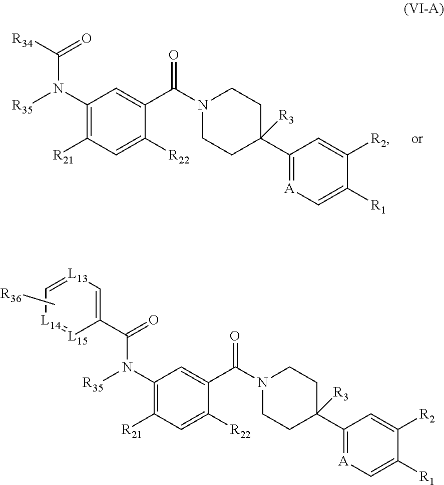

(VI-B), or a pharmaceutically acceptable salt thereof, wherein: L₁₃, L₁₄, L₁₅, and A are each independently CH or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₄ is hydrogen, C₁₋₆ alkyl, C₁₋₆ alkoxy, cycloalkyl, hydroxyl, hydroxyalkyl, aryl, heterocyclyl, heteroaryl, alkylamino, CF₃, —OCF₃, —S(═O)₂R₂₀, or —N(R₁₅R₁₆); R₃₅ is hydrogen, C₁₋₆ alkyl, or C₁₋₆ alkoxy; R₃₆ is hydrogen, C₁₋₆ alkyl, C₁₋₆ alkoxy, —N(R₁₅R₁₆), heterocyclyl, or heteroaryl; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; and R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino.

In certain embodiments, the FASN inhibitor may be compounds of Formula (VI) having one of the following Formulas (VI-C) or (VI-D):

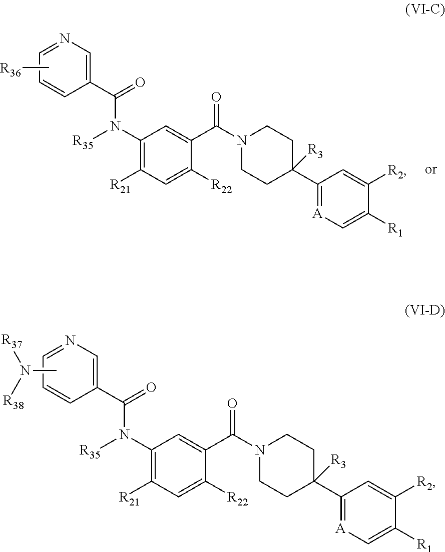

or a pharmaceutically acceptable salt thereof, wherein: R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₅ is hydrogen, C₁₋₆ alkyl, or C₁₋₆ alkoxy; R₃₆ is hydrogen, C₁₋₆ alkyl, C₁₋₆ alkoxy, —N(R₁₅R₁₆), heterocyclyl, or heteroaryl; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; R₁₅ and R₁₆ are each independently hydrogen, C₁₋₆ alkyl, C₁₋₆ alkoxy, cycloalkyl, aryl, heterocyclyl, heteroaryl, hydroxyalkyl, or alkylamino; and R₃₇ and R₃₈ are each independently hydrogen, C₁₋₆ alkyl, C₁₋₆ alkoxy, hydroxyalkyl, heteroaryl, heterocyclyl, or R₃₇ and R₃₈ taken together with the atoms to which they are attached join together to form a heteroaryl or heterocyclyl.

In certain embodiments of Formula (VI), R₁ is hydrogen, cyano, C₁₋₆ alkyl, C₁₋₆ alkoxy, or —C(═O)N(R₁₃)(R₁₄).

In certain embodiments of Formula (VI), R₁ is cyano.

In certain embodiments of Formula (VI), R₂ is hydrogen or halo.

In certain embodiments of Formula (VI), R₂ is hydrogen.

In certain embodiments of Formula (VI), R₃ is fluorine.

In certain embodiments of Formula (VI), R₂₁ and R₂₂ are each independently hydrogen or C₁₋₆ alkyl.

In certain embodiments of Formula (VI), R₂₁ and R₂₂ are each independently C₁₋₆ alkyl.

In certain embodiments of Formula (VI), R₃₅ is hydrogen.

In certain embodiments of Formula (VI), R₃₄ is heteroaryl;

In certain embodiments of Formula (VI), R₃₄ is thienyl, pyrryl, furyl, pyridyl, pyrimidyl, pyrazinyl, pyrazolyl, oxazolyl, isoxazolyl, imidazolyl, thiazolyl, pyranyl, tetrazolyl, pyrrolyl, pyrrolinyl, pyridazinyl, triazolyl, indolyl, isoindolyl, indolizinyl, benzimidazolyl, quinolyl, isoquinolyl, indazolyl, benzotriazolyl, tetrazolopyridazinyl, oxadiazolyl, benzoxazolyl, benzoxadiazolyl, thiadiazolyl, benzothiazolyl, or benzothiadiazolyl.

In certain embodiments of Formula (VI), L₁₃ is N.

In certain embodiments of Formula (VI), L₁₄ and L₁₅ are each independently CH.

In certain embodiments of Formula (VI), A is N.

In certain embodiments of Formula (VI), A is CH.

In certain embodiments, the fatty acid synthase inhibitor is a compound selected from Compounds 001-42, 001-43, 001-48, 001-62, and 001-322 of Table C-1, or a pharmaceutically acceptable salt thereof.

In some embodiments, the fatty acid synthase inhibitor may be a compound of Formula (VI-J):

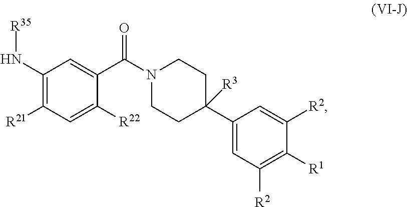

or a pharmaceutically acceptable salt thereof, wherein: R¹ is H, —CN, halogen, C₁-C₄ straight or branched alkyl, —O—(C₃-C₅ cycloalkyl), or —O—(C₁-C₄ straight or branched alkyl) wherein: the C₃-C₅ cycloalkyl optionally includes an oxygen or nitrogen heteroatom; and when R¹ is not H, —CN or halogen, it is optionally substituted with one or more halogens; each R² is independently H, halogen or C₁-C₄ straight or branched alkyl; R³ is H, —OH, or halogen; R²¹ is cyclobutyl, azetidin-1-yl, or cyclopropyl; R²² is H, halogen, or C₁-C₂ alkyl; R³⁵ is —C(O)—R³⁵¹, —C(O)—NHR³⁵¹, —C(O)—O—R³⁵¹ or S(O)₂R³⁵¹; and R³⁵¹ is C₁-C₆ straight or branched alkyl, cycloalkyl, heterocyclyl, aryl or heteroaryl.

In some embodiments of the compound of Formula (VI-J), R³ is H or halogen.

In some embodiments of the compound of Formula (VI-J), R¹ is halogen, —CN or C₁-C₂ haloalkyl.

In some embodiments of the compound of Formula (VI-J), R²² is C₁-C₂ alkyl.

In some embodiments of the compound of Formula (VI-J), R²¹ is cyclobutyl and R²² is C₁-C₂ alkyl.

In some embodiments of the compound of Formula (VI-J), R²¹ is cyclobutyl.

In some embodiments of the compound of Formula (VI-J), R³ is H or F.

In some embodiments of the compound of Formula (VI-J), R¹ is —CN.

In some embodiments of the compound of Formula (VI-J), R¹ is —CF₃.

In some embodiments of the compound of Formula (VI-J), R²² is H, methyl or ethyl.

In some embodiments of the compound of Formula (VI-J), R²² is H.

In some embodiments of the compound of Formula (VI-J), R²² is methyl.

In some embodiments of the compound of Formula (VI-J), R³⁵ is —C(O)—NHR³⁵¹.

In some embodiments of the compound of Formula (VI-J), R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl or (S)-tetrahydro-2H-pyran-3-yl.

In some embodiments of the compound of Formula (VI-J), R³⁵¹ is (R)-(tetrahydrofuran-2-yl)methyl or (S)-(tetrahydrofuran-2-yl)methyl.

In some embodiments of the compound of Formula (VI-J), R¹ is —CN, each R² is hydrogen, R³ is H or F, R²¹ is C₃-C₄ cycloalkyl, R²² is H, R³⁵ is —C(O)—NHR³⁵¹ where R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl.

In some embodiments of the compound of Formula (VI-J), R³⁵ is —C(O)—O—R³⁵¹.

In some embodiments of the compound of Formula (VI-J), R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl.

In some embodiments of the compound of Formula (VI-J), R¹ is —CN, each R² is H, R³ is H or F, R²¹ is C₃-C₄ cycloalkyl, R²² is H, R³⁵ is —C(O)—O—R³⁵¹ where R³⁵¹ is isopropyl, isobutyl, (R)-3-tetrahydrofuranyl, (S)-3-tetrahydrofuranyl, (R)-(tetrahydrofuran-2-yl)methyl, (S)-(tetrahydrofuran-2-yl)methyl, (R)-tetrahydro-2H-pyran-3-yl, or (S)-tetrahydro-2H-pyran-3-yl.

In some embodiments of the compound of Formula (VI-J), R³⁵¹ is (R)-3-tetrahydrofuranyl or (S)-3-tetrahydrofuranyl.

In certain embodiments, the fatty acid synthase inhibitor is a compound selected from Compounds 001-443, 001-444, 001-490, 001-491, 001-492, and 001-493 of Table C-1, or a pharmaceutically acceptable salt thereof.

In some embodiments, the fatty acid synthase inhibitor may be a compound of Formulas (VII-A) or (VII-B):

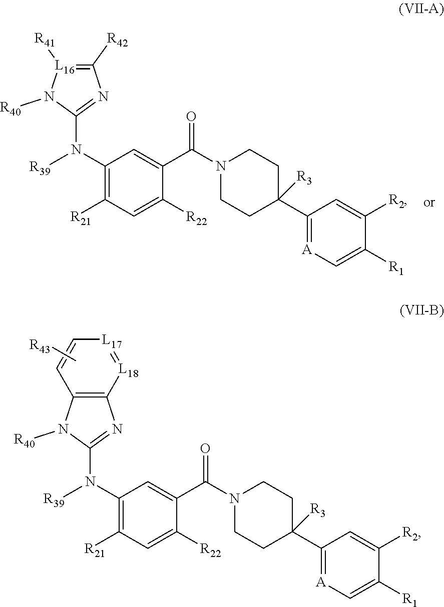

or a pharmaceutically acceptable salt thereof, wherein: L₁₆ is C or N, wherein R₄₁ is absent if L₁₆ is N; L₁₇, Lis, and A are each independently CH or N; R₁ is hydrogen, cyano, halo, —C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₄₀, R₄₂, and R₄₃ are each independently hydrogen, C₁₋₆ alkyl, C₁₋₆ alkoxy, —S(═O)₂R₂₀, —C(═O)R, hydroxyalkyl, hydroxyl, or —N(R₁₃R₁₄), or R₄₁ and R₄₂ taken together with the atoms to which they are attached join together to form a heteroaryl or heterocyclyl; R₄₁ is hydrogen, C₁₋₆ alkyl, C₁₋₆ alkoxy, —S(═O)₂R₂₀, —C(═O)R, hydroxyalkyl, hydroxyl, or —N(R₁₃R₁₄), or R₄₁ is absent if L₁₆ is N, or R₄₁ and R₄₂ taken together with the atoms to which they are attached join together to form a heteroaryl or heterocyclyl; R is hydrogen, C₁₋₆ alkyl, cycloalkyl, aryl, heterocyclyl, heteroaryl, hydroxyalkyl, —N(R₁₅R₁₆), or —S(═O)₂R₂₀; R₃₉ is hydrogen, C₁₋₆ alkyl, or C₁₋₆ alkoxy; R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; and R₁₅ and R₁₆ are each independently hydrogen, C₁₋₆ alkyl, cycloalkyl, aryl, heterocyclyl, heteroaryl, hydroxyalkyl, or alkylamino.

In certain embodiments of Formula (VII), R₁ is hydrogen, cyano, C₁₋₆ alkyl, C₁₋₆ alkoxy, or —C(═O)N(R₁₃)(R₁₄).

In certain embodiments of Formula (VII), R₁ is cyano.

In certain embodiments of Formula (VII), R₂ is hydrogen or halo.

In certain embodiments of Formula (VII), R₂ is hydrogen.

In certain embodiments of Formula (VII), R₃ is hydrogen.

In certain embodiments of Formula (VII), R₂₁ and R₂₂ are each independently hydrogen or C₁₋₆ alkyl.

In certain embodiments of Formula (VII), R₂₁ and R₂₂ are each independently C₁₋₆ alkyl.

In certain embodiments of Formula (VII), R₃₉ is hydrogen.

In certain embodiments of Formula (VII), R₄₀ is hydrogen.

In certain embodiments of Formula (VII), L₁₆ is N.

In certain embodiments of Formula (VII), L₁₇ is N.

In certain embodiments of Formula (VII), L₁₈ is CH.

In certain embodiments of Formula (VII), L₁₈ is N.

In certain embodiments of Formula (VII), A is N.

In certain embodiments of Formula (VII), A is CH.

In certain embodiments of Formula (VII), R₄₂ is C₁₋₆ alkyl.

In certain embodiments of Formula (VII), R₄₁ is C₁₋₆ alkyl.

In certain embodiments, the fatty acid synthase inhibitor is Compound 001-161 or 001-499 of Table C-1, or a pharmaceutically acceptable salt thereof.

In some embodiments, the fatty acid synthase inhibitor may be a compound of one of Formulae (VIII-A), (VIII-B), and (VIII-C):

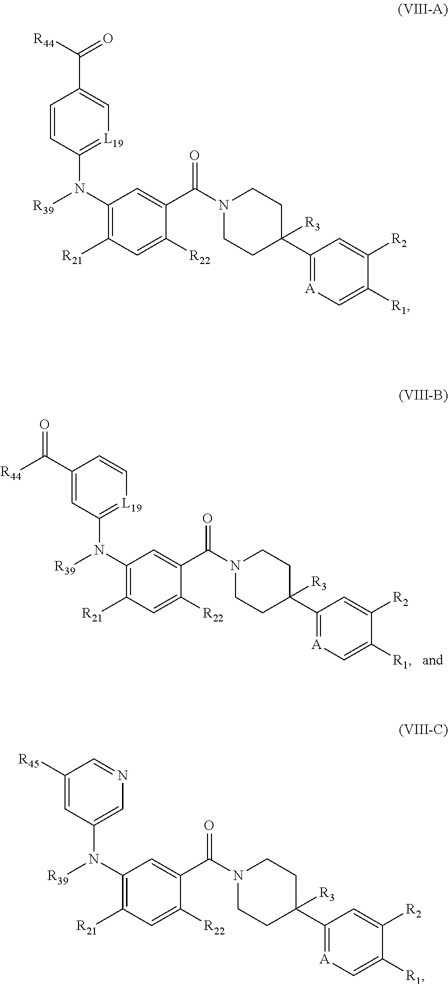

or a pharmaceutically acceptable salt thereof, wherein: L₁₉ and A are each independently CH or N; R₁ is hydrogen, cyano, halo, C₁₋₆ alkyl, C₁₋₆ alkoxy, —C(═O)N(R₁₃)(R₁₄), —(CH₂)_{q}C(═O)N(R₁₃)(R₁₄), CF₃, —OCF₃, or —S(═O)₂R₂₀; q is 0, 1, 2, 3, or 4; R₂₀ is hydrogen or C₁₋₆ alkyl, C₁₋₆ alkoxy, or —N(R₁₃)(R₁₄); R₂ is hydrogen, halo, C₁₋₆ alkoxy, or C₁₋₆ alkyl; R₃ is hydrogen, hydroxyl, halo, C₁₋₆ alkyl, or C₁₋₆ alkoxy; R₂₁ and R₂₂ are each independently hydrogen, halo, cyano, C₁₋₆ alkyl, C₁₋₆ alkoxy, CF₃, —OCF₃, or —S(═O)₂R₂₀; R₃₉ is hydrogen, C₁₋₆ alkyl, or C₁₋₆ alkoxy; R₄₄ and R₄₅ are each independently hydrogen, C₁₋₆ alkyl, C₁₋₆ alkoxy, cycloalkyl, hydroxyalkyl, aryl, heterocyclyl, heteroaryl, alkylamino, —S(═O)₂R₂₀, —C(═O)R, or —N(R₁₃R₁₄); and R₁₃ and R₁₄ are each independently hydrogen, C₁₋₆ alkyl, cycloalkyl, aryl, heterocyclyl, heteroaryl, hydroxyalkyl, alkylamino, —N(R₁₅R₁₆), or —S(═O)₂R₂₀; and R₁₅ and R₁₆ are each independently hydrogen, C₁₋₆ alkyl, cycloalkyl, aryl, heterocyclyl, heteroaryl, hydroxyalkyl, or alkylamino.

In certain embodiments of Formula (VIII), R₁ is cyano.

In certain embodiments of Formula (VIII), R₂ is hydrogen or halo.

In certain embodiments of Formula (VIII), R₂ is hydrogen.

In certain embodiments of Formula (VIII), R₃ is hydrogen.

In certain embodiments of Formula (VIII), R₂₁ and R₂₂ are each independently hydrogen or C₁₋₆ alkyl.

In certain embodiments of Formula (VIII), R₂₁ and R₂₂ are each independently C₁₋₆ alkyl.

In certain embodiments of Formula (VIII), R₃₉ is hydrogen.

In certain embodiments of Formula (VIII), L₁₉ is N.

In certain embodiments of Formula (VIII), A is N.

In certain embodiments of Formula (VIII), A is CH.

In certain embodiments, the fatty acid synthase inhibitor is Compound 001-498 of Table C-1, or a pharmaceutically acceptable salt thereof.

In some embodiments, the fatty acid synthase inhibitor may be a compound of Formula (IX):

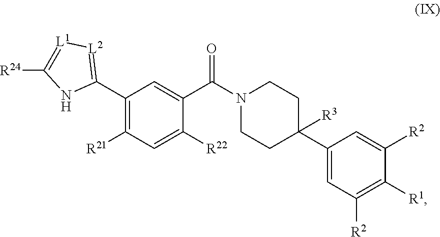

or a pharmaceutically acceptable salt thereof, wherein: R¹ is H, —CN, halogen, C₁-C₄ straight or branched alkyl, —O—(C₃-C₅ cycloalkyl), or —O—(C₁-C₄ straight or branched alkyl) wherein: C₃-C₅ cycloalkyl optionally includes an oxygen or nitrogen heteroatom; and when R¹ is not H, —CN or halogen, it is optionally substituted with one or more halogens; each R² is independently hydrogen, halogen or C₁-C₄ straight or branched alkyl; R³ is H, —OH, or halogen; R₂₁ is H, halogen, C₁-C₄ straight or branched alkyl, or C₃-C₅ cycloalkyl wherein the C₃-C₅ cycloalkyl optionally includes an oxygen or nitrogen heteroatom; R²² is H, halogen, or C₁-C₂ alkyl; R²⁴ is H, C₁-C₄ straight or branched alkyl, —(C₁-C₄ alkyl)ₜ-OH, —(C₁-C₄ alkyl)ₜ-Oₜ—(C₃-C₅ cycloalkyl), or —(C₁-C₄ alkyl)ₜ-O—(C₁-C₄ straight or branched alkyl) wherein: t is 0 or 1; the C₃-C₅ cycloalkyl optionally includes an oxygen or nitrogen heteroatom; L¹ is CR²³ or N; L² is CH or N; at least one of L¹ or L² is N; and R²³ is H or C₁-C₄ straight or branched alkyl.

In some embodiments of the compound of Formula (IX), R²⁴ is C₁-C₄ straight or branched alkyl or —(C₁-C₄ alkyl)ₜ-O—(C₁-C₄ straight or branched alkyl) wherein t is 0 or 1.

In some embodiments of the compound of Formula (IX), R²¹ is halogen, C₁-C₄ straight or branched alkyl, C₃-C₅ cycloalkyl wherein the C₃-C₅ cycloalkyl optionally includes an oxygen or nitrogen heteroatom, or —S(O)ᵤ—(C₁-C₄ straight or branched alkyl) wherein u is 0 or 2, or —S(O)ᵤ—(C₃-C₅ cycloalkyl) wherein u is 0 or 2;

In some embodiments of the compound of Formula (IX), R³ is H or halogen.

In some embodiments of the compound of Formula (IX), R¹ is halogen, —CN or C₁-C₂ haloalkyl.

In some embodiments of the compound of Formula (IX), both L¹ and L² are N.

In some embodiments of the compound of Formula (IX), R²¹ is C₁-C₂ alkyl or C₃-C₅ cycloalkyl and R²² is C₁-C₂ alkyl.

In some embodiments of the compound of Formula (IX), R²¹ is C₃-C₅ cycloalkyl and R²² is C₁-C₂ alkyl.

In some embodiments of the compound of Formula (IX), R²⁴ is —(C₁-C₂ alkyl)ₜ-O—(C₁-C₂ alkyl) wherein t is 0 or 1.

In some embodiments of the compound of Formula (IX), R²¹ is C₃-C₅ cycloalkyl, R²² is C₁-C₂ alkyl and R²⁴ is C₁-C₂ alkyl.

In some embodiments of the compound of Formula (IX), R²¹ is cyclobutyl, R²² is C₁-C₂ alkyl and R²⁴ is C₁-C₂ alkyl.

In some embodiments of the compound of Formula (IX), R²¹ is cyclobutyl.

In some embodiments of the compound of Formula (IX), R³ is H or F.

In some embodiments of the compound of Formula (IX), R¹ is —CN.

In some embodiments of the compound of Formula (IX), R¹ is —CF₃.

In some embodiments of the compound of Formula (IX), R²² is H, methyl or ethyl.

In some embodiments of the compound of Formula (IX), R²² is H.

In some embodiments of the compound of Formula (IX), R²² is methyl.

In some embodiments of the compound of Formula (IX), R¹ is —CN, each R² is H, R³ is H or F, R²¹ is C₃-C₄ cycloalkyl, R²² is methyl, L¹ and L² are N, and R²⁴ is methyl, ethyl, hydroxymethyl, methoxymethyl, 2-methoxyethyl.

In some embodiments of the compound of Formula (IX), R¹ is —CN, each R² is H, R³ is H or F, R²¹ is C₃-C₄ cycloalkyl, R²² is methyl, L¹ and L² are N, and R²⁴ is methoxy or ethoxy.

In some embodiments of the compound of Formula (IX), R¹ is —CN, each R² is H, R³ is H or F, R²¹ is C₃-C₄ cycloalkyl, R²² is methyl, L¹ is CH, L² is N, and R²⁴ is methyl, ethyl, hydroxymethyl, methoxymethyl, or 2-methoxyethyl.

In some embodiments of the compound of Formula (IX), R¹ is —CN, each R² is H, R³ is H or F, R²¹ is C₃-C₄ cycloalkyl, R²² is methyl, L¹ is N, L² is CH, and R²⁴ is methyl, ethyl, hydroxymethyl, methoxymethyl, or 2-methoxyethyl.

In certain embodiments, the fatty acid synthase inhibitor is a compound selected from Compounds 001-152, 001-154, 001-156, and 001-246 of Table C-1, or a pharmaceutically acceptable salt thereof.

In some embodiments, the fatty acid synthase inhibitor may be a compound of Formula (X):

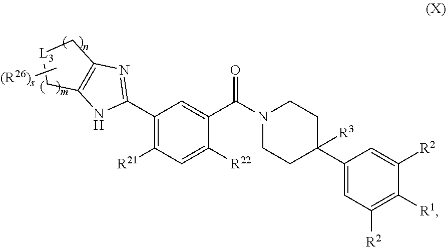

or a pharmaceutically acceptable salt thereof, wherein: R¹ is H, —CN, halogen, C₁-C₄ straight or branched alkyl, —O—(C₃-C₅ cycloalkyl), or —O—(C₁-C₄ straight or branched alkyl) wherein: the C₃-C₅ cycloalkyl optionally includes an oxygen or nitrogen heteroatom; and when R¹ is not H, —CN or halogen, it is optionally substituted with one or more halogens; each R² is independently hydrogen, halogen or C₁-C₄ straight or branched alkyl; R³ is H, —OH or halogen; L³ is C(R⁶⁰)₂, O or NR⁵⁰; each R⁶⁰ is independently H, —OH, —CN, —Oₜ—(C₃-C₅ cycloalkyl), —O—(C₁-C₄ straight or branched alkyl), or —C(O)—N(R⁶⁰¹)₂ wherein: t is 0 or 1, and the C₃-C₅ cycloalkyl optionally includes an oxygen or nitrogen heteroatom; each R⁵⁰ is independently H, —C(O)—Oₜ—(C₁-C₄ straight or branched alkyl), —C(O)—Oₜ—(C₃-C₅ cyclic alkyl), —C₃-C₅ cyclic alkyl optionally containing an oxygen or nitrogen heteroatom, —C(O)—N(R⁵⁰¹)₂, or C₁-C₄ straight or branched alkyl wherein: t is 0 or 1, and the C₃-C₅ cycloalkyl optionally includes an oxygen or nitrogen heteroatom; n is 1, 2 or 3; m is 1 or 2; R²¹ is H, halogen, C₁-C₄ straight or branched alkyl, or C₃-C₅ cycloalkyl wherein the C₃-C₅ cycloalkyl optionally includes an oxygen or nitrogen heteroatom R²² is H, halogen, or C₁-C₂ alkyl; each R²⁶ is independently —OH, —CN, halogen, C₁-C₄ straight or branched alkyl, —(C₁-C₄ alkyl)ₜ-Oₜ—(C₃-C₅ cycloalkyl), —(C₁-C₄ alkyl)ₜ-O—(C₁-C₄ straight or branched alkyl), —C(O)—Oₜ—(C₁-C₄ alkyl), or —C(O)—N(R⁵⁰¹)₂ wherein: t is 0 or 1, and the C₃-C₅ cycloalkyl optionally includes an oxygen or nitrogen heteroatom; s is 0, 1 or 2; and each R⁶⁰¹ and R⁵⁰¹ is independently H or C₁-C₄ straight or branched alkyl; and wherein two of R²⁶, R⁶⁰, R⁵⁰, R⁵⁰¹ and R⁶⁰¹ optionally join to form a ring wherein the two of R²⁶, R⁶⁰, R⁵⁰, R⁵⁰¹ and R⁶⁰¹ may be two R²⁶, two R⁶⁰, two R⁵⁰, two R⁵⁰¹ or two R⁶⁰¹.

In some embodiments of the compound of Formula (X), R²¹ is halogen, C₁-C₄ straight or branched alkyl or C₃-C₅ cycloalkyl.

In some embodiments of the compound of Formula (X), R³ is H or halogen.

In some embodiments of the compound of Formula (X), R¹ is —CN or C₁-C₂ haloalkyl.

In some embodiments of the compound of Formula (X), R³ is H or F.

In some embodiments of the compound of Formula (X), R¹ is —CN.

In some embodiments of the compound of Formula (X), R¹ is —CF₃.

In some embodiments of the compound of Formula (X), n is 1.

In some embodiments of the compound of Formula (X), n is 2.

In some embodiments of the compound of Formula (X), m is 1

In some embodiments of the compound of Formula (X), m is 2.

In some embodiments of the compound of Formula (X), R²¹ is C₁-C₂ alkyl or C₃-C₅ cycloalkyl and R²² is C₁-C₂ alkyl.

In some embodiments of the compound of Formula (X), R²¹ is C₃-C₅ cycloalkyl and R²² is C₁-C₂ alkyl.

In some embodiments of the compound of Formula (X), n is 2, m is 1, L³ is —N—C(O)—O—(C₁-C₂ alkyl).

In some embodiments of the compound of Formula (X), L³ is NR⁵⁰; R⁵⁰ is C₁-C₂ alkyl; R²¹ is cyclobutyl; R²² is H or methyl; R³ is H; R¹ is —CN; m is 2 and n is 1 or 2.

In some embodiments of the compound of Formula (X), n is 2, m is 1, L³ is O and s is 0.

In some embodiments of the compound of Formula (X), R²² is H, methyl or ethyl.

In some embodiments of the compound of Formula (X), R²² is methyl.

In some embodiments of the compound of Formula (X), R²² is H.

In some embodiments of the compound of Formula (X), R¹ is —CN, each R² is H, R³ is H or F, R²¹ is C₃-C₄ cycloalkyl, R²² is methyl, n is 2 and L³ is NR⁵⁰ where R⁵⁰ is methyl or ethyl.

In some embodiments of the compound of Formula (X), R¹ is —CN, each R² is H, R³ is H or F, R²¹ is C₃-C₄ cycloalkyl, R²² is methyl, n is 2 and L³ is O.

In certain embodiments, the fatty acid synthase inhibitor is a compound selected from Compounds 001-406, 001-440, and 001-439 of Table C-1, or a pharmaceutically acceptable salt thereof.

In some embodiments, the fatty acid synthase inhibitor may be a compound of Formula (XI):

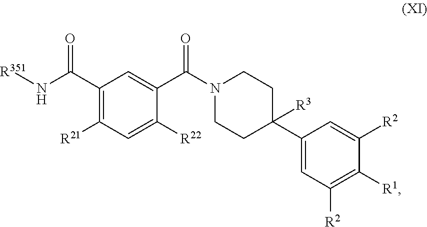

or a pharmaceutically acceptable salt thereof, wherein: R¹ is H, —CN, halogen, C₁-C₄ straight or branched alkyl, —O—(C₃-C₅ cycloalkyl), or —O—(C₁-C₄ straight or branched alkyl) wherein: the C₃-C₅ cycloalkyl optionally includes an oxygen or nitrogen heteroatom; and when R¹ is not H, —CN or halogen, it is optionally substituted with one or more halogens; each R² is independently H, halogen or C₁-C₄ straight or branched alkyl; R³ is H, —OH, or halogen; R²¹ is cyclobutyl, azetidin-1-yl, or cyclopropyl; R²² is H, halogen, C₁-C₂ alkyl; and R³⁵¹ is C₁-C₂ alkyl or C₂—O—(C₁ or C₂ alkyl).

In some embodiments of the compound of Formula (XI), R³ is H or halogen.

In some embodiments of the compound of Formula (XI), R¹ is halogen, —CN or C₁-C₂ haloalkyl.

In some embodiments of the compound of Formula (XI), R²¹ is C₃-C₄ cycloalkyl and R²² is C₁-C₂ alkyl.

In some embodiments of the compound of Formula (XI), R²¹ is cyclobutyl and R²² is C₁-C₂ alkyl.

In some embodiments of the compound of Formula (XI), R²¹ is cyclobutyl.

In some embodiments of the compound of Formula (XI), R³ is H or F.

In some embodiments of the compound of Formula (XI), R¹ is —CN.

In some embodiments of the compound of Formula (XI), R¹ is —CF₃.

In some embodiments of the compound of Formula (XI), R²² is H, methyl or ethyl.

In some embodiments of the compound of Formula (XI), R²² is H.

In some embodiments of the compound of Formula (XI), R²² is methyl.

In some embodiments of the compound of Formula (XI), R¹ is —CN, each R² is H, R³ is H or F, R²¹ is cyclobutyl, R²² is methyl and R³⁵¹ is methyl or ethyl.

In certain embodiments, the fatty acid synthase inhibitor is Compound 001-254 or 001-256 of Table C-1, or a pharmaceutically acceptable salt thereof.

In some embodiments, the fatty acid synthase inhibitor may be a compound of Formula (XII):

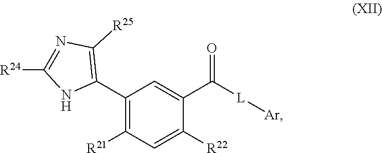

or pharmaceutically acceptable salts thereof, wherein:
L-Ar is

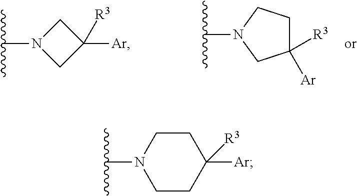

Ar is

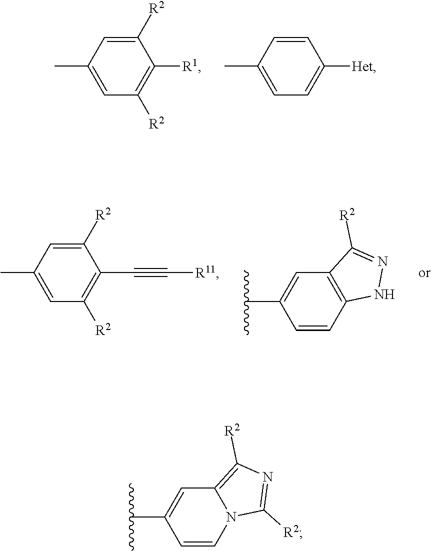

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; R²⁴ is H, —CN, —(C₁-C₄ alkyl)-CN, C₁-C₄ alkyl, C₁-C₄ haloalkyl, —(C₁-C₄ alkyl)-OH, —(C₁-C₄ alkyl)-N(R²⁴¹)₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl), wherein: t is 0 or 1; u is 0 or 1; with the proviso that when u is 1, t is 1; and each R²⁴¹ is independently H or C₁-C₂ alkyl; and R²⁵ is halogen, —CN, —(C₁-C₄ alkyl)-CN, C₁-C₂ alkyl, C₁-C₄ haloalkyl, —(C₁-C₄ alkyl)-O—(C₁-C₄ alkyl), or cyclopropyl.

In some embodiments of the compound of Formula (XII), L-Ar is

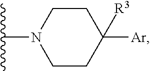

### Ar is not

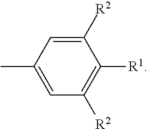

In some embodiments of the compound of Formula (XII), L-Ar is

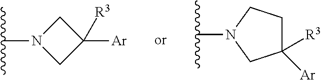

### and Ar is

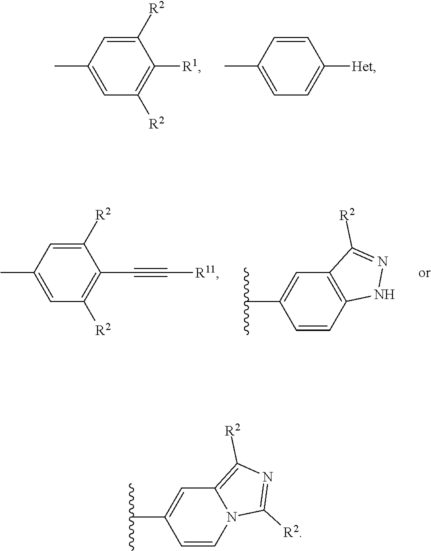

In some embodiments of the compound of Formula (XII), L-Ar is

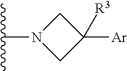

### and Ar is

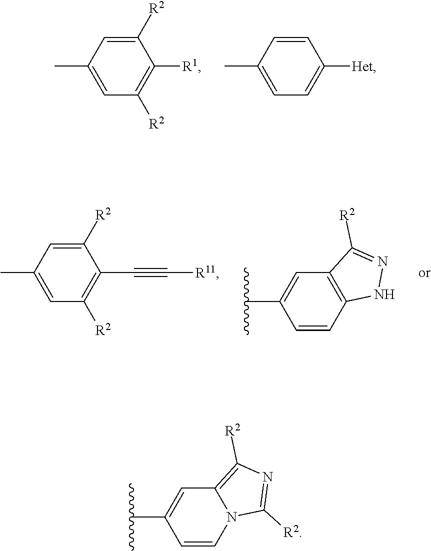

In some embodiments of the compound of Formula (XII), Ar is

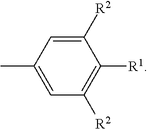

In some embodiments of the compound of Formula (XII), R¹ is halogen, —CN or C₁-C₂ haloalkyl.

In some embodiments of the compound of Formula (XII), R¹ is —CN.

In some embodiments of the compound of Formula (XII), R² is H.

In some embodiments of the compound of Formula (XII), R²¹ is halogen, C₁-C₄ alkyl or C₃-C₅ cycloalkyl.

In some embodiments of the compound of Formula (XII), R²¹ is C₁-C₄ alkyl or C₃-C₅ cycloalkyl.

In some embodiments of the compound of Formula (XII), R²¹ is C₁-C₂ alkyl or C₃-C₅ cycloalkyl.

In some embodiments of the compound of Formula (XII), R²¹ is C₁-C₂ alkyl.

In some embodiments of the compound of Formula (XII), R²¹ is —CH₃.

In some embodiments of the compound of Formula (XII), R²² is H or C₁-C₂ alkyl.

In some embodiments of the compound of Formula (XII), R²² is H or —CH₃.

In some embodiments of the compound of Formula (XII), R²² is —CH₃.

In some embodiments of the compound of Formula (XII), R²⁴ is H, —CN, —(C₁-C₄ alkyl)-CN, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl).

In some embodiments of the compound of Formula (XII), R²⁴ is H, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₆ cycloalkyl), —(C₁-C₄ alkyl)ₜ-Oᵤ-(4- to 6-membered heterocycle) or —(C₁-C₄ alkyl)-O—(C₁-C₄ alkyl).

In some embodiments of the compound of Formula (XII), R²⁴ is C₁-C₄ alkyl or —(C₁-C₄ alkyl)-O—(C₁-C₄ alkyl).

In some embodiments, the fatty acid synthase inhibitor is a compound of Formula (XII) wherein R²⁴ is —(C₁-C₂ alkyl)-O—(C₁-C₂ alkyl).

In some embodiments of the compound of Formula (XII), R²⁴ is —CH₂—O—CH₃.

In some embodiments of the compound of Formula (XII), R²⁴ is C₁-C₂ alkyl.

In some embodiments of the compound of Formula (XII), R²⁴ is —CH₃.

In some embodiments of the compound of Formula (XII), R²⁴ is C₃-C₆ cycloalkyl.

In some embodiments of the compound of Formula (XII), R²⁴ is —CN or —(C₁-C₂ alkyl)-CN.

In some embodiments of the compound of Formula (XII), R²⁴ is —CN.

In some embodiments of the compound of Formula (XII), R²⁴ is —(C₁-C₂ alkyl)-CN.

In some embodiments of the compound of Formula (XII), R²⁴ is H, —CH₃, —CH₂OH, —CH₂OCH₃, —(CH₂)₂OH, —(CH₂)₂OCH₃ or —(CH₂)₂N(CH₃)₂.

In some embodiments of the compound of Formula (XII), R²⁴ is methyl, isopropyl, cyclopropyl, —CN, or —(C₁-C₂ alkyl)-CN.

In some embodiments of the compound of Formula (XII), R²⁴ is substituted with one or more substituents selected from C₁-C₂ alkyl, oxo, —CN, halogen, alkanoyl, alkoxycarbonyl, —OH and C₁-C₂ alkoxy.

In some embodiments of the compound of Formula (XII), R²⁴ is substituted with one or more substituents selected from methyl, —F, methoxy, —C(═O)CH₃ and —C(═O)—OCH₃.

In some embodiments of the compound of Formula (XII), R²⁴ is substituted with two substituents that are the same or different.

In some embodiments of the compound of Formula (XII), R²⁴ is substituted with three substituents that are the same or different.

In some embodiments of the compound of Formula (XII), R²⁵ is halogen, —CN, C₁-C₂ alkyl or cyclopropyl.

In some embodiments of the compound of Formula (XII), R²⁵ is halogen, C₁-C₂ alkyl or cyclopropyl.

In some embodiments of the compound of Formula (XII), R²⁵ is —CN, —Cl or —CH₃.

In some embodiments of the compound of Formula (XII), R²⁵ is —Cl.

In some embodiments of the compound of Formula (XII), R²⁵ is —CH₃.

In some embodiments of the compound of Formula (XII), R²⁵ is substituted with one or more substituents selected from —OH, halogen, C₁-C₂ alkyl and alkylcarbonyloxy.

In some embodiments of the compound of Formula (XII), R²⁵ is substituted with one or more substituents selected from —F, methyl and —O—C(═O)—CH₃.

In some embodiments of the compound of Formula (XII), R²⁵ is substituted with two substituents that are the same or different.

In some embodiments of the compound of Formula (XII), R²⁵ is substituted with three substituents that are the same or different.

In some embodiments, the fatty acid synthase inhibitor may be a compound of Formula (XIII):

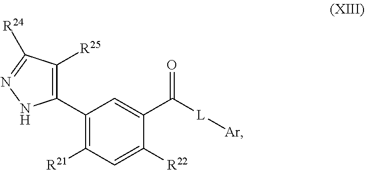

or pharmaceutically acceptable salts thereof, wherein:
L-Ar is

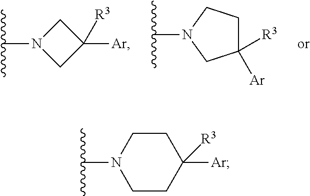

Ar is

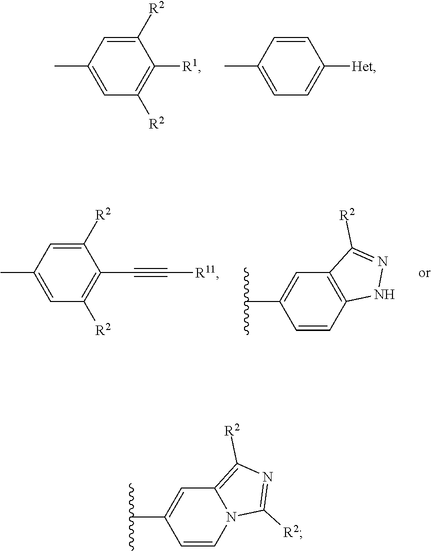

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and each R²⁴ and R²⁵ is independently H, halogen, —CN, —(C₁-C₄ alkyl)-CN, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₅ cycloalkyl), —(C₁-C₄ alkyl)ₜ-Oᵤ—(4- to 6-membered heterocycle) or —(C₁-C₄ alkyl)ₜ-O—(C₁-C₄ alkyl), wherein: each t is independently 0 or 1; each u is independently 0 or 1; and each R²⁴¹ is independently H or C₁-C₂ alkyl.

In some embodiments of the compound of Formula (XIII), when L-Ar is

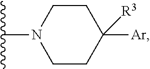

### Ar is not

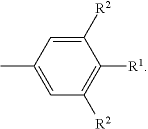

In some embodiments of the compound of Formula (XIII), L-Ar is

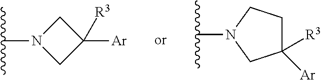

### and Ar is

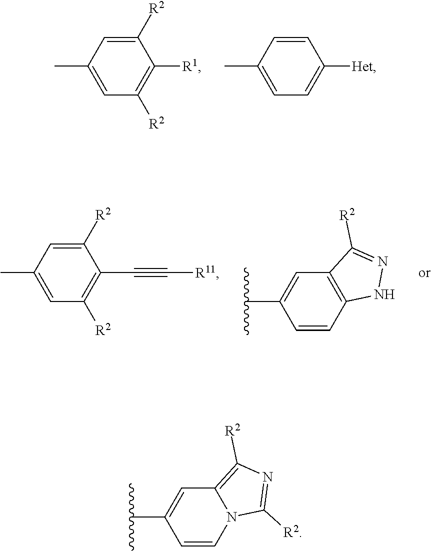

In some embodiments of the compound of Formula (XIII), L-Ar is

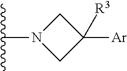

### and Ar is

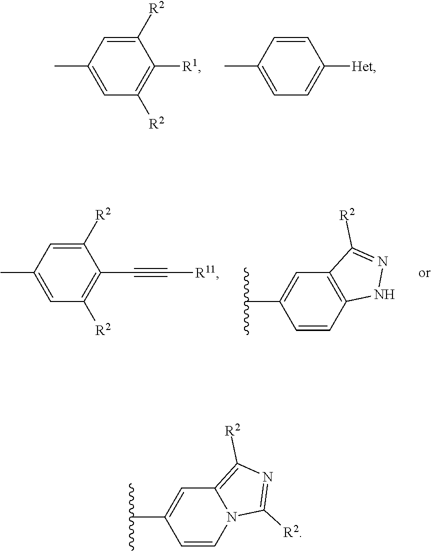

In some embodiments of the compound of Formula (XIII), Ar is

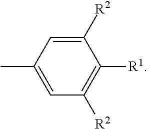

In some embodiments of the compound of Formula (XIII), R¹ is halogen, —CN or C₁-C₂ haloalkyl.

In some embodiments of the compound of Formula (XIII), R¹ is —CN.

In some embodiments of the compound of Formula (XIII), R² is H.

In some embodiments of the compound of Formula (XIII), R²¹ is halogen, C₁-C₄ alkyl, C₃-C₅ cycloalkyl or 4- to 6-membered heterocycle.

In some embodiments of the compound of Formula (XIII), R²¹ is C₁-C₄ alkyl, C₃-C₅ cycloalkyl or 4- to 6-membered heterocycle.

In some embodiments of the compound of Formula (XIII), R²¹ is C₁-C₂ alkyl or C₃-C₅ cycloalkyl.

In some embodiments of the compound of Formula (XIII), R²¹ is C₁-C₂ alkyl.

In some embodiments of the compound of Formula (XIII), R²¹ is —CH₃.

In some embodiments of the compound of Formula (XIII), R²² is H or C₁-C₂ alkyl.

In some embodiments of the compound of Formula (XIII), R²² is H or —CH₃.

In some embodiments of the compound of Formula (XIII), R²² is —CH₃.

In some embodiments of the compound of Formula (XIII), each R²⁴ and R²⁵ is independently H, —CN, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₅ cycloalkyl), —(C₁-C₄ alkyl)ₜ-Oᵤ—(4- to 6-membered heterocycle) or —(C₁-C₄ alkyl)-O—(C₁-C₄ alkyl).

In some embodiments of the compound of Formula (XIII), each R²⁴ and R²⁵ is independently H, C₁-C₄ alkyl, —(C₁-C₄ alkyl)ₜ-Oᵤ—(4- to 6-membered heterocycle) or —(C₁-C₄ alkyl)-O—(C₁-C₄ alkyl).

In some embodiments of the compound of Formula (XIII), R²⁴ is H, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₅ cycloalkyl), —(C₁-C₄ alkyl)ₜ-Oᵤ-(4- to 6-membered heterocycle) or —(C₁-C₄ alkyl)-O—(C₁-C₄ alkyl).

In some embodiments of the compound of Formula (XIII), R²⁴ is —CN, —Cl, C₁-C₄ alkyl or —(C₁-C₄ alkyl)-O—(C₁-C₄ alkyl).

In some embodiments of the compound of Formula (XIII), R²⁴ is C₁-C₄ alkyl or —(C₁-C₄ alkyl)-O—(C₁-C₄ alkyl).

In some embodiments of the compound of Formula (XIII), R²⁴ is —(C₁-C₂ alkyl)-O—(C₁-C₂ alkyl).

In some embodiments of the compound of Formula (XIII), R²⁴ is C₁-C₄ alkyl.

In some embodiments of the compound of Formula (XIII), R²⁴ is —CH₃.

In some embodiments of the compound of Formula (XIII), R²⁴ is hydrogen.

In some embodiments of the compound of Formula (XIII), R²⁴ is substituted with one or more substituents selected from halogen, C₃-C₅ cycloalkyl and C₁-C₂ alkoxy.

In some embodiments of the compound of Formula (XIII), R²⁴ is substituted with one or more substituents selected from —F, cyclopropyl and —OCH₃.

In some embodiments of the compound of Formula (XIII), R²⁴ is substituted with two substituents that are the same or different.

In some embodiments of the compound of Formula (XIII), R²⁴ is substituted with three substituents that are the same or different.

In some embodiments of the compound of Formula (XIII), R²⁵ is halogen, methyl, ethyl or cyclopropyl.

In some embodiments of the compound of Formula (XIII), R²⁵ is —CN, —Cl, C₁-C₄ alkyl, —(C₁-C₄ alkyl)ₜ-O—(C₃-C₅ cycloalkyl) or —(C₁-C₄ alkyl)ₜ-O—(C₁-C₄ alkyl).

In some embodiments of the compound of Formula (XIII), R²⁵ is —CN, —Cl, —CH₃, —O—(C₃-C₅ cycloalkyl) or —O—(C₁-C₂ alkyl).

In some embodiments of the compound of Formula (XIII), R²⁵ is —CN, —Cl or C₁-C₄ alkyl.

In some embodiments of the compound of Formula (XIII), R²⁵ is —CH₃.

In some embodiments of the compound of Formula (XIII), R²⁵ is —Cl.

In some embodiments of the compound of Formula (XIII), R²⁵ is substituted with one or more halogen.

In some embodiments of the compound of Formula (XIII), R²⁵ is substituted with one or more —F.

In some embodiments of the compound of Formula (XIII), R²⁵ is substituted by two substituents.

In some embodiments of the compound of Formula (XIII), R²⁵ is substituted by three substituents.

In some embodiments, the fatty acid synthase inhibitor may be a compound of Formula (XIV):

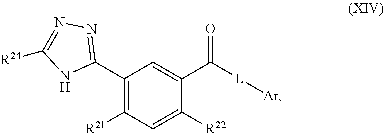

or pharmaceutically acceptable salts thereof, wherein:
L-Ar is

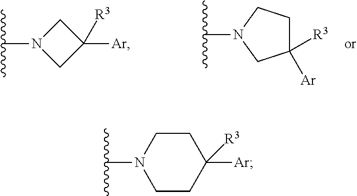

Ar is

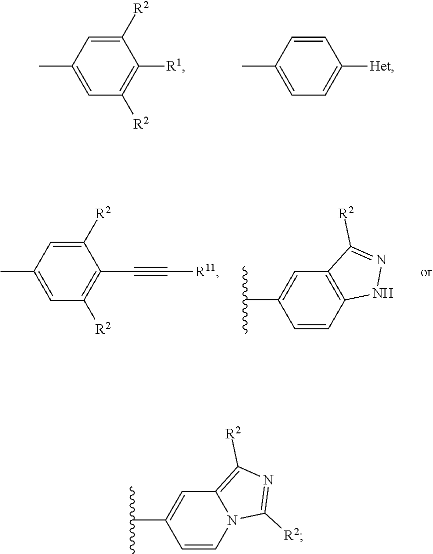

with the proviso that when L-Ar is

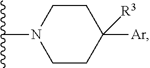

### Ar is not

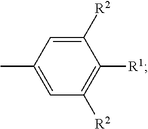

Het is a 5- to 6-membered heteroaryl; R is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; and R²⁴ is H, C₁-C₄ alkyl, C₁-C₄ haloalkyl, —(C₁-C₄ alkyl)-OH, —(C₁-C₄ alkyl)ₜ-N(R²⁴¹)₂, —(C₁-C₄ alkyl)ₜ-Oₜ—(C₃-C₅ cycloalkyl), —(C₁-C₄ alkyl)ₜ-Oₜ-(4- to 6-membered heterocycle) or —(C₁-C₄ alkyl)ₜ-O—(C₁-C₄ alkyl), wherein: each t is independently 0 or 1; and each R²⁴¹ is independently H or C₁-C₂ alkyl.

In some embodiments of the compound of Formula (XIV), L-Ar is

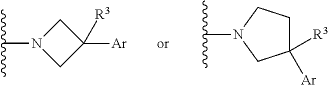

### and Ar is

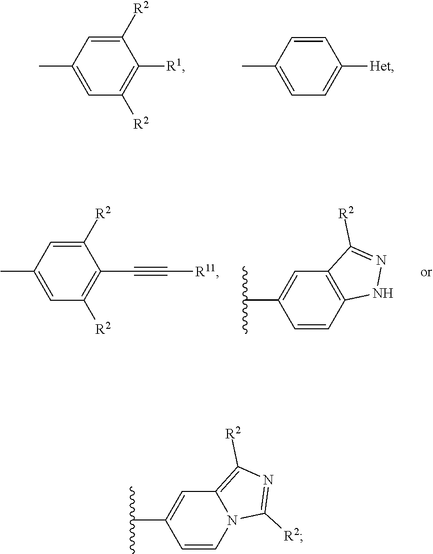

In some embodiments of the compound of Formula (XIV), L-Ar is

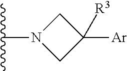

### and Ar is

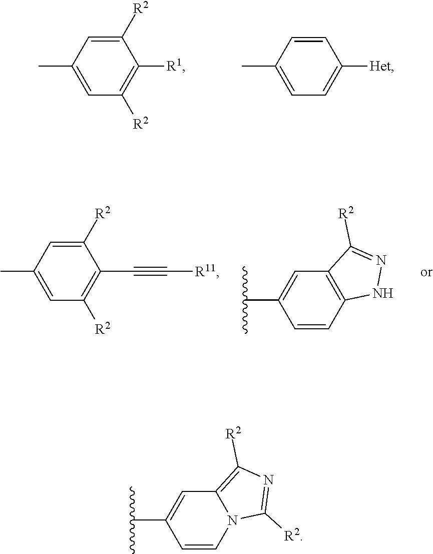

In some embodiments of the compound of Formula (XIV), Ar is

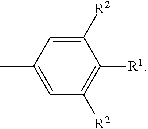

In some embodiments of the compound of Formula (XIV), R¹ is halogen, —CN or C₁-C₂ haloalkyl.

In some embodiments of the compound of Formula (XIV), R¹ is —CN.

In some embodiments of the compound of Formula (XIV), R² is H.

In some embodiments of the compound of Formula (XIV), R²¹ is halogen, C₁-C₄ alkyl, C₃-C₅ cycloalkyl or 4- to 6-membered heterocycle.

In some embodiments of the compound of Formula (XIV), R²¹ is H, C₁-C₄ alkyl, C₃-C₅ cycloalkyl or 4- to 6-membered heterocycle.

In some embodiments of the compound of Formula (XIV), R²¹ is C₁-C₂ alkyl or C₃-C₅ cycloalkyl.

In some embodiments of the compound of Formula (XIV), R²¹ is C₁-C₂ alkyl.

In some embodiments of the compound of Formula (XIV), R²¹ is C₃-C₅ cycloalkyl.

In some embodiments of the compound of Formula (XIV), R²² is H or C₁-C₂ alkyl.

In some embodiments of the compound of Formula (XIV), R²² is H.

In some embodiments of the compound of Formula (XIV), R²² is C₁-C₂ alkyl.

In some embodiments of the compound of Formula (XIV), R²² is —CH₃.

In some embodiments of the compound of Formula (XIV), R²⁴ is C₁-C₄ alkyl or —(C₁-C₄ alkyl)ₜ-O—(C₁-C₄ alkyl).

In some embodiments of the compound of Formula (XIV), R²⁴ is —(C₁-C₂ alkyl)ₜ-O—(C₁-C₂ alkyl).

In some embodiments, the fatty acid synthase inhibitor may be a compound of Formula (XV):

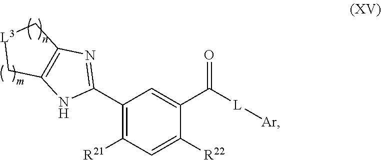

or pharmaceutically acceptable salts thereof, wherein: L³ is —CH₂—, —CHR⁵⁰—, —O—, —NR⁵⁰—, —NC(O)R⁵⁰— or —NC(O)OR⁵⁰—, wherein R⁵⁰ is C₁-C₆ alkyl, C₃-C₅ cycloalkyl, or 4- to 6-membered heterocycle; n is 1, 2, or 3; m is 1 or 2 with the proviso that n+m≥3;
L-Ar is

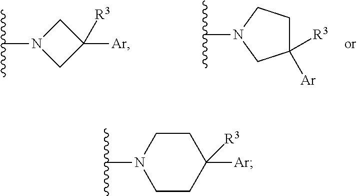

Ar is

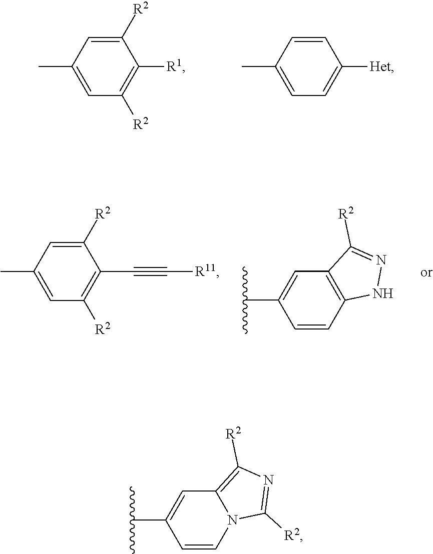

with the proviso that when L-Ar is

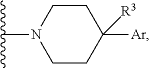

Ar is not

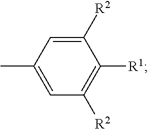

Het is a 5- to 6-membered heteroaryl; R¹ is H, —CN, halogen, C₁-C₄ alkyl, —O—(C₃-C₅ cycloalkyl), —O-(4- to 6-membered heterocycle), or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl, or a 4- to 6-membered heterocycle; and R²² is H, halogen, or C₁-C₂ alkyl.

In some embodiments of the compound of Formula (XV), L-Ar is

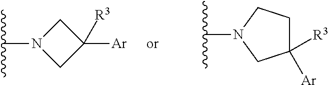

### and Ar is

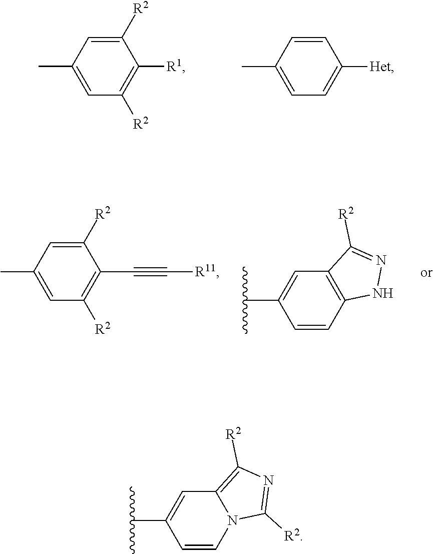

In some embodiments of the compound of Formula (XV), L-Ar is

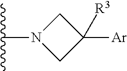

### and Ar is

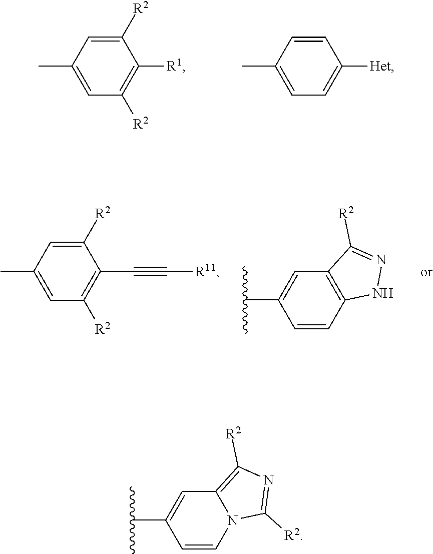

In some embodiments of the compound of Formula (XV), R¹ is H, —CN, —C₁-C₄ alkyl, —O—(C₃-C₅ cycloalkyl), —O-(4- to 6-membered heterocycle) or —O—(C₁-C₄ alkyl) wherein when R¹ is not H or —CN, R¹ is optionally substituted with one or more halogens.

In some embodiments of the compound of Formula (XV), R¹ is halogen, —CN or C₁-C₂ haloalkyl.

In some embodiments of the compound of Formula (XV), R¹ is —CN or C₁-C₂ haloalkyl.

In some embodiments of the compound of Formula (XV), R¹ is —CN.

In some embodiments of the compound of Formula (XV), R¹ is —Cl.

In some embodiments of the compound of Formula (XV), R² is H.

In some embodiments of the compound of Formula (XV), R²¹ is halogen, C₁-C₄ alkyl, C₃-C₅ cycloalkyl or 4- to 6-membered heterocycle.

In some embodiments of the compound of Formula (XV), R²¹ is C₁-C₂ alkyl or C₃-C₅ cycloalkyl.

In some embodiments of the compound of Formula (XV), R²¹ is C₃-C₅ cycloalkyl.

In some embodiments of the compound of Formula (XV), R²² is H or C₁-C₂ alkyl.

In some embodiments of the compound of Formula (XV), R²² is H.

In some embodiments of the compound of Formula (XV), R²² is C₁-C₂ alkyl.

In some embodiments of the compound of Formula (XV), R²² is —CH₃.

In some embodiments of the compound of Formula (XV), L³ is —N(CH₃)—.

In some embodiments of the compound of Formula (XV), n is 2 and m is 2.

In some embodiments of the compound of Formula (XV), n is 1 or 2.

In some embodiments of the compound of Formula (XV), n is 1 and m is 2.

In some embodiments, the fatty acid synthase inhibitor may be a compound of Formula (XVI):

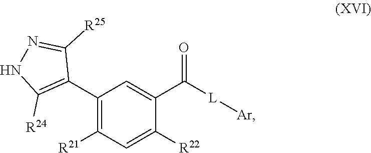

or pharmaceutically acceptable salts thereof, wherein:
L-Ar is

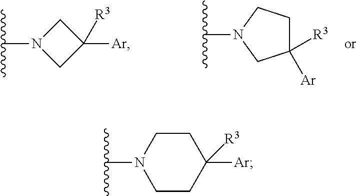

Ar is

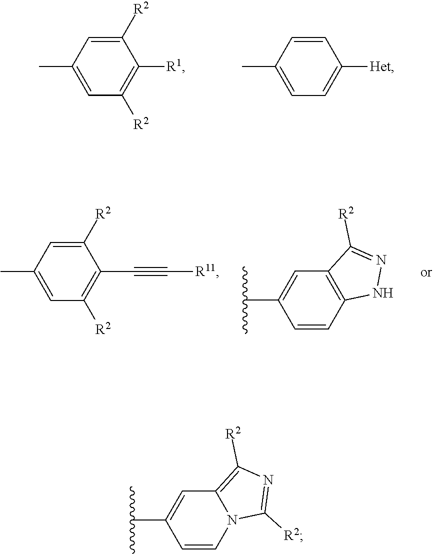

Het is a 5- to 6-membered heteroaryl; R¹ is H, —CN, halogen, C₁-C₄ alkyl, —O—(C₃-C₅ cycloalkyl), —O-(4- to 6-membered heterocycle) or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen or C₁-C₂ alkyl; and each of R²⁴ and R²⁵ is independently H, —C₁-C₄ alkyl, or halogen.

In some embodiments of the compound of Formula (XVI), L-Ar is

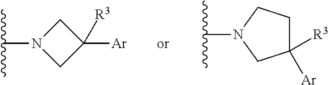

### and Ar is

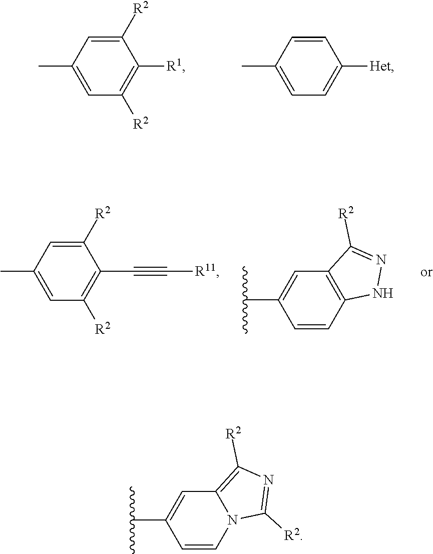

In some embodiments of the compound of Formula (XVI), L-Ar is

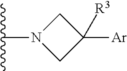

### and Ar is

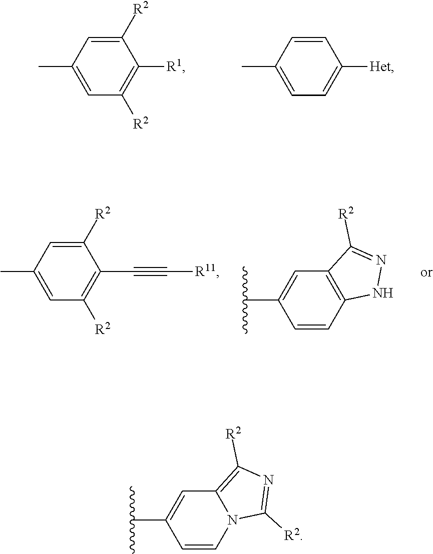

In some embodiments of the compound of Formula (XVI), R¹ is halogen, —CN or C₁-C₂ haloalkyl.

In some embodiments of the compound of Formula (XVI), R²¹ is halogen, C₁-C₄ alkyl, C₃-C₅ cycloalkyl or 4- to 6-membered heterocycle.

In some embodiments of the compound of Formula (XVI), R²¹ is —CH₃.

In some embodiments of the compound of Formula (XVI), R²² is H.

In some embodiments, the fatty acid synthase inhibitor may be a compound of Formula (XVII):

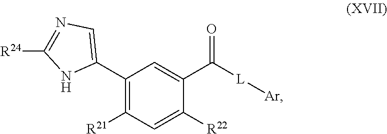

or pharmaceutically acceptable salts thereof, wherein:
L-Ar is

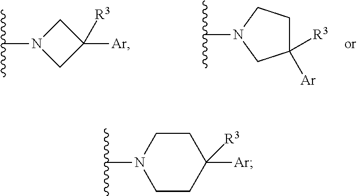

Ar is

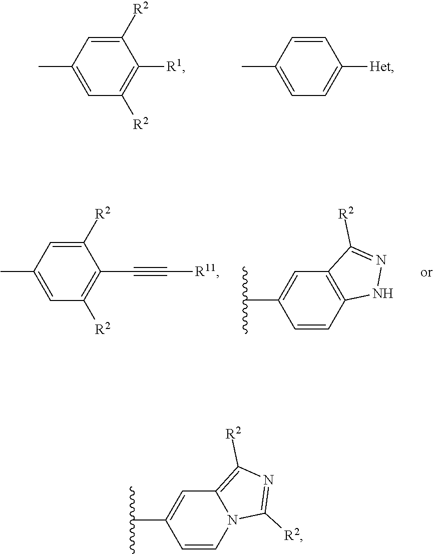

with the proviso that when L-Ar is

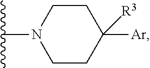

### Ar is not

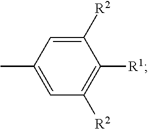

Het is a 5- to 6-membered heteroaryl; R¹ is H, —CN, halogen, C₁-C₄ alkyl, —O—(C₃-C₅ cycloalkyl), —O-(4- to 6-membered heterocycle), or —O—(C₁-C₄ alkyl), wherein when R¹ is not H, —CN or halogen, R¹ is optionally substituted with one or more halogens; each R² is independently hydrogen, halogen, or C₁-C₄ alkyl; R³ is H or F; R¹¹ is H or —CH₃; R²¹ is H, halogen, C₁-C₄ alkyl, C₃-C₅ cycloalkyl, or 4- to 6-membered heterocycle; R²² is H, halogen, or C₁-C₂ alkyl; and R²⁴ is H, C₁-C₄ alkyl, —(C₁-C₄ alkyl)-OH, —(C₁-C₄ alkyl)-N(R²⁴¹)₂, —(C₁-C₄ alkyl)ₜ-Oᵤ—(C₃-C₅ cycloalkyl), —(C₁-C₄ alkyl)ₜ-Oᵤ-(4- to 6-membered heterocycle), or —(C₁-C₄ alkyl)-O—(C₁-C₄ alkyl), wherein: t is 0 or 1; u is 0 or 1; with the proviso that when u is 1, t is 1; and R²⁴¹ is H or C₁-C₂ alkyl.

In some embodiments of the compound of Formula (XVII), L-Ar is

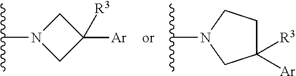

### and Ar is

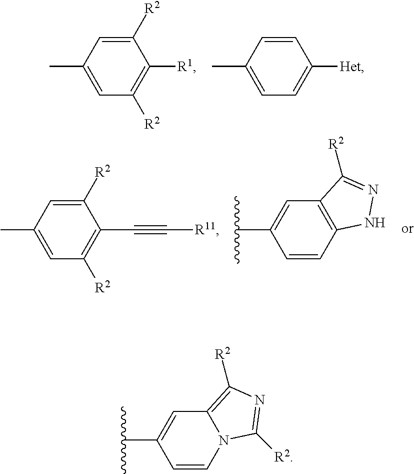

In some embodiments of the compound of Formula (XVII), R¹ is halogen, —CN or C₁-C₂ haloalkyl.

In some embodiments of the compound of Formula (XVII), R¹ is —CN.

In some embodiments of the compound of Formula (XVII), R² is H.

In some embodiments of the compound of Formula (XVII), R²¹ is halogen, C₁-C₄ alkyl, C₃-C₅ cycloalkyl or 4- to 6-membered heterocycle.

In some embodiments of the compound of Formula (XVII), R²¹ is C₁-C₂ alkyl or C₃-C₅ cycloalkyl.

In some embodiments of the compound of Formula (XVII), R²¹ is C₁-C₂ alkyl.

In some embodiments of the compound of Formula (XVII), R²¹ is C₃-C₅ cycloalkyl.

In some embodiments of the compound of Formula (XVII), R²² is H or C₁-C₂ alkyl.

In some embodiments of the compound of Formula (XVII), R²² is H.

In some embodiments of the compound of Formula (XVII), R²² is C₁-C₂ alkyl.

In some embodiments of the compound of Formula (XVII), R²² is —CH₃.

In some embodiments of the compound of Formula (XVII), R²⁴ is C₁-C₄ alkyl or —(C₁-C₄ alkyl)-O—(C₁-C₄ alkyl).

In some embodiments of the compound of Formula (XVII), R²⁴ is —(C₁-C₂ alkyl)-O—(C₁-C₂ alkyl).

In some embodiments, the fatty acid synthase inhibitor may be a compound of Formula (XVIII):

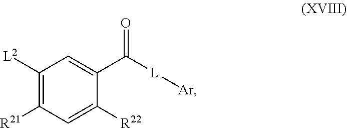

or pharmaceutically acceptable salts thereof, wherein:
L-Ar is

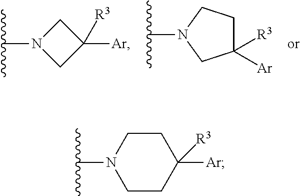

Ar is

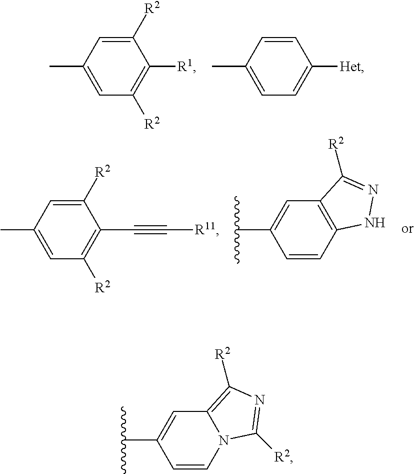

with the proviso that when L-Ar is

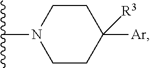

Ar is not

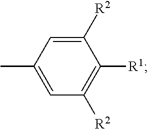

L² is —NHR³⁵ or —C(O)NHR³⁵¹, wherein R³⁵¹ is C₁-C₆ alkyl, C₃-C₅ cycloalkyl, 4- to 6-membered heterocycle, aryl, or heteroaryl; Het is a 5- to 6-membered heteroaryl; R¹ is H, —CN, halogen, C₁-C₄ alkyl, —O—(C₃-C₅ cycloalkyl), —O-(4- to 6-membered heterocycle), or —O—(C₁-C₄ alkyl), wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl or 4- to 6-membered heterocycle; R²² is H, halogen, or C₁-C₂ alkyl; and R³⁵ is —C(O)R³⁵¹, —C(O)NHR³⁵¹, C(O)OR³⁵¹ or S(O)₂R³⁵¹, wherein R³⁵¹ is C₁-C₆ alkyl, C₃-C₅ cycloalkyl, 4- to 6-membered heterocycle, aryl, or heteroaryl.

In some embodiments, the fatty acid synthase inhibitor is a compound of Formula (XVIII) wherein L-Ar is

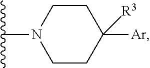

### Ar is not

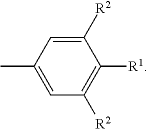

In some embodiments, the fatty acid synthase inhibitor is a compound of Formula (XVIII) wherein L² is —NHR³⁵.

In some embodiments, the fatty acid synthase inhibitor is a compound of Formula (XVIII) wherein L² is —C(O)NHR³⁵¹.

In some embodiments, the fatty acid synthase inhibitor may be a compound of Formula (XIX):

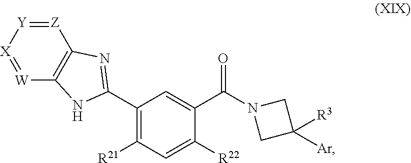

or pharmaceutically acceptable salts thereof, wherein: each W, X, Y and Z is independently —N— or —CR²⁶— with the proviso that not more than 2 of W, X, Y and Z are —N—; each R²⁶ is independently H, C₁-C₄ alkyl, —O—(C₁-C₄ alkyl), —N(R²⁷)₂, —S(O)₂—(C₁-C₄ alkyl), or —C(O)—(C₁-C₄ alkyl); each R²⁷ is independently H or C₁-C₄alkyl or both R²⁷ are C₁-C₄ alkyl and join to form a 3- to 6-membered ring together with the N to which they are attached and wherein the ring optionally includes one oxygen atom as one of the members of the ring;
Ar is

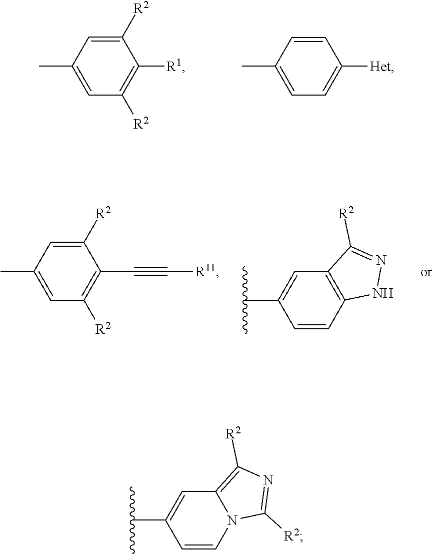

Het is a 5- to 6-membered heteroaryl; R¹ is H, —CN, halogen, C₁-C₄ alkyl, —O—(C₃-C₅ cycloalkyl), —O-(4- to 6-membered heterocycle), or —O—(C₁-C₄ alkyl) wherein when R¹ is not H, —CN or halogen, R¹ is optionally substituted with one or more halogens; each R² is independently hydrogen, halogen or C₁-C₄ alkyl; R³ is H or F; R¹¹ is H or —CH₃; R²¹ is H, halogen, C₁-C₄ alkyl, C₃-C₅ cycloalkyl, or a 4- to 6-membered heterocycle; and R²² is H, halogen or C₁-C₂ alkyl.

In some embodiments of the compound of Formula (XIX), Ar is

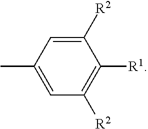

In some embodiments of the compound of Formula (XIX), Y is —CR²⁶— wherein R²⁶ is —N(R²⁷)₂.

In some embodiments of the compound of Formula (XIX), X is —N—.

In some embodiments, the fatty acid synthase inhibitor may be a compound of Formula (XX):

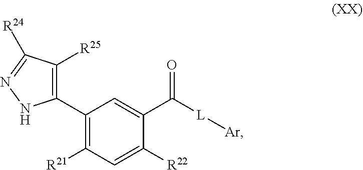

or a pharmaceutically acceptable salt thereof, wherein:
L-Ar is

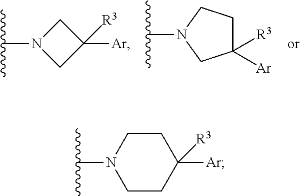

Ar is

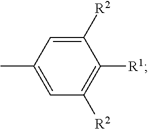

R¹ is H, —CN, halogen, C₁-C₄ alkyl, —O—(C₃-C₅ cycloalkyl), —O-(4- to 6-membered heterocycle), or —O—(C₁-C₄ alkyl), wherein when R¹ is not H, —CN or halogen, R¹ is optionally substituted with one or more halogen; each R² is independently hydrogen, halogen or C₁-C₄ alkyl; R³ is H or F; R²¹ is H, halogen, C₁-C₄ alkyl, C₃-C₅ cycloalkyl or 4- to 6-membered heterocycle; R²² is H, halogen, or C₁-C₂ alkyl; R²⁴ is —O—(C₁-C₄ alkyl), —O—(C₁-C₄ alkyl)-O—(C₁-C₄ alkyl), —O—(C₃-C₅ cycloalkyl), or —O-(4- to 6-membered heterocycle), wherein R²⁴ is optionally substituted with one or more hydroxyl or halogen; and R²⁵ is H, halogen, C₁-C₄ alkyl or C₃-C₅ cycloalkyl, wherein R²⁵ is optionally substituted with one or more halogen.

In some embodiments of the compound of Formula (XX), L-Ar is

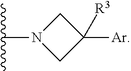

In some embodiments of the compound of Formula (XX), L-Ar is

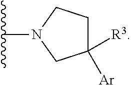

In some embodiments of the compound of Formula (XX), L-Ar is

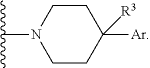

In some embodiments of the compound of Formula (XX), R³ is H.

In some embodiments of the compound of Formula (XX), R¹ is —CN or —O—(C₁-C₄ alkyl), wherein when R¹ is not —CN, R¹ is optionally substituted with one or more halogen.

In some embodiments of the compound of Formula (XX), R¹ is —CN.

In some embodiments of the compound of Formula (XX), R¹ is —O—(C₁-C₄ alkyl) optionally substituted with one or more halogen.

In some embodiments of the compound of Formula (XX), each R² is hydrogen.

In some embodiments of the compound of Formula (XX), R²¹ is C₁-C₄ alkyl.

In some embodiments of the compound of Formula (XX), R²² is H or C₁-C₂ alkyl.

In some embodiments of the compound of Formula (XX), R²⁴ is —O—(C₁-C₄ alkyl) optionally substituted with one or more hydroxyl or halogen.

In some embodiments of the compound of Formula (XX), R²⁴ is —O—(C₁-C₄ alkyl) optionally substituted with one or more hydroxyl.

In some embodiments of the compound of Formula (XX), R²⁵ is —CH₃.

In some embodiments, the present disclosure provides pharmaceutical compositions comprising any one of the compounds of Formulae (IX-1), (XII-1), (XIII-1), (XX-1), (I), (II), (III), (IV), (V), (VI), (VII), (VIII), (IX), (X), (XI), (XII), (XIII), (XIV), (XV), (XVI), (XVII), (XVIII), (XIV), or (XX) and a pharmaceutically acceptable carrier, excipient, or diluent.

Embodiments of the subject matter and the functional operations described in this specification can be implemented in digital electronic circuitry, in tangibly embodied computer Software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus’ refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, Software, a software application, a module, a software module, a script, or code, can be written in any form of programming language, including compiled or interpreted languages, or declarative or procedural languages, and it can be deployed in any form, including as a stand-alone program or as a module, component, Subroutine, or other unit Suitable for use in a computing environment. A computer program may, but need not, correspond to a file in a file system. A program can be stored in a portion of a file that holds other programs or data, e.g., one or more Scripts stored in a markup language document, in a single file dedicated to the program in question, or in multiple coordinated files, e.g., files that store one or more modules, Sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. Elements of a computer include a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have Such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD-ROM and DVD-ROM disks. The processor and the memory can be Supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more Such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

An example of one such type of computer is shown in FIG. 8, which is a block diagram of computing devices 800 that may be used to implement the systems and methods described above, as either a client or as a server or a plurality of servers. Computing device 800 is intended to represent various forms of digital computers, such as laptops, desktops, workstations, personal digital assistants, servers, blade servers, mainframes, and other appropriate computers. Additionally, computing device 500 can include Universal Serial Bus (USB) flash drives. The USB flash drives may store operating systems and other applications. The USB flash drives can include input/output components, such as a wireless transmitter or USB connector that may be inserted into a USB port of another computing device. The components shown here, their connections and relationships, and their functions, are meant to be exemplary only, and are not meant to limit implementations of the inventions described and/or claimed in this document.

Computing device 800 includes a processor 802, memory 804, a storage device 806, a high-speed interface 808 connecting to memory 804 and high-speed expansion ports 810, and a low-speed interface 812 connecting to low-speed bus 814 and storage device 806. Each of the components 802, 804, 806, 808, 810, and 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as display 816 coupled to high-speed interface 808. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 804 stores information within the computing device 800. In one implementation, the memory 804 is a volatile memory unit or units. In another implementation, the memory 804 is a non-volatile memory unit or units. The memory 804 may also be another form of computer-readable medium, such as a magnetic or optical disk.

The storage device 806 is capable of providing mass storage for the computing device 800. In one implementation, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 504, the storage device 806, or memory on processor 802.

The high-speed controller 808 manages bandwidth-intensive operations for the computing device 800, while the low-speed controller 812 manages lower bandwidth-intensive operations. Such allocation of functions is exemplary only. In one implementation, the high-speed controller 808 is coupled to memory 804, display 816 (e.g., through a graphics processor or accelerator), and to high-speed expansion ports 810, which may accept various expansion cards (not shown). In the implementation, low-speed controller 812 is coupled to storage device 806 and low-speed expansion port 814. The low-speed expansion port, which may include various communication ports (e.g., USB, Bluetooth, Ethernet, wireless Ethernet) may be coupled to one or more input/output devices, such as a keyboard, a pointing device, microphone/speaker pair, a scanner, or a networking device such as a switch or router, e.g., through a network adapter.

The computing device 800 may be implemented in a number of different forms, as shown in the figure. For example, it may be implemented as a standard server 820, or multiple times in a group of such servers. It may also be implemented as part of a rack server system 824. In addition, it may be implemented in a personal computer such as a laptop computer 822. Alternatively, components from computing device 800 may be combined with other components in a mobile device (not shown). Each of such devices may contain one or more of computing device 800, and an entire system may be made up of multiple computing devices 800 communicating with each other.

### EXAMPLES

In view of the preceding Figures, the remaining Figures will be referred to during description of development of the machine learning models in view of experimental data on subjects having or suspected of having NASH.

### Example 1: Modeling Techniques to Develop Algorithms for Baseline Determination of % LF Change Between Responder and Non-Responder Subjects

The compounds described herein are orally bioavailable inhibitors. In particular, Compound 001-152 of Table C-1 is a first-in-class FASN inhibitor. FASN is a key enzyme in the DNL pathway and is the only enzyme in the human body capable of converting metabolized sugar into a fatty, palmitate (16:0). The safety and efficacy of Compound 001-152 has been studied in subjects with NASH in FASCINATE-1, a Phase 2 multi-center, randomized, single-blind, placebo-controlled clinical trial. Subjects were randomly assigned to an active group (administered Compound 001-152) or a placebo group (2:1). Two doses (25 mg and 50 mg) of Compound 001-152 were evaluated in the active group (once-daily dosing of Compound 001-152 for 12 weeks). The percentage of liver fat (% LF) or hepatic fat fraction (HFF) measured via MRI-Proton density fat fraction (PDFF) were assessed as the treatment metric at baseline and at week 12.

Motivating the present study, a previously developed prototype model was deployed in an effort to discriminate, in a binary fashion, between responder and non-responder subjects (or patients) based on a relative reduction of 30% in % LF. Although preliminary, the promising results of from this classification approach indicated that metabolomics data (i.e., biochemical data) are potentially able to discriminate between responders and non-responders to Compound 001-152. As will be described below, different modeling techniques were used to develop algorithms for baseline determination of % LF change as a continuous variable, instead of the categorical classification previously established.

The predictive models described below were developed considering samples taken at baseline from patients allocated in a Compound 001-152-50 mg arm. Five patients were classified as ET (early termination) and EXT (patients who had week 12 between week 12 and week 16 due to COVID). Analyses described below were performed excluding patients classified as ET and/or EXT.

To obtain values of biochemical data for each patient, blood samples were collected. It can be appreciated that, in another instance, a serum sample could be collected into a serum collection tube. Plasma was prepared by incubation of venous blood from each patient in plasma separator tubes with ethylenediaminetetraacetic acid (EDTA) for 30 minutes before centrifugation at 2500 g for 15 minutes. Supernatants were aliquoted into microtubes and stored at −80° C. until metabolomic analysis.

Plasma samples were divided into aliquots and analyzed in three different metabolomic platforms.

### Metabolic Platforms

Platform 1: Methanol extract. Proteins were precipitated from defrosted plasma samples (75 μL) by adding 300 μL of methanol in 1.5 mL microtubes on ice. The extraction solvent was spiked with the following compounds not detected in non-spiked human plasma extracts: NEFA (19:0); tryptophan-d5 (indole-d5); dehydrocholic acid; and PC (13:0/0:0). After brief vortex mixing, the samples were incubated overnight at −20° C. Supernatants (300 μL) were collected after centrifugation at 16000 g for 15 minutes and solvent was removed. The dried extracts were then reconstituted in 120 μL of methanol, centrifuged (16000 g for 5 minutes), and transferred to vials for ultra performance liquid chromatography—tandem mass spectrometer (UPLC-MS/MS) analysis.

Platform 2: Chloroform/methanol extract. Proteins were precipitated from the defrosted plasma samples (10 μL) by adding 10 μL of sodium chloride (50 mM) and 110 μL of chloroform/methanol (2:1) in 1.5 mL microtubes on ice. The extraction solvent was spiked with the following compounds not detected in non-spiked human plasma extracts: SM (d18:1/6:0); PE (17:0/17:0); PC (19:0/19:0); TAG (13:0/13:0/13:0); TAG (17:0/17:0/17:0); Cer (d18:1/17:0); and ChoE (12:0). After brief vortex mixing, the samples were incubated for 1 hour at −20° C. After centrifugation at 16000 g for 15 minutes, 70 μL of the lower organic phase was collected and the solvent was removed. The dried extracts were then reconstituted in 100 μL acetronitrile/isopropanol (50:50), centrifuged (16000 g for 5 minutes), and transferred to vials for UPLC-MS analysis.

Platform 3: Amino acids. 10 μL aliquots of the extracts prepared according to Platform 1 were transferred to microtubes and derivatized for amino acid analysis.

### Liquid Chromatography—Mass Spectrometry Platforms

A UPLC-single quadrupole-MS amino acid analysis system was combined with two separate UPLC-time-of-flight (TOF)-MS based platforms analyzing methanol and chloroform/methanol plasma extracts. Each platform involves the use of a different UPLC-MS method.

Platform A: Chromatography was performed on a 1.0 mm i.d.×100 mm ACQUITY 1.7 μm C18 BEH column (Waters Corp., Milford, MA) using an ACQUITY UPLC system (Waters Corp., Milford, MA). The column was maintained at 40° C. and eluted with an 18 minute gradient. The mobile phase, at a flow rate of 140 μL/min, initially consisted of 100% solvent A (0.05% formic acid), with a linear increase of solvent B (acetonitrile containing 0.05% formic acid) up to 50% over two minutes, and a linear increase to 100% B over the next 11 minutes before returning to the initial composition in readiness for the subsequent injection which preceded a 45 second system recycle time. The volume of sample injected onto the column was 2 μL. The eluent was introduced into the mass spectrometer (LCT-PremierXE, Waters Corp., Milford, MA) by electrospray ionization, with capillary and cone voltages set in the negative ion mode to 2800 V and 50 V, respectively. The nebulization gas was set to 600 L/h at a temperature of 350° C. The cone gas was set to 30 L/h, and the source temperature was set to 120° C. Centroid data were acquired from m/z 50-1000 using an accumulation time of 0.2 seconds per spectrum.

Platform B: Chromatography was performed on a 2.1 mm i.d.×100 mm ACQUITY 1.7 μm C18 BEH column (Waters Corp., Milford, MA) using an ACQUITY UPLC system (Waters Corp., Milford, MA). The column was maintained at 60° C. and eluted with a 17 minute linear gradient of solvents A (water, acetonitrile and 10 mM ammonium formate) and B (acetonitrile, isopropanol and 10 mM ammonium formate). The mobile phase, at a flow rate of 400 μL/min, initially consisted of 40% solvent B, increasing up to 100% at 10 minutes. After 5 minutes the mobile phase was reset to the initial composition in readiness for the subsequent injection which preceded a 45 second system recycle time. The volume of sample injected onto the column was 3 μL. The eluent was introduced into the mass spectrometer (Acquity-Xevo G2 QTof, Waters Corp., Milford, MA) by electrospray ionization, with capillary and cone voltages set in the positive ion mode to 3200 V and 30 V, respectively. The nebulization gas was set to 1000 L/h at a temperature of 500° C. The cone gas was set to 30 L/h, and the source temperature was set to 120° C. Centroid data were acquired from m/z 50-1000 using an accumulation time of 0.2 seconds per spectrum.

Platform C: Analytes were separated by means of a gradient of solvents A (water 10 mM NH4HCO3, pH=8.8 adjusted with ammonium hydroxide 28% in water) and B (acetonitrile). Flow was 0.140 μL/min. Gradient started with 98% of A that decreased linearly to 92% at minute 6.5, 80% at minute 10, 70% at minute 11, and 0% at minute 12. After 2 minutes the mobile phase was reset to the initial composition in readiness for the subsequent injection which preceded a 45 second system recycle time. The eluent was introduced into the mass spectrometer (SQD, Waters Corp., Milford, MA) by electrospray ionization, with capillary and cone voltages set in the positive ion mode to 3200 V and 30 V, respectively. The nebulization gas was set to 600 L/h at a temperature of 350° C. The cone gas was set to 10 L/h, and the source temperature was set to 120° C. Selected ion recording (SIR) was used to analyze desired metabolites. An appropriate test mixture of standard compounds may be analyzed along the entire set of randomized sample injections in order to examine the retention time stability, mass accuracy and sensitivity of the system throughout the course of the run which lasted a maximum of 48 hour per batch of samples injected.

Online tandem mass spectrometry (MS/MS) experiments for metabolite identification were performed on a Acquity-SYNAPT G2 system and a Xevo G2 QTof system (Waters Corp., Milford, MA) operating in both the positive and negative ion electrospray modes. Source parameters were identical to those employed in the profiling experiments, except for the cone voltage which was increased (30-70 V) when pseudo MS/MS/MS data was required. During retention time, windows corresponding to the elution of the compounds under investigation, the quadrupole was set to resolve and transmit ions with appropriate mass-to-charge values. The selected ions then traversed an argon-pressurized cell, with a collision energy voltage (typically between 5 V and 50 V) applied in accordance with the extent of ion fragmentation required. Subsequent TOF analysis of the fragment ions generated accurate mass generally <3 ppm MS/MS or pseudo MS/MS/MS spectra corrected in real time by reference to leucine enkephalin, infused at 10 μL/min through an independent reference electrospray, sampled every 10 seconds.

LC-MS features (as defined by retention time, mass-to-charge ratio pairs; Rt-m/z), were associated with fatty acids (FA), N-acylethanolamines (NAE), and oxidized fatty acids by comparison of their accurate mass spectra and chromatographic retention times in the extracts with those obtained using available reference standards. A metabolic feature with m/z value between 400 and 1000 Da was considered unambiguously identified when Rt difference with respect to the standard was smaller than 3 s and the deviation from its m/z value (δm/z) smaller than 3 ppm. For metabolites with m/z values smaller than 400 Da, the criterion followed with respect to Rt was the same but setting the δm/z limit to 1.2 mDa. The ion features considered for the following data analysis were the adducts [M−CO2H]−, [M−CO2H]− and [M−H]− for FA, NAE and oxidized FA, respectively. The nomenclature C:Dn-x is used for these fatty acyl species, where C is the number of carbon atoms, and D is the number of double bonds in the fatty acid chains. A double bond is located on the xth carbon-carbon union, counting from the terminal methyl group towards the carbonyl carbon. However, since the physiological properties of unsaturated fatty acids largely depend on the position of the first unsaturation relative to the end position and not the carboxylate, the location is signified by ω or “n” minus the unsaturation location, i.e., 20:5 (n-3). An “x” in the name of some FA indicates the unknown position the double bounds, as reference standards for full identification were not available.

For diglycerides (DG), triglycerides (TG), cholesteryl esters (ChoE), glycerophosphatidylcholines (PC), glycerophosphatidylethanolamines (PE), glycerophosphatidylinositols (PI), sphingomyelins (SM), ceramides (Cer), and monohexosylceramides (CMH) species, a theoretical m/z database was first generated for all possible combinations of fatty acid derived moieties. The association of detected Rt-m/z pairs with lipid species contained in the theoretical database was subsequently established either by comparison of their accurate mass spectra and chromatographic retention times with those obtained using available reference standards or, where these were not available, by accurate mass MS/MS fragment ion analysis, as described in detail previously (Barr 2010, Murphy, R C, Fiedler J, Hevko J. 2001. Analysis of Nonvolatile Lipids by Mass Spectrometry. Chem. Rev. 101: 479-526).

The ion features considered for the following data analysis of these lipid classes were the adducts [M−CO2H]− for monoacyl- and monoether-PC; [M−H]− for monoacyl- and monoether-PE and PI; [M+H]+ for diacyl- or 1-ether,2-acyl-PC, PE and PI, CMH and SM; [M+H—H2O]+ for Cer; [M+Na]+ for DG; [M+NH4]+ for ChoE and TG. The nomenclature for these lipid species follows the rules: (i) Glycerolipids species (DG and TG): C:D nomenclature is used, where C is the number of carbon atoms and D is the number of double bonds considering all the acyl chains esterified to glycerol. The position of the double bonds in the acyl chains is not considered, as well as the position of the acyl chains; (ii) ChoE: C:D nomenclature is used, where C is the number of carbon atoms and D is the number of double bonds in the acyl chains esterified to the cholesterol. The position of the double bonds in the acyl chains is not considered; (iii) Sphingolipids species (SM, Cer and CMH): sA:B/C:D nomenclature is used, where sA:B represents the sphingoid base: s18:1, sphingosine; s18:2, sphingadiene; s18:0, sphinganine. C:D indicates the number of carbon atoms C, and double bonds D, contained in the N-linked fatty acid. The position of the double bonds in the acyl chains is not considered; and (iv) Glycerophospholipids (PC, PE, and PI) as diacyl, 1-acyl, 2-acyl, 1-ether or 1-ether, 2acyl-linked species: A:B/C:D nomenclature is used, where A:B and C:D refer to the number of carbon atoms and number of double bonds contained in the sn-1 and sn-2 side chains, respectively. For glycerophospholipids containing an ether moiety the prefix, O—, denotes the presence of an alkyl ether (plasmanyl) substituent, i.e., PC(O-16:0/16:0), whereas the prefix P—refers to a vinyl ether (alkenyl or plasmenyl) substituent, i.e., PC(P-16:0/16:0). The suffix, e, indicates the presence of an ether linked substituent, although plasmanyl or plasmenyl classification has not been confirmed. X:Y nomenclature (where X=A+C and Y=B+D) is used where evidence was found for the contribution of multiple species to a single chromatographic peak. The position of the double bonds in the acyl chains is not considered.

### Data Pre-Processing

All data were pre-processed using the TarkerLynx application manager for MassLynx 4.1 software (Waters Corp., Milford, MA). The complete set of predefined Rt-m/z pairs was fed into the software, which generated associated extracted ion chromatograms (mass tolerance window=0.05 Da), peak-detected and noise-reduced in both the LC and MS domains. A list of intensities (chromatographic peak areas) was then generated for each sample injection, using the Rt-m/z pairs (retention time tolerance=6 s) as identifiers. Intra- and inter-batch normalization was performed. This process involved (i) internal standard response correction (intra-batch normalization) and (ii) variable specific inter-batch single point external calibration using repeat extracts of a commercial serum sample (inter-batch normalization).

### Data Analysis

All analyses were performed with JupyterLab 2.2.8 and Python. For reproducible research purposes, the hardware information and libraries used are described in Table 2.

TABLE 2
Hardware information and principal libraries.
Hardware Information

| Hardware | Version |
|---|---|
| CPython | 3.7.8 |
| IPython | 7.18.1 |
| JupyterLab | 2.2.8 |

Principal Libraries

| Library | Version |
|---|---|
| pandas | 1.1.3 |
| numpy | 1.20.3 |
| scipy | 1.5.2 |
| matplotlib | 3.3.2 |
| seaborn | 0.11.0 |
| sklearn | 0.24.2 |
| pingouin | 0.3.8 |
| joblib | 0.17.0 |

### Correlation Analysis

In statistics, the correlation is a statistical relationship between two random variables. Correlations are useful because they can show a predictive relationship. However, it must be remembered that the presence of a significant correlation is not sufficient to ensure causation. There are several correlation coefficients measuring the degree of correlation. The most common is the Pearson's correlation coefficient, which is sensitive only to a linear relationship between two variables. The Spearman's rank correlation is less sensitive to nonlinear relationships and this coefficient can be considered more robust than Pearson's correlation coefficient. Pearson's correlation coefficient is defined as

$$ρ ( X , Y ) = corr ( X , Y ) = \frac{cov ( X , Y )}{σ X σ Y}$$

while Spearman's rank correlation is defined as

$$rs = 1 - \frac{6 \sum {d}_{i}^{2}}{n ( {n}^{2} - 1 )} ,$$

where dᵢ is the difference between the two ranks of each observation, or dᵢ=rg(Xᵢ) −rg(Yᵢ), and the parameter n is the number of observations.

Both coefficients have a rank from −1 (perfect inverse correlation) to 1 (perfect correlation), but while Pearson's correlation assesses linear relationships, Spearman's correlation assesses monotonic relationships (whether linear or not). The significance of both coefficients has been calculated. A value p<0.05 means a significant relationship.

In this study, a series of correlations of % LF and biochemical data were calculated. These correlations were calculated considering raw data at baseline time.

### Correlation Versus Percentage of Change

In addition, the change between baseline and week 12 was calculated per variable as

$$\% = \frac{Week 12 - Baseline}{Baseline} \times 100$$

Further correlation analyses with % LF change were performed using this percentage of change.

### Data Modeling

In view of methods 100, 200, and 250, a predictive model for the determination of the % LF change was developed based on the biochemical data to obtain a companion diagnostic of patients that will respond to Compound 001-152-50 mg treatment.

Two different machine learning regression algorithms, a Random Forest algorithm and a Support Vector Machine algorithm, were tested in the study. A random cross-validation procedure was used to evaluate the estimator performance. In other words, models were built with a certain percentage of samples and then they were validated with the remainder of the samples, a process that was repeated 50 times. The performance of the models was assessed and optimized by calculating (and/or minimizing) the Root Mean Square Error (RMSE) of the residuals, the difference between the predicted values and the measured values. RMSE is an absolute measure of fit, being in the same units as the response variable. The lower the values of RMSE the better the fit is. RMSE is defined as

$$RMSE = \sqrt{\frac{{\sum }_{i = 1}^{N} {( {E}_{i} - {O}_{i} )}^{2}}{N}}$$

where Eᵢ is the expected value for i sample and Oᵢ is the observed value for i sample.

In order to deploy a Support Vector Machine algorithm, the data must be scaled. This is, individual features were standardized (centered and scaled) by removing the mean and scaling to unit variance,

$$\tilde{X} = \frac{X - mean ( X )}{SD ( X )}$$

Feature importance was calculated using an adapted permutation importance algorithm. The permutation importance algorithm is described in Breiman, L. Random Forests. Machine Learning 45, 5-32 (2001). https://doi.org/10.1023/A:1010933404324.

In order to compute a reference score s (RMSE) of a model m on data D, the following are performed for each feature fⱼ (metabolite or biochemical parameter). For each repetition k in 1, 2, 3, . . . , K, variable fⱼ of data D is randomly shuffled (new version of D named D_{k,j}) and a score S_{k,j} of the model is calculated over the D_{k,j}. Importance iⱼ for each feature fⱼ can then be computed as

$${i}_{j} = S - \frac{1}{K} ( {\sum }_{k = 1}^{K} {S}_{k , j} ) .$$

### Results and Discussion

### Correlation Results

Results of Pearson and Spearman correlation coefficients for the best 10 features are listed in Table 3 and Table 4, respectively. n is the number of paired values in the correlation analysis, r is the Pearson's or Spearman's correlation coefficient, CI95% is a 95% confidence interval for the Pearson's or Spearman's r, r² is the square of the Pearson's or Spearman's correlation coefficient, adj_r² is the r² that has been adjusted for the number of predictors in the model, and p-val is the vp-value or significance of the correlation and represents the probability that the correlation occurred by chance. Each of the Features of Table 3 and Table 4 can be understood with reference to Table 1.

TABLE 3
Pearson's Coefficient Correlation (r²) and related p-values (p-val) for the correlation
between each feature and % liver fat change. The 10 best features are shown.

| Feature | n | r | CI95% | r² | p-val |
|---|---|---|---|---|---|
| D(−)-2-Aminobutyric | 28 | −0.422059 | [−0.69, −0.06] | 0.178134 | 0.025273 |
| acid (AA53) | | | | | |
| DL-2-Aminocaprylic | 28 | −0.390845 | [−0.67, −0.02] | 0.152760 | 0.039736 |
| acid (AA35) | | | | | |
| PE(18:3/0:0) (MAPE22) | 28 | 0.388044 | [0.02, 0.66] | 0.150578 | 0.041304 |
| isomer androsterone | 28 | 0.383133 | [0.01, 0.66] | 0.146791 | 0.044172 |
| sulfate (ST09) | | | | | |
| 18:2n-6 (FFA11) | 28 | −0.362019 | [−0.65, 0.01] | 0.131058 | 0.058349 |
| 20:2n-6 (FFA19) | 28 | −0.358735 | [−0.65, 0.02] | 0.128691 | 0.060841 |
| 20:4n-3 (FFA25) | 28 | −0.347846 | [−0.64, 0.03] | 0.120997 | 0.069704 |
| 20:1n-6 (FFA17) | 28 | −0.332696 | [−0.63, 0.05] | 0.110687 | 0.083654 |
| 19:1n-x (FFA35) | 28 | −0.330669 | [−0.63, 0.05] | 0.109342 | 0.085671 |
| Taurocholic acid (BA09) | 28 | 0.324886 | [−0.05, 0.62] | 0.105551 | 0.091629 |

TABLE 4
Spearman's Coefficient Correlation (r²) and related p-values (p-val) for the correlation
between each feature and % liver fat change. The 10 best features are shown.

| Feature | n | r | CI95% | r² | adj_r² |
|---|---|---|---|---|---|
| TG(40:0) (TG121) | 28 | 0.454297 | [0.1, 0.71] | 0.206385 | 0.015159 |
| AC(8:1n-x) (AC07) | 28 | −0.443897 | [−0.7, −0.08] | 0.197045 | 0.017969 |
| Taurochenodeoxycholic | 28 | 0.427477 | [0.06, 0.69] | 0.182736 | 0.023268 |
| acid (BA11) | | | | | |
| PC(O-22:0/20:4) | 28 | 0.417625 | [0.05, 0.68] | 0.174410 | 0.027017 |
| (MEMAPC37) | | | | | |
| PE(34:2) (DAPE03) | 28 | 0.414888 | [0.05, 0.68] | 0.172132 | 0.028141 |
| TG(42:1) (TG82) | 28 | 0.403941 | [0.04, 0.68] | 0.163168 | 0.033023 |
| PC(O-20:0/20:4) | 28 | 0.397373 | [0.03, 0.67] | 0.157905 | 0.036266 |
| (MEMAPC31) | | | | | |
| PC(O-22:1/20:4) | 28 | 0.390805 | [0.02, 0.67] | 0.152728 | 0.039758 |
| (MEMAPC35) | | | | | |
| PC(28:0) (DAPC46) | 28 | 0.388068 | [0.02, 0.66] | 0.150597 | 0.041291 |
| DL-2-Aminocaprylic acid | 28 | −0.386973 | [−0.66, −0.02] | 0.149748 | 0.041917 |
| (AA35) | | | | | |

### Feature Selection

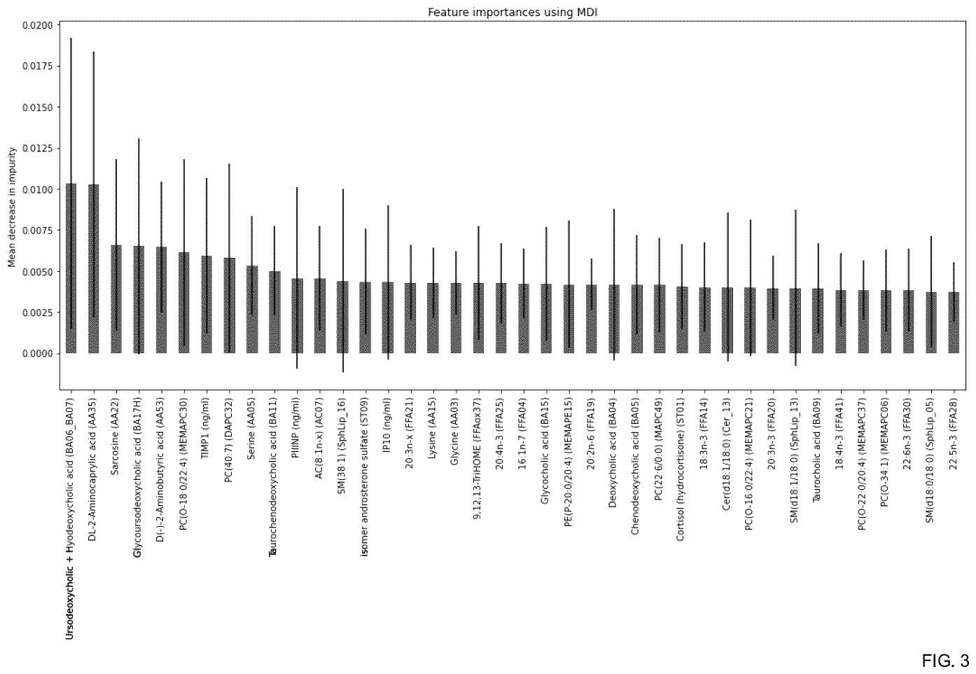

A feature selection step was performed in order to select the best set of variables to predict % LF change, and, therefore, to be included in a final machine learning model. “mean decrease in impurity” (MDI), a built-in method of a Random Forest algorithm, was used to evaluate the importance of features. In this method, feature importance is computed as the mean decrease in impurity, or the sum over the number of splits (across all trees) that include the feature, proportionally to the number of samples it splits. The most important features are shown in FIG. 3A, wherein the blue bars indicate the average of importance and black lines indicate the deviation of the measure.

To find the best features, a Random Forest regressor with 10,000 estimators (number of trees in the forest) and the importance (as mean decrease in impurity score) was evaluated. This process was repeated 10 times. As a result, it was determined that the two important features are ursodeoxycholic+hyodeoxycholic acid (Variable ID: BA06_BA07) and DL-2-Aminocaprylic acid (Variable ID: AA35).

### Regression Models

Regression is a supervised machine learning process. Regression models are similar to classification models, but rather than predicting a label, the algorithm is developed to predict a continuous value. Based on the previously selected features, two different machine learning algorithms, a Random Forest algorithm and a Support Vector Machine (SVM) algorithm were tested to predict % LF change, using a random cross-validation procedure. A recursive feature aggregation was used to fit a model to find the best model.

i. Random Forest

A Random Forest algorithm is a meta estimator that fits a number of individual classifying decision trees, each trained with a random sub-sample extracted from the original training data by bootstrapping.

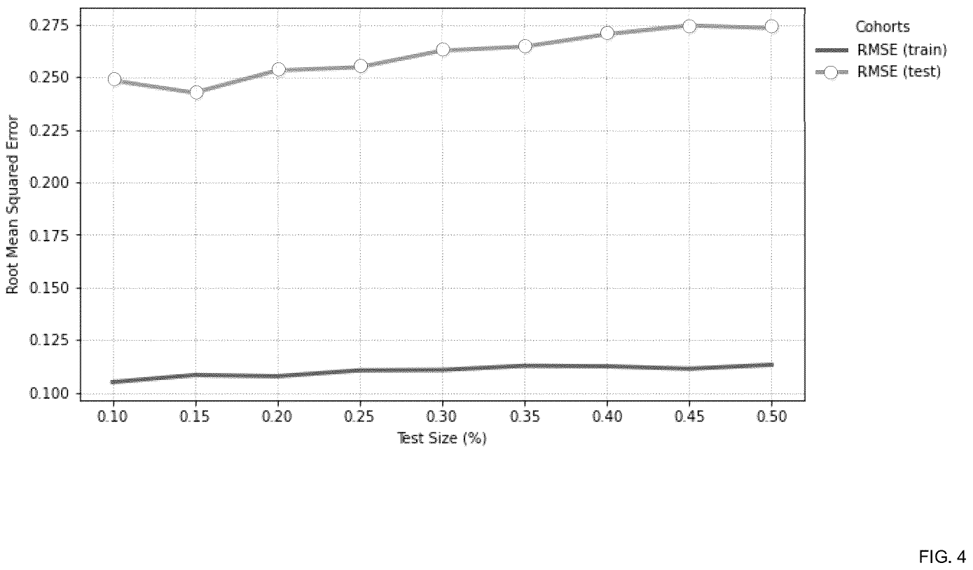

A first step of model selection is analysis of possible effects produced by a number of samples used in training and validation. As mentioned, models were built with a certain percentage of samples and then they were validated with the rest of the samples (i.e., cross-validation). FIG. 4 shows the estimated RMSE value obtained in a training cohort and a validation cohort as a function of the test size. When the size of the validation cohort, or the test size, is 10%, that the model has been trained with 90% of the cohort. The observed effect is the expected one—a lower value of RMSE in the training cohort and a higher value in the validation cohort. Due to these results, a 50%/50% training cohort to validation cohort is selected as the best approach to avoid bias. Models were then constructed with 50% of the samples and then validated with the remaining 50%. The process was repeated 50 times.

In order to select the best results, the search of the optimum model was performed including different number of features. In some embodiments, the search of the optimum model started from the five most important features and reached up to 50 features. RMSE was evaluated once a feature was added, being always added one feature at a time.

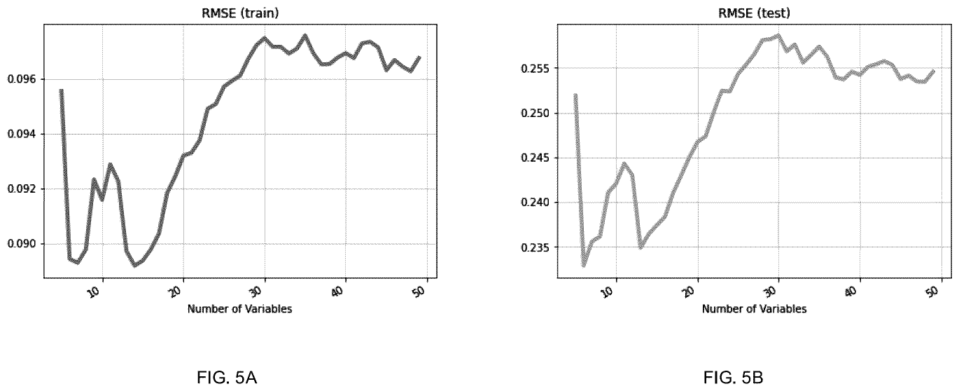

The mean values of RMSE in the training cohort (or train cohort) and the validation cohort (or test cohort) for the obtained models per number of variables considered in the model are shown in FIG. 5A and FIG. 5B. Best results were obtained with six features. There is an overfitting of the model once over 15 features were added.

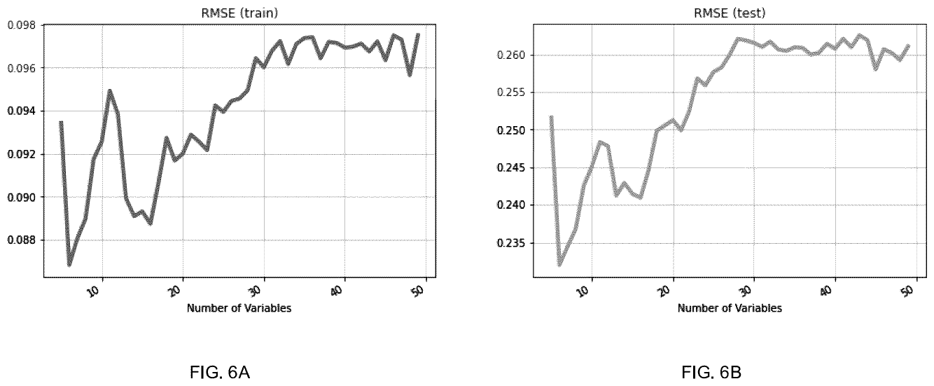

As the median is a robust measure of central tendency, the same graphical representations were generated for the median values of RMSE, as ween in FIG. 6A and FIG. 6B. As in the previous representation, best results were obtained considering six features (see Table 5).

TABLE 5
List of biomarkers included in the model.

| Metabolite | Variable ID | Class | HMDB code |
|---|---|---|---|
| Ursodeoxycholic + | BA06_BA07 | Bile acids | HMDB00946 |
| Hyodeoxycholic acid | | | |
| DL-2-Aminocaprylic acid | AA35 | Amino Acids | HMDB00991 |
| | | Derivative | |
| Sarcosine | AA22 | Amino Acids | HDB00271 |
| Glycoursodeoxycholic acid | BA17H | Bile acids | HMDB00708 |
| D(−)-2-Aminobutryic acid | AA53 | Amino Acid derivative | HMDB00650 |
| PC(O-18:0/22:4) | MEMAPC30 | Glycerophospholipids | — |

The distribution of RMSE values for each number of variables is shown in FIG. 7A and FIG. 7B.

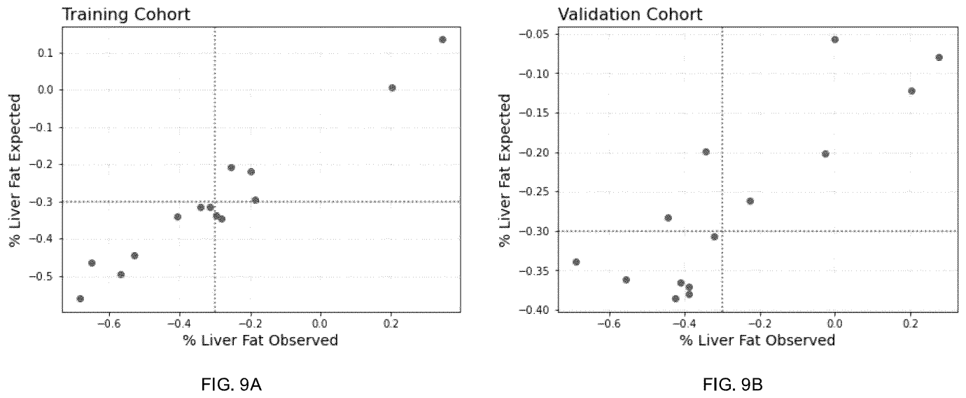

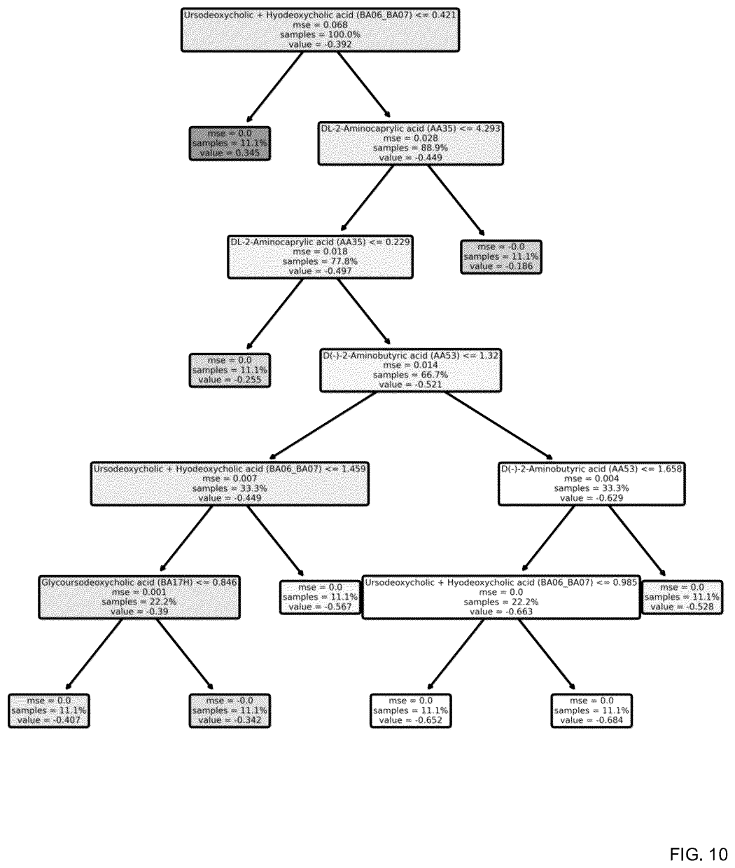

After development, the Random Forest algorithm was implemented to predict % LF. The RMSE was the metric used to optimize the models, and a correlation analysis was performed to evaluate the relationship between % LF change observed and the % LF change expected (i.e., model predicted value). This correlation is shown in Table 6 and the same are graphically represented in FIG. 9A and FIG. 9B. FIG. 10 shows an exemplary decision structure of a Random Forest algorithm used in the present disclosure.

TABLE 6
Correlation between % Liver Fat Observed and % Liver
Fat Expected for training and validation cohort.

| Cohort | n | r | CI95% | r² | adj_r² | p-val |
|---|---|---|---|---|---|---|
| Training | 14 | 0.978 | [0.93, | 0.956 | 0.948 | 1.497e−09 |
| | | | 0.99] | | | |
| Validation | 14 | 0.854 | [0.59, | 0.730 | 0.681 | 0.000099 |
| | | | 0.95] | | | |

ii. Support Vector Machine

Support Vector Machine (SVM) algorithms try to find a line/hyperplane in multidimensional space that separates two classes. Support Vector Regression (SVR) uses the same principle as SVM, but for regression problems.

Standardization of a dataset is a common requirement for machine learning estimators to avoid undesired behavior when individual features are not normally distributed. Here, individual features were standardized (centered and scaled) by removing the mean and scaling to unit variance. Three kernels have been used: linear, polynomial, and radial. The polynomial kernel has been applied considering three different degrees: degree 2, degree 3, and degree 4.

As a first step, the five most important variables for building the algorithm were used as a starting point. Then, subsequent variables were added one by one to the algorithm according to the order of importance (see FIG. 3), up to a maximum of 50 variables. As performed in the Random Forest algorithm-based model, SVM models were built in a training cohort and test with the remaining samples comprising a validation cohort. RMSE values were calculated in both cohorts. A 50%/50% cross-validation was used and each analysis was repeated 50 times to have a robust estimate of the RMSE value.

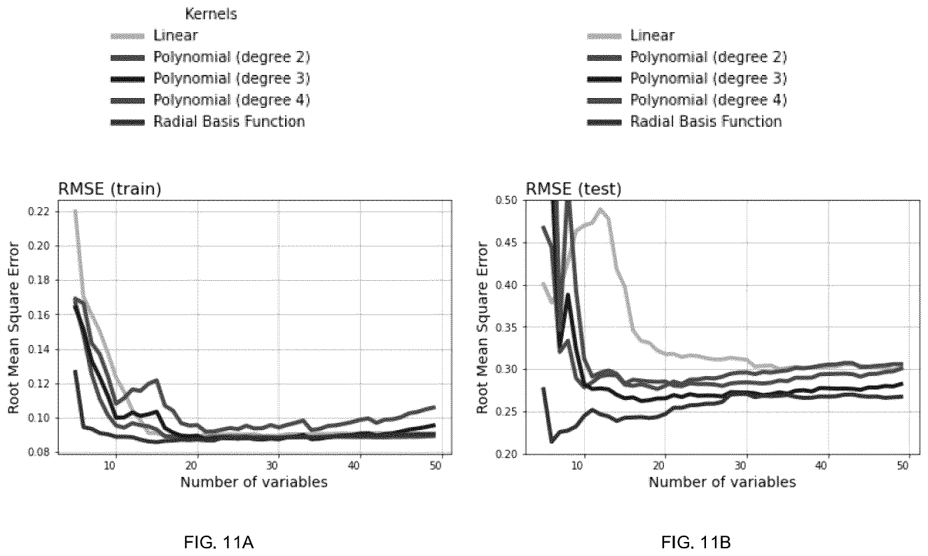

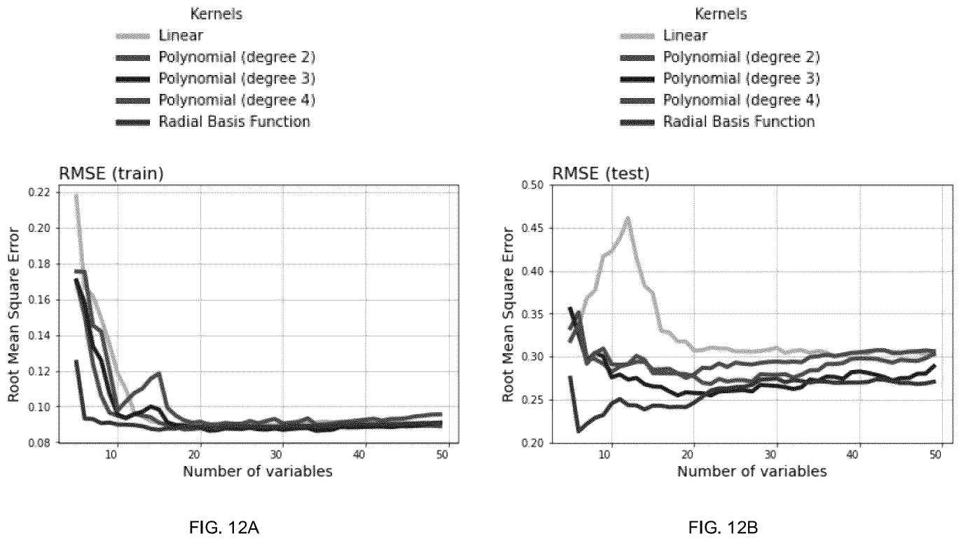

Analysis of the RMSE value by cross-validation based on the number of variables was carried out by kernel. The results of these analyses as mean and median values are shown in FIGS. 11A, 11B, 12A, and 12B. In each case, an optimal value (minimum value of the RMSE in the validation cohort) can be observed for six variables. The best results (minimum values for the RMSE) were achieved for the radial kernel. As expected, the RMSE for the validation cohort decreases for a low number of variables (when the algorithm is learning) and increases for a high number of variables (when the algorithm is overfitting).

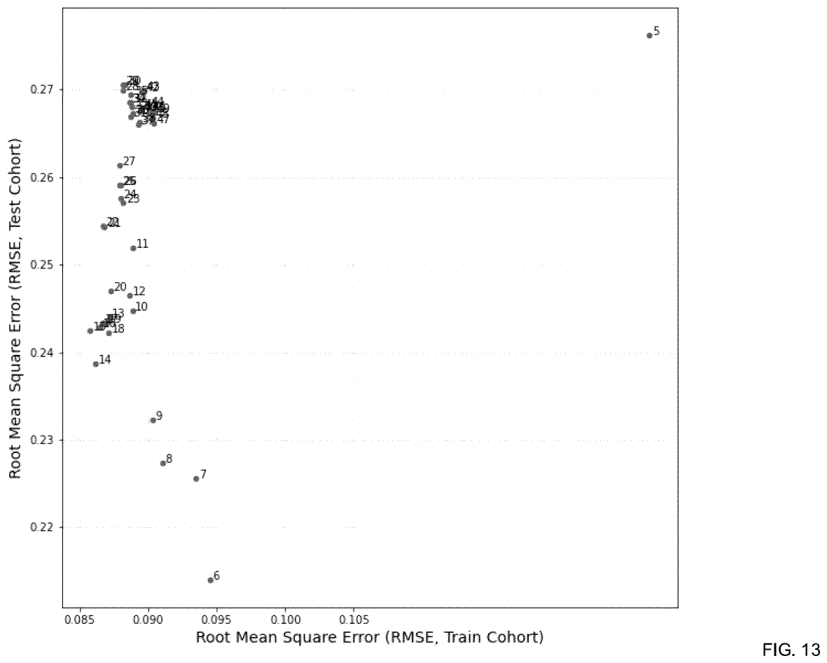

FIG. 13 shows the relationship between RMSE values in the training cohort and in the validation cohort. The model built with six features achieved the minimum RMSE in the training cohort. In FIG. 13, the training cohort is on the x-axis and validation cohort is on the y-axis. Each point on the graph indicates results obtained in one model constructed with N variables (label).

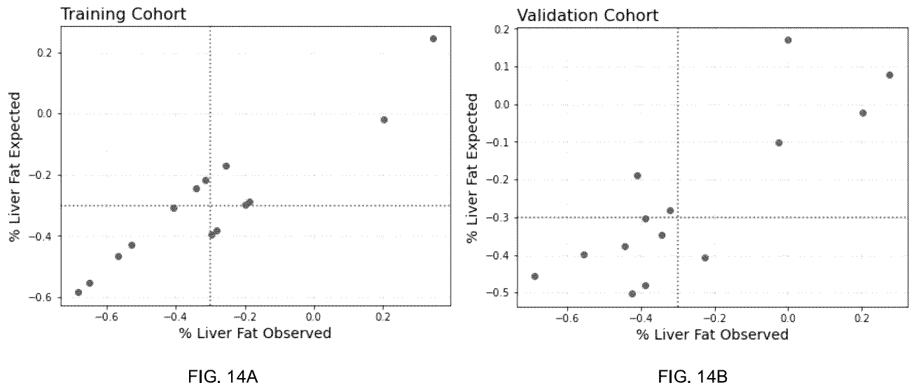

Using RMSE as an optimization metric, a correlation analysis was performed to evaluate the relationship between % LF observed and the % LF expected (as predicted by the SVM model). These results are shown in Table 7. The relationship between observed values and predicted values of % LF change (in the training cohort and in the validation cohort) are shown in FIG. 14A and FIG. 14B.

TABLE 7
Correlation between % Liver Fat Observed and % Liver
Fat Expected for training and validation cohort.

| Cohort | n | r | CI95% | r² | adj_r² | p-val |
|---|---|---|---|---|---|---|
| Training | 14 | 0.937 | [0.81, | 0.879 | 0.857 | 7.412e−07 |
| | | | 0.98] | | | |
| Validation | 14 | 0.835 | [0.55, | 0.698 | 0.643 | 0.197 |
| | | | 0.95] | | | |

### Common Results

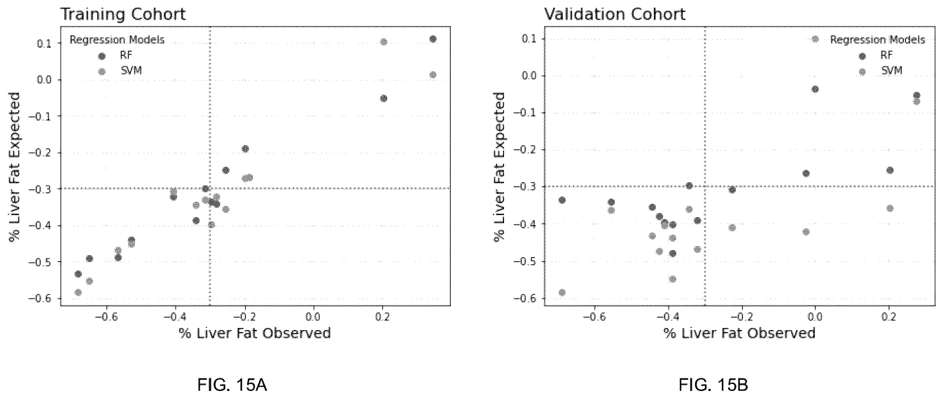

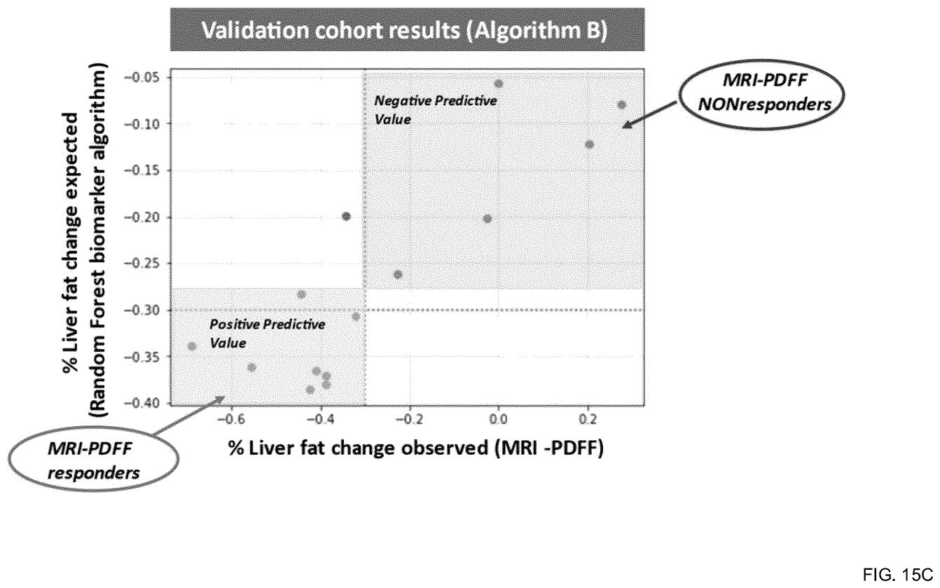

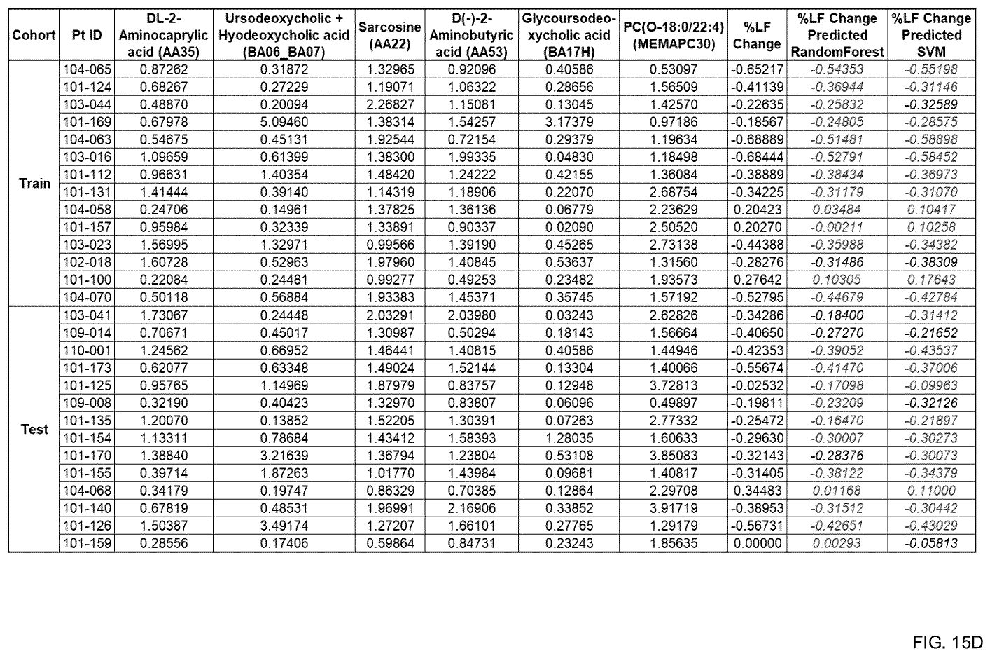

Predictions made by both of the Random Forest algorithm-based model and the SVM algorithm-based model are displayed for visual comparison in FIG. 15A and FIG. 15B. Tabular representations of a portion of these results are shown in FIG. 15D. For instance, in FIG. 15D, for each training cohort member and each validation cohort member, a % LF change was predicted using each of the Random Forest algorithm-based model and the SVM algorithm-based model. Columns between ‘Pt ID’ (i.e., Patient ID) and ‘% LF Change’ (i.e., observed % LF change) list the variables, and values thereof, deployed for each model. These variables are the same biochemical markers described above with reference to Table 5.

In FIG. 15A, correlation between % LF observed and % LF expected (model predicted value) for the training cohort is shown. Each observation is plotted twice −one from the Random Forest prediction (blue dots) and the other from the SVM prediction (orange dots). In FIG. 15B, a correlation between % LF observed and % LF expected (model predicted value) for the validation cohort is shown. Each observation is plotted twice −one from the Random Forest prediction (blue dots) and the other from the SVM prediction (orange dots). In FIG. 15C, a correlation between % LF observed and % LF expected, as predicted by a Random Forest algorithm-based model (“Algorithm B”), is shown. As in FIG. 15C, a lower left quadrant of the correlation indicates points where the expected and observed % LF suggest a responsive patient, while an upper right quadrant of the correlation indicates points where the expected and observed % LF suggest a non-responsive patient.

### ROC Analysis

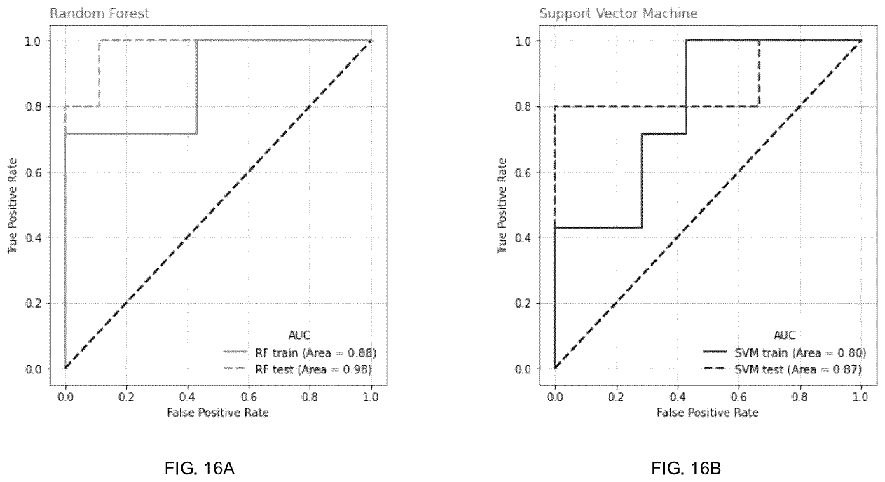

The Random Forest algorithm-based model and the SVM algorithm-based model were evaluated through a receiver-operating characteristic (ROC) analysis considering % LF as a categorical variable with a cutoff at −0.3 (considering responders those patients whose relative reduction in % LF is greater than 30%). Four ROC curves (each model in the training cohort and the validation cohort) are shown in FIG. 16A and FIG. 16B.

### Comparison of Results Against Previous Models

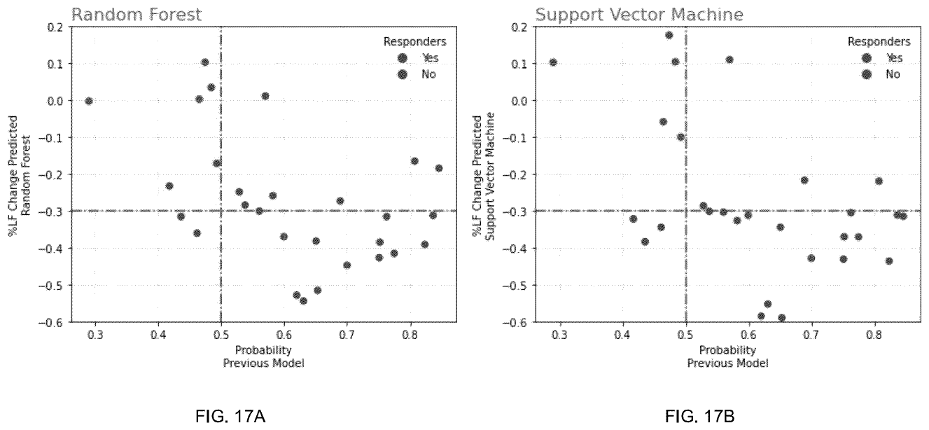

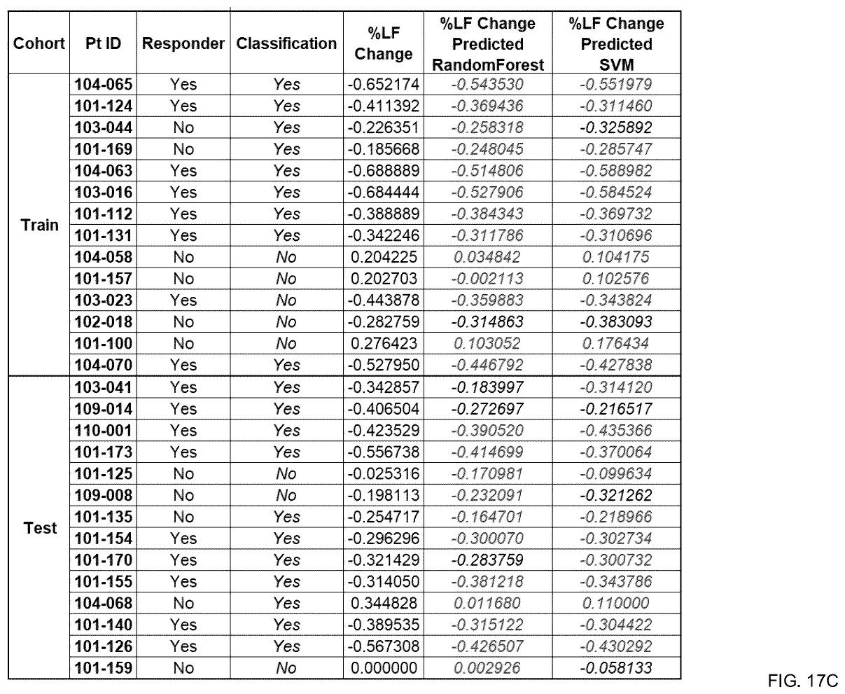

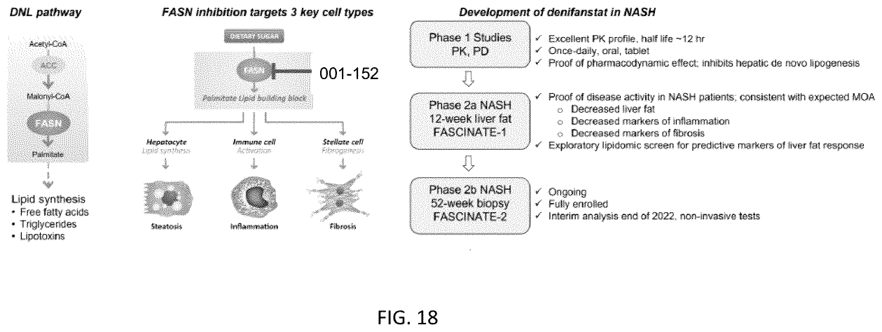

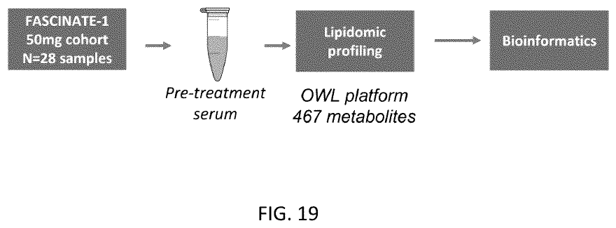

Introduced above, a classification-based model was previously developed in order to discriminate between responder and non-responder patients based on a relative reduction of 30% on % LF. As shown in FIG. 17A and FIG. 17B, and as tabulated in FIG. 17C, predictions of this classification-based model were compared to predictions of the Random Forest algorithm-based model and the SVM algorithm-based model. In other words, FIG. 17C shows a tabulated comparison between the continuous value-based model predictions generated via the methods of the present disclosure and classification-based model predictions. FIG. 17A illustrates % LF predicted by the Random Forest algorithm-based model and probability from the classification-based model (categorical response based on a 30% reduction in % LF). Each point is an observation and are colored according to the response to treatment (responders: green; non-responders: red). FIG. 17B illustrates % LF predicted by the SVM algorithm-based model and probability from the classification-based model (categorical response based on a 30% reduction in % LF). Each point is an observation and are colored according to the response to treatment (responders: green; non-responders: red). The same process was applied for placebo samples and samples treated with 25 mg.

### Conclusions

Two predictive models for baseline determination of % LF change in patients receiving 50 mg of Compound 001-152 in the Phase 2a Study FASCINATE-1 were developed with two different methodologies: Random Forest and Support Vector Machine. The development of the algorithms has been based on optimizing the RMSE value. A cross-validation process was performed in view of the sample size. This process demonstrated that there is no bias for the sample size used in training. A rigorous evaluation process of the importance of the different variables was considered and models were generated through a variable aggregation process.

### Example 2: A Baseline Metabolomic Signature Predicts Liver Fat Response to Compound 001-152, a First-In-Class, Fatty Acid Synthase (Fasn) Inhibitor: Analysis in Fascinate-1 and Fascinate-2 Clinical Studies

The study described herein was conducted to evaluate baseline lipidomic markers that predict liver fat response to FASN inhibitors of the disclosure and to explore the effect of FASN inhibitors of the disclosure on the circulating lipidome.

### Methods

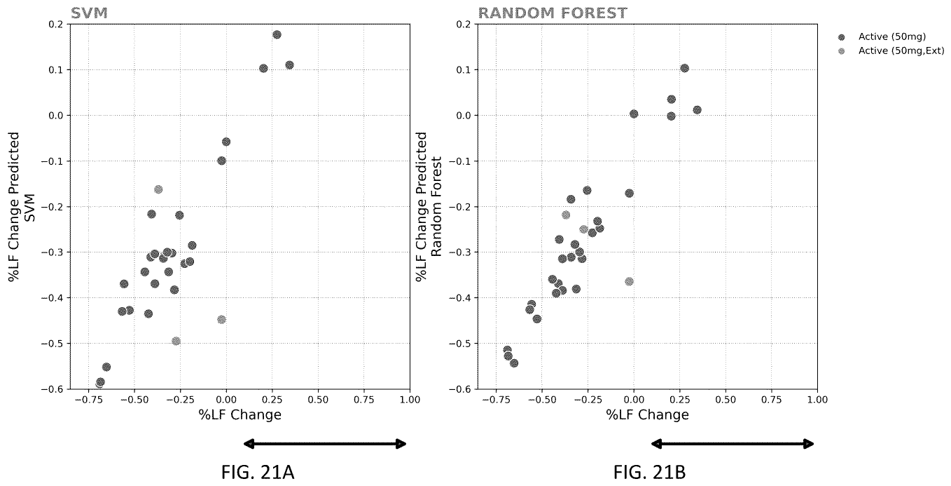

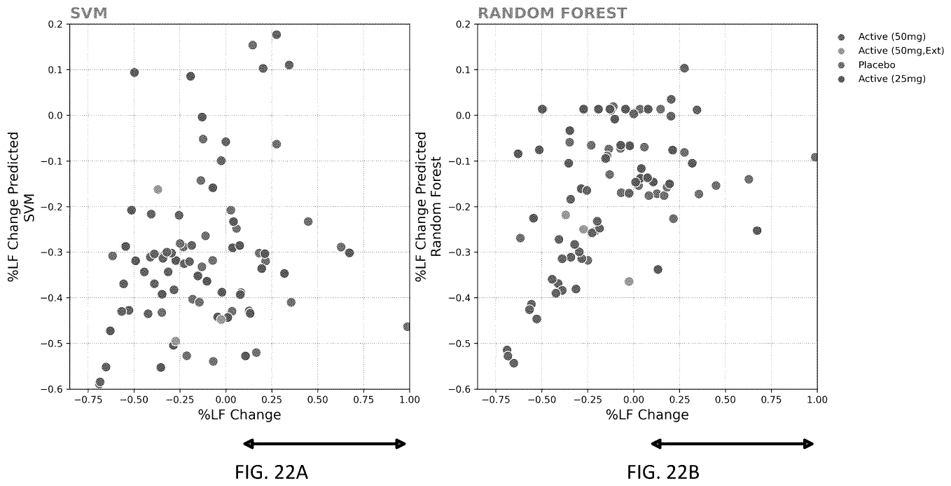

Baseline blood samples from FASCINATE-1 were profiled for ˜470 metabolites by LC/MS/MS. Metabolomic results from the 50 mg compound 001-152 group (n=34) were analyzed using two different nonlinear regression machine learning algorithms (Random Forest (RF) and Support Vector Machine (SVM)) (FIG. 21 and FIG. 22), to identify a biomarker panel that predicted liver fat response as measured by Magnetic Resonance Imaging Proton Density Fat Fraction (MRI-PDFF). Samples were split into training and validation cohorts with 50× cross-validation using liver fat (LF) as a continuous variable. Blood samples collected at 4 and 12 weeks of compound 001-152 treatment were profiled to evaluate changes in the lipidome with compound 001-152 treatment.

### Results

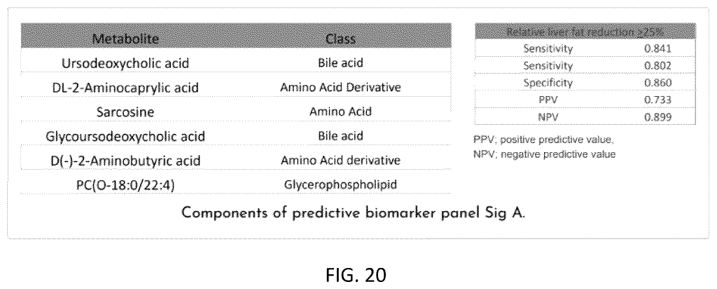

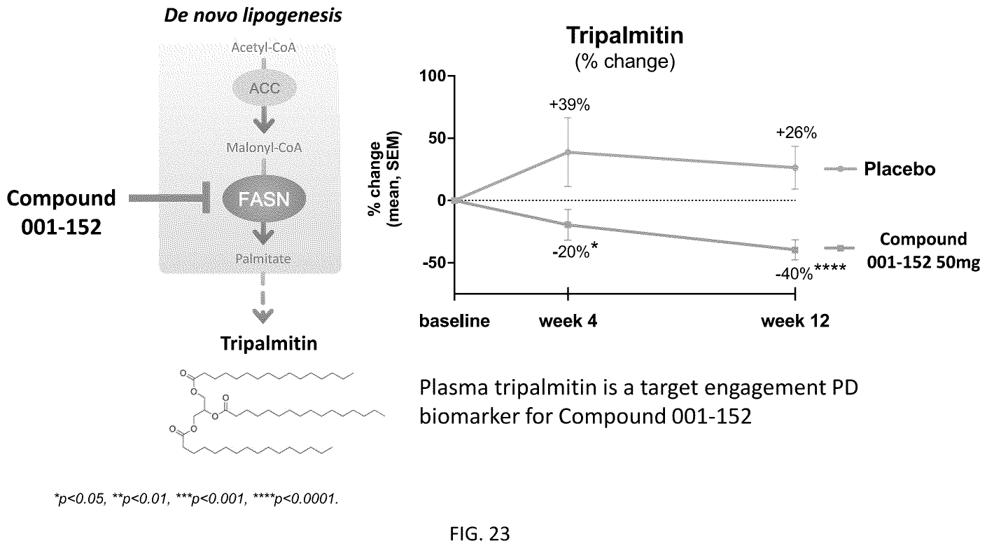

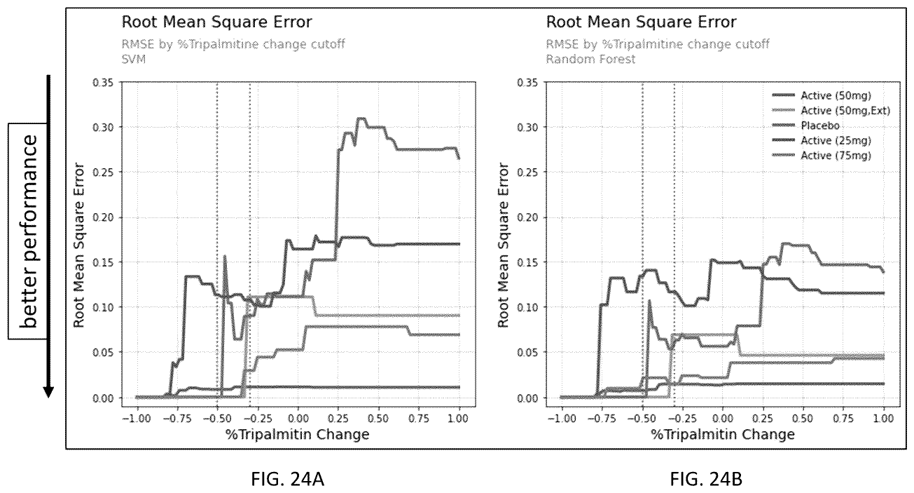

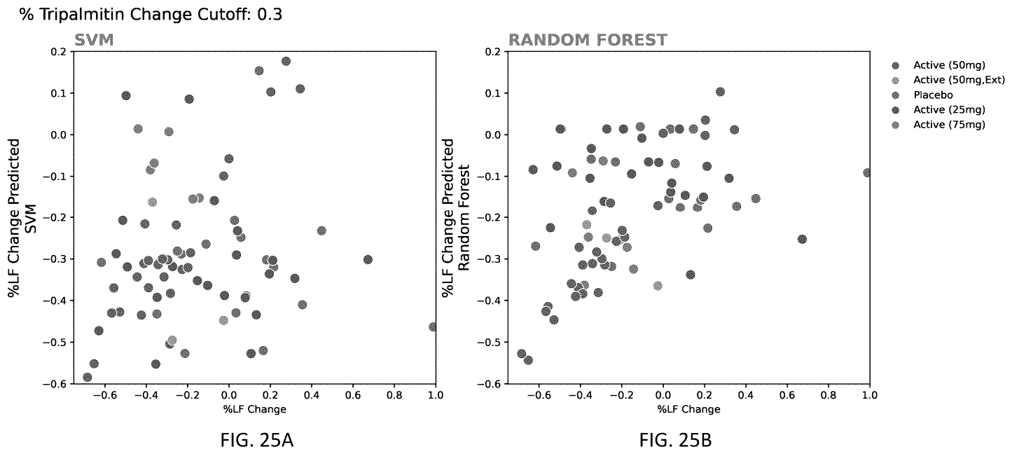

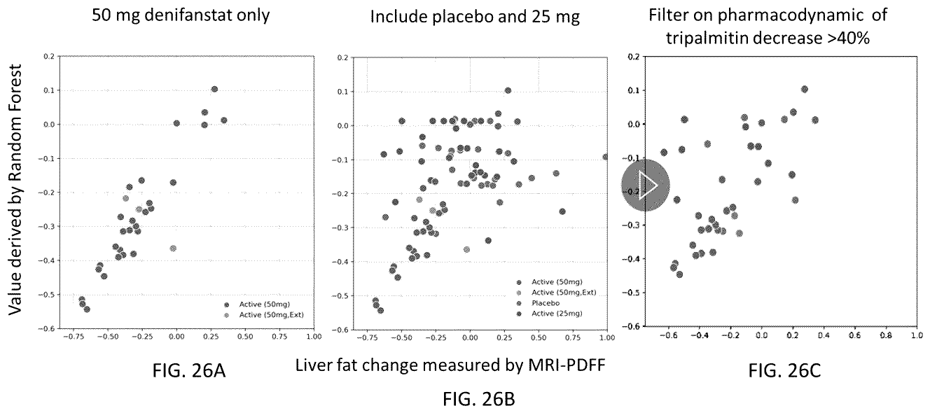

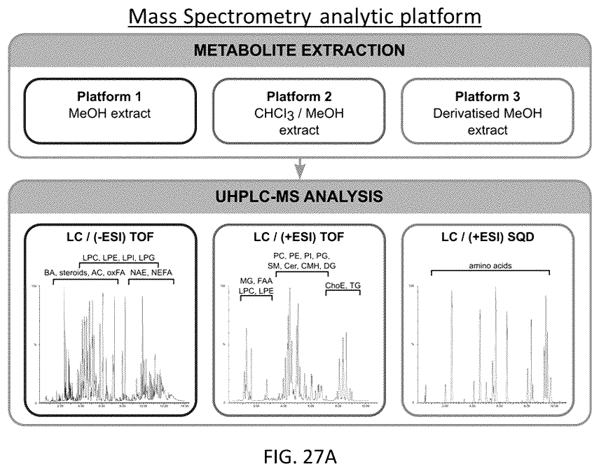

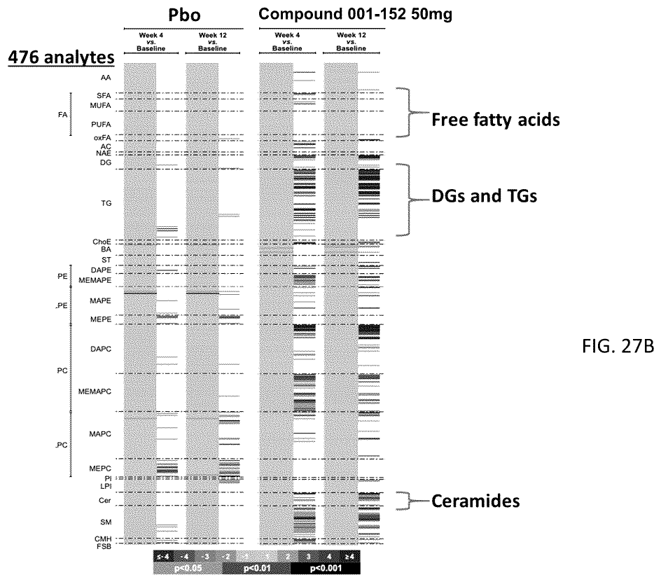

A baseline biomarker signature (Sig-A) was identified to predict the de novo lipogenesis LF response at 50 mg compound 001-152 using the RF algorithm (FIG. 20). Sig-A consists of 6 serum metabolites; ursodeoxycholic acid (UDCA), DL-2-aminocaprylic acid, sarcosine, glyco-UDCA, D(−)-2-aminobutyric acid, phosphatidylcholine (0-18:0/22:4). Sig-A gave accuracy of 84%, PPV of 73% and NPV 90% for LF decrease of >25% at 50 mg Compound 001-152. To expand the dataset, 25 mg and 75 mg FASCINATE-1 dose groups were included (n=64 total). To account for sub-optimal FASN inhibition, a filter for tripalmitin (TP) decrease was incorporated. TP, a target engagement marker for compound 001-152 showed a dose-dependent decrease in FASCINATE-1 (FIG. 23). Root Mean Square Difference analysis (FIGS. 24A and 24B) showed that the model improved performance when patients with >25% decrease in TP were included, and further improved with a 50% decrease threshold. Sig-A predicted LF response independent of PNPLA3 rs738409 status. 2 of 3 pts homozygous for PNPLA3 rs738409 had >30% LF decrease.

Baseline samples from FASCINATE-2 will be profiled. Sig-A will be used prospectively to predict LF reduction, and subsequently compared to actual LF results, to enable validation of Sig-A. Biopsy results at 52 wk will be incorporated to evaluate translation to liver histology and refine Sig-A and other biomarker panels.

### Conclusion

A biomarker signature that strongly predicts liver fat response to compound 001-152 has been identified. FASN inhibition is a promising therapeutic approach in NASH, even in patients homozygous for PNPLA3 rs738409. SNP analysis will be used to test impact of variants known to be associated with common NAFLD/NASH.

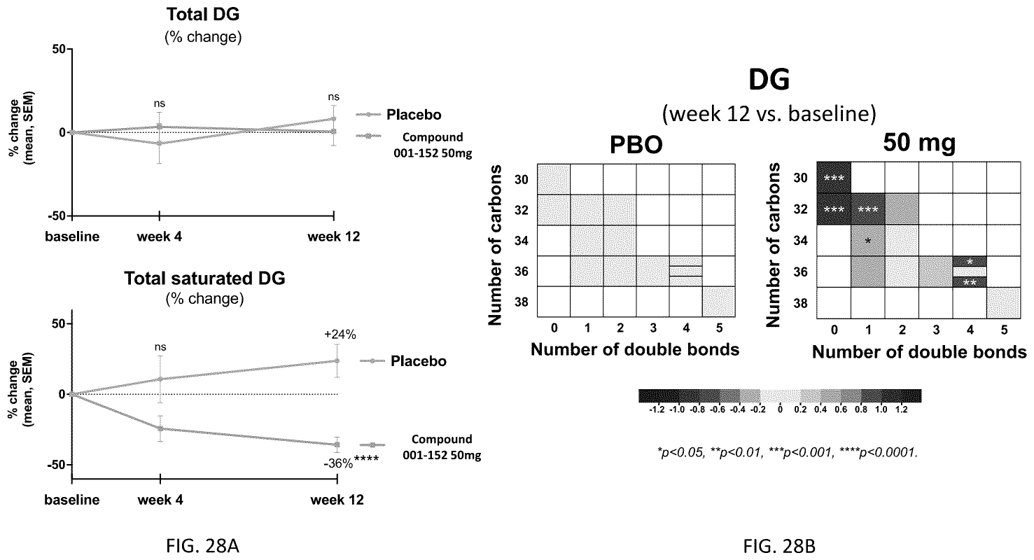

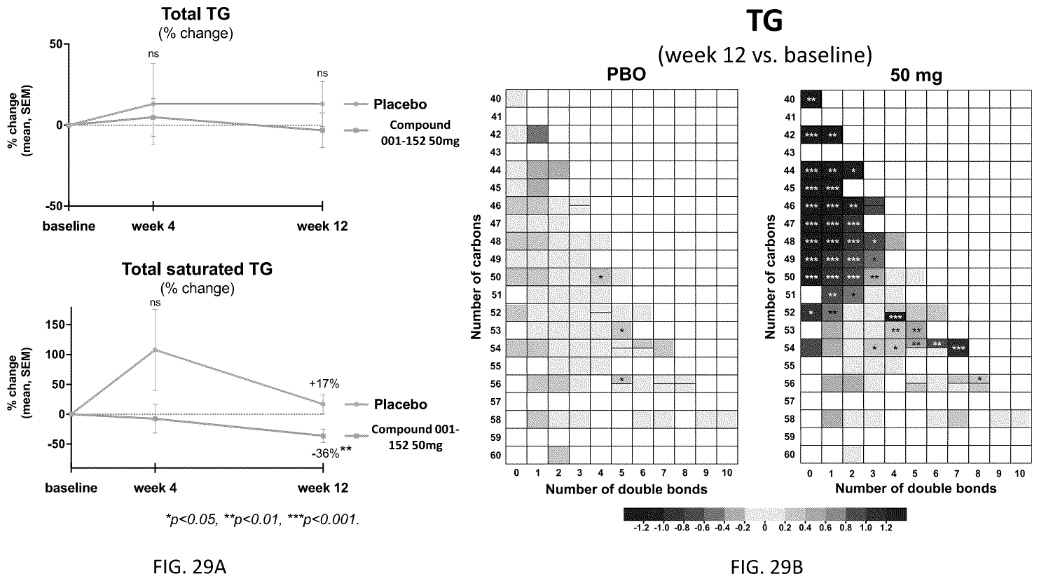

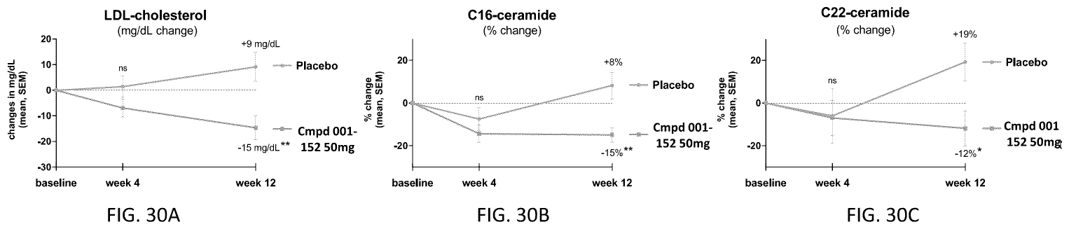

Compound 001-152 has a favorable effect on fatty acid composition (FIG. 28 and FIG. 29) and cholesterol (FIG. 30A), indicative of cardiovascular benefit, decreased lipotoxic saturated di- and tri-acylglycerols (FIG. 28A and FIG. 29A), increased long chain pufa (FIG. 28B and FIG. 29B), and reduced LDL cholesterol (FIG. 30A).

### Data Set and Methodology

All analyses were performed with JupyterLab 3.2.0 and Python. For reproducible research purpose, the hardware information and libraries used are described: scipy 1.5.2 matplotlib 3.3.2 seaborn 0.11.0 sklearn 0.24.2 pandas 1.1.3 and numpy 1.20.3.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring Such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### SUPPLEMENTARY TABLES

TABLE C-1
Fatty Acid Synthase Inhibitors

| | | FASN |
|---|---|---|
| Cmpd | | IC₅₀ |
| No. | Structure | (μM) |
| 001-1 | 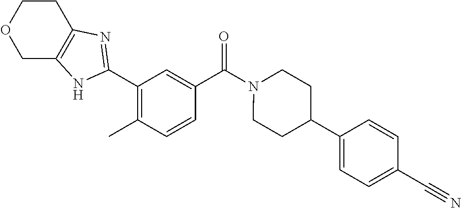 [SMILES: Cc1ccc(C(=O)N2CCC(c3ccc(C#N)cc3)CC2)cc1-c1nc2c([nH]1)COCC2] | 0.230 |
| 001-2 | 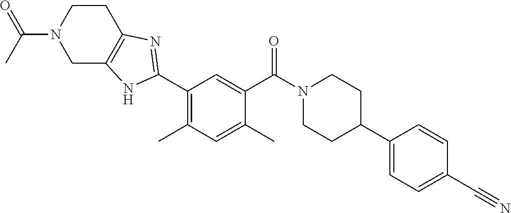 [SMILES: CC(=O)N1CCc2nc(-c3cc(C(=O)N4CCC(c5ccc(C#N)cc5)CC4)c(C)cc3C)[nH]c2C1] | 0.065 |
| 001-2.9 | 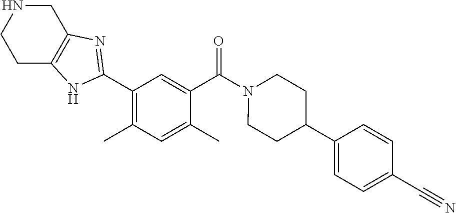 [SMILES: Cc1cc(C)c(-c2nc3c([nH]2)CCNC3)cc1C(=O)N1CCC(c2ccc(C#N)cc2)CC1] | 0.060 |
| 001-3 | 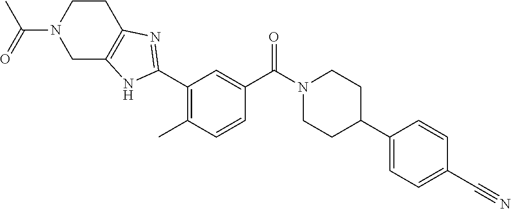 [SMILES: CC(=O)N1CCc2nc(-c3cc(C(=O)N4CCC(c5ccc(C#N)cc5)CC4)ccc3C)[nH]c2C1] | 0.146 |
| 001-4 | 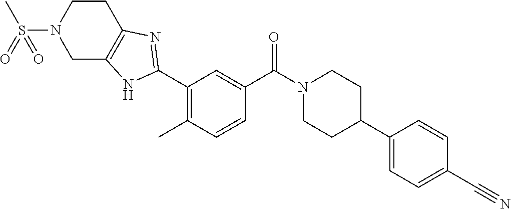 [SMILES: Cc1ccc(C(=O)N2CCC(c3ccc(C#N)cc3)CC2)cc1-c1nc2c([nH]1)CN(S(C)(=O)=O)CC2] | 0.200 |
| 001-5 | 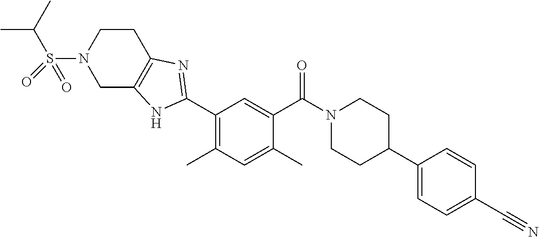 [SMILES: Cc1cc(C)c(-c2nc3c([nH]2)CN(S(=O)(=O)C(C)C)CC3)cc1C(=O)N1CCC(c2ccc(C#N)cc2)CC1] | 0.055 |
| 001-6 | 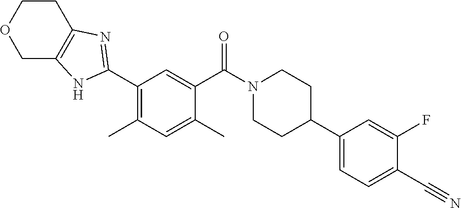 [SMILES: Cc1cc(C)c(-c2nc3c([nH]2)COCC3)cc1C(=O)N1CCC(c2ccc(C#N)c(F)c2)CC1] | 0.360 |
| 001-7 | 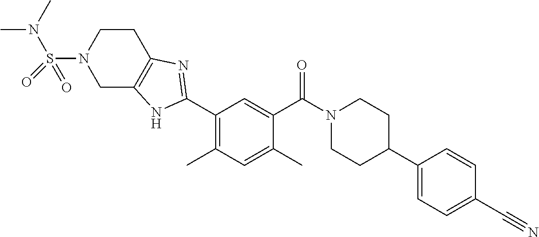 [SMILES: Cc1cc(C)c(-c2nc3c([nH]2)CN(S(=O)(=O)N(C)C)CC3)cc1C(=O)N1CCC(c2ccc(C#N)cc2)CC1] | 0.050 |
| 001-8 | 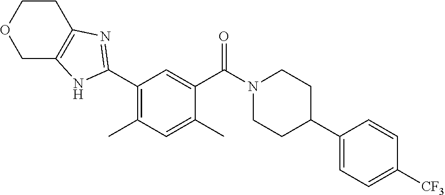 [SMILES: Cc1cc(C)c(-c2nc3c([nH]2)COCC3)cc1C(=O)N1CCC(c2ccc(C(F)(F)F)cc2)CC1] | 0.125 |
| 001-251 | 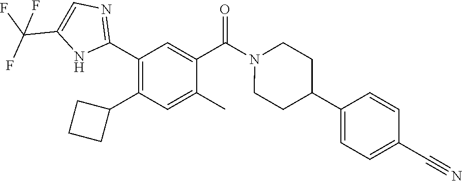 [SMILES: Cc1cc(C2CCC2)c(-c2ncc(C(F)(F)F)[nH]2)cc1C(=O)N1CCC(c2ccc(C#N)cc2)CC1] | 0.580 |
| 001-252 | 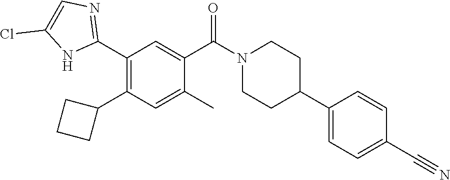 [SMILES: Cc1cc(C2CCC2)c(-c2ncc(Cl)[nH]2)cc1C(=O)N1CCC(c2ccc(C#N)cc2)CC1] | 0.180 |
| 001-253 | 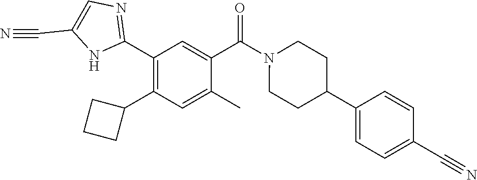 [SMILES: Cc1cc(C2CCC2)c(-c2ncc(C#N)[nH]2)cc1C(=O)N1CCC(c2ccc(C#N)cc2)CC1] | 0.090 |
| 001-254 | 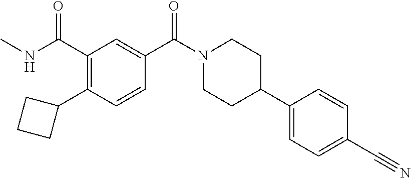 [SMILES: CNC(=O)c1cc(C(=O)N2CCC(c3ccc(C#N)cc3)CC2)ccc1C1CCC1] | 0.090 |
| 001-255 | 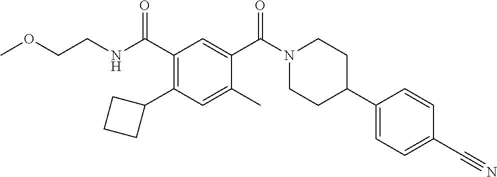 [SMILES: COCCNC(=O)c1cc(C(=O)N2CCC(c3ccc(C#N)cc3)CC2)c(C)cc1C1CCC1] | 0.410 |
| 001-256 | 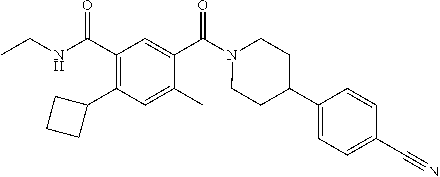 [SMILES: CCNC(=O)c1cc(C(=O)N2CCC(c3ccc(C#N)cc3)CC2)c(C)cc1C1CCC1] | 0.090 |
| 001-257 | 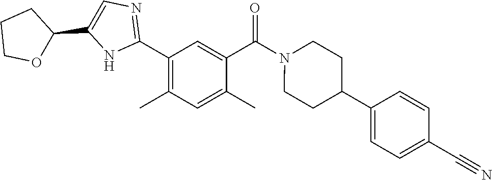 [SMILES: Cc1cc(C)c(-c2ncc([C@@H]3CCCO3)[nH]2)cc1C(=O)N1CCC(c2ccc(C#N)cc2)CC1] | 0.580 |
| 001-258 | 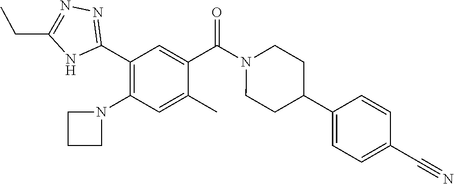 [SMILES: CCc1nnc(-c2cc(C(=O)N3CCC(c4ccc(C#N)cc4)CC3)c(C)cc2N2CCC2)[nH]1] | 0.150 |
| 001-259 | 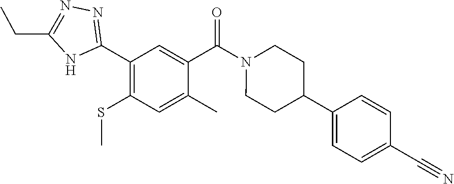 [SMILES: CCc1nnc(-c2cc(C(=O)N3CCC(c4ccc(C#N)cc4)CC3)c(C)cc2SC)[nH]1] | 1.330 |
| 001-9 | 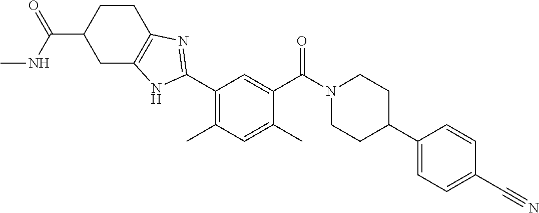 [SMILES: CNC(=O)C1CCc2nc(-c3cc(C(=O)N4CCC(c5ccc(C#N)cc5)CC4)c(C)cc3C)[nH]c2C1] | 0.040 |
| 001-11 | 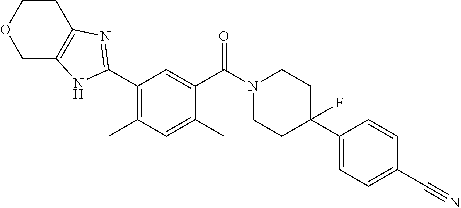 [SMILES: Cc1cc(C)c(-c2nc3c([nH]2)COCC3)cc1C(=O)N1CCC(F)(c2ccc(C#N)cc2)CC1] | 0.170 |
| 001-12 | 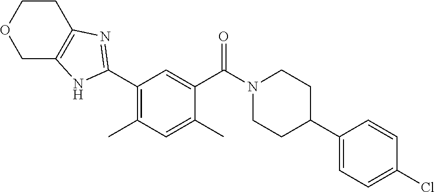 [SMILES: Cc1cc(C)c(-c2nc3c([nH]2)COCC3)cc1C(=O)N1CCC(c2ccc(Cl)cc2)CC1] | 0.370 |
| 001-13 | 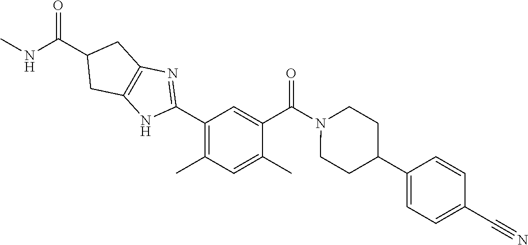 [SMILES: CNC(=O)C1Cc2nc(-c3cc(C(=O)N4CCC(c5ccc(C#N)cc5)CC4)c(C)cc3C)[nH]c2C1] | 0.260 |
| 001-14 | 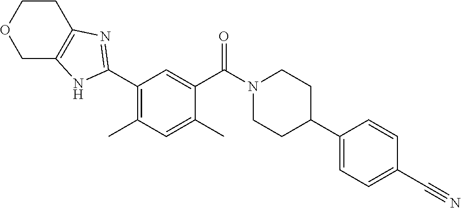 [SMILES: Cc1cc(C)c(-c2nc3c([nH]2)COCC3)cc1C(=O)N1CCC(c2ccc(C#N)cc2)CC1] | 0.263 |
| 001-15 | 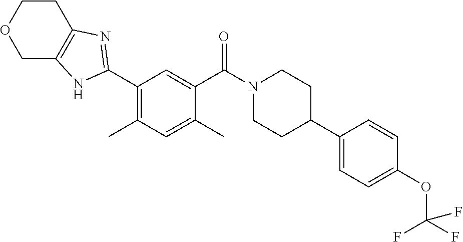 [SMILES: Cc1cc(C)c(-c2nc3c([nH]2)COCC3)cc1C(=O)N1CCC(c2ccc(OC(F)(F)F)cc2)CC1] | 0.180 |
| 001-16 | 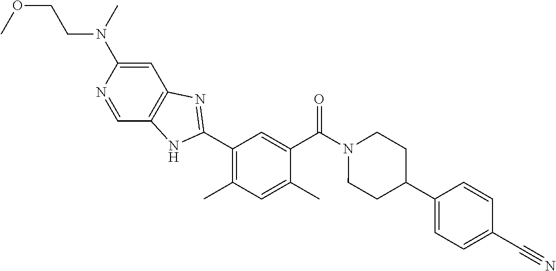 [SMILES: COCCN(C)c1cc2nc(-c3cc(C(=O)N4CCC(c5ccc(C#N)cc5)CC4)c(C)cc3C)[nH]c2cn1] | 0.058 |
| 001-17 | 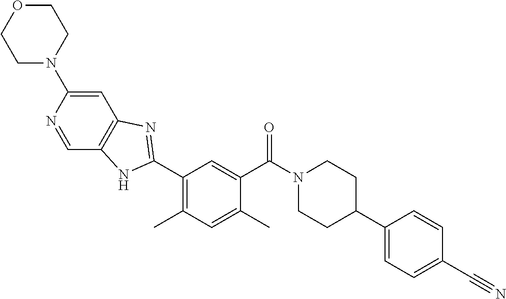 [SMILES: Cc1cc(C)c(-c2nc3cc(N4CCOCC4)ncc3[nH]2)cc1C(=O)N1CCC(c2ccc(C#N)cc2)CC1] | 0.073 |
| 001-260 | 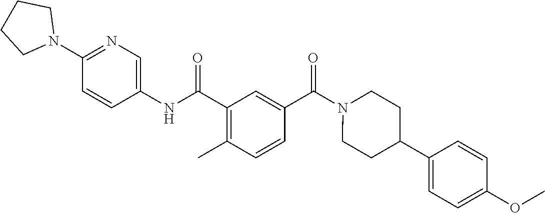 [SMILES: COc1ccc(C2CCN(C(=O)c3ccc(C)c(C(=O)Nc4ccc(N5CCCC5)nc4)c3)CC2)cc1] | 3.500 |
| 001-261 | 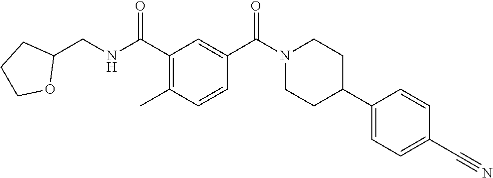 [SMILES: Cc1ccc(C(=O)N2CCC(c3ccc(C#N)cc3)CC2)cc1C(=O)NCC1CCCO1] | 21.750 |
| 001-262 | 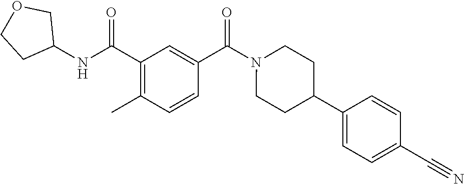 [SMILES: Cc1ccc(C(=O)N2CCC(c3ccc(C#N)cc3)CC2)cc1C(=O)NC1CCOC1] | 5.343 |
| 001-263 | 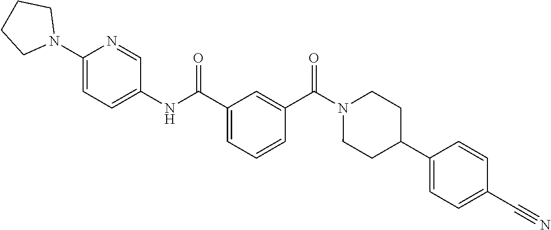 [SMILES: N#Cc1ccc(C2CCN(C(=O)c3cccc(C(=O)Nc4ccc(N5CCCC5)nc4)c3)CC2)cc1] | 0.250 |
| 001-264 | 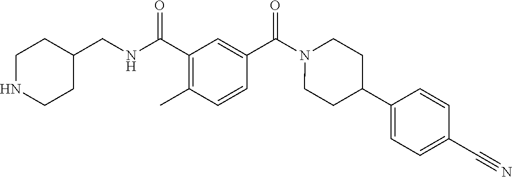 [SMILES: Cc1ccc(C(=O)N2CCC(c3ccc(C#N)cc3)CC2)cc1C(=O)NCC1CCNCC1] | >50 |
| 001-265 | 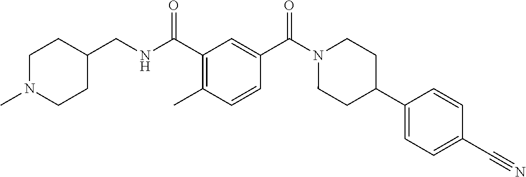 [SMILES: Cc1ccc(C(=O)N2CCC(c3ccc(C#N)cc3)CC2)cc1C(=O)NCC1CCN(C)CC1] | >50 |
| 001-266 | 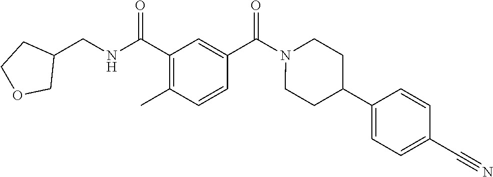 [SMILES: Cc1ccc(C(=O)N2CCC(c3ccc(C#N)cc3)CC2)cc1C(=O)NCC1CCOC1] | >50 |
| 001-267 | 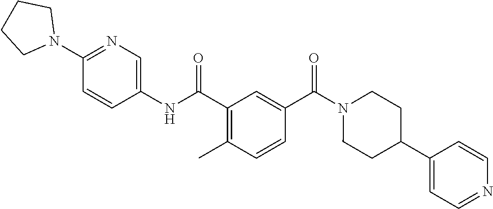 [SMILES: Cc1ccc(C(=O)N2CCC(c3ccncc3)CC2)cc1C(=O)Nc1ccc(N2CCCC2)nc1] | 30.260 |
| 001-18 | 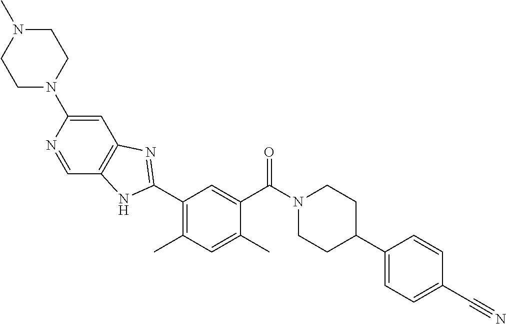 [SMILES: Cc1cc(C)c(-c2nc3cc(N4CCN(C)CC4)ncc3[nH]2)cc1C(=O)N1CCC(c2ccc(C#N)cc2)CC1] | 0.990 |
| 001-19 | 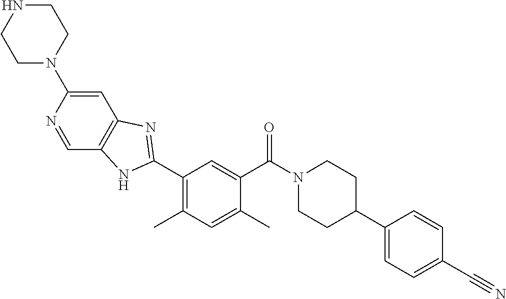 [SMILES: Cc1cc(C)c(-c2nc3cc(N4CCNCC4)ncc3[nH]2)cc1C(=O)N1CCC(c2ccc(C#N)cc2)CC1] | 0.870 |
| 001-20 | 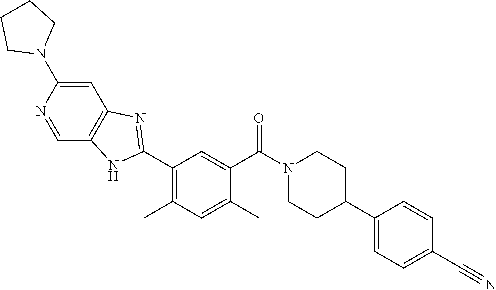 [SMILES: Cc1cc(C)c(-c2nc3cc(N4CCCC4)ncc3[nH]2)cc1C(=O)N1CCC(c2ccc(C#N)cc2)CC1] | 0.022 |
| 001-21 | 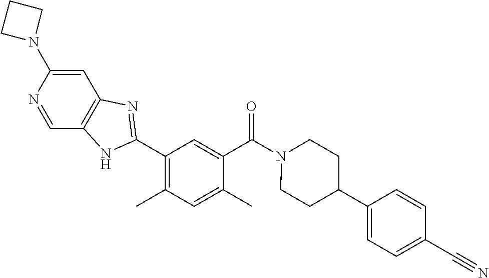 [SMILES: Cc1cc(C)c(-c2nc3cc(N4CCC4)ncc3[nH]2)cc1C(=O)N1CCC(c2ccc(C#N)cc2)CC1] | 0.045 |
| 001-22 | 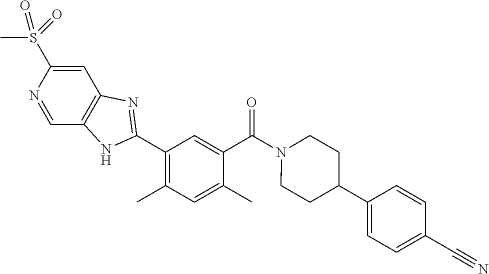 [SMILES: Cc1cc(C)c(-c2nc3cc(S(C)(=O)=O)ncc3[nH]2)cc1C(=O)N1CCC(c2ccc(C#N)cc2)CC1] | 0.085 |
| 001-23 | 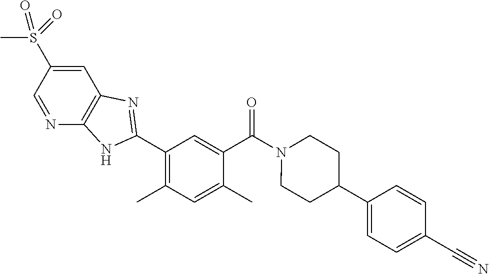 [SMILES: Cc1cc(C)c(-c2nc3cc(S(C)(=O)=O)cnc3[nH]2)cc1C(=O)N1CCC(c2ccc(C#N)cc2)CC1] | 0.070 |
| 001-268 | 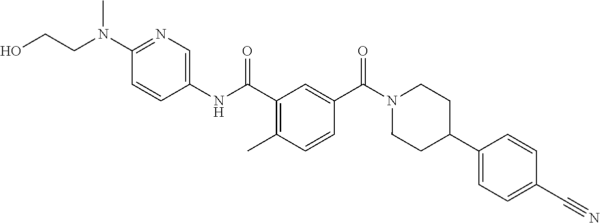 [SMILES: Cc1ccc(C(=O)N2CCC(c3ccc(C#N)cc3)CC2)cc1C(=O)Nc1ccc(N(C)CCO)nc1] | 0.390 |
| 001-269 | 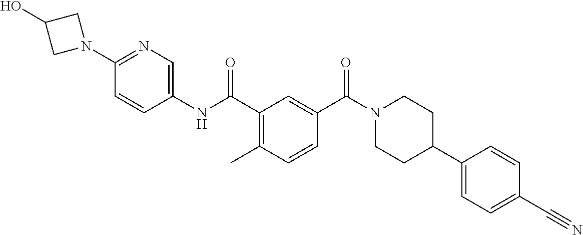 [SMILES: Cc1ccc(C(=O)N2CCC(c3ccc(C#N)cc3)CC2)cc1C(=O)Nc1ccc(N2CC(O)C2)nc1] | 1.350 |
| 001-270 | 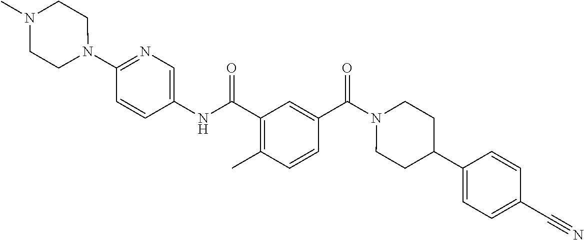 [SMILES: Cc1ccc(C(=O)N2CCC(c3ccc(C#N)cc3)CC2)cc1C(=O)Nc1ccc(N2CCN(C)CC2)nc1] | 1.980 |
| 001-271 | 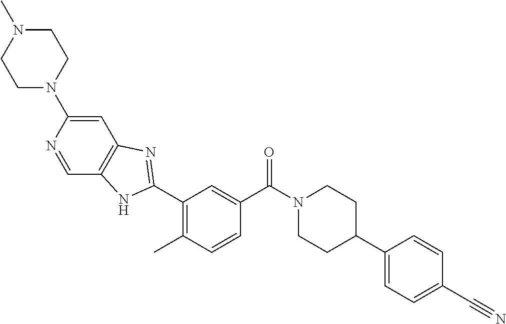 [SMILES: Cc1ccc(C(=O)N2CCC(c3ccc(C#N)cc3)CC2)cc1-c1nc2cc(N3CCN(C)CC3)ncc2[nH]1] | 0.520 |
| 001-272 | 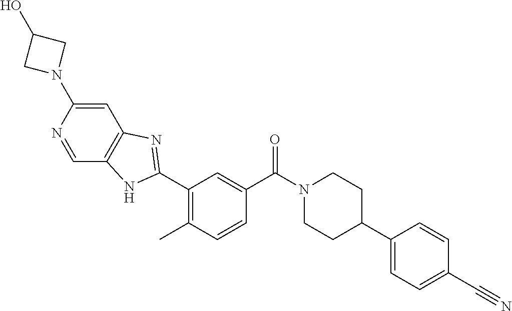 [SMILES: Cc1ccc(C(=O)N2CCC(c3ccc(C#N)cc3)CC2)cc1-c1nc2cc(N3CC(O)C3)ncc2[nH]1] | 0.300 |
| 001-273 | 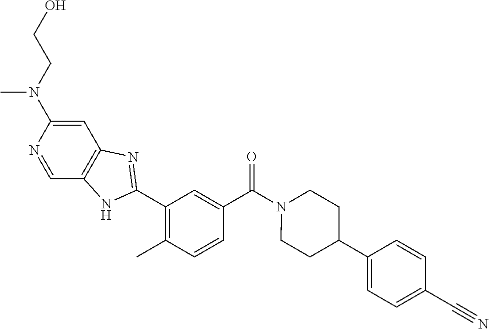 [SMILES: Cc1ccc(C(=O)N2CCC(c3ccc(C#N)cc3)CC2)cc1-c1nc2cc(N(C)CCO)ncc2[nH]1] | 0.060 |
| 001-24 | 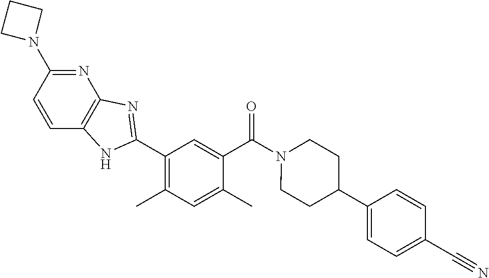 [SMILES: Cc1cc(C)c(-c2nc3nc(N4CCC4)ccc3[nH]2)cc1C(=O)N1CCC(c2ccc(C#N)cc2)CC1] | 0.070 |
| 001-25 | 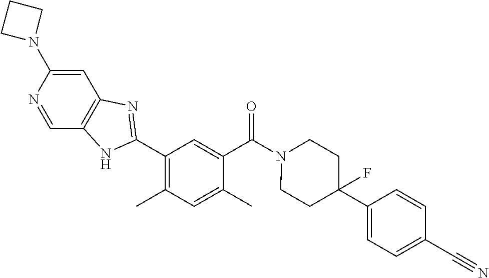 [SMILES: Cc1cc(C)c(-c2nc3cc(N4CCC4)ncc3[nH]2)cc1C(=O)N1CCC(F)(c2ccc(C#N)cc2)CC1] | 0.150 |
| 001-26 | 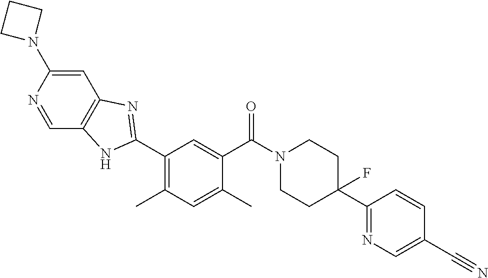 [SMILES: Cc1cc(C)c(-c2nc3cc(N4CCC4)ncc3[nH]2)cc1C(=O)N1CCC(F)(c2ccc(C#N)cn2)CC1] | 7.195 |
| 001-27 | 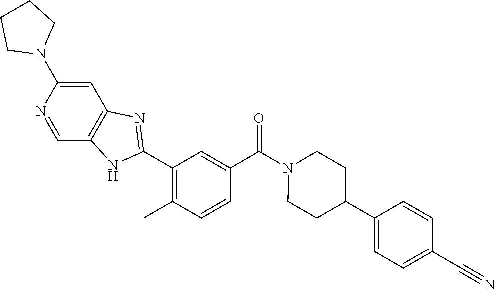 [SMILES: Cc1ccc(C(=O)N2CCC(c3ccc(C#N)cc3)CC2)cc1-c1nc2cc(N3CCCC3)ncc2[nH]1] | 0.107 |
| 001-28 | 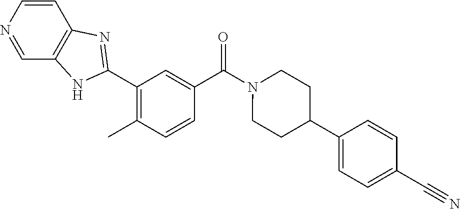 [SMILES: Cc1ccc(C(=O)N2CCC(c3ccc(C#N)cc3)CC2)cc1-c1nc2ccncc2[nH]1] | 0.490 |
| 001-29 | 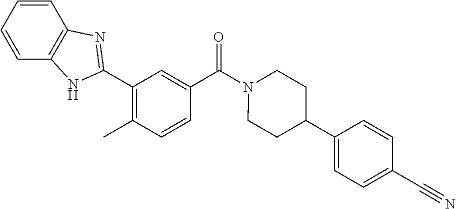 [SMILES: Cc1ccc(C(=O)N2CCC(c3ccc(C#N)cc3)CC2)cc1-c1nc2ccccc2[nH]1] | 0.980 |
| 001-30 | 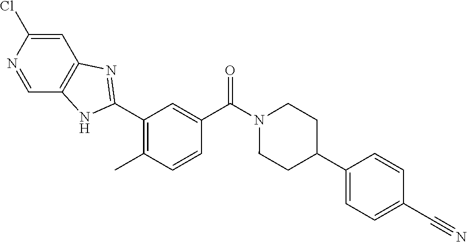 [SMILES: Cc1ccc(C(=O)N2CCC(c3ccc(C#N)cc3)CC2)cc1-c1nc2cc(Cl)ncc2[nH]1] | 0.320 |
| 001-274 | 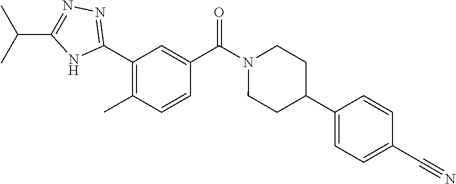 [SMILES: Cc1ccc(C(=O)N2CCC(c3ccc(C#N)cc3)CC2)cc1-c1nnc(C(C)C)[nH]1] | 2.345 |
| 001-275 | 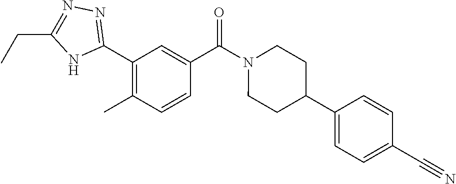 [SMILES: CCc1nnc(-c2cc(C(=O)N3CCC(c4ccc(C#N)cc4)CC3)ccc2C)[nH]1] | 2.245 |
| 001-276 | 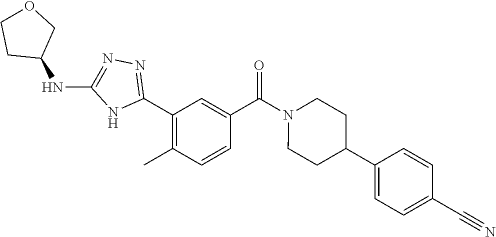 [SMILES: Cc1ccc(C(=O)N2CCC(c3ccc(C#N)cc3)CC2)cc1-c1nnc(N[C@H]2CCOC2)[nH]1] | 9.060 |
| 001-277 | 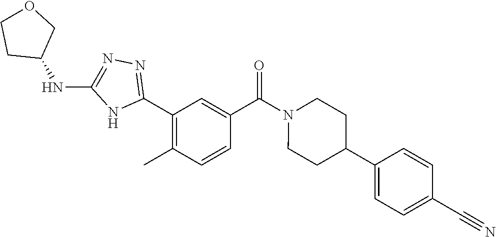 [SMILES: Cc1ccc(C(=O)N2CCC(c3ccc(C#N)cc3)CC2)cc1-c1nnc(N[C@@H]2CCOC2)[nH]1] | 11.680 |
| 001-278 | 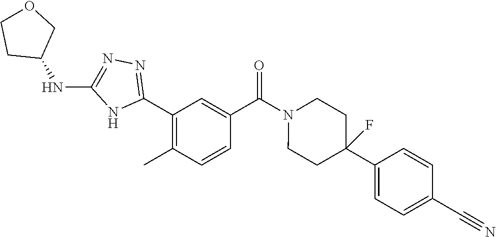 [SMILES: Cc1ccc(C(=O)N2CCC(F)(c3ccc(C#N)cc3)CC2)cc1-c1nnc(N[C@@H]2CCOC2)[nH]1] | 1.750 |
| 001-279 | 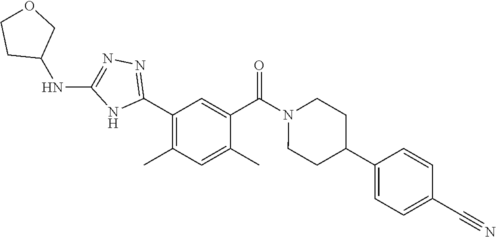 [SMILES: Cc1cc(C)c(-c2nnc(NC3CCOC3)[nH]2)cc1C(=O)N1CCC(c2ccc(C#N)cc2)CC1] | 0.330 |
| 001-280 | 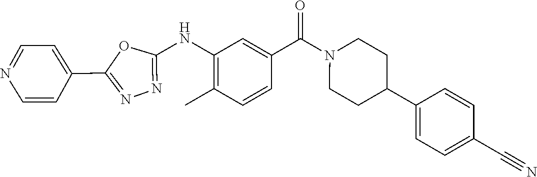 [SMILES: Cc1ccc(C(=O)N2CCC(c3ccc(C#N)cc3)CC2)cc1Nc1nnc(-c2ccncc2)o1] | 0.700 |
| 001-31 | 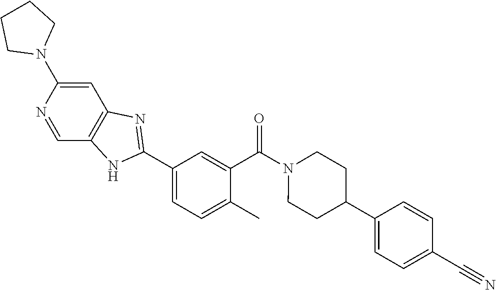 [SMILES: Cc1ccc(-c2nc3cc(N4CCCC4)ncc3[nH]2)cc1C(=O)N1CCC(c2ccc(C#N)cc2)CC1] | 0.060 |
| 001-32 | 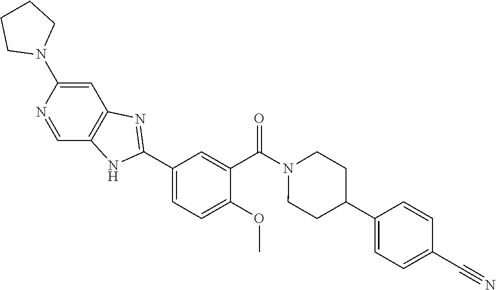 [SMILES: COc1ccc(-c2nc3cc(N4CCCC4)ncc3[nH]2)cc1C(=O)N1CCC(c2ccc(C#N)cc2)CC1] | 0.215 |
| 001-33 | 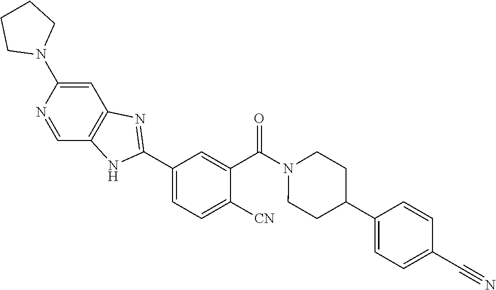 [SMILES: N#Cc1ccc(C2CCN(C(=O)c3cc(-c4nc5cc(N6CCCC6)ncc5[nH]4)ccc3C#N)CC2)cc1] | 7.500 |
| 001-34 | 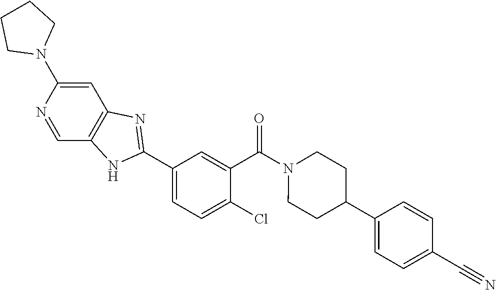 [SMILES: N#Cc1ccc(C2CCN(C(=O)c3cc(-c4nc5cc(N6CCCC6)ncc5[nH]4)ccc3Cl)CC2)cc1] | 0.080 |
| 001-35 | 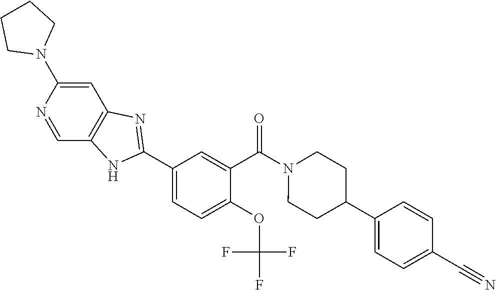 [SMILES: N#Cc1ccc(C2CCN(C(=O)c3cc(-c4nc5cc(N6CCCC6)ncc5[nH]4)ccc3OC(F)(F)F)CC2)cc1] | 25.083 |
| 001-36 | 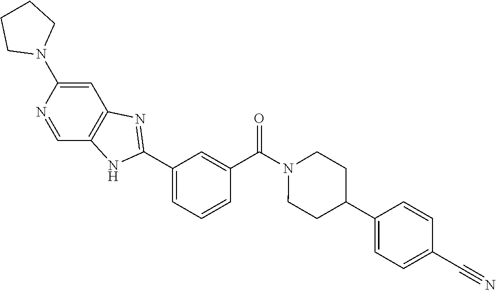 [SMILES: N#Cc1ccc(C2CCN(C(=O)c3cccc(-c4nc5cc(N6CCCC6)ncc5[nH]4)c3)CC2)cc1] | 0.130 |
| 001-37 | 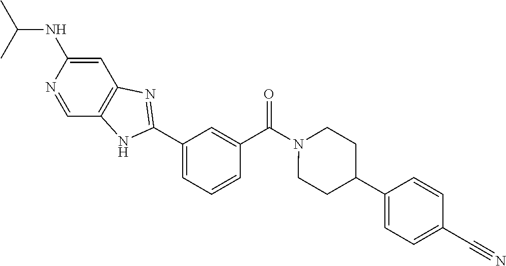 [SMILES: CC(C)Nc1cc2nc(-c3cccc(C(=O)N4CCC(c5ccc(C#N)cc5)CC4)c3)[nH]c2cn1] | 0.675 |
| 001-281 | 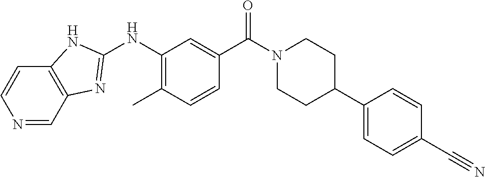 [SMILES: Cc1ccc(C(=O)N2CCC(c3ccc(C#N)cc3)CC2)cc1Nc1nc2cnccc2[nH]1] | 42.300 |
| 001-282 | 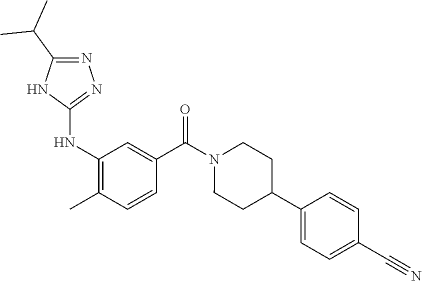 [SMILES: Cc1ccc(C(=O)N2CCC(c3ccc(C#N)cc3)CC2)cc1Nc1nnc(C(C)C)[nH]1] | 30.090 |
| 001-283 | 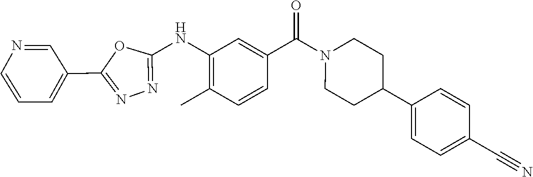 [SMILES: Cc1ccc(C(=O)N2CCC(c3ccc(C#N)cc3)CC2)cc1Nc1nnc(-c2cccnc2)o1] | 1.000 |
| 001-284 | 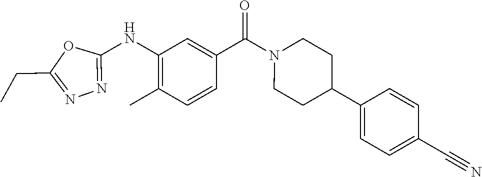 [SMILES: CCc1nnc(Nc2cc(C(=O)N3CCC(c4ccc(C#N)cc4)CC3)ccc2C)o1] | 1.005 |
| 001-285 | 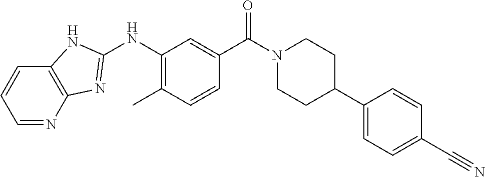 [SMILES: Cc1ccc(C(=O)N2CCC(c3ccc(C#N)cc3)CC2)cc1Nc1nc2ncccc2[nH]1] | >50 |
| 001-286 | 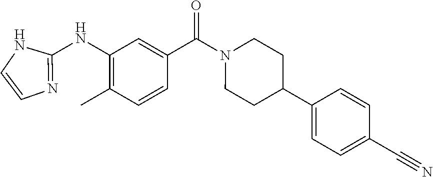 [SMILES: Cc1ccc(C(=O)N2CCC(c3ccc(C#N)cc3)CC2)cc1Nc1ncc[nH]1] | >50 |
| 001-287 | 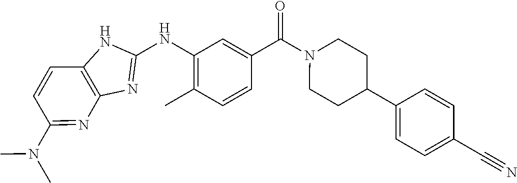 [SMILES: Cc1ccc(C(=O)N2CCC(c3ccc(C#N)cc3)CC2)cc1Nc1nc2nc(N(C)C)ccc2[nH]1] | >40 |
| 001-38 | 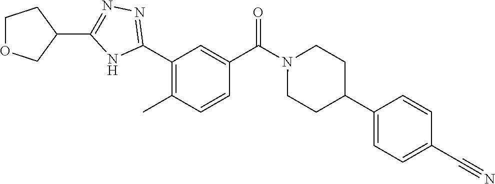 [SMILES: Cc1ccc(C(=O)N2CCC(c3ccc(C#N)cc3)CC2)cc1-c1nnc(C2CCOC2)[nH]1] | 2.345 |
| 001-39 | 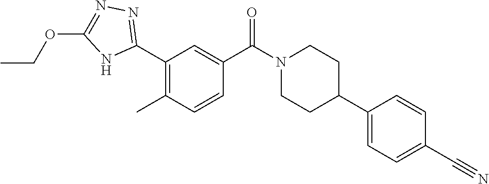 [SMILES: CCOc1nnc(-c2cc(C(=O)N3CCC(c4ccc(C#N)cc4)CC3)ccc2C)[nH]1] | 1.710 |
| 001-40 | 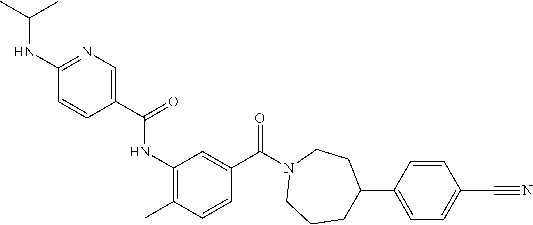 [SMILES: Cc1ccc(C(=O)N2CCCC(c3ccc(C#N)cc3)CC2)cc1NC(=O)c1ccc(NC(C)C)nc1] | 0.050 |
| 001-41 | 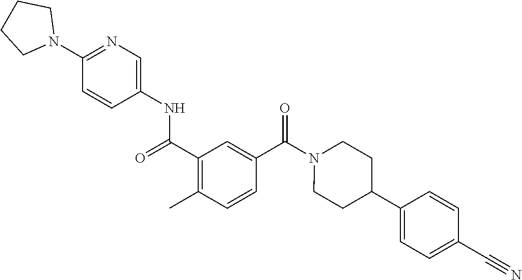 [SMILES: Cc1ccc(C(=O)N2CCC(c3ccc(C#N)cc3)CC2)cc1C(=O)Nc1ccc(N2CCCC2)nc1] | 0.035 |
| 001-42 | 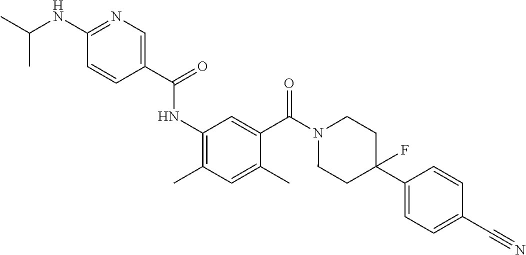 [SMILES: Cc1cc(C)c(C(=O)N2CCC(F)(c3ccc(C#N)cc3)CC2)cc1NC(=O)c1ccc(NC(C)C)nc1] | 0.067 |
| 001-43 | 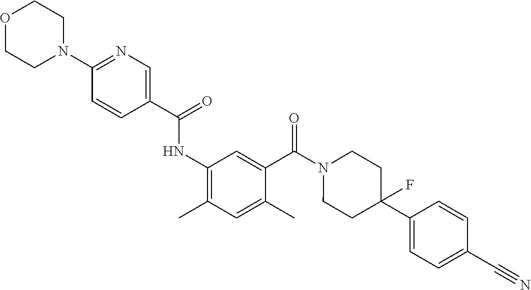 [SMILES: Cc1cc(C)c(C(=O)N2CCC(F)(c3ccc(C#N)cc3)CC2)cc1NC(=O)c1ccc(N2CCOCC2)nc1] | 0.110 |
| 001-288 | 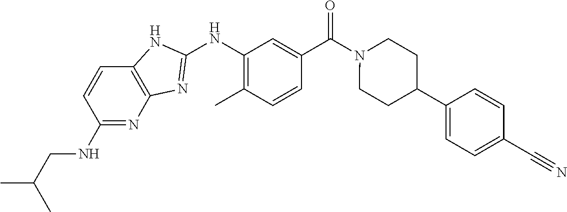 [SMILES: Cc1ccc(C(=O)N2CCC(c3ccc(C#N)cc3)CC2)cc1Nc1nc2nc(NCC(C)C)ccc2[nH]1] | |
| 001-289 | 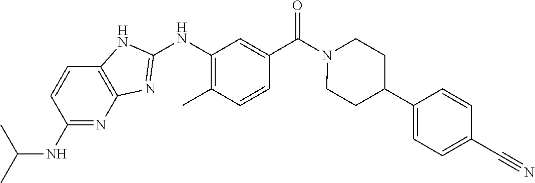 [SMILES: Cc1ccc(C(=O)N2CCC(c3ccc(C#N)cc3)CC2)cc1Nc1nc2nc(NC(C)C)ccc2[nH]1] | 32.733 |
| 001-290 | 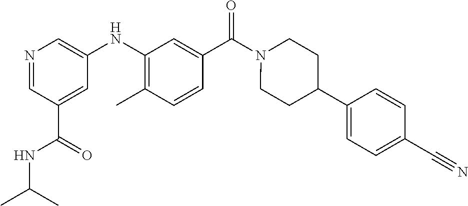 [SMILES: Cc1ccc(C(=O)N2CCC(c3ccc(C#N)cc3)CC2)cc1Nc1cncc(C(=O)NC(C)C)c1] | 0.470 |
| 001-291 | 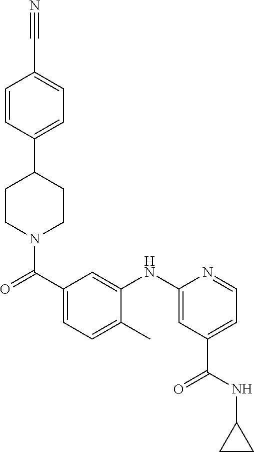 [SMILES: Cc1ccc(C(=O)N2CCC(c3ccc(C#N)cc3)CC2)cc1Nc1cc(C(=O)NC2CC2)ccn1] | 0.370 |
| 001-292 | 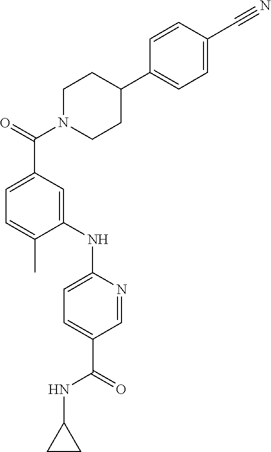 [SMILES: Cc1ccc(C(=O)N2CCC(c3ccc(C#N)cc3)CC2)cc1Nc1ccc(C(=O)NC2CC2)cn1] | 0.270 |
| 001-293 | 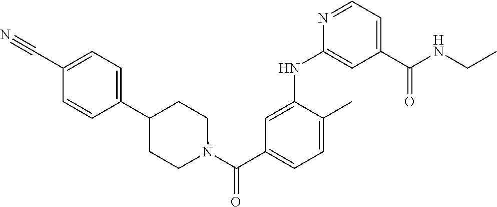 [SMILES: CCNC(=O)c1ccnc(Nc2cc(C(=O)N3CCC(c4ccc(C#N)cc4)CC3)ccc2C)c1] | 1.080 |
| 001-44 | 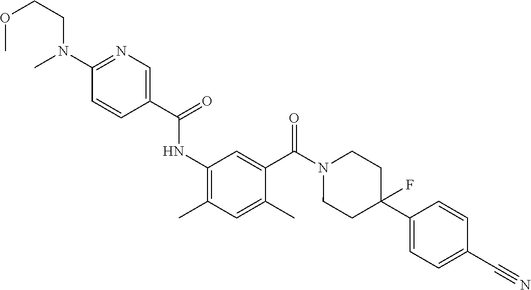 [SMILES: COCCN(C)c1ccc(C(=O)Nc2cc(C(=O)N3CCC(F)(c4ccc(C#N)cc4)CC3)c(C)cc2C)cn1] | 0.180 |
| 001-45 | 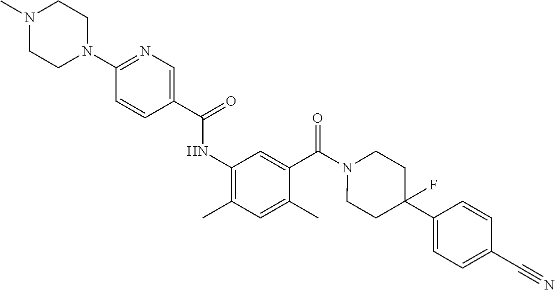 [SMILES: Cc1cc(C)c(C(=O)N2CCC(F)(c3ccc(C#N)cc3)CC2)cc1NC(=O)c1ccc(N2CCN(C)CC2)nc1] | 0.147 |
| 001-46 | 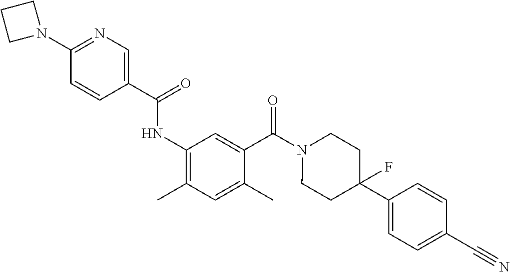 [SMILES: Cc1cc(C)c(C(=O)N2CCC(F)(c3ccc(C#N)cc3)CC2)cc1NC(=O)c1ccc(N2CCC2)nc1] | 0.050 |
| 001-47 | 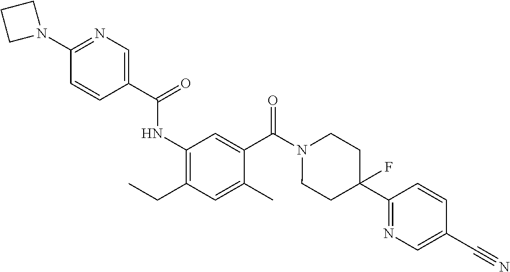 [SMILES: CCc1cc(C)c(C(=O)N2CCC(F)(c3ccc(C#N)cn3)CC2)cc1NC(=O)c1ccc(N2CCC2)nc1] | 0.230 |
| 001-48 | 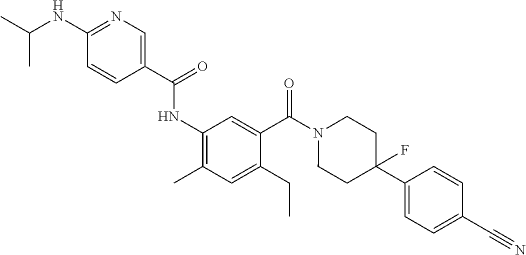 [SMILES: CCc1cc(C)c(NC(=O)c2ccc(NC(C)C)nc2)cc1C(=O)N1CCC(F)(c2ccc(C#N)cc2)CC1] | 0.170 |
| 001-49 | 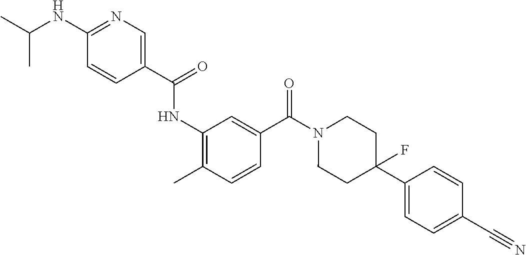 [SMILES: Cc1ccc(C(=O)N2CCC(F)(c3ccc(C#N)cc3)CC2)cc1NC(=O)c1ccc(NC(C)C)nc1] | 0.040 |
| 001-294 | 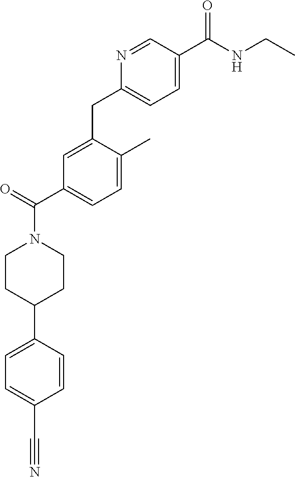 [SMILES: CCNC(=O)c1ccc(Cc2cc(C(=O)N3CCC(c4ccc(C#N)cc4)CC3)ccc2C)nc1] | |
| 001-295 | 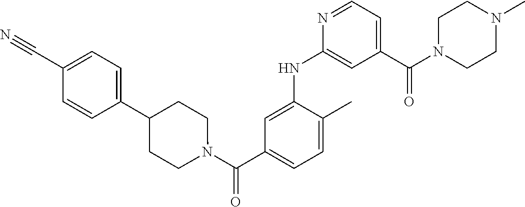 [SMILES: Cc1ccc(C(=O)N2CCC(c3ccc(C#N)cc3)CC2)cc1Nc1cc(C(=O)N2CCN(C)CC2)ccn1] | 3.820 |
| 001-296 | 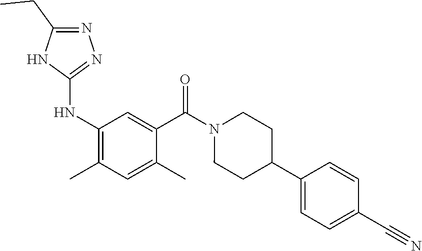 [SMILES: CCc1nnc(Nc2cc(C(=O)N3CCC(c4ccc(C#N)cc4)CC3)c(C)cc2C)[nH]1] | 3.250 |
| 001-297 | 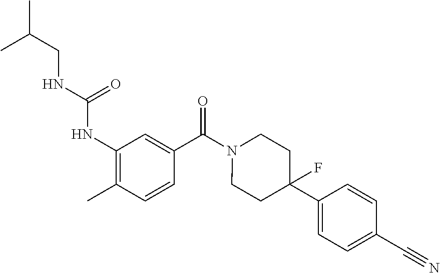 [SMILES: Cc1ccc(C(=O)N2CCC(F)(c3ccc(C#N)cc3)CC2)cc1NC(=O)NCC(C)C] | 0.025 |
| 001-298 | 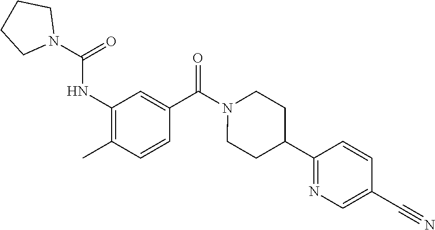 [SMILES: Cc1ccc(C(=O)N2CCC(c3ccc(C#N)cn3)CC2)cc1NC(=O)N1CCCC1] | |
| 001-299 | 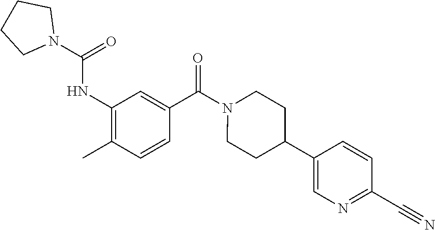 [SMILES: Cc1ccc(C(=O)N2CCC(c3ccc(C#N)nc3)CC2)cc1NC(=O)N1CCCC1] | |
| 001-50 | 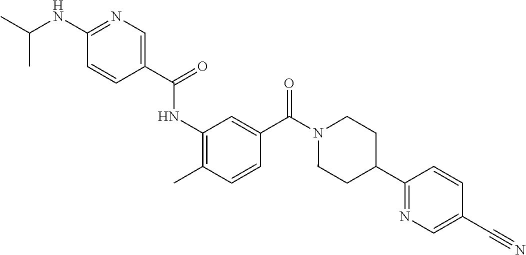 [SMILES: Cc1ccc(C(=O)N2CCC(c3ccc(C#N)cn3)CC2)cc1NC(=O)c1ccc(NC(C)C)nc1] | 0.520 |
| 001-51 | 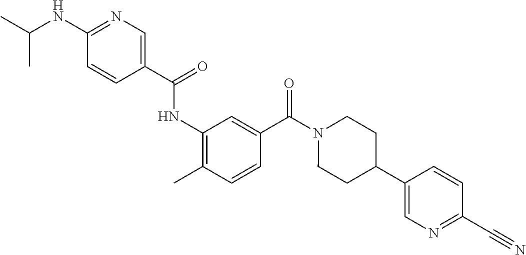 [SMILES: Cc1ccc(C(=O)N2CCC(c3ccc(C#N)nc3)CC2)cc1NC(=O)c1ccc(NC(C)C)nc1] | 1.700 |
| 001-52 | 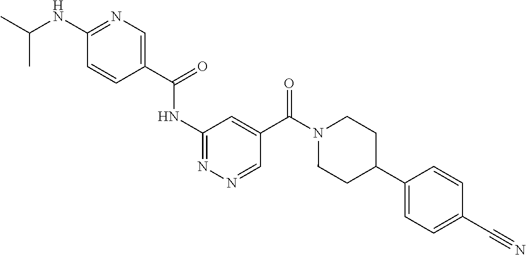 [SMILES: CC(C)Nc1ccc(C(=O)Nc2cc(C(=O)N3CCC(c4ccc(C#N)cc4)CC3)cnn2)cn1] | 31.650 |
| 001-53 | 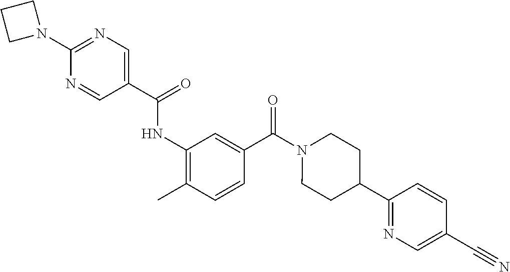 [SMILES: Cc1ccc(C(=O)N2CCC(c3ccc(C#N)cn3)CC2)cc1NC(=O)c1cnc(N2CCC2)nc1] | 1.970 |
| 001-54 | 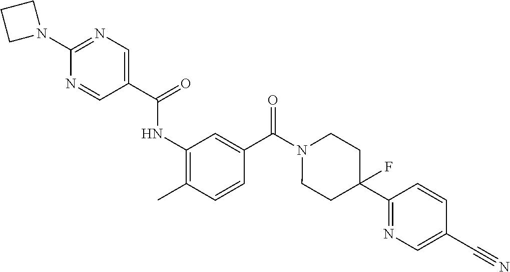 [SMILES: Cc1ccc(C(=O)N2CCC(F)(c3ccc(C#N)cn3)CC2)cc1NC(=O)c1cnc(N2CCC2)nc1] | 0.310 |
| 001-300 | 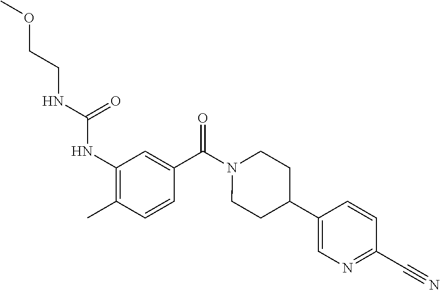 [SMILES: COCCNC(=O)Nc1cc(C(=O)N2CCC(c3ccc(C#N)nc3)CC2)ccc1C] | |
| 001-301 | 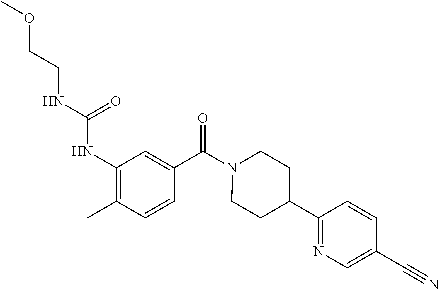 [SMILES: COCCNC(=O)Nc1cc(C(=O)N2CCC(c3ccc(C#N)cn3)CC2)ccc1C] | |
| 001-302 | 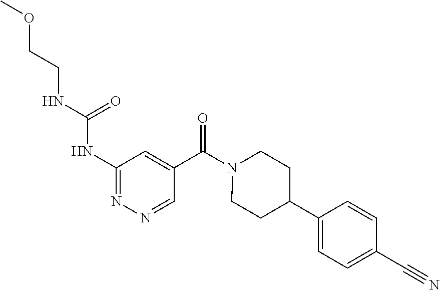 [SMILES: COCCNC(=O)Nc1cc(C(=O)N2CCC(c3ccc(C#N)cc3)CC2)cnn1] | >50 |
| 001-303 | 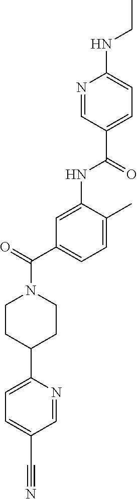 [SMILES: CCNc1ccc(C(=O)Nc2cc(C(=O)N3CCC(c4ccc(C#N)cn4)CC3)ccc2C)cn1] | |
| 001-304 | 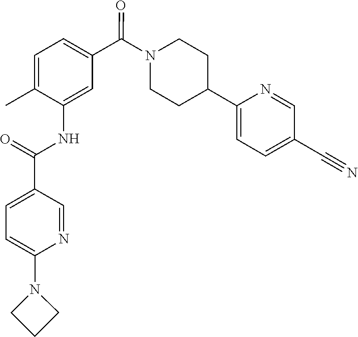 [SMILES: Cc1ccc(C(=O)N2CCC(c3ccc(C#N)cn3)CC2)cc1NC(=O)c1ccc(N2CCC2)nc1] | 0.680 |
| 001-55 | 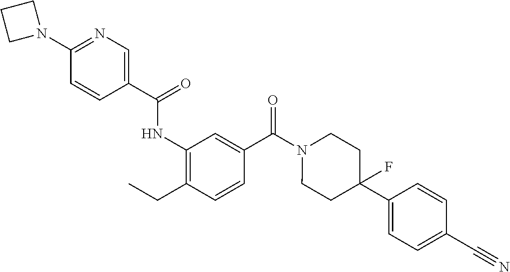 [SMILES: CCc1ccc(C(=O)N2CCC(F)(c3ccc(C#N)cc3)CC2)cc1NC(=O)c1ccc(N2CCC2)nc1] | 0.035 |
| 001-56 | 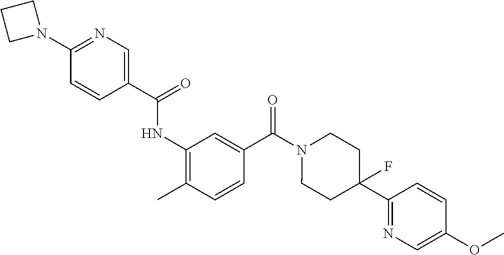 [SMILES: COc1ccc(C2(F)CCN(C(=O)c3ccc(C)c(NC(=O)c4ccc(N5CCC5)nc4)c3)CC2)nc1] | 2.800 |
| 001-57 | 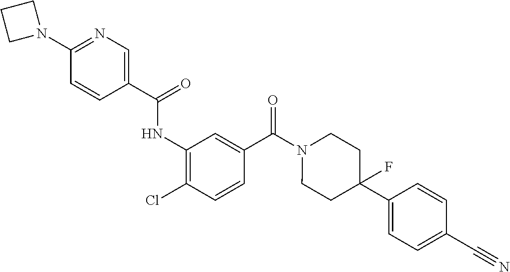 [SMILES: N#Cc1ccc(C2(F)CCN(C(=O)c3ccc(Cl)c(NC(=O)c4ccc(N5CCC5)nc4)c3)CC2)cc1] | 0.030 |
| 001-58 | 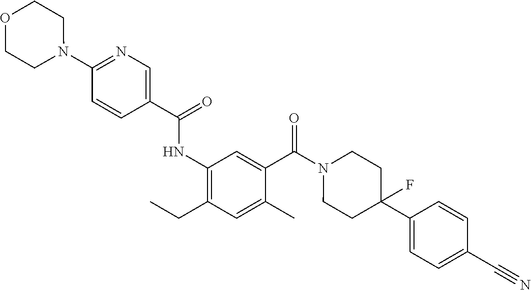 [SMILES: CCc1cc(C)c(C(=O)N2CCC(F)(c3ccc(C#N)cc3)CC2)cc1NC(=O)c1ccc(N2CCOCC2)nc1] | 0.025 |
| 001-60 | 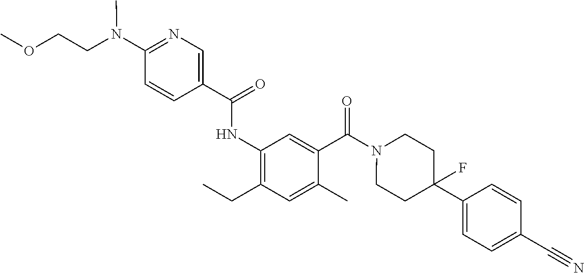 [SMILES: CCc1cc(C)c(C(=O)N2CCC(F)(c3ccc(C#N)cc3)CC2)cc1NC(=O)c1ccc(N(C)CCOC)nc1] | 0.045 |
| 001-61 | 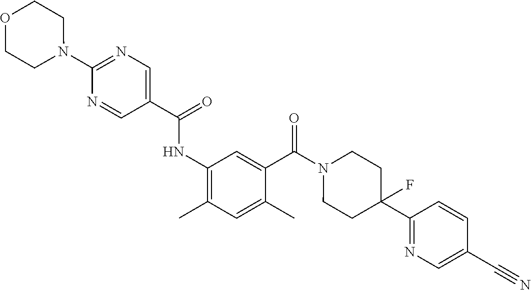 [SMILES: Cc1cc(C)c(C(=O)N2CCC(F)(c3ccc(C#N)cn3)CC2)cc1NC(=O)c1cnc(N2CCOCC2)nc1] | 0.230 |
| 001-62 | 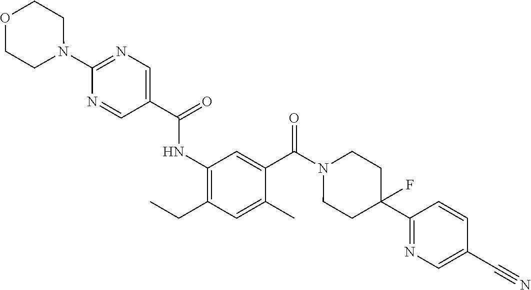 [SMILES: CCc1cc(C)c(C(=O)N2CCC(F)(c3ccc(C#N)cn3)CC2)cc1NC(=O)c1cnc(N2CCOCC2)nc1] | 0.093 |
| 001-305 | 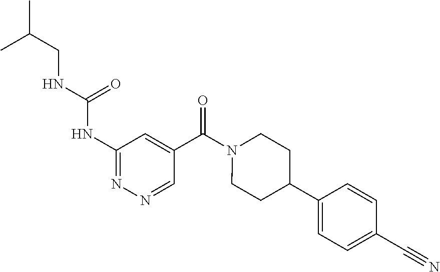 [SMILES: CC(C)CNC(=O)Nc1cc(C(=O)N2CCC(c3ccc(C#N)cc3)CC2)cnn1] | >50 |
| 001-306 | 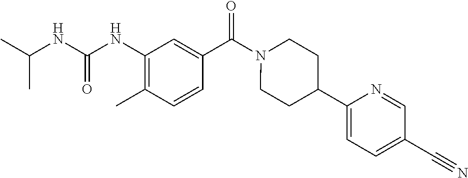 [SMILES: Cc1ccc(C(=O)N2CCC(c3ccc(C#N)cn3)CC2)cc1NC(=O)NC(C)C] | 2.900 |
| 001-307 | 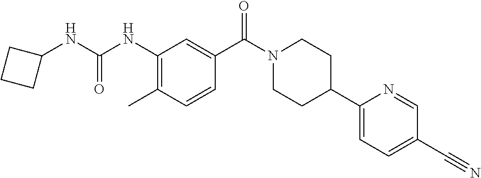 [SMILES: Cc1ccc(C(=O)N2CCC(c3ccc(C#N)cn3)CC2)cc1NC(=O)NC1CCC1] | 5.360 |
| 001-308 | 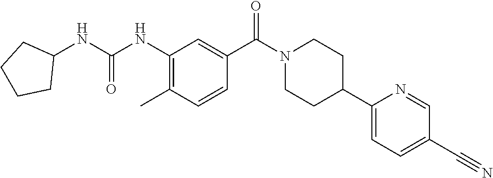 [SMILES: Cc1ccc(C(=O)N2CCC(c3ccc(C#N)cn3)CC2)cc1NC(=O)NC1CCCC1] | 2.750 |
| 001-309 | 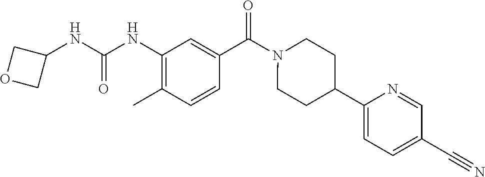 [SMILES: Cc1ccc(C(=O)N2CCC(c3ccc(C#N)cn3)CC2)cc1NC(=O)NC1COC1] | |
| 001-310 | 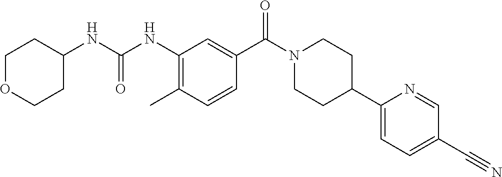 [SMILES: Cc1ccc(C(=O)N2CCC(c3ccc(C#N)cn3)CC2)cc1NC(=O)NC1CCOCC1] | 8.780 |
| 001-311 | 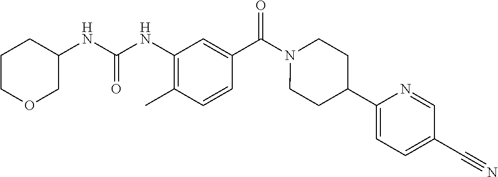 [SMILES: Cc1ccc(C(=O)N2CCC(c3ccc(C#N)cn3)CC2)cc1NC(=O)NC1CCCOC1] | |
| 001-63 | 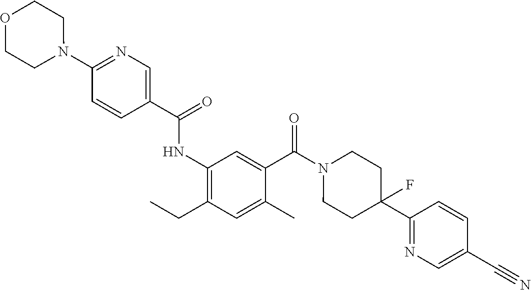 [SMILES: CCc1cc(C)c(C(=O)N2CCC(F)(c3ccc(C#N)cn3)CC2)cc1NC(=O)c1ccc(N2CCOCC2)nc1] | 0.510 |
| 001-64 | 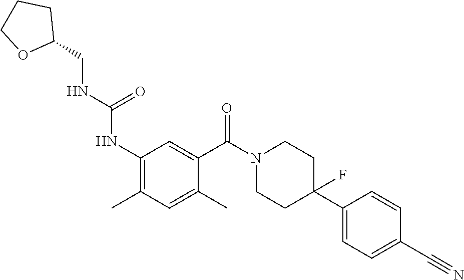 [SMILES: Cc1cc(C)c(C(=O)N2CCC(F)(c3ccc(C#N)cc3)CC2)cc1NC(=O)NC[C@H]1CCCO1] | 0.170 |
| 001-65 | 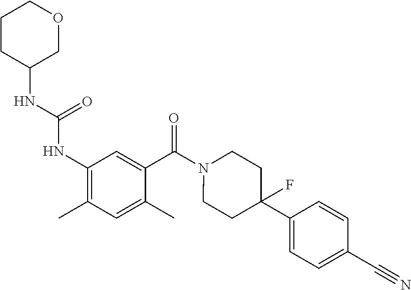 [SMILES: Cc1cc(C)c(C(=O)N2CCC(F)(c3ccc(C#N)cc3)CC2)cc1NC(=O)NC1CCCOC1] | 0.090 |
| 001-66 | 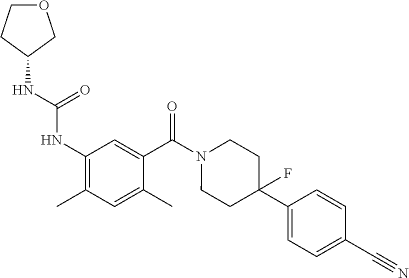 [SMILES: Cc1cc(C)c(C(=O)N2CCC(F)(c3ccc(C#N)cc3)CC2)cc1NC(=O)N[C@@H]1CCOC1] | 0.230 |
| 001-67 | 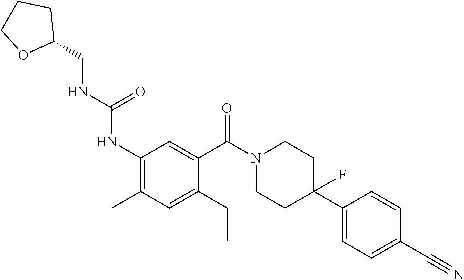 [SMILES: CCc1cc(C)c(NC(=O)NC[C@H]2CCCO2)cc1C(=O)N1CCC(F)(c2ccc(C#N)cc2)CC1] | 0.090 |
| 001-68 | 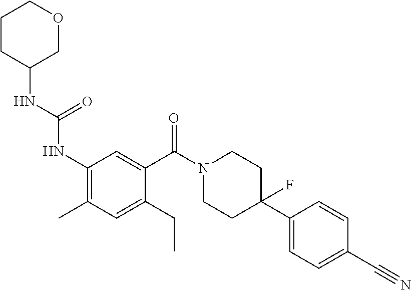 [SMILES: CCc1cc(C)c(NC(=O)NC2CCCOC2)cc1C(=O)N1CCC(F)(c2ccc(C#N)cc2)CC1] | 0.100 |
| 001-312 | 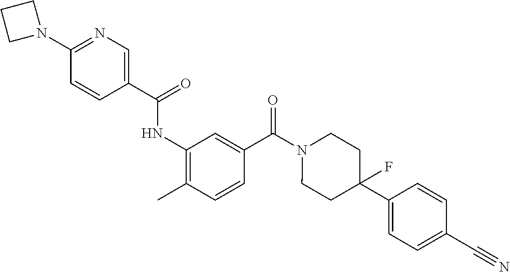 [SMILES: Cc1ccc(C(=O)N2CCC(F)(c3ccc(C#N)cc3)CC2)cc1NC(=O)c1ccc(N2CCC2)nc1] | 0.025 |
| 001-313 | 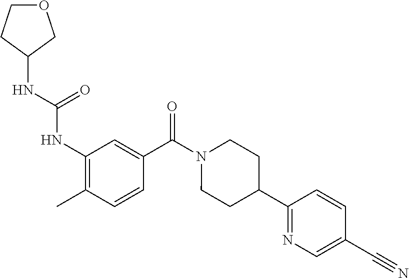 [SMILES: Cc1ccc(C(=O)N2CCC(c3ccc(C#N)cn3)CC2)cc1NC(=O)NC1CCOC1] | 1.630 |
| 001-314 | 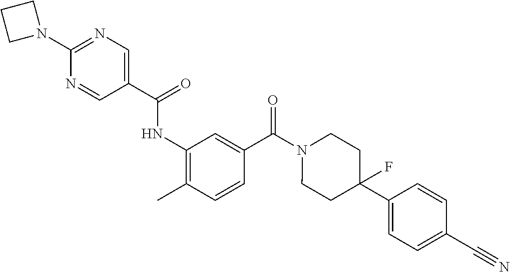 [SMILES: Cc1ccc(C(=O)N2CCC(F)(c3ccc(C#N)cc3)CC2)cc1NC(=O)c1cnc(N2CCC2)nc1] | 0.047 |
| 001-315 | 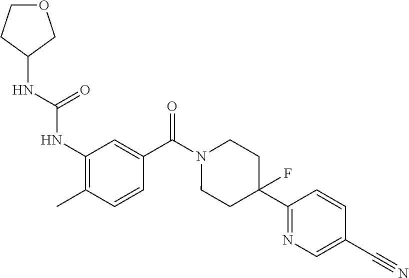 [SMILES: Cc1ccc(C(=O)N2CCC(F)(c3ccc(C#N)cn3)CC2)cc1NC(=O)NC1CCOC1] | 0.267 |
| 001-316 | 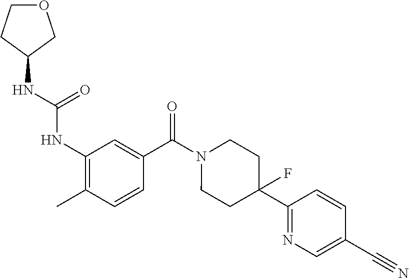 [SMILES: Cc1ccc(C(=O)N2CCC(F)(c3ccc(C#N)cn3)CC2)cc1NC(=O)N[C@H]1CCOC1] | 0.690 |
| 001-317 | 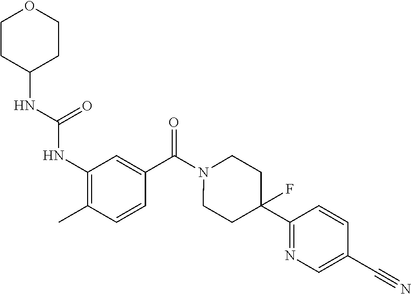 [SMILES: Cc1ccc(C(=O)N2CCC(F)(c3ccc(C#N)cn3)CC2)cc1NC(=O)NC1CCOCC1] | 1.170 |
| 001-69 | 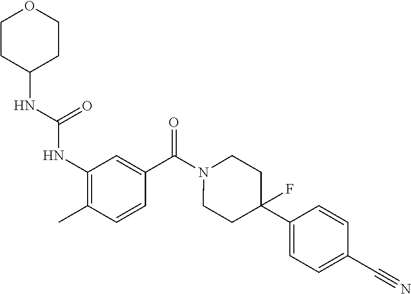 [SMILES: Cc1ccc(C(=O)N2CCC(F)(c3ccc(C#N)cc3)CC2)cc1NC(=O)NC1CCOCC1] | 0.065 |
| 001-70 | 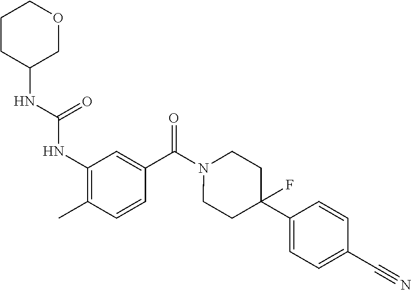 [SMILES: Cc1ccc(C(=O)N2CCC(F)(c3ccc(C#N)cc3)CC2)cc1NC(=O)NC1CCCOC1] | 0.037 |
| 001-71 | 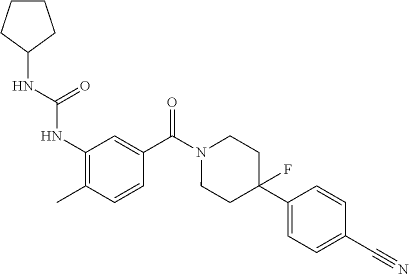 [SMILES: Cc1ccc(C(=O)N2CCC(F)(c3ccc(C#N)cc3)CC2)cc1NC(=O)NC1CCCC1] | 0.030 |
| 001-72 | 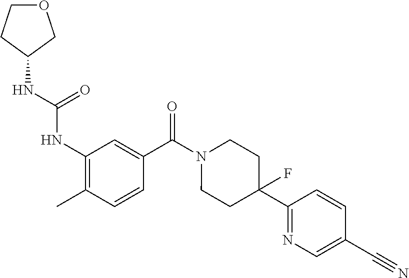 [SMILES: Cc1ccc(C(=O)N2CCC(F)(c3ccc(C#N)cn3)CC2)cc1NC(=O)N[C@@H]1CCOC1] | 0.200 |
| 001-73 | 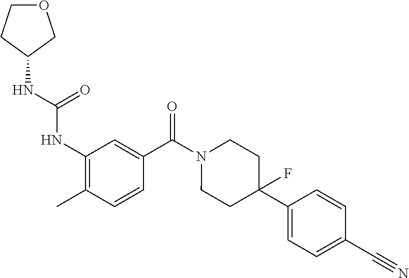 [SMILES: Cc1ccc(C(=O)N2CCC(F)(c3ccc(C#N)cc3)CC2)cc1NC(=O)N[C@@H]1CCOC1] | 0.040 |
| 001-318 | 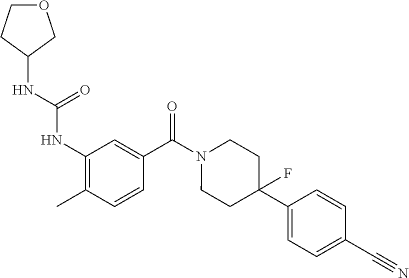 [SMILES: Cc1ccc(C(=O)N2CCC(F)(c3ccc(C#N)cc3)CC2)cc1NC(=O)NC1CCOC1] | 0.037 |
| 001-319 | 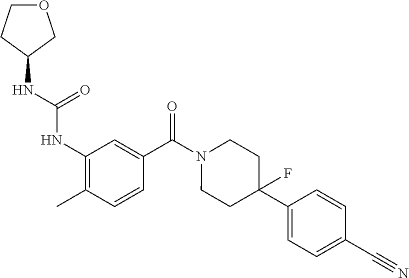 [SMILES: Cc1ccc(C(=O)N2CCC(F)(c3ccc(C#N)cc3)CC2)cc1NC(=O)N[C@H]1CCOC1] | 0.195 |
| 001-320 | 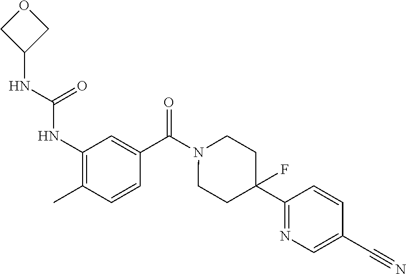 [SMILES: Cc1ccc(C(=O)N2CCC(F)(c3ccc(C#N)cn3)CC2)cc1NC(=O)NC1COC1] | 1.020 |
| 001-321 | 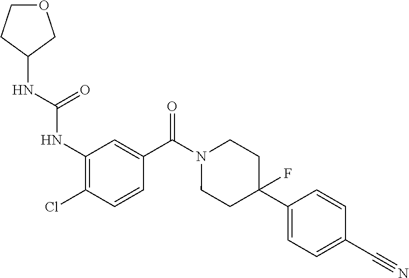 [SMILES: N#Cc1ccc(C2(F)CCN(C(=O)c3ccc(Cl)c(NC(=O)NC4CCOC4)c3)CC2)cc1] | 0.030 |
| 001-322 | 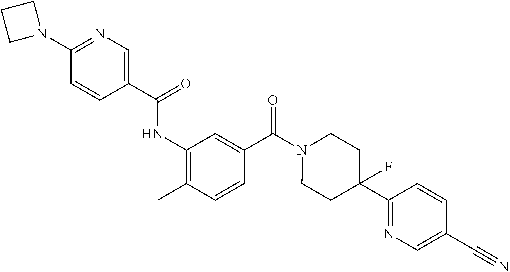 [SMILES: Cc1ccc(C(=O)N2CCC(F)(c3ccc(C#N)cn3)CC2)cc1NC(=O)c1ccc(N2CCC2)nc1] | 0.180 |
| 001-74 | 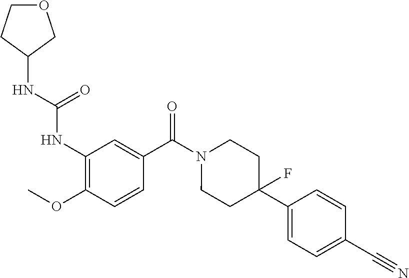 [SMILES: COc1ccc(C(=O)N2CCC(F)(c3ccc(C#N)cc3)CC2)cc1NC(=O)NC1CCOC1] | 0.120 |
| 001-75 | 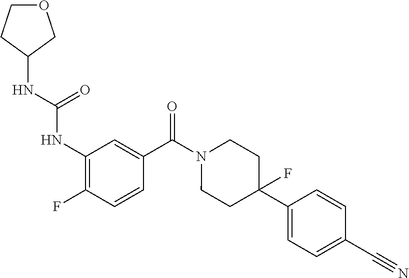 [SMILES: N#Cc1ccc(C2(F)CCN(C(=O)c3ccc(F)c(NC(=O)NC4CCOC4)c3)CC2)cc1] | 0.130 |
| 001-76 | 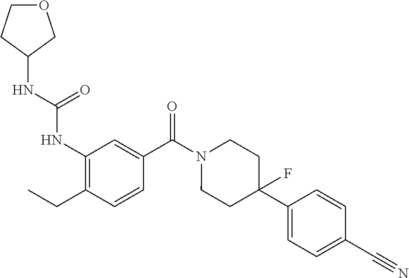 [SMILES: CCc1ccc(C(=O)N2CCC(F)(c3ccc(C#N)cc3)CC2)cc1NC(=O)NC1CCOC1] | 0.035 |
| 001-77 | 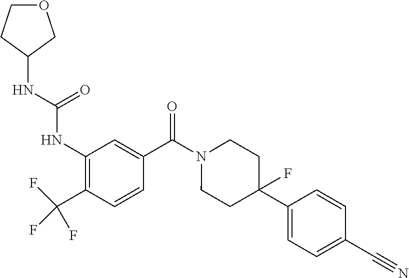 [SMILES: N#Cc1ccc(C2(F)CCN(C(=O)c3ccc(C(F)(F)F)c(NC(=O)NC4CCOC4)c3)CC2)cc1] | 0.080 |
| 001-78 | 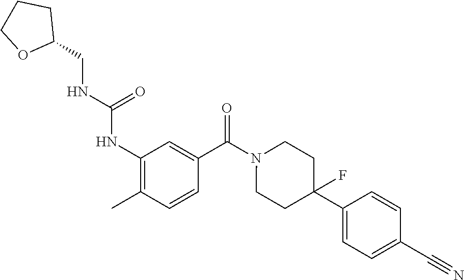 [SMILES: Cc1ccc(C(=O)N2CCC(F)(c3ccc(C#N)cc3)CC2)cc1NC(=O)NC[C@H]1CCCO1] | 0.035 |
| 001-79 | 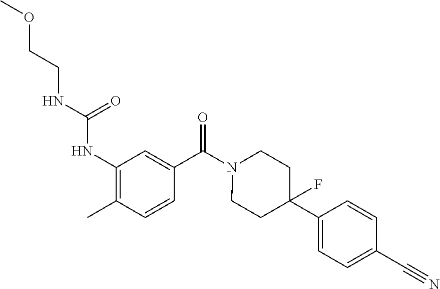 [SMILES: COCCNC(=O)Nc1cc(C(=O)N2CCC(F)(c3ccc(C#N)cc3)CC2)ccc1C] | 0.045 |
| 001-323 | 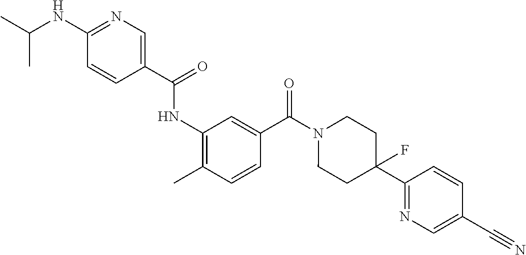 [SMILES: Cc1ccc(C(=O)N2CCC(F)(c3ccc(C#N)cn3)CC2)cc1NC(=O)c1ccc(NC(C)C)nc1] | 0.360 |
| 001-324 | 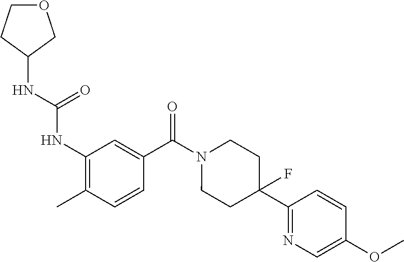 [SMILES: COc1ccc(C2(F)CCN(C(=O)c3ccc(C)c(NC(=O)NC4CCOC4)c3)CC2)nc1] | 1.175 |
| 001-325 | 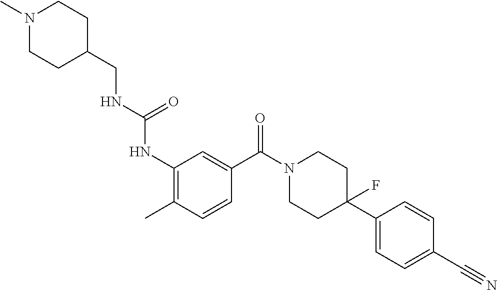 [SMILES: Cc1ccc(C(=O)N2CCC(F)(c3ccc(C#N)cc3)CC2)cc1NC(=O)NCC1CCN(C)CC1] | 0.340 |
| 001-326 | 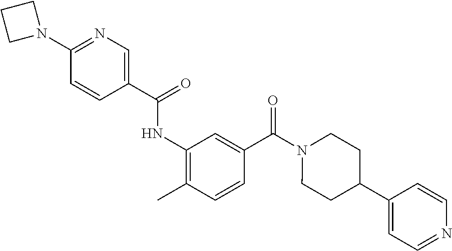 [SMILES: Cc1ccc(C(=O)N2CCC(c3ccncc3)CC2)cc1NC(=O)c1ccc(N2CCC2)nc1] | 2.750 |
| 001-327 | 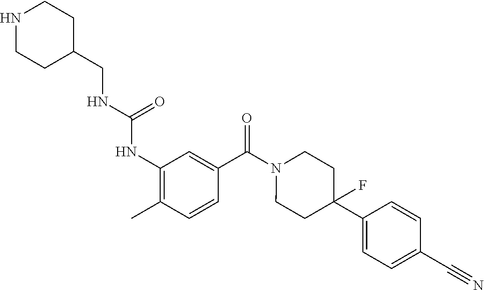 [SMILES: Cc1ccc(C(=O)N2CCC(F)(c3ccc(C#N)cc3)CC2)cc1NC(=O)NCC1CCNCC1] | 0.610 |
| 001-328 | 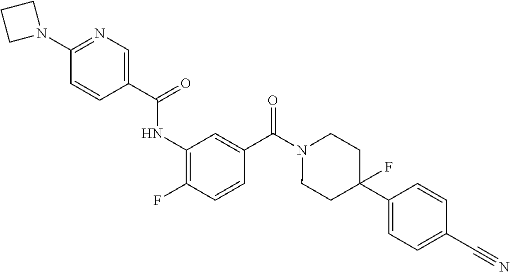 [SMILES: N#Cc1ccc(C2(F)CCN(C(=O)c3ccc(F)c(NC(=O)c4ccc(N5CCC5)nc4)c3)CC2)cc1] | 0.220 |
| 001-80 | 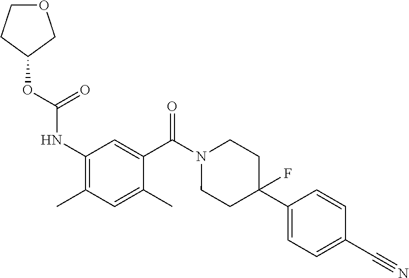 [SMILES: Cc1cc(C)c(C(=O)N2CCC(F)(c3ccc(C#N)cc3)CC2)cc1NC(=O)O[C@@H]1CCOC1] | 0.400 |
| 001-81 | 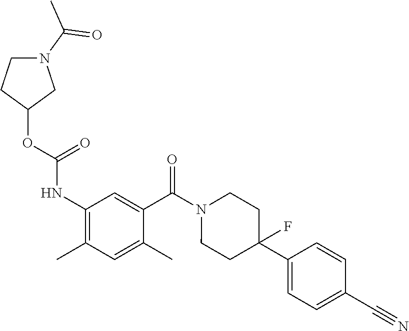 [SMILES: CC(=O)N1CCC(OC(=O)Nc2cc(C(=O)N3CCC(F)(c4ccc(C#N)cc4)CC3)c(C)cc2C)C1] | 0.200 |
| 001-82 | 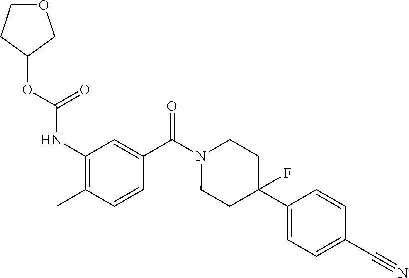 [SMILES: Cc1ccc(C(=O)N2CCC(F)(c3ccc(C#N)cc3)CC2)cc1NC(=O)OC1CCOC1] | 0.077 |
| 001-83 | 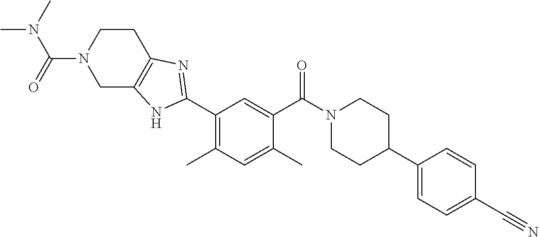 [SMILES: Cc1cc(C)c(-c2nc3c([nH]2)CN(C(=O)N(C)C)CC3)cc1C(=O)N1CCC(c2ccc(C#N)cc2)CC1] | 0.090 |
| 001-84 | 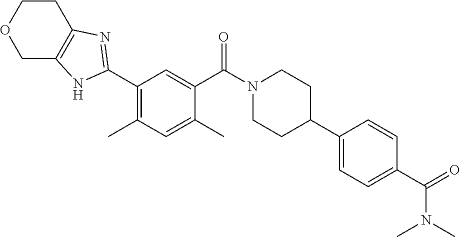 [SMILES: Cc1cc(C)c(-c2nc3c([nH]2)COCC3)cc1C(=O)N1CCC(c2ccc(C(=O)N(C)C)cc2)CC1] | 5.320 |
| 001-85 | 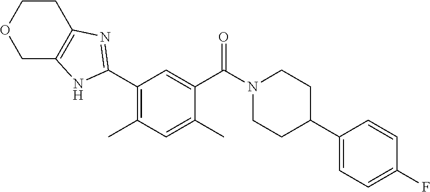 [SMILES: Cc1cc(C)c(-c2nc3c([nH]2)COCC3)cc1C(=O)N1CCC(c2ccc(F)cc2)CC1] | 1.450 |
| 001-329 | 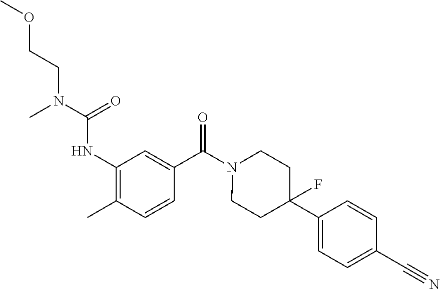 [SMILES: COCCN(C)C(=O)Nc1cc(C(=O)N2CCC(F)(c3ccc(C#N)cc3)CC2)ccc1C] | 8.870 |
| 001-330 | 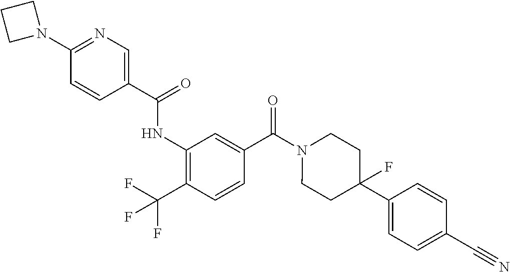 [SMILES: N#Cc1ccc(C2(F)CCN(C(=O)c3ccc(C(F)(F)F)c(NC(=O)c4ccc(N5CCC5)nc4)c3)CC2)cc1] | 0.120 |
| 001-331 | 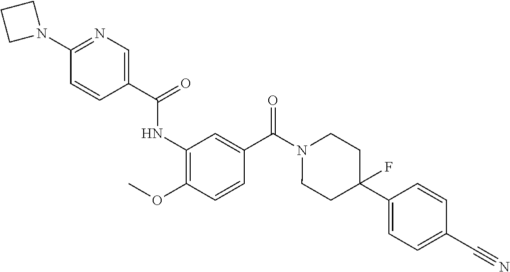 [SMILES: COc1ccc(C(=O)N2CCC(F)(c3ccc(C#N)cc3)CC2)cc1NC(=O)c1ccc(N2CCC2)nc1] | 0.085 |
| 001-332 | 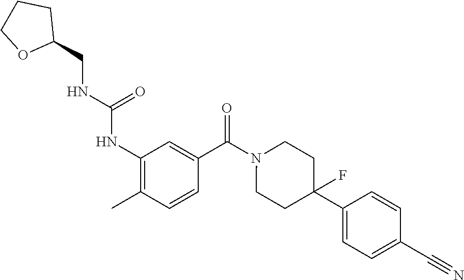 [SMILES: Cc1ccc(C(=O)N2CCC(F)(c3ccc(C#N)cc3)CC2)cc1NC(=O)NC[C@@H]1CCCO1] | 0.050 |
| 001-333 | 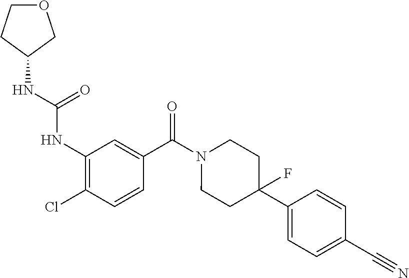 [SMILES: N#Cc1ccc(C2(F)CCN(C(=O)c3ccc(Cl)c(NC(=O)N[C@@H]4CCOC4)c3)CC2)cc1] | |
| 001-334 | 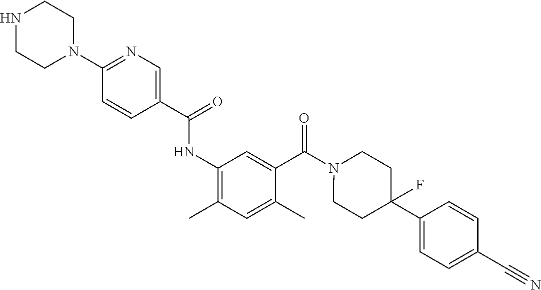 [SMILES: Cc1cc(C)c(C(=O)N2CCC(F)(c3ccc(C#N)cc3)CC2)cc1NC(=O)c1ccc(N2CCNCC2)nc1] | 0.140 |
| 001-86 | 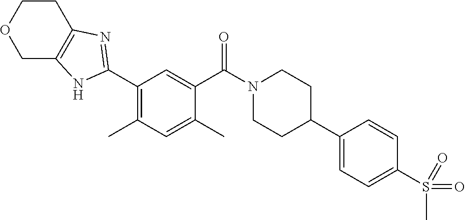 [SMILES: Cc1cc(C)c(-c2nc3c([nH]2)COCC3)cc1C(=O)N1CCC(c2ccc(S(C)(=O)=O)cc2)CC1] | 5.420 |
| 001-87 | 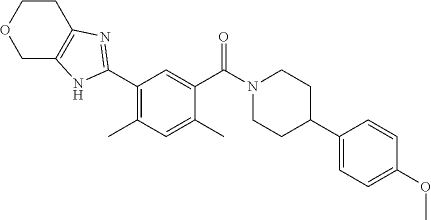 [SMILES: COc1ccc(C2CCN(C(=O)c3cc(-c4nc5c([nH]4)COCC5)c(C)cc3C)CC2)cc1] | 0.310 |
| 001-88 | 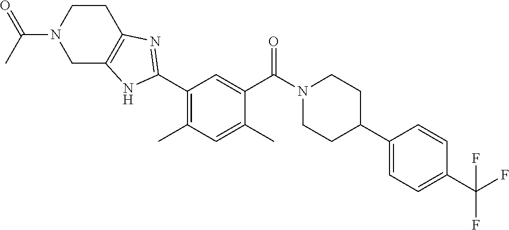 [SMILES: CC(=O)N1CCc2nc(-c3cc(C(=O)N4CCC(c5ccc(C(F)(F)F)cc5)CC4)c(C)cc3C)[nH]c2C1] | 0.040 |
| 001-89 | 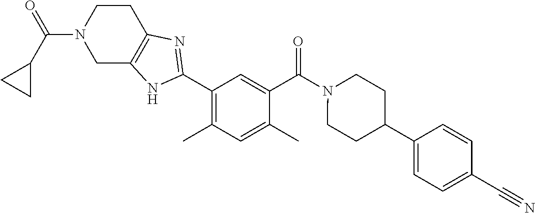 [SMILES: Cc1cc(C)c(-c2nc3c([nH]2)CN(C(=O)C2CC2)CC3)cc1C(=O)N1CCC(c2ccc(C#N)cc2)CC1] | 0.030 |
| 001-90 | 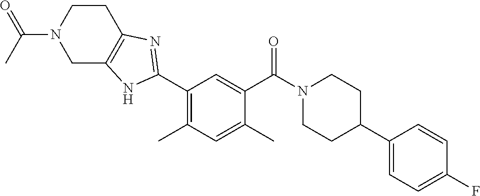 [SMILES: CC(=O)N1CCc2nc(-c3cc(C(=O)N4CCC(c5ccc(F)cc5)CC4)c(C)cc3C)[nH]c2C1] | 0.380 |
| 001-91 | 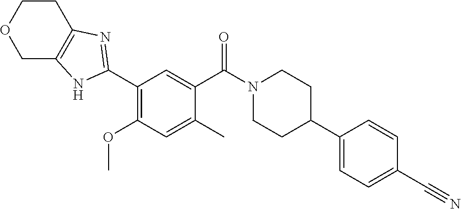 [SMILES: COc1cc(C)c(C(=O)N2CCC(c3ccc(C#N)cc3)CC2)cc1-c1nc2c([nH]1)COCC2] | 2.210 |
| 001-335 | 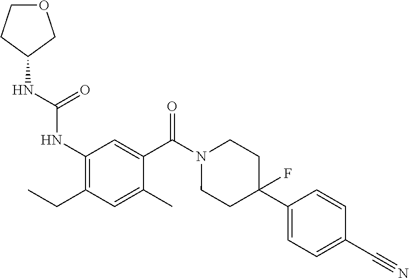 [SMILES: CCc1cc(C)c(C(=O)N2CCC(F)(c3ccc(C#N)cc3)CC2)cc1NC(=O)N[C@@H]1CCOC1] | 0.490 |
| 001-336 | 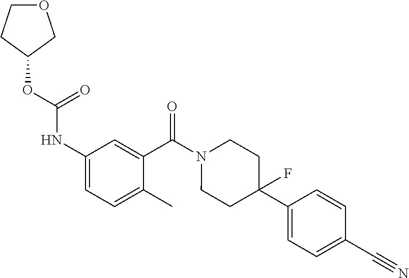 [SMILES: Cc1ccc(NC(=O)O[C@@H]2CCOC2)cc1C(=O)N1CCC(F)(c2ccc(C#N)cc2)CC1] | 0.420 |
| 001-337 | 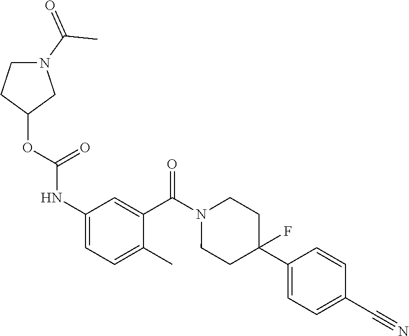 [SMILES: CC(=O)N1CCC(OC(=O)Nc2ccc(C)c(C(=O)N3CCC(F)(c4ccc(C#N)cc4)CC3)c2)C1] | 0.165 |
| 001-338 | 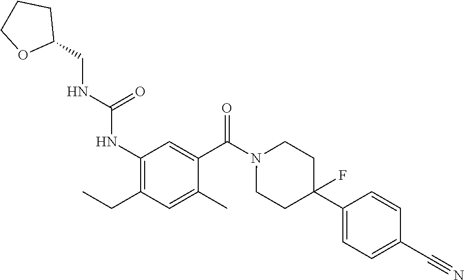 [SMILES: CCc1cc(C)c(C(=O)N2CCC(F)(c3ccc(C#N)cc3)CC2)cc1NC(=O)NC[C@H]1CCCO1] | 0.035 |
| 001-339 | 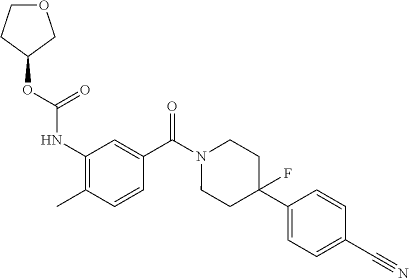 [SMILES: Cc1ccc(C(=O)N2CCC(F)(c3ccc(C#N)cc3)CC2)cc1NC(=O)O[C@H]1CCOC1] | 0.093 |
| 001-340 | 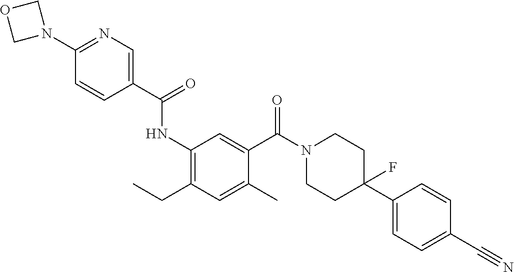 [SMILES: CCc1cc(C)c(C(=O)N2CCC(F)(c3ccc(C#N)cc3)CC2)cc1NC(=O)c1ccc(N2COC2)nc1] | 0.080 |
| 001-92 | 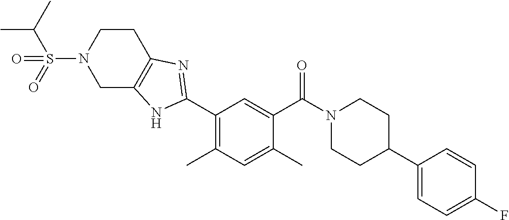 [SMILES: Cc1cc(C)c(-c2nc3c([nH]2)CN(S(=O)(=O)C(C)C)CC3)cc1C(=O)N1CCC(c2ccc(F)cc2)CC1] | 0.940 |
| 001-93 | 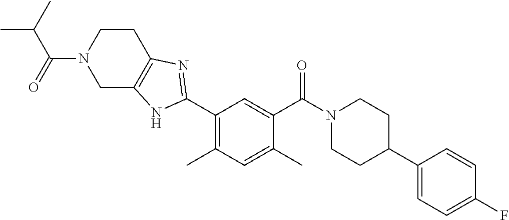 [SMILES: Cc1cc(C)c(-c2nc3c([nH]2)CN(C(=O)C(C)C)CC3)cc1C(=O)N1CCC(c2ccc(F)cc2)CC1] | 0.550 |
| 001-94 | 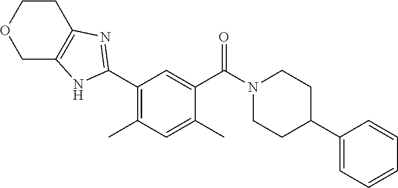 [SMILES: Cc1cc(C)c(-c2nc3c([nH]2)COCC3)cc1C(=O)N1CCC(c2ccccc2)CC1] | 5.260 |
| 001-95 | 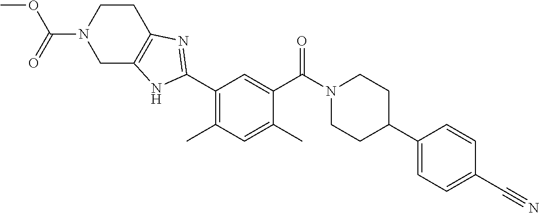 [SMILES: COC(=O)N1CCc2nc(-c3cc(C(=O)N4CCC(c5ccc(C#N)cc5)CC4)c(C)cc3C)[nH]c2C1] | 0.030 |
| 001-96 | 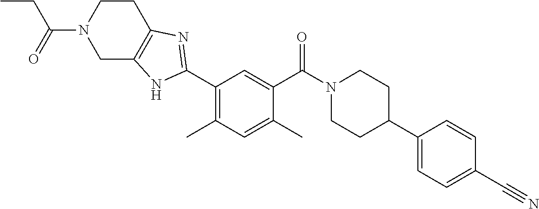 [SMILES: CCC(=O)N1CCc2nc(-c3cc(C(=O)N4CCC(c5ccc(C#N)cc5)CC4)c(C)cc3C)[nH]c2C1] | 0.030 |
| 001-97 | 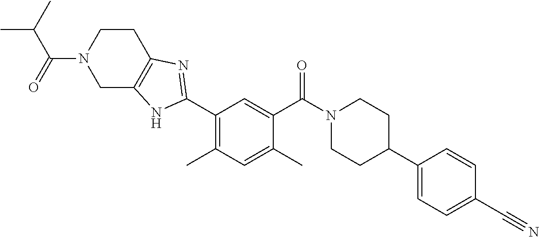 [SMILES: Cc1cc(C)c(-c2nc3c([nH]2)CN(C(=O)C(C)C)CC3)cc1C(=O)N1CCC(c2ccc(C#N)cc2)CC1] | 0.025 |
| 001-98 | 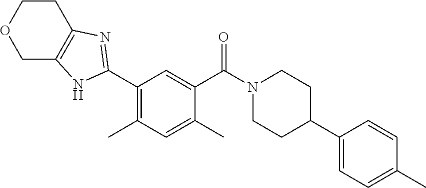 [SMILES: Cc1ccc(C2CCN(C(=O)c3cc(-c4nc5c([nH]4)COCC5)c(C)cc3C)CC2)cc1] | 0.900 |
| 001-341 | 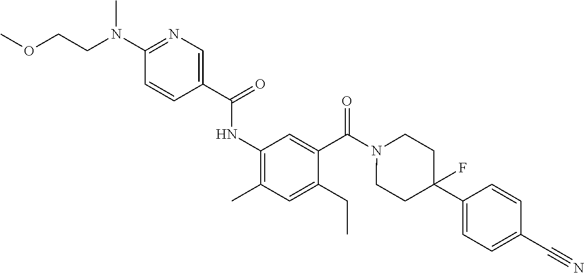 [SMILES: CCc1cc(C)c(NC(=O)c2ccc(N(C)CCOC)nc2)cc1C(=O)N1CCC(F)(c2ccc(C#N)cc2)CC1] | 0.085 |
| 001-342 | 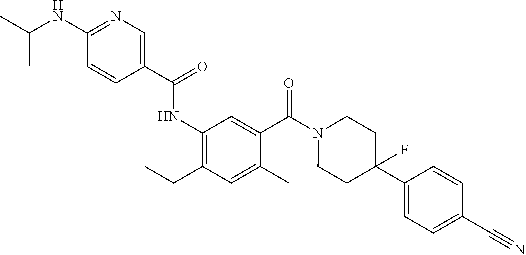 [SMILES: CCc1cc(C)c(C(=O)N2CCC(F)(c3ccc(C#N)cc3)CC2)cc1NC(=O)c1ccc(NC(C)C)nc1] | 0.090 |
| 001-343 | 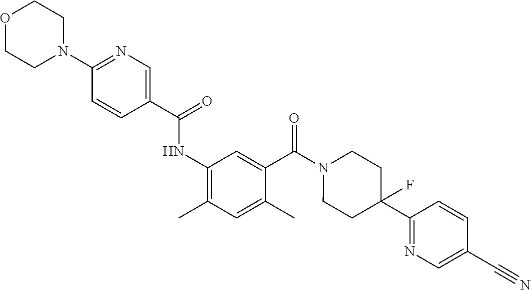 [SMILES: Cc1cc(C)c(C(=O)N2CCC(F)(c3ccc(C#N)cn3)CC2)cc1NC(=O)c1ccc(N2CCOCC2)nc1] | 0.600 |
| 001-344 | 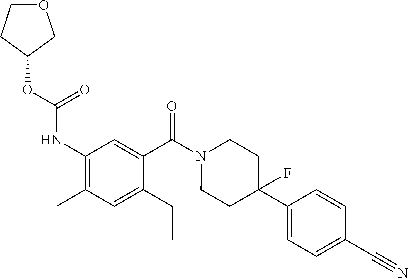 [SMILES: CCc1cc(C)c(NC(=O)O[C@@H]2CCOC2)cc1C(=O)N1CCC(F)(c2ccc(C#N)cc2)CC1] | 0.250 |
| 001-345 | 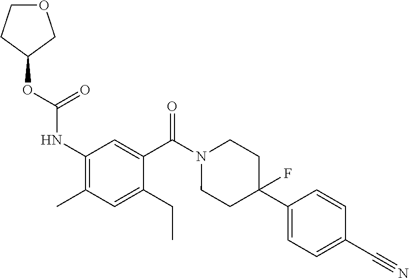 [SMILES: CCc1cc(C)c(NC(=O)O[C@H]2CCOC2)cc1C(=O)N1CCC(F)(c2ccc(C#N)cc2)CC1] | 0.140 |
| 001-346 | 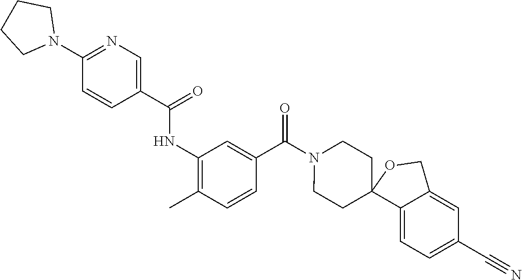 [SMILES: Cc1ccc(C(=O)N2CCC3(CC2)OCc2cc(C#N)ccc23)cc1NC(=O)c1ccc(N2CCCC2)nc1] | 0.330 |
| 001-347 | 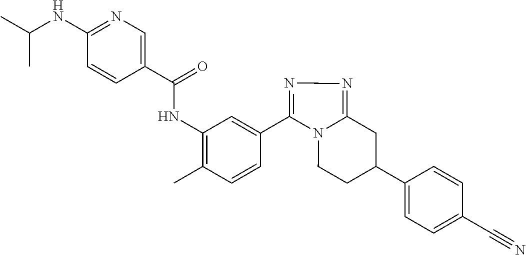 [SMILES: Cc1ccc(-c2nnc3n2CCC(c2ccc(C#N)cc2)C3)cc1NC(=O)c1ccc(NC(C)C)nc1] | 0.300 |
| 001-99 | 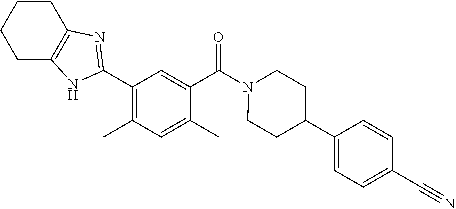 [SMILES: Cc1cc(C)c(-c2nc3c([nH]2)CCCC3)cc1C(=O)N1CCC(c2ccc(C#N)cc2)CC1] | 0.220 |
| 001-100 | 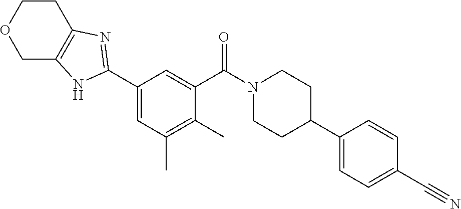 [SMILES: Cc1cc(-c2nc3c([nH]2)COCC3)cc(C(=O)N2CCC(c3ccc(C#N)cc3)CC2)c1C] | >50 |
| 001-101 | 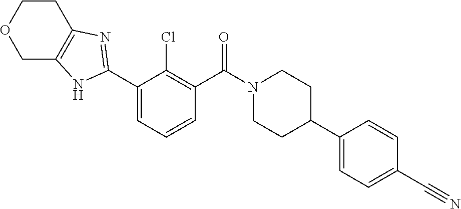 [SMILES: N#Cc1ccc(C2CCN(C(=O)c3cccc(-c4nc5c([nH]4)COCC5)c3Cl)CC2)cc1] | >50 |
| 001-102 | 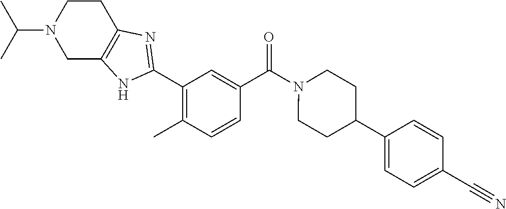 [SMILES: Cc1ccc(C(=O)N2CCC(c3ccc(C#N)cc3)CC2)cc1-c1nc2c([nH]1)CN(C(C)C)CC2] | 0.085 |
| 001-103 | 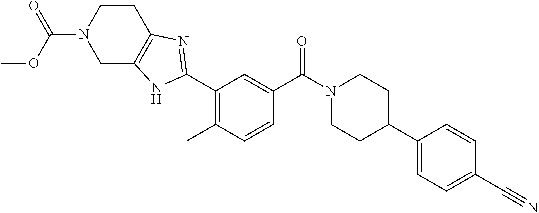 [SMILES: COC(=O)N1CCc2nc(-c3cc(C(=O)N4CCC(c5ccc(C#N)cc5)CC4)ccc3C)[nH]c2C1] | 0.035 |
| 001-104 | 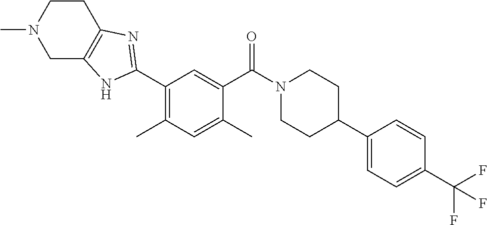 [SMILES: Cc1cc(C)c(-c2nc3c([nH]2)CN(C)CC3)cc1C(=O)N1CCC(c2ccc(C(F)(F)F)cc2)CC1] | 0.130 |
| 001-105 | 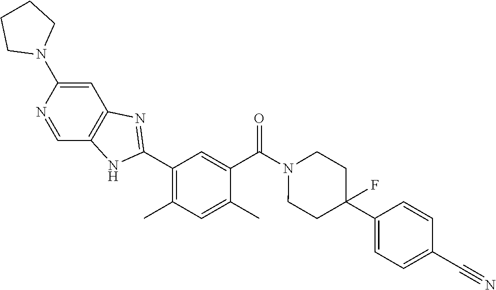 [SMILES: Cc1cc(C)c(-c2nc3cc(N4CCCC4)ncc3[nH]2)cc1C(=O)N1CCC(F)(c2ccc(C#N)cc2)CC1] | 0.015 |
| 001-348 | 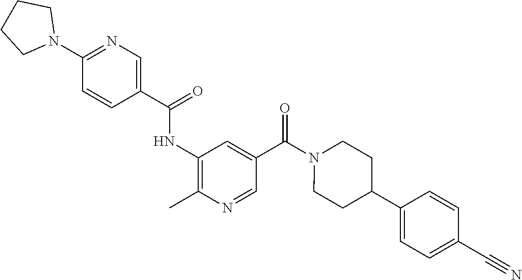 [SMILES: Cc1ncc(C(=O)N2CCC(c3ccc(C#N)cc3)CC2)cc1NC(=O)c1ccc(N2CCCC2)nc1] | 0.455 |
| 001-349 | 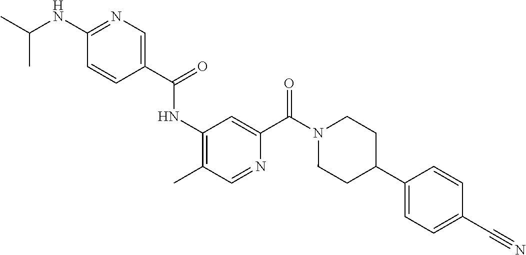 [SMILES: Cc1cnc(C(=O)N2CCC(c3ccc(C#N)cc3)CC2)cc1NC(=O)c1ccc(NC(C)C)nc1] | >50 |
| 001-350 | 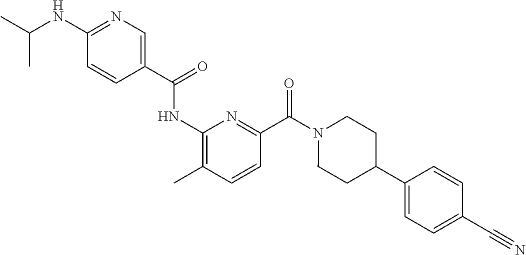 [SMILES: Cc1ccc(C(=O)N2CCC(c3ccc(C#N)cc3)CC2)nc1NC(=O)c1ccc(NC(C)C)nc1] | 32.600 |
| 001-351 | 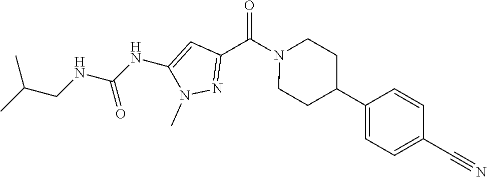 [SMILES: CC(C)CNC(=O)Nc1cc(C(=O)N2CCC(c3ccc(C#N)cc3)CC2)nn1C] | >50 |
| 001-352 | 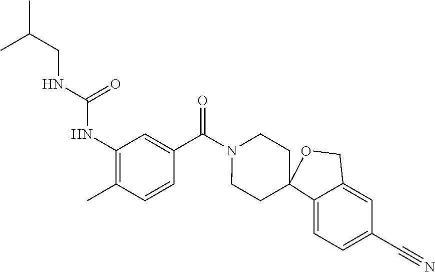 [SMILES: Cc1ccc(C(=O)N2CCC3(CC2)OCc2cc(C#N)ccc23)cc1NC(=O)NCC(C)C] | 0.880 |
| 001-353 | 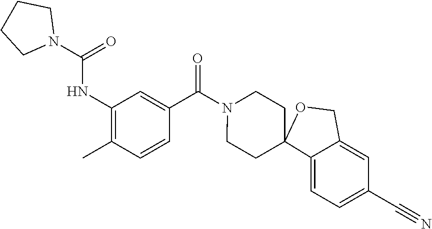 [SMILES: Cc1ccc(C(=O)N2CCC3(CC2)OCc2cc(C#N)ccc23)cc1NC(=O)N1CCCC1] | 18.500 |
| 001-354 | 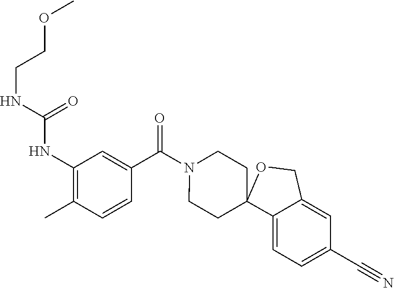 [SMILES: COCCNC(=O)Nc1cc(C(=O)N2CCC3(CC2)OCc2cc(C#N)ccc23)ccc1C] | 0.380 |
| 001-106 | 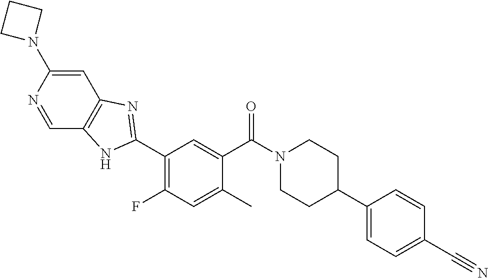 [SMILES: Cc1cc(F)c(-c2nc3cc(N4CCC4)ncc3[nH]2)cc1C(=O)N1CCC(c2ccc(C#N)cc2)CC1] | 0.900 |
| 001-107 | 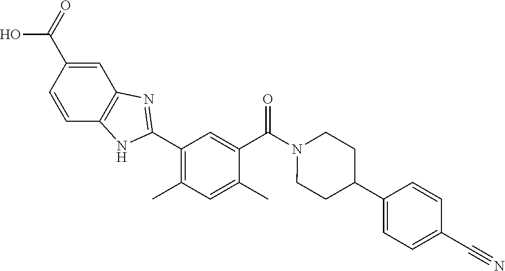 [SMILES: Cc1cc(C)c(-c2nc3cc(C(=O)O)ccc3[nH]2)cc1C(=O)N1CCC(c2ccc(C#N)cc2)CC1] | 1.490 |
| 001-108 | 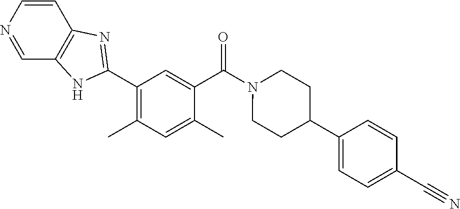 [SMILES: Cc1cc(C)c(-c2nc3ccncc3[nH]2)cc1C(=O)N1CCC(c2ccc(C#N)cc2)CC1] | 1.050 |
| 001-109 | 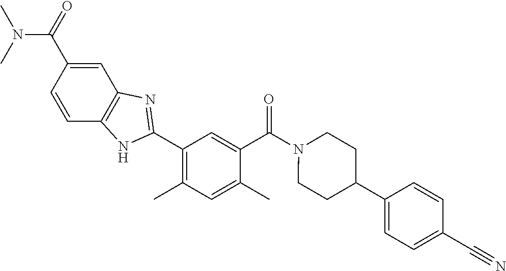 [SMILES: Cc1cc(C)c(-c2nc3cc(C(=O)N(C)C)ccc3[nH]2)cc1C(=O)N1CCC(c2ccc(C#N)cc2)CC1] | 0.250 |
| 001-110 | 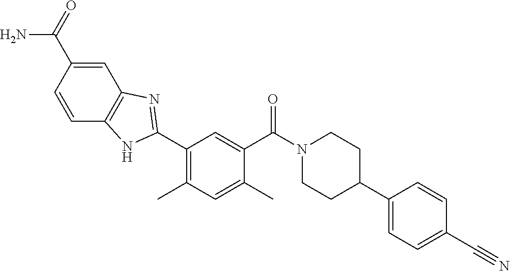 [SMILES: Cc1cc(C)c(-c2nc3cc(C(N)=O)ccc3[nH]2)cc1C(=O)N1CCC(c2ccc(C#N)cc2)CC1] | 0.280 |
| 001-111 | 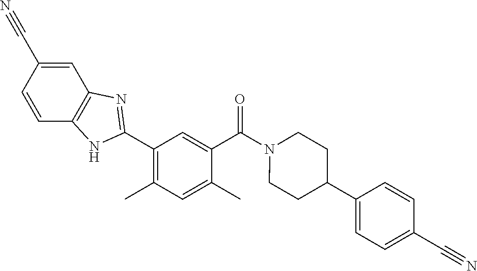 [SMILES: Cc1cc(C)c(-c2nc3cc(C#N)ccc3[nH]2)cc1C(=O)N1CCC(c2ccc(C#N)cc2)CC1] | 0.135 |
| 001-112 | 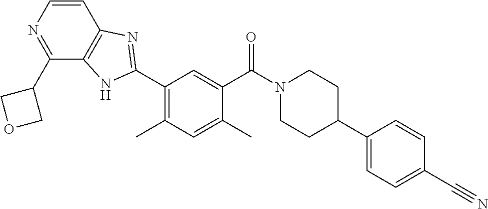 [SMILES: Cc1cc(C)c(-c2nc3ccnc(C4COC4)c3[nH]2)cc1C(=O)N1CCC(c2ccc(C#N)cc2)CC1] | 1.295 |
| 001-355 | 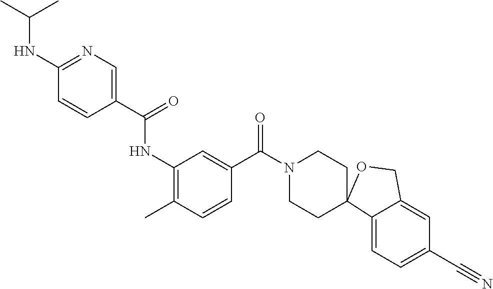 [SMILES: Cc1ccc(C(=O)N2CCC3(CC2)OCc2cc(C#N)ccc23)cc1NC(=O)c1ccc(NC(C)C)nc1] | 0.140 |
| 001-356 | 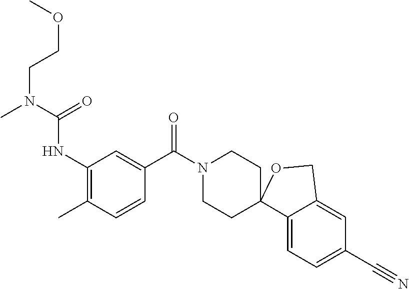 [SMILES: COCCN(C)C(=O)Nc1cc(C(=O)N2CCC3(CC2)OCc2cc(C#N)ccc23)ccc1C] | >50 |
| 001-357 | 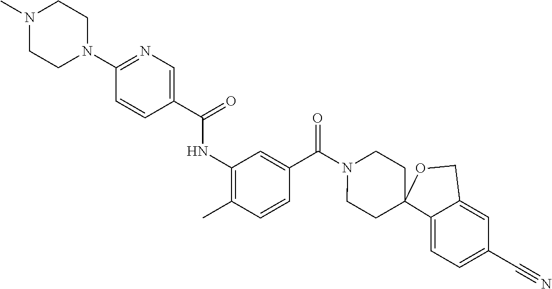 [SMILES: Cc1ccc(C(=O)N2CCC3(CC2)OCc2cc(C#N)ccc23)cc1NC(=O)c1ccc(N2CCN(C)CC2)nc1] | 0.230 |
| 358 | 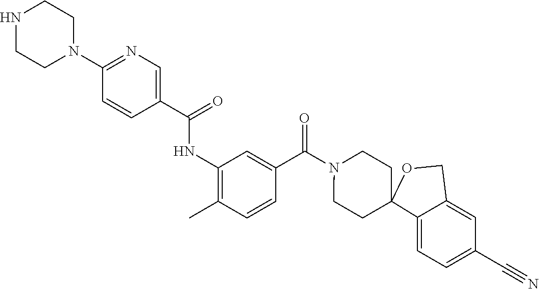 [SMILES: Cc1ccc(C(=O)N2CCC3(CC2)OCc2cc(C#N)ccc23)cc1NC(=O)c1ccc(N2CCNCC2)nc1] | 0.805 |
| 001-359 | 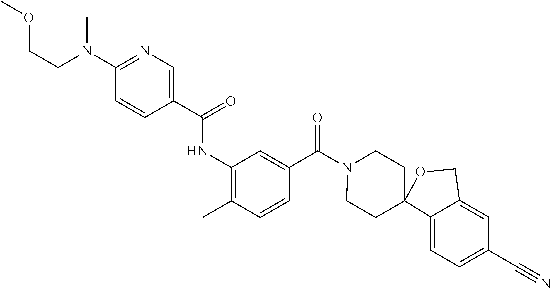 [SMILES: COCCN(C)c1ccc(C(=O)Nc2cc(C(=O)N3CCC4(CC3)OCc3cc(C#N)ccc34)ccc2C)cn1] | 0.105 |
| 001-360 | 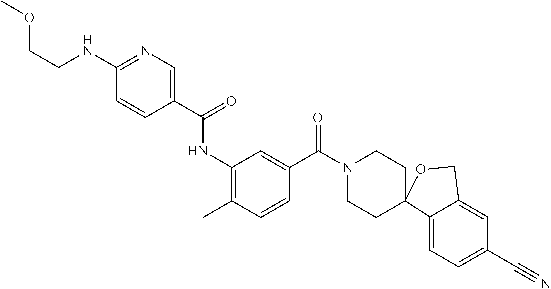 [SMILES: COCCNc1ccc(C(=O)Nc2cc(C(=O)N3CCC4(CC3)OCc3cc(C#N)ccc34)ccc2C)cn1] | 0.280 |
| 001-361 | 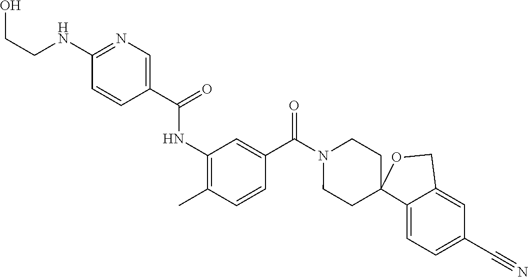 [SMILES: Cc1ccc(C(=O)N2CCC3(CC2)OCc2cc(C#N)ccc23)cc1NC(=O)c1ccc(NCCO)nc1] | 0.360 |
| 001-113 | 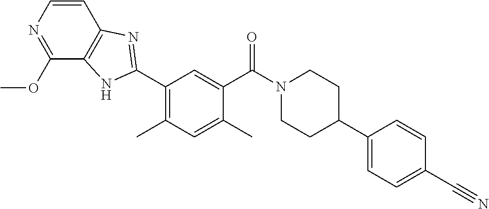 [SMILES: COc1nccc2nc(-c3cc(C(=O)N4CCC(c5ccc(C#N)cc5)CC4)c(C)cc3C)[nH]c12] | 0.130 |
| 001-114 | 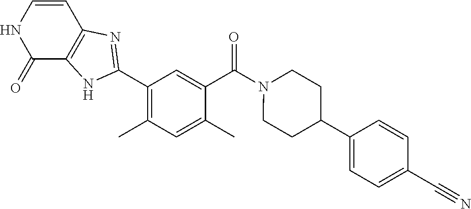 [SMILES: Cc1cc(C)c(-c2nc3cc[nH]c(=O)c3[nH]2)cc1C(=O)N1CCC(c2ccc(C#N)cc2)CC1] | 0.245 |
| 001-115 | 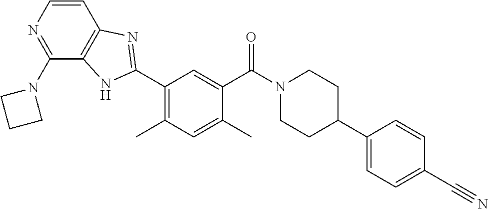 [SMILES: Cc1cc(C)c(-c2nc3ccnc(N4CCC4)c3[nH]2)cc1C(=O)N1CCC(c2ccc(C#N)cc2)CC1] | 0.480 |
| 001-116 | 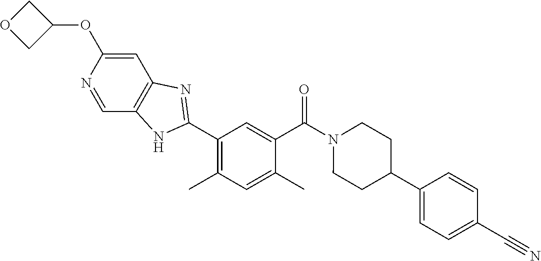 [SMILES: Cc1cc(C)c(-c2nc3cc(OC4COC4)ncc3[nH]2)cc1C(=O)N1CCC(c2ccc(C#N)cc2)CC1] | 0.090 |
| 001-117 | 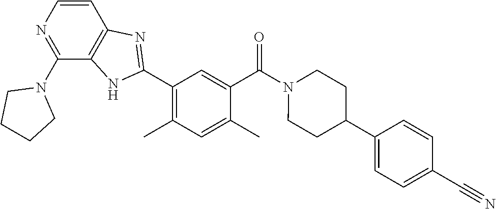 [SMILES: Cc1cc(C)c(-c2nc3ccnc(N4CCCC4)c3[nH]2)cc1C(=O)N1CCC(c2ccc(C#N)cc2)CC1] | 0.090 |
| 001-118 | 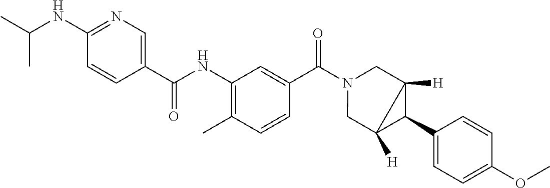 [SMILES: COc1ccc([C@H]2[C@@H]3CN(C(=O)c4ccc(C)c(NC(=O)c5ccc(NC(C)C)nc5)c4)C[C@@H]32)cc1] | 0.350 |
| 001-362 | 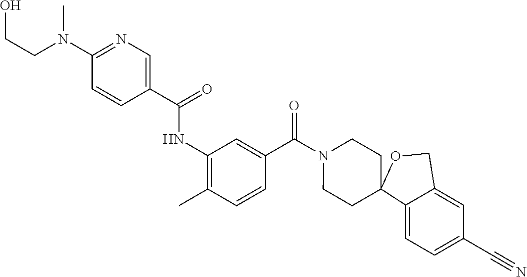 [SMILES: Cc1ccc(C(=O)N2CCC3(CC2)OCc2cc(C#N)ccc23)cc1NC(=O)c1ccc(N(C)CCO)nc1] | 0.247 |
| 001-363 | 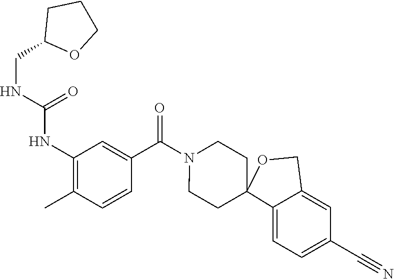 [SMILES: Cc1ccc(C(=O)N2CCC3(CC2)OCc2cc(C#N)ccc23)cc1NC(=O)NC[C@@H]1CCCO1] | 0.050 |
| 001-364 | 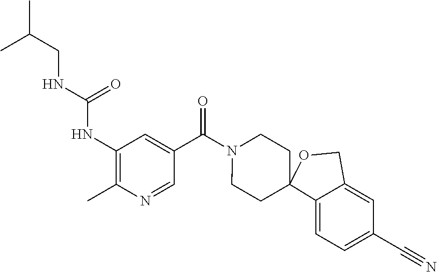 [SMILES: Cc1ncc(C(=O)N2CCC3(CC2)OCc2cc(C#N)ccc23)cc1NC(=O)NCC(C)C] | 37.000 |
| 001-365 | 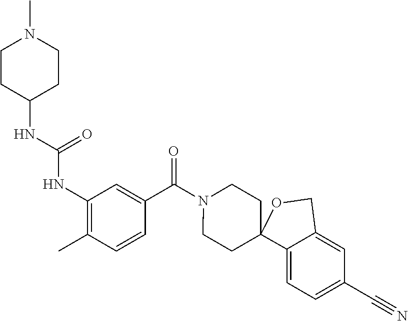 [SMILES: Cc1ccc(C(=O)N2CCC3(CC2)OCc2cc(C#N)ccc23)cc1NC(=O)NC1CCN(C)CC1] | 0.310 |
| 001-366 | 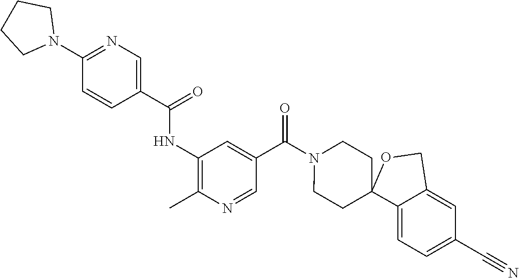 [SMILES: Cc1ncc(C(=O)N2CCC3(CC2)OCc2cc(C#N)ccc23)cc1NC(=O)c1ccc(N2CCCC2)nc1] | 1.100 |
| 001-367 | 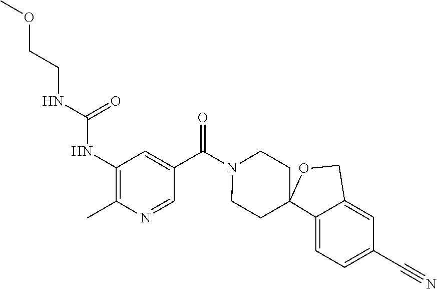 [SMILES: COCCNC(=O)Nc1cc(C(=O)N2CCC3(CC2)OCc2cc(C#N)ccc23)cnc1C] | |
| 001-119 | 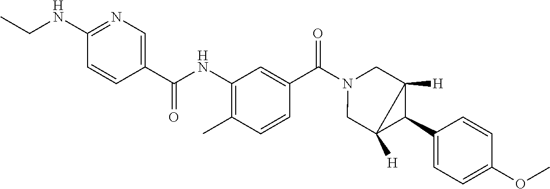 [SMILES: CCNc1ccc(C(=O)Nc2cc(C(=O)N3C[C@@H]4[C@H](C3)[C@H]4c3ccc(OC)cc3)ccc2C)cn1] | |
| 001-120 | 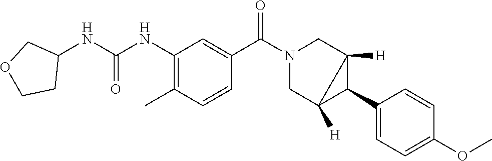 [SMILES: COc1ccc([C@H]2[C@@H]3CN(C(=O)c4ccc(C)c(NC(=O)NC5CCOC5)c4)C[C@@H]32)cc1] | 0.460 |
| 001-121 | 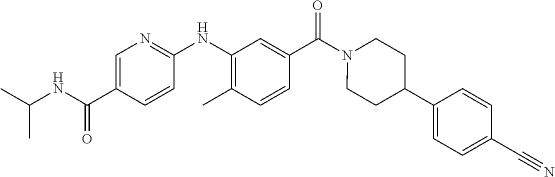 [SMILES: Cc1ccc(C(=O)N2CCC(c3ccc(C#N)cc3)CC2)cc1Nc1ccc(C(=O)NC(C)C)cn1] | 2.300 |
| 001-122 | 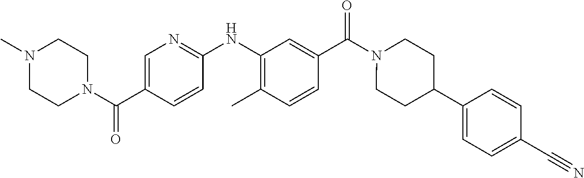 [SMILES: Cc1ccc(C(=O)N2CCC(c3ccc(C#N)cc3)CC2)cc1Nc1ccc(C(=O)N2CCN(C)CC2)cn1] | 0.890 |
| 001-123 | 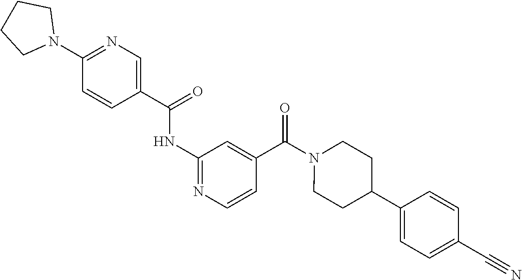 [SMILES: N#Cc1ccc(C2CCN(C(=O)c3ccnc(NC(=O)c4ccc(N5CCCC5)nc4)c3)CC2)cc1] | 0.690 |
| 001-124 | 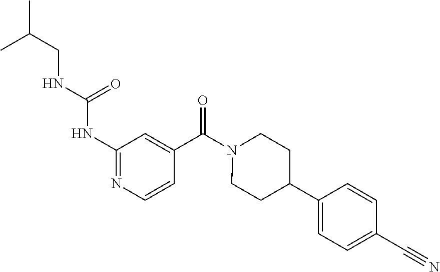 [SMILES: CC(C)CNC(=O)Nc1cc(C(=O)N2CCC(c3ccc(C#N)cc3)CC2)ccn1] | 32.880 |
| 001-125 | 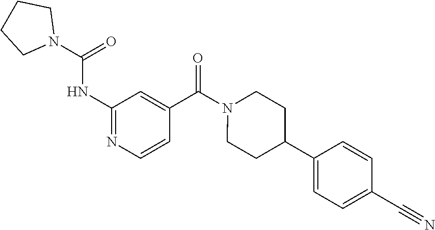 [SMILES: N#Cc1ccc(C2CCN(C(=O)c3ccnc(NC(=O)N4CCCC4)c3)CC2)cc1] | >50 |
| 001-368 | 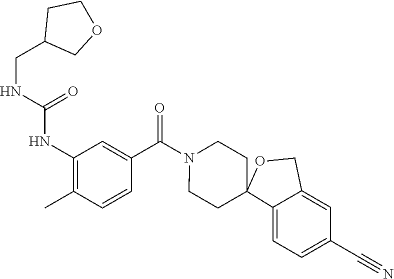 [SMILES: Cc1ccc(C(=O)N2CCC3(CC2)OCc2cc(C#N)ccc23)cc1NC(=O)NCC1CCOC1] | 0.310 |
| 001-369 | 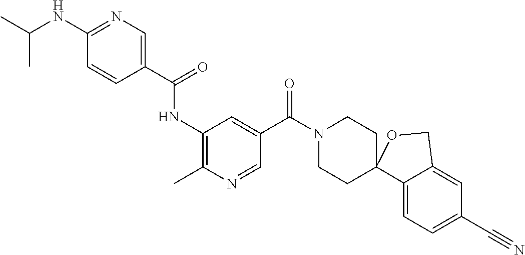 [SMILES: Cc1ncc(C(=O)N2CCC3(CC2)OCc2cc(C#N)ccc23)cc1NC(=O)c1ccc(NC(C)C)nc1] | 7.150 |
| 001-370 | 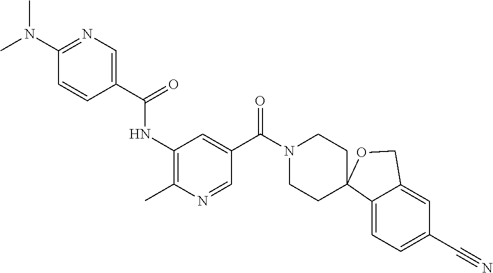 [SMILES: Cc1ncc(C(=O)N2CCC3(CC2)OCc2cc(C#N)ccc23)cc1NC(=O)c1ccc(N(C)C)nc1] | |
| 001-371 | 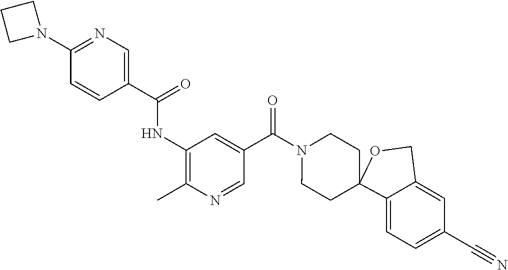 [SMILES: Cc1ncc(C(=O)N2CCC3(CC2)OCc2cc(C#N)ccc23)cc1NC(=O)c1ccc(N2CCC2)nc1] | |
| 001-372 | 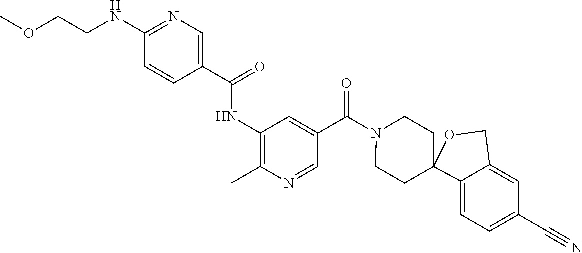 [SMILES: COCCNc1ccc(C(=O)Nc2cc(C(=O)N3CCC4(CC3)OCc3cc(C#N)ccc34)cnc2C)cn1] | |
| 001-373 | 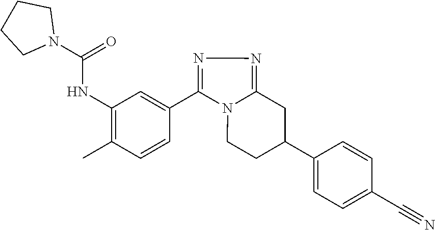 [SMILES: Cc1ccc(-c2nnc3n2CCC(c2ccc(C#N)cc2)C3)cc1NC(=O)N1CCCC1] | >50 |
| 001-374 | 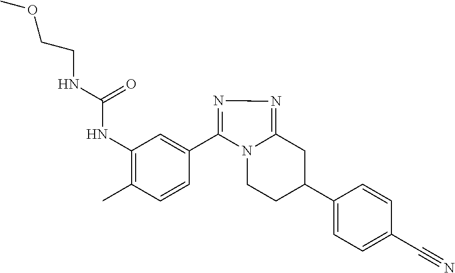 [SMILES: COCCNC(=O)Nc1cc(-c2nnc3n2CCC(c2ccc(C#N)cc2)C3)ccc1C] | 3.580 |
| 001-126 | 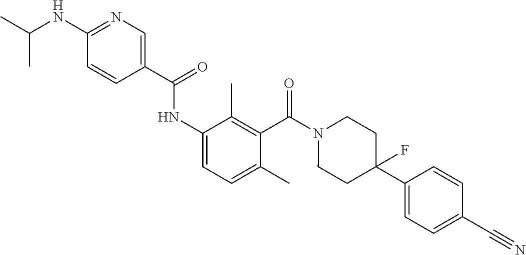 [SMILES: Cc1ccc(NC(=O)c2ccc(NC(C)C)nc2)c(C)c1C(=O)N1CCC(F)(c2ccc(C#N)cc2)CC1] | >50 |
| 001-127 | 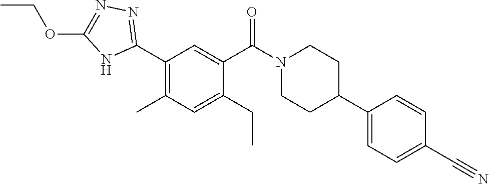 [SMILES: CCOc1nnc(-c2cc(C(=O)N3CCC(c4ccc(C#N)cc4)CC3)c(CC)cc2C)[nH]1] | 0.730 |
| 001-128 | 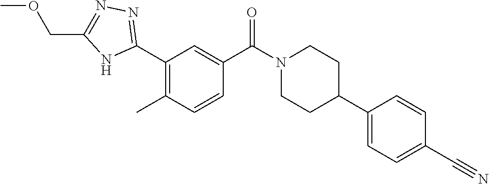 [SMILES: COCc1nnc(-c2cc(C(=O)N3CCC(c4ccc(C#N)cc4)CC3)ccc2C)[nH]1] | 2.260 |
| 001-129 | 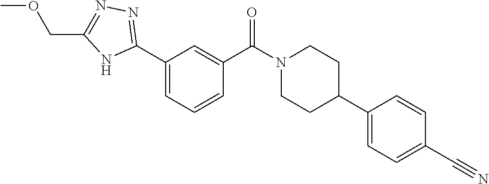 [SMILES: COCc1nnc(-c2cccc(C(=O)N3CCC(c4ccc(C#N)cc4)CC3)c2)[nH]1] | >50 |
| 001-130 | 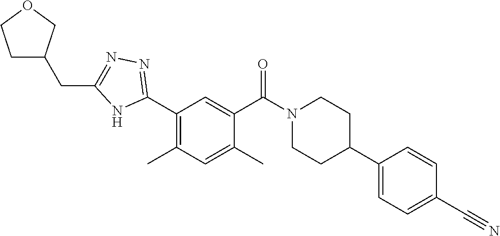 [SMILES: Cc1cc(C)c(-c2nnc(CC3CCOC3)[nH]2)cc1C(=O)N1CCC(c2ccc(C#N)cc2)CC1] | 0.300 |
| 001-131 | 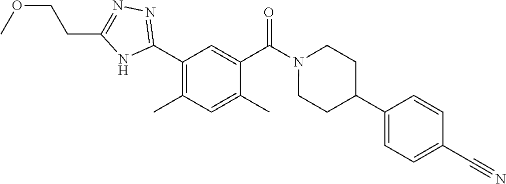 [SMILES: COCCc1nnc(-c2cc(C(=O)N3CCC(c4ccc(C#N)cc4)CC3)c(C)cc2C)[nH]1] | 0.220 |
| 001-375 | 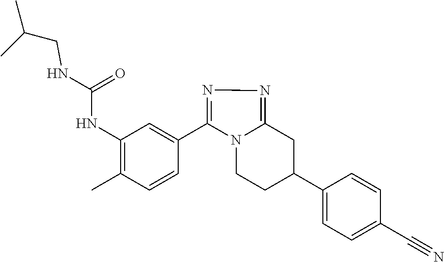 [SMILES: Cc1ccc(-c2nnc3n2CCC(c2ccc(C#N)cc2)C3)cc1NC(=O)NCC(C)C] | 6.360 |
| 001-376 | 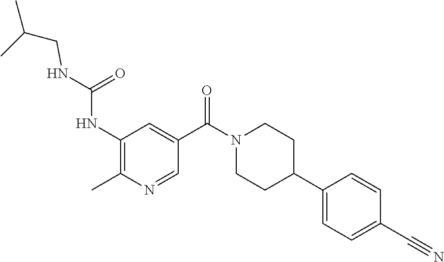 [SMILES: Cc1ncc(C(=O)N2CCC(c3ccc(C#N)cc3)CC2)cc1NC(=O)NCC(C)C] | 6.070 |
| 001-377 | 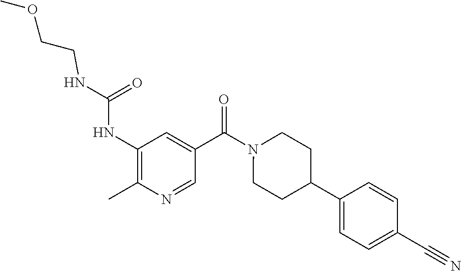 [SMILES: COCCNC(=O)Nc1cc(C(=O)N2CCC(c3ccc(C#N)cc3)CC2)cnc1C] | 2.550 |
| 001-378 | 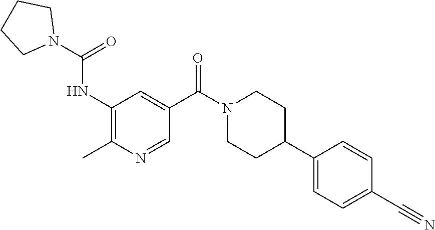 [SMILES: Cc1ncc(C(=O)N2CCC(c3ccc(C#N)cc3)CC2)cc1NC(=O)N1CCCC1] | |
| 001-379 | 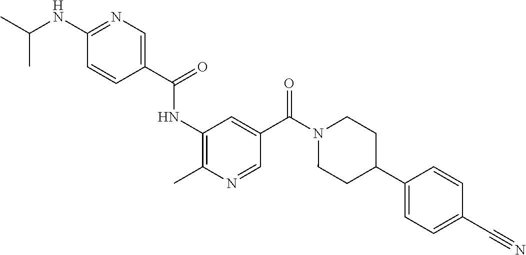 [SMILES: Cc1ncc(C(=O)N2CCC(c3ccc(C#N)cc3)CC2)cc1NC(=O)c1ccc(NC(C)C)nc1] | 0.705 |
| 001-380 | 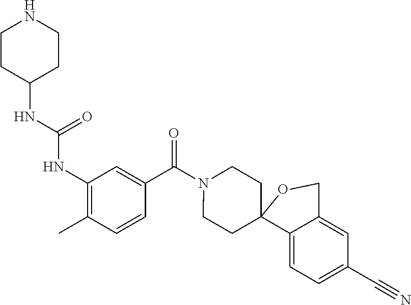 [SMILES: Cc1ccc(C(=O)N2CCC3(CC2)OCc2cc(C#N)ccc23)cc1NC(=O)NC1CCNCC1] | 0.420 |
| 001-132 | 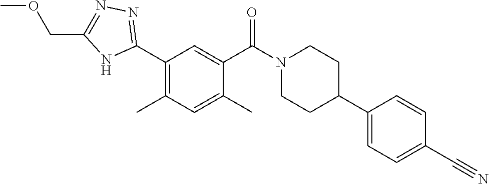 [SMILES: COCc1nnc(-c2cc(C(=O)N3CCC(c4ccc(C#N)cc4)CC3)c(C)cc2C)[nH]1] | 0.450 |
| 001-133 | 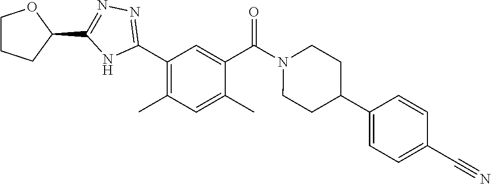 [SMILES: Cc1cc(C)c(-c2nnc([C@H]3CCCO3)[nH]2)cc1C(=O)N1CCC(c2ccc(C#N)cc2)CC1] | 0.790 |
| 001-134 | 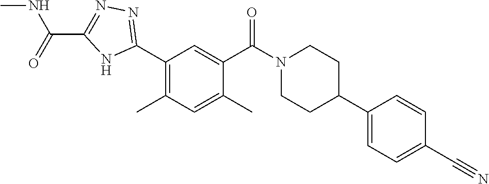 [SMILES: CNC(=O)c1nnc(-c2cc(C(=O)N3CCC(c4ccc(C#N)cc4)CC3)c(C)cc2C)[nH]1] | 1.330 |
| 001-135 | 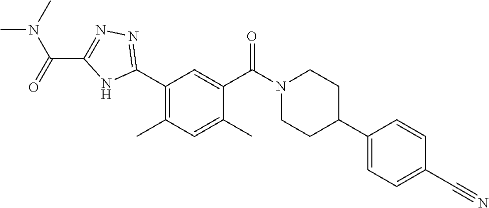 [SMILES: Cc1cc(C)c(-c2nnc(C(=O)N(C)C)[nH]2)cc1C(=O)N1CCC(c2ccc(C#N)cc2)CC1] | 2.040 |
| 001-136 | 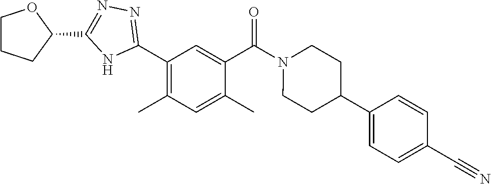 [SMILES: Cc1cc(C)c(-c2nnc([C@@H]3CCCO3)[nH]2)cc1C(=O)N1CCC(c2ccc(C#N)cc2)CC1] | 0.240 |
| 001-137 | 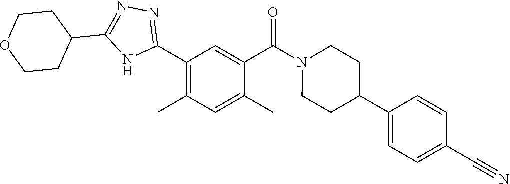 [SMILES: Cc1cc(C)c(-c2nnc(C3CCOCC3)[nH]2)cc1C(=O)N1CCC(c2ccc(C#N)cc2)CC1] | 0.500 |
| 001-138 | 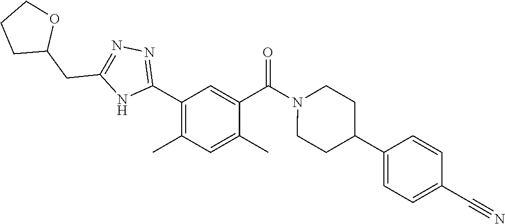 [SMILES: Cc1cc(C)c(-c2nnc(CC3CCCO3)[nH]2)cc1C(=O)N1CCC(c2ccc(C#N)cc2)CC1] | 0.640 |
| 001-381 | 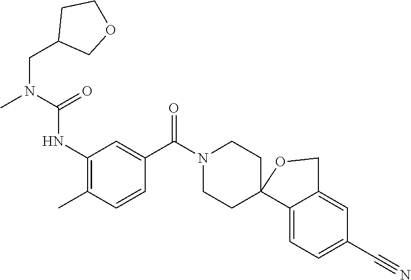 [SMILES: Cc1ccc(C(=O)N2CCC3(CC2)OCc2cc(C#N)ccc23)cc1NC(=O)N(C)CC1CCOC1] | >50 |
| 001-382 | 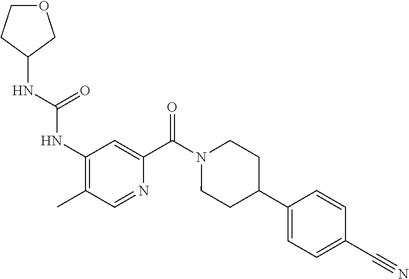 [SMILES: Cc1cnc(C(=O)N2CCC(c3ccc(C#N)cc3)CC2)cc1NC(=O)NC1CCOC1] | >50 |
| 001-383 | 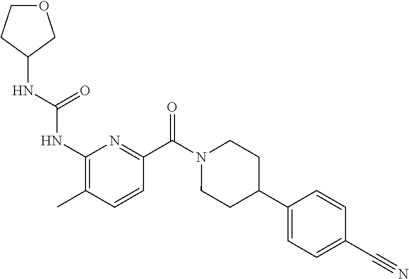 [SMILES: Cc1ccc(C(=O)N2CCC(c3ccc(C#N)cc3)CC2)nc1NC(=O)NC1CCOC1] | >50 |
| 001-384 | 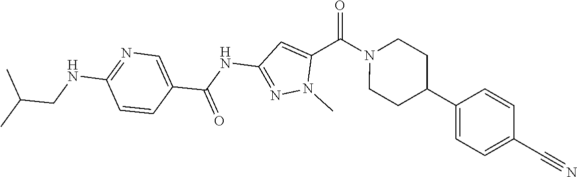 [SMILES: CC(C)CNc1ccc(C(=O)Nc2cc(C(=O)N3CCC(c4ccc(C#N)cc4)CC3)n(C)n2)cn1] | >50 |
| 001-385 | 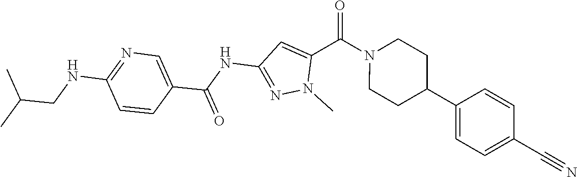 [SMILES: CC(C)CNc1ccc(C(=O)Nc2cc(C(=O)N3CCC(c4ccc(C#N)cc4)CC3)n(C)n2)cn1] | 8.867 |
| 001-386 | 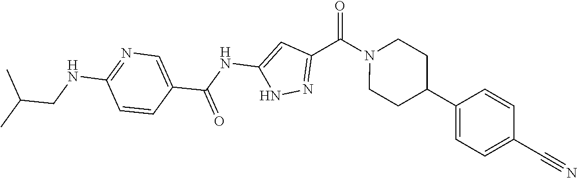 [SMILES: CC(C)CNc1ccc(C(=O)Nc2cc(C(=O)N3CCC(c4ccc(C#N)cc4)CC3)n[nH]2)cn1] | |
| 001-387 | 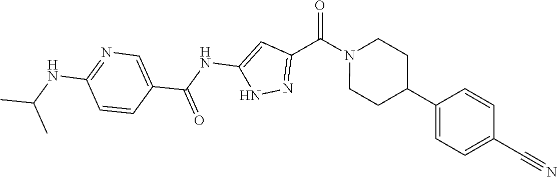 [SMILES: CC(C)Nc1ccc(C(=O)Nc2cc(C(=O)N3CCC(c4ccc(C#N)cc4)CC3)n[nH]2)cn1] | >50 |
| 001-139 | 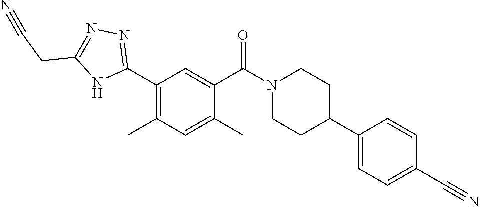 [SMILES: Cc1cc(C)c(-c2nnc(CC#N)[nH]2)cc1C(=O)N1CCC(c2ccc(C#N)cc2)CC1] | 1.210 |
| 001-140 | 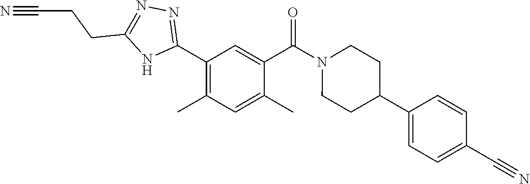 [SMILES: Cc1cc(C)c(-c2nnc(CCC#N)[nH]2)cc1C(=O)N1CCC(c2ccc(C#N)cc2)CC1] | 0.380 |
| 001-141 | 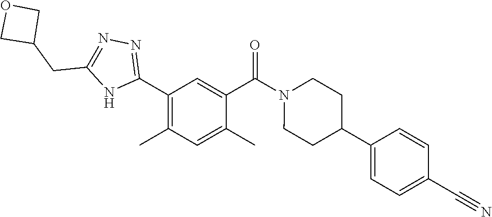 [SMILES: Cc1cc(C)c(-c2nnc(CC3COC3)[nH]2)cc1C(=O)N1CCC(c2ccc(C#N)cc2)CC1] | >50 |
| 001-142 | 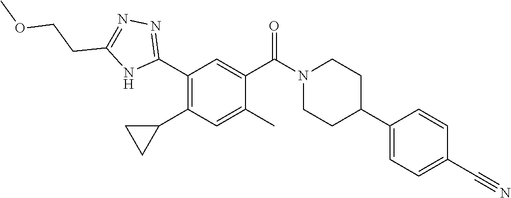 [SMILES: COCCc1nnc(-c2cc(C(=O)N3CCC(c4ccc(C#N)cc4)CC3)c(C)cc2C2CC2)[nH]1] | 0.140 |
| 001-143 | 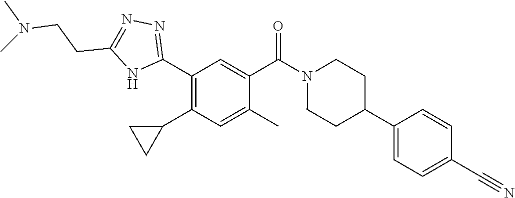 [SMILES: Cc1cc(C2CC2)c(-c2nnc(CCN(C)C)[nH]2)cc1C(=O)N1CCC(c2ccc(C#N)cc2)CC1] | 0.175 |
| 001-144 | 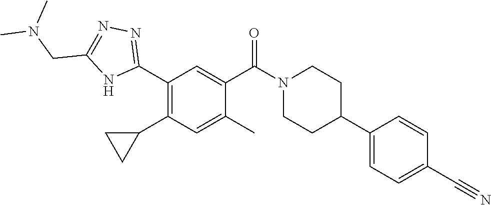 [SMILES: Cc1cc(C2CC2)c(-c2nnc(CN(C)C)[nH]2)cc1C(=O)N1CCC(c2ccc(C#N)cc2)CC1] | 0.320 |
| 001-145 | 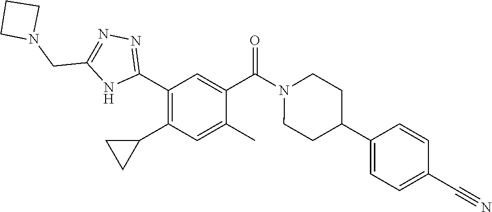 [SMILES: Cc1cc(C2CC2)c(-c2nnc(CN3CCC3)[nH]2)cc1C(=O)N1CCC(c2ccc(C#N)cc2)CC1] | 0.820 |
| 001-146 | 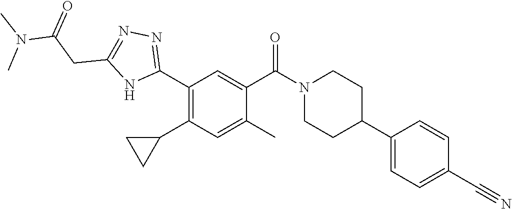 [SMILES: Cc1cc(C2CC2)c(-c2nnc(CC(=O)N(C)C)[nH]2)cc1C(=O)N1CCC(c2ccc(C#N)cc2)CC1] | 0.260 |
| 001-147 | 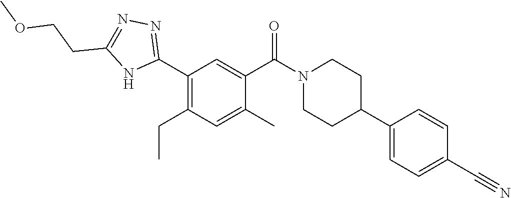 [SMILES: CCc1cc(C)c(C(=O)N2CCC(c3ccc(C#N)cc3)CC2)cc1-c1nnc(CCOC)[nH]1] | 0.250 |
| 001-388 | 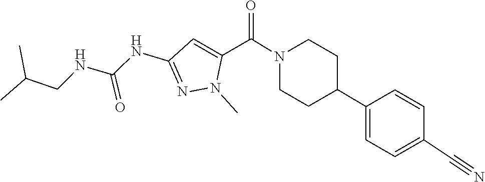 [SMILES: CC(C)CNC(=O)Nc1cc(C(=O)N2CCC(c3ccc(C#N)cc3)CC2)n(C)n1] | 13.380 |
| 001-389 | 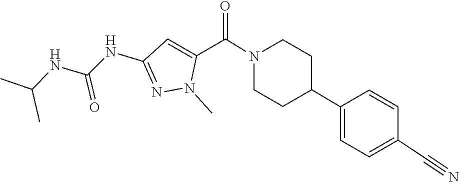 [SMILES: CC(C)NC(=O)Nc1cc(C(=O)N2CCC(c3ccc(C#N)cc3)CC2)n(C)n1] | 16.600 |
| 001-390 | 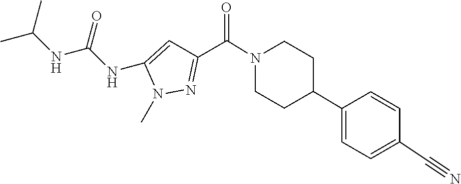 [SMILES: CC(C)NC(=O)Nc1cc(C(=O)N2CCC(c3ccc(C#N)cc3)CC2)nn1C] | >50 |
| 001-391 | 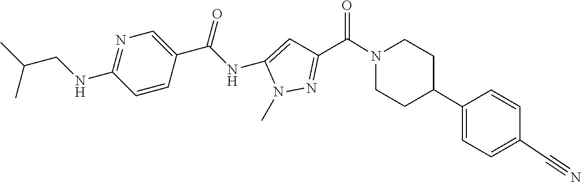 [SMILES: CC(C)CNc1ccc(C(=O)Nc2cc(C(=O)N3CCC(c4ccc(C#N)cc4)CC3)nn2C)cn1] | >50 |
| 001-392 | 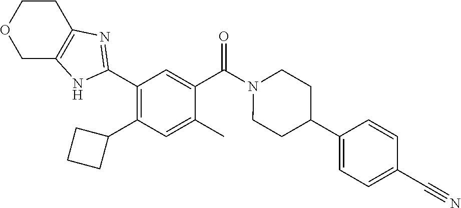 [SMILES: Cc1cc(C2CCC2)c(-c2nc3c([nH]2)COCC3)cc1C(=O)N1CCC(c2ccc(C#N)cc2)CC1] | 0.025 |
| 001-393 | 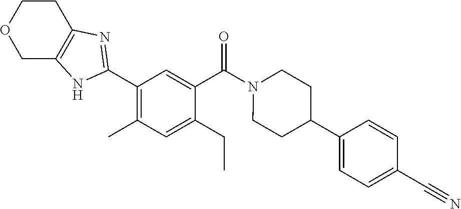 [SMILES: CCc1cc(C)c(-c2nc3c([nH]2)COCC3)cc1C(=O)N1CCC(c2ccc(C#N)cc2)CC1] | 0.065 |
| 001-394 | 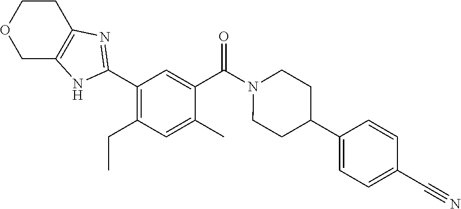 [SMILES: CCc1cc(C)c(C(=O)N2CCC(c3ccc(C#N)cc3)CC2)cc1-c1nc2c([nH]1)COCC2] | 0.135 |
| 001-395 | 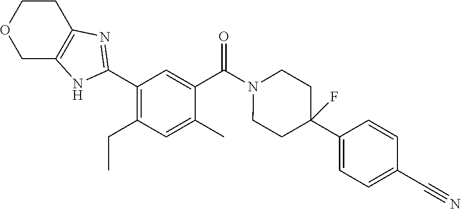 [SMILES: CCc1cc(C)c(C(=O)N2CCC(F)(c3ccc(C#N)cc3)CC2)cc1-c1nc2c([nH]1)COCC2] | 0.260 |
| 001-396 | 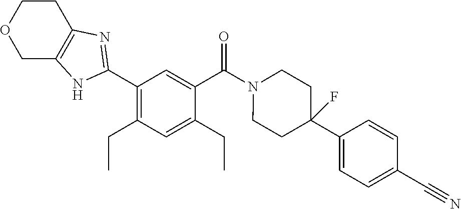 [SMILES: CCc1cc(CC)c(-c2nc3c([nH]2)COCC3)cc1C(=O)N1CCC(F)(c2ccc(C#N)cc2)CC1] | 0.050 |
| 001-148 | 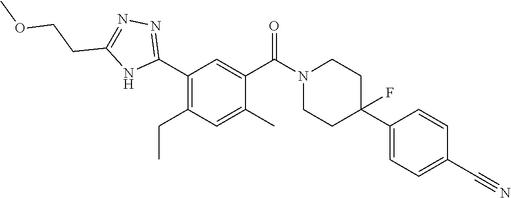 [SMILES: CCc1cc(C)c(C(=O)N2CCC(F)(c3ccc(C#N)cc3)CC2)cc1-c1nnc(CCOC)[nH]1] | 0.180 |
| 001-149 | 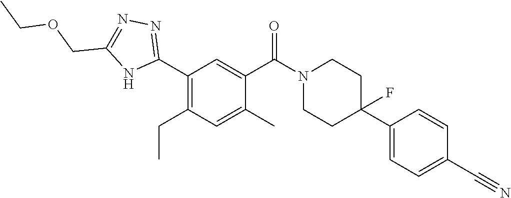 [SMILES: CCOCc1nnc(-c2cc(C(=O)N3CCC(F)(c4ccc(C#N)cc4)CC3)c(C)cc2CC)[nH]1] | 0.240 |
| 001-150 | 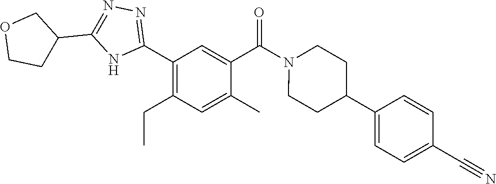 [SMILES: CCc1cc(C)c(C(=O)N2CCC(c3ccc(C#N)cc3)CC2)cc1-c1nnc(C2CCOC2)[nH]1] | 0.300 |
| 001-151 | 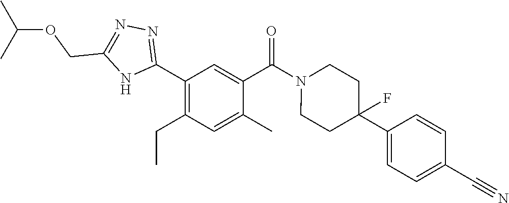 [SMILES: CCc1cc(C)c(C(=O)N2CCC(F)(c3ccc(C#N)cc3)CC2)cc1-c1nnc(COC(C)C)[nH]1] | 0.420 |
| 001-152 | 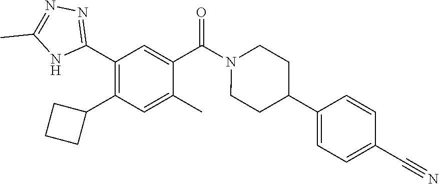 [SMILES: Cc1nnc(-c2cc(C(=O)N3CCC(c4ccc(C#N)cc4)CC3)c(C)cc2C2CCC2)[nH]1] | 0.052 |
| 001-153 | 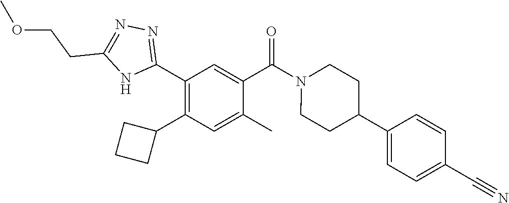 [SMILES: COCCc1nnc(-c2cc(C(=O)N3CCC(c4ccc(C#N)cc4)CC3)c(C)cc2C2CCC2)[nH]1] | 0.055 |
| 001-154 | 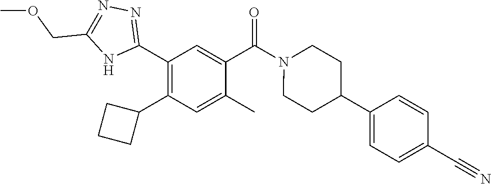 [SMILES: COCc1nnc(-c2cc(C(=O)N3CCC(c4ccc(C#N)cc4)CC3)c(C)cc2C2CCC2)[nH]1] | 0.105 |
| 001-155 | 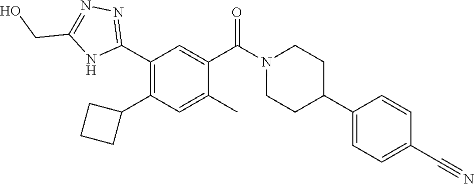 [SMILES: Cc1cc(C2CCC2)c(-c2nnc(CO)[nH]2)cc1C(=O)N1CCC(c2ccc(C#N)cc2)CC1] | 0.20 |
| 001-156 | 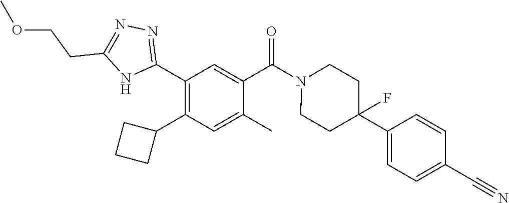 [SMILES: COCCc1nnc(-c2cc(C(=O)N3CCC(F)(c4ccc(C#N)cc4)CC3)c(C)cc2C2CCC2)[nH]1] | 0.030 |
| 001-397 | 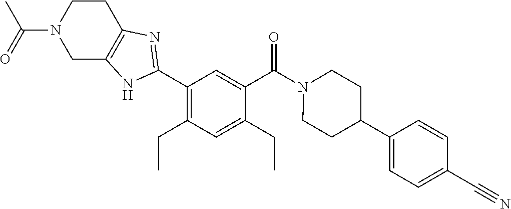 [SMILES: CCc1cc(CC)c(-c2nc3c([nH]2)CN(C(C)=O)CC3)cc1C(=O)N1CCC(c2ccc(C#N)cc2)CC1] | 0.040 |
| 001-398 | 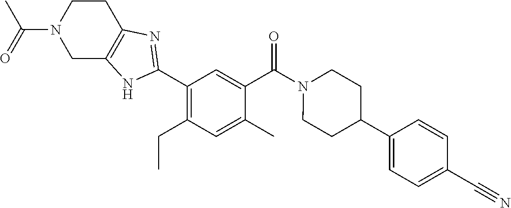 [SMILES: CCc1cc(C)c(C(=O)N2CCC(c3ccc(C#N)cc3)CC2)cc1-c1nc2c([nH]1)CN(C(C)=O)CC2] | 0.035 |
| 001-399 | 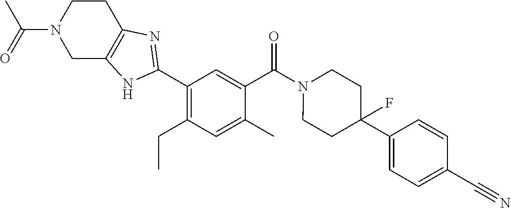 [SMILES: CCc1cc(C)c(C(=O)N2CCC(F)(c3ccc(C#N)cc3)CC2)cc1-c1nc2c([nH]1)CN(C(C)=O)CC2] | 0.030 |
| 001-400 | 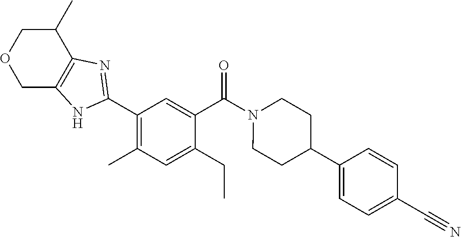 [SMILES: CCc1cc(C)c(-c2nc3c([nH]2)COCC3C)cc1C(=O)N1CCC(c2ccc(C#N)cc2)CC1] | 0.260 |
| 001-401 | 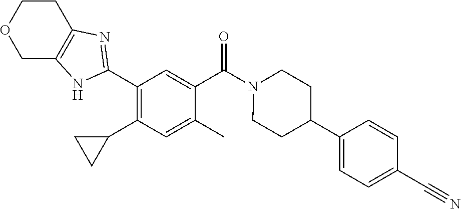 [SMILES: Cc1cc(C2CC2)c(-c2nc3c([nH]2)COCC3)cc1C(=O)N1CCC(c2ccc(C#N)cc2)CC1] | 0.065 |
| 001-402 | 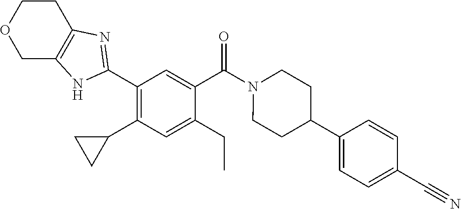 [SMILES: CCc1cc(C2CC2)c(-c2nc3c([nH]2)COCC3)cc1C(=O)N1CCC(c2ccc(C#N)cc2)CC1] | 0.077 |
| 001-403 | 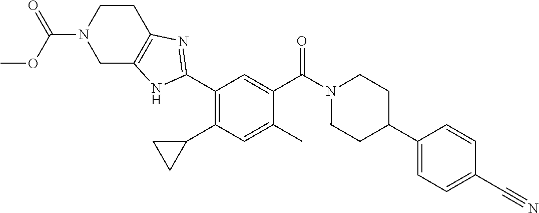 [SMILES: COC(=O)N1CCc2nc(-c3cc(C(=O)N4CCC(c5ccc(C#N)cc5)CC4)c(C)cc3C3CC3)[nH]c2C1] | 0.045 |
| 001-404 | 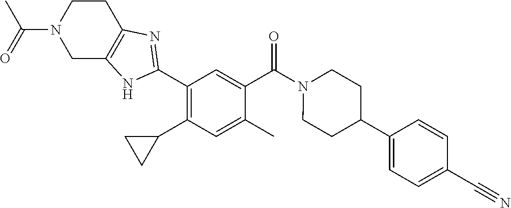 [SMILES: CC(=O)N1CCc2nc(-c3cc(C(=O)N4CCC(c5ccc(C#N)cc5)CC4)c(C)cc3C3CC3)[nH]c2C1] | 0.040 |
| 001-405 | 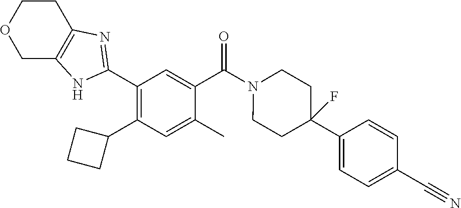 [SMILES: Cc1cc(C2CCC2)c(-c2nc3c([nH]2)COCC3)cc1C(=O)N1CCC(F)(c2ccc(C#N)cc2)CC1] | 0.010 |
| 001-157 | 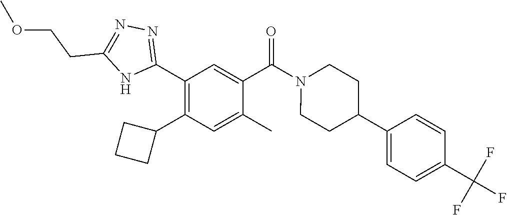 [SMILES: COCCc1nnc(-c2cc(C(=O)N3CCC(c4ccc(C(F)(F)F)cc4)CC3)c(C)cc2C2CCC2)[nH]1] | 0.090 |
| 001-158 | 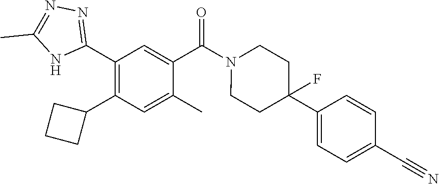 [SMILES: Cc1nnc(-c2cc(C(=O)N3CCC(F)(c4ccc(C#N)cc4)CC3)c(C)cc2C2CCC2)[nH]1] | 0.057 |
| 001-159 | 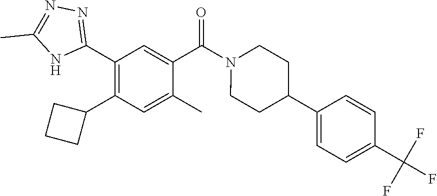 [SMILES: Cc1nnc(-c2cc(C(=O)N3CCC(c4ccc(C(F)(F)F)cc4)CC3)c(C)cc2C2CCC2)[nH]1] | 0.070 |
| 001-160 | 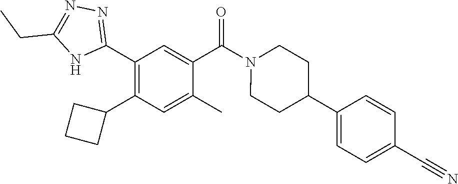 [SMILES: CCc1nnc(-c2cc(C(=O)N3CCC(c4ccc(C#N)cc4)CC3)c(C)cc2C2CCC2)[nH]1] | 0.055 |
| 001-161 | 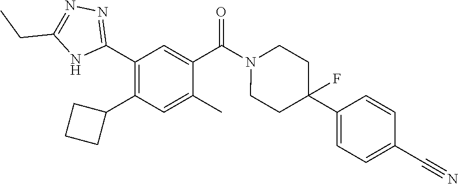 [SMILES: CCc1nnc(-c2cc(C(=O)N3CCC(F)(c4ccc(C#N)cc4)CC3)c(C)cc2C2CCC2)[nH]1] | 0.040 |
| 001-162 | 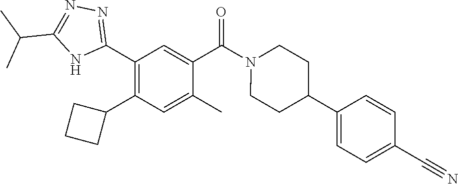 [SMILES: Cc1cc(C2CCC2)c(-c2nnc(C(C)C)[nH]2)cc1C(=O)N1CCC(c2ccc(C#N)cc2)CC1] | 0.115 |
| 001-163 | 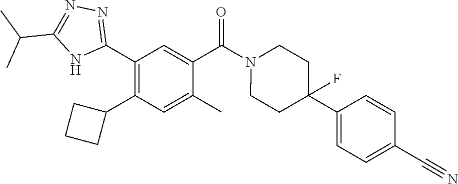 [SMILES: Cc1cc(C2CCC2)c(-c2nnc(C(C)C)[nH]2)cc1C(=O)N1CCC(F)(c2ccc(C#N)cc2)CC1] | 0.135 |
| 001-164 | 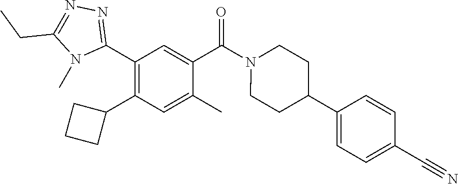 [SMILES: CCc1nnc(-c2cc(C(=O)N3CCC(c4ccc(C#N)cc4)CC3)c(C)cc2C2CCC2)n1C] | >50 |
| 001-165 | 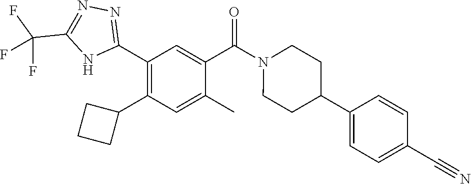 [SMILES: Cc1cc(C2CCC2)c(-c2nnc(C(F)(F)F)[nH]2)cc1C(=O)N1CCC(c2ccc(C#N)cc2)CC1] | 1.000 |
| 001-406 | 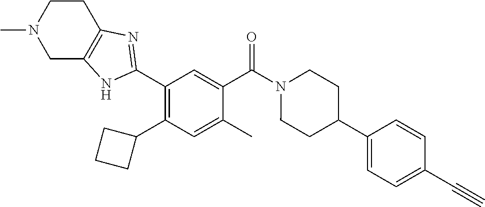 [SMILES: C#Cc1ccc(C2CCN(C(=O)c3cc(-c4nc5c([nH]4)CN(C)CC5)c(C4CCC4)cc3C)CC2)cc1] | 0.020 |
| 001-407 | 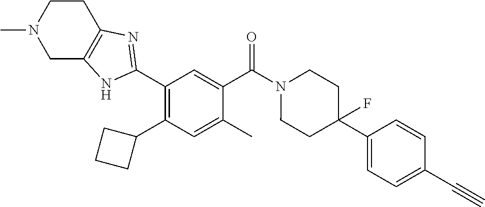 [SMILES: C#Cc1ccc(C2(F)CCN(C(=O)c3cc(-c4nc5c([nH]4)CN(C)CC5)c(C4CCC4)cc3C)CC2)cc1] | 0.040 |
| 001-408 | 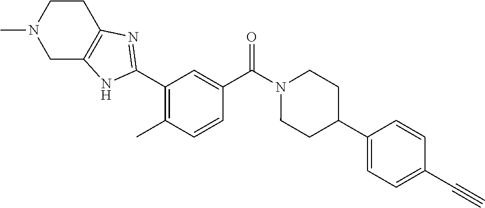 [SMILES: C#Cc1ccc(C2CCN(C(=O)c3ccc(C)c(-c4nc5c([nH]4)CN(C)CC5)c3)CC2)cc1] | 0.145 |
| 001-409 | 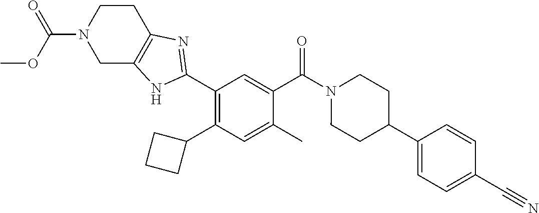 [SMILES: COC(=O)N1CCc2nc(-c3cc(C(=O)N4CCC(c5ccc(C#N)cc5)CC4)c(C)cc3C3CCC3)[nH]c2C1] | 0.020 |
| 001-410 | 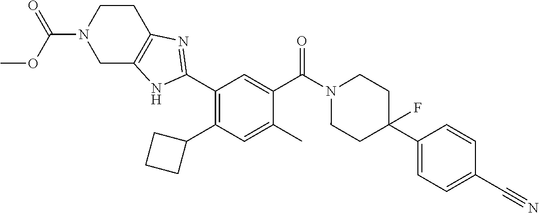 [SMILES: COC(=O)N1CCc2nc(-c3cc(C(=O)N4CCC(F)(c5ccc(C#N)cc5)CC4)c(C)cc3C3CCC3)[nH]c2C1] | 0.010 |
| 001-411 | 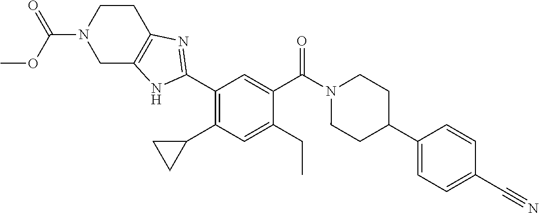 [SMILES: CCc1cc(C2CC2)c(-c2nc3c([nH]2)CN(C(=O)OC)CC3)cc1C(=O)N1CCC(c2ccc(C#N)cc2)CC1] | 0.035 |
| 001-412 | 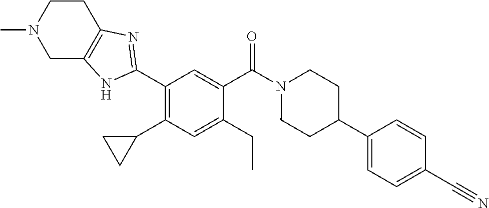 [SMILES: CCc1cc(C2CC2)c(-c2nc3c([nH]2)CN(C)CC3)cc1C(=O)N1CCC(c2ccc(C#N)cc2)CC1] | 0.035 |
| 001-413 | 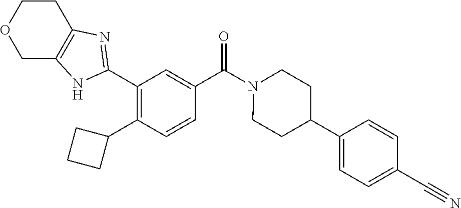 [SMILES: N#Cc1ccc(C2CCN(C(=O)c3ccc(C4CCC4)c(-c4nc5c([nH]4)COCC5)c3)CC2)cc1] | 0.065 |
| 001-414 | 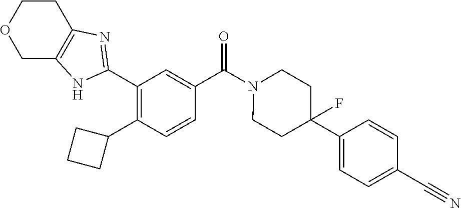 [SMILES: N#Cc1ccc(C2(F)CCN(C(=O)c3ccc(C4CCC4)c(-c4nc5c([nH]4)COCC5)c3)CC2)cc1] | 0.033 |
| 001-166 | 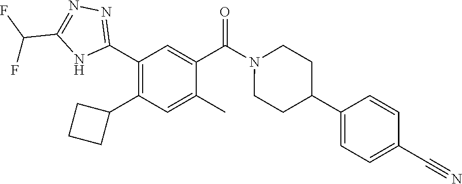 [SMILES: Cc1cc(C2CCC2)c(-c2nnc(C(F)F)[nH]2)cc1C(=O)N1CCC(c2ccc(C#N)cc2)CC1] | 0.160 |
| 001-167 | 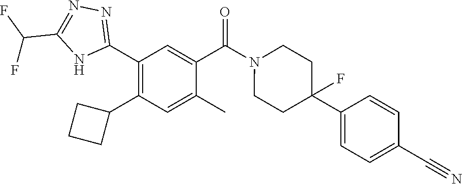 [SMILES: Cc1cc(C2CCC2)c(-c2nnc(C(F)F)[nH]2)cc1C(=O)N1CCC(F)(c2ccc(C#N)cc2)CC1] | 0.125 |
| 001-168 | 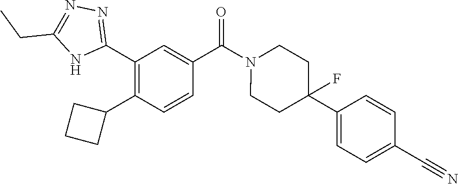 [SMILES: CCc1nnc(-c2cc(C(=O)N3CCC(F)(c4ccc(C#N)cc4)CC3)ccc2C2CCC2)[nH]1] | 0.060 |
| 001-169 | 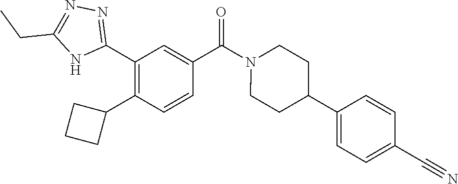 [SMILES: CCc1nnc(-c2cc(C(=O)N3CCC(c4ccc(C#N)cc4)CC3)ccc2C2CCC2)[nH]1] | 0.090 |
| 001-170 | 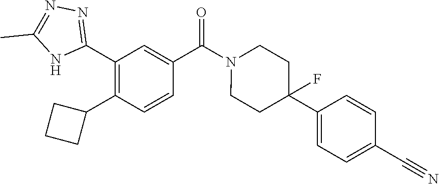 [SMILES: Cc1nnc(-c2cc(C(=O)N3CCC(F)(c4ccc(C#N)cc4)CC3)ccc2C2CCC2)[nH]1] | 0.090 |
| 001-171 | 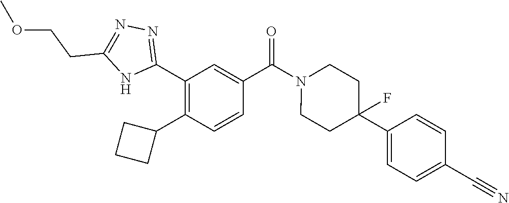 [SMILES: COCCc1nnc(-c2cc(C(=O)N3CCC(F)(c4ccc(C#N)cc4)CC3)ccc2C2CCC2)[nH]1] | 0.070 |
| 001-172 | 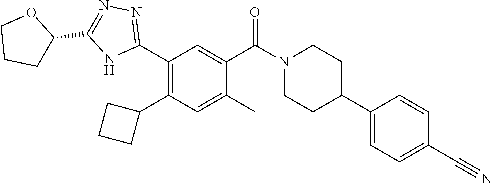 [SMILES: Cc1cc(C2CCC2)c(-c2nnc([C@@H]3CCCO3)[nH]2)cc1C(=O)N1CCC(c2ccc(C#N)cc2)CC1] | 0.140 |
| 001-173 | 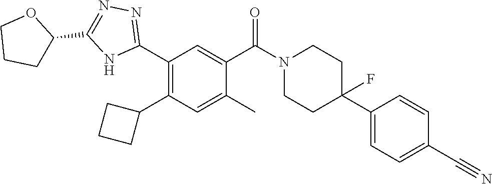 [SMILES: Cc1cc(C2CCC2)c(-c2nnc([C@@H]3CCCO3)[nH]2)cc1C(=O)N1CCC(F)(c2ccc(C#N)cc2)CC1] | 0.240 |
| 001-415 | 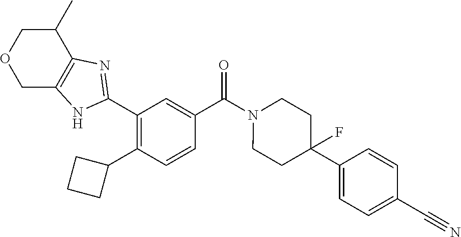 [SMILES: CC1COCc2[nH]c(-c3cc(C(=O)N4CCC(F)(c5ccc(C#N)cc5)CC4)ccc3C3CCC3)nc21] | 0.040 |
| 001-416 | 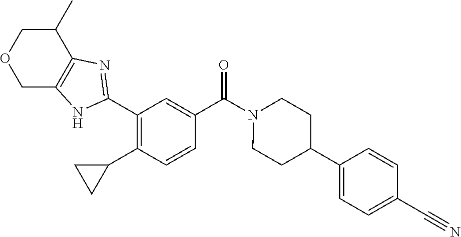 [SMILES: CC1COCc2[nH]c(-c3cc(C(=O)N4CCC(c5ccc(C#N)cc5)CC4)ccc3C3CC3)nc21] | 0.055 |
| 001-417 | 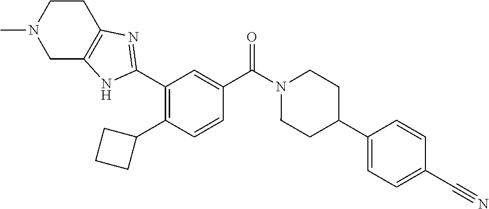 [SMILES: CN1CCc2nc(-c3cc(C(=O)N4CCC(c5ccc(C#N)cc5)CC4)ccc3C3CCC3)[nH]c2C1] | 0.020 |
| 001-418 | 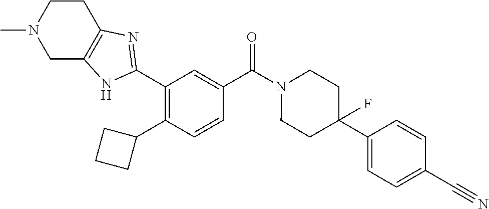 [SMILES: CN1CCc2nc(-c3cc(C(=O)N4CCC(F)(c5ccc(C#N)cc5)CC4)ccc3C3CCC3)[nH]c2C1] | 0.050 |
| 001-419 | 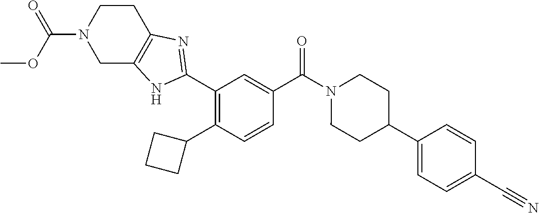 [SMILES: COC(=O)N1CCc2nc(-c3cc(C(=O)N4CCC(c5ccc(C#N)cc5)CC4)ccc3C3CCC3)[nH]c2C1] | 0.010 |
| 001-420 | 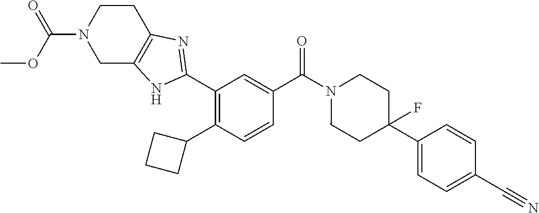 [SMILES: COC(=O)N1CCc2nc(-c3cc(C(=O)N4CCC(F)(c5ccc(C#N)cc5)CC4)ccc3C3CCC3)[nH]c2C1] | 0.020 |
| 001-421 | 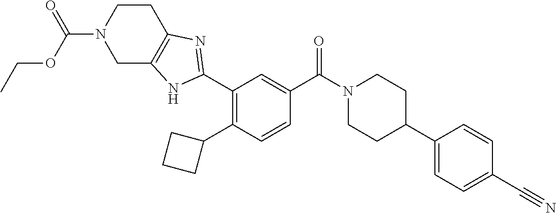 [SMILES: CCOC(=O)N1CCc2nc(-c3cc(C(=O)N4CCC(c5ccc(C#N)cc5)CC4)ccc3C3CCC3)[nH]c2C1] | 0.050 |
| 001-422 | 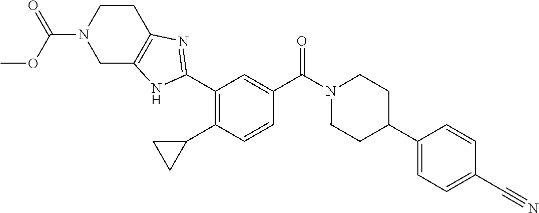 [SMILES: COC(=O)N1CCc2nc(-c3cc(C(=O)N4CCC(c5ccc(C#N)cc5)CC4)ccc3C3CC3)[nH]c2C1] | 0.020 |
| 001-174 | 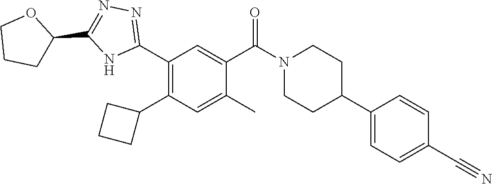 [SMILES: Cc1cc(C2CCC2)c(-c2nnc([C@H]3CCCO3)[nH]2)cc1C(=O)N1CCC(c2ccc(C#N)cc2)CC1] | 0.200 |
| 001-175 | 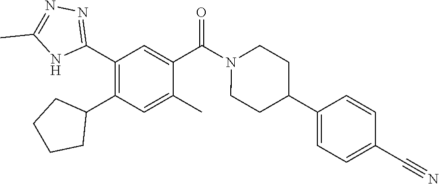 [SMILES: Cc1nnc(-c2cc(C(=O)N3CCC(c4ccc(C#N)cc4)CC3)c(C)cc2C2CCCC2)[nH]1] | 0.310 |
| 001-176 | 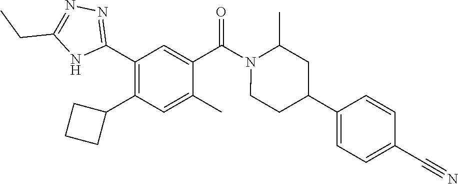 [SMILES: CCc1nnc(-c2cc(C(=O)N3CCC(c4ccc(C#N)cc4)CC3C)c(C)cc2C2CCC2)[nH]1] | 5.990 |
| 001-177 | 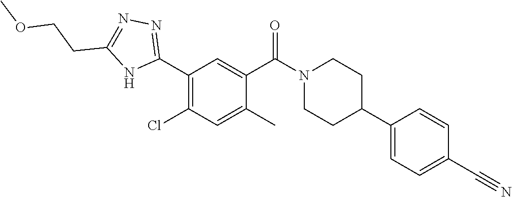 [SMILES: COCCc1nnc(-c2cc(C(=O)N3CCC(c4ccc(C#N)cc4)CC3)c(C)cc2Cl)[nH]1] | 2.110 |
| 001-178 | 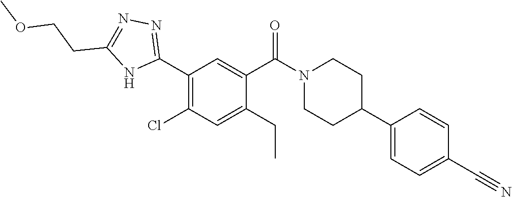 [SMILES: CCc1cc(Cl)c(-c2nnc(CCOC)[nH]2)cc1C(=O)N1CCC(c2ccc(C#N)cc2)CC1] | 0.555 |
| 001-179 | 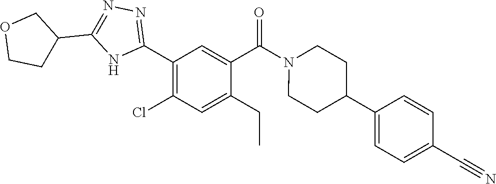 [SMILES: CCc1cc(Cl)c(-c2nnc(C3CCOC3)[nH]2)cc1C(=O)N1CCC(c2ccc(C#N)cc2)CC1] | 1.260 |
| 001-180 | 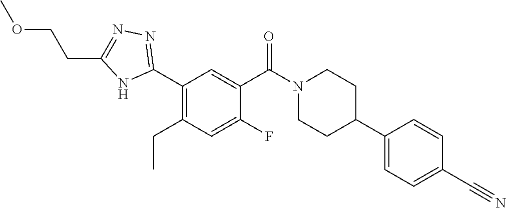 [SMILES: CCc1cc(F)c(C(=O)N2CCC(c3ccc(C#N)cc3)CC2)cc1-c1nnc(CCOC)[nH]1] | 8.230 |
| 001-423 | 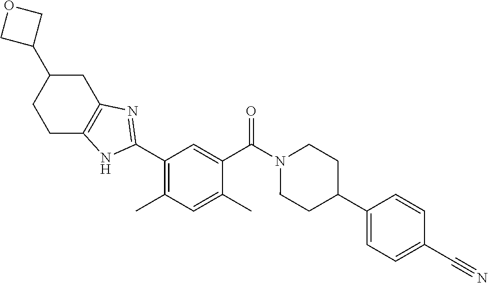 [SMILES: Cc1cc(C)c(-c2nc3c([nH]2)CCC(C2COC2)C3)cc1C(=O)N1CCC(c2ccc(C#N)cc2)CC1] | 0.175 |
| 001-424 | 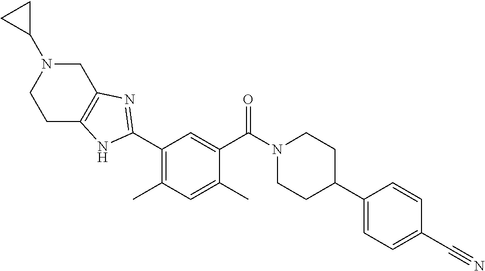 [SMILES: Cc1cc(C)c(-c2nc3c([nH]2)CCN(C2CC2)C3)cc1C(=O)N1CCC(c2ccc(C#N)cc2)CC1] | 0.165 |
| 001-425 | 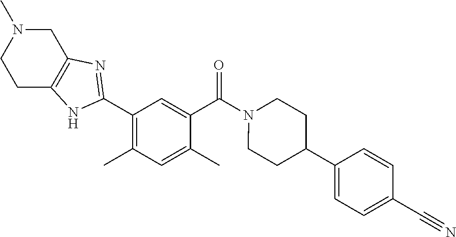 [SMILES: Cc1cc(C)c(-c2nc3c([nH]2)CCN(C)C3)cc1C(=O)N1CCC(c2ccc(C#N)cc2)CC1] | 0.075 |
| 001-426 | 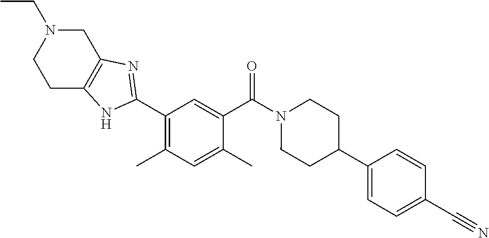 [SMILES: CCN1CCc2[nH]c(-c3cc(C(=O)N4CCC(c5ccc(C#N)cc5)CC4)c(C)cc3C)nc2C1] | 0.060 |
| 001-427 | 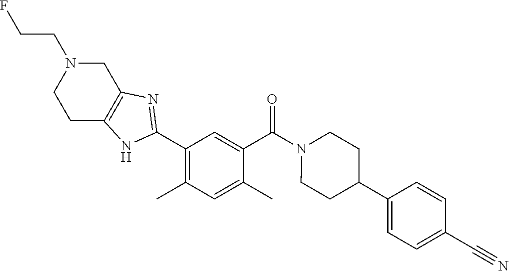 [SMILES: Cc1cc(C)c(-c2nc3c([nH]2)CCN(CCF)C3)cc1C(=O)N1CCC(c2ccc(C#N)cc2)CC1] | 0.080 |
| 001-428 | 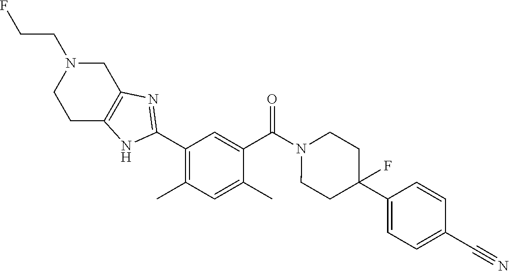 [SMILES: Cc1cc(C)c(-c2nc3c([nH]2)CCN(CCF)C3)cc1C(=O)N1CCC(F)(c2ccc(C#N)cc2)CC1] | 0.040 |
| 001-429 | 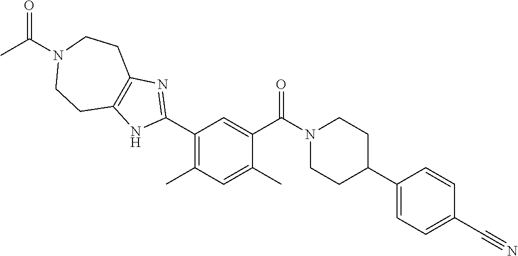 [SMILES: CC(=O)N1CCc2nc(-c3cc(C(=O)N4CCC(c5ccc(C#N)cc5)CC4)c(C)cc3C)[nH]c2CC1] | 0.060 |
| 001-181 | 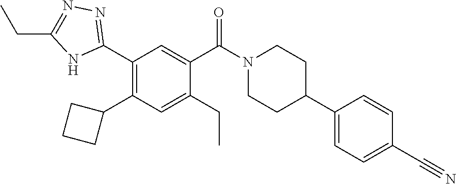 [SMILES: CCc1nnc(-c2cc(C(=O)N3CCC(c4ccc(C#N)cc4)CC3)c(CC)cc2C2CCC2)[nH]1] | 0.115 |
| 001-182 | 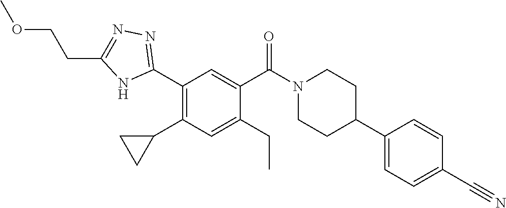 [SMILES: CCc1cc(C2CC2)c(-c2nnc(CCOC)[nH]2)cc1C(=O)N1CCC(c2ccc(C#N)cc2)CC1] | 0.145 |
| 001-183 | 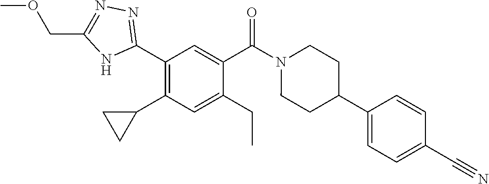 [SMILES: CCc1cc(C2CC2)c(-c2nnc(COC)[nH]2)cc1C(=O)N1CCC(c2ccc(C#N)cc2)CC1] | 0.170 |
| 001-184 | 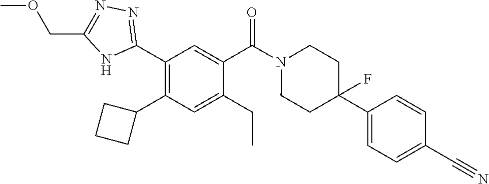 [SMILES: CCc1cc(C2CCC2)c(-c2nnc(COC)[nH]2)cc1C(=O)N1CCC(F)(c2ccc(C#N)cc2)CC1] | 0.085 |
| 001-185 | 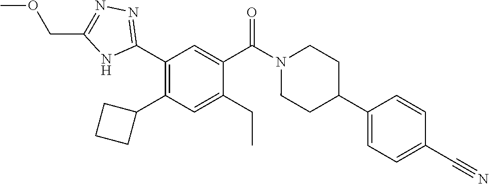 [SMILES: CCc1cc(C2CCC2)c(-c2nnc(COC)[nH]2)cc1C(=O)N1CCC(c2ccc(C#N)cc2)CC1] | 0.080 |
| 001-186 | 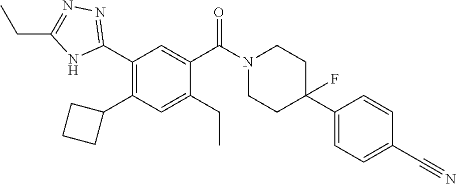 [SMILES: CCc1nnc(-c2cc(C(=O)N3CCC(F)(c4ccc(C#N)cc4)CC3)c(CC)cc2C2CCC2)[nH]1] | 0.055 |
| 001-187 | 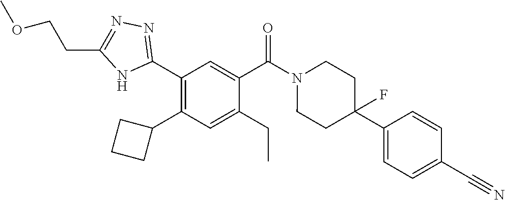 [SMILES: CCc1cc(C2CCC2)c(-c2nnc(CCOC)[nH]2)cc1C(=O)N1CCC(F)(c2ccc(C#N)cc2)CC1] | 0.065 |
| 001-188 | 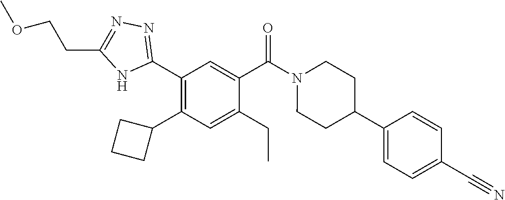 [SMILES: CCc1cc(C2CCC2)c(-c2nnc(CCOC)[nH]2)cc1C(=O)N1CCC(c2ccc(C#N)cc2)CC1] | 0.070 |
| 001-430 | 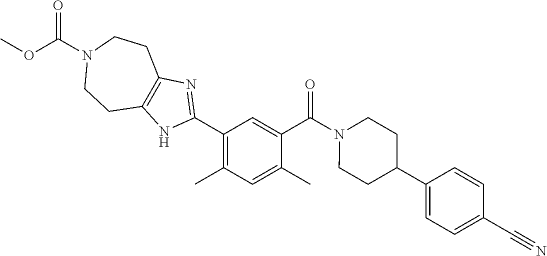 [SMILES: COC(=O)N1CCc2nc(-c3cc(C(=O)N4CCC(c5ccc(C#N)cc5)CC4)c(C)cc3C)[nH]c2CC1] | 0.035 |
| 001-431 | 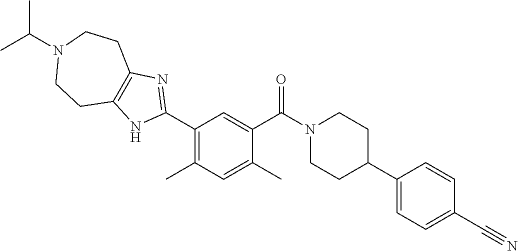 [SMILES: Cc1cc(C)c(-c2nc3c([nH]2)CCN(C(C)C)CC3)cc1C(=O)N1CCC(c2ccc(C#N)cc2)CC1] | 0.025 |
| 001-432 | 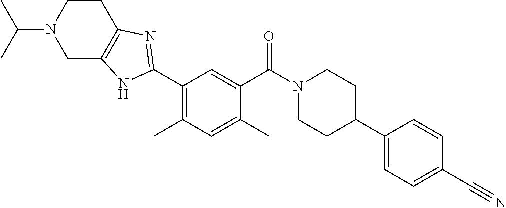 [SMILES: Cc1cc(C)c(-c2nc3c([nH]2)CN(C(C)C)CC3)cc1C(=O)N1CCC(c2ccc(C#N)cc2)CC1] | 0.065 |
| 001-433 | 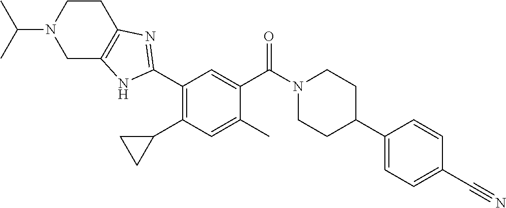 [SMILES: Cc1cc(C2CC2)c(-c2nc3c([nH]2)CN(C(C)C)CC3)cc1C(=O)N1CCC(c2ccc(C#N)cc2)CC1] | 0.040 |
| 001-434 | 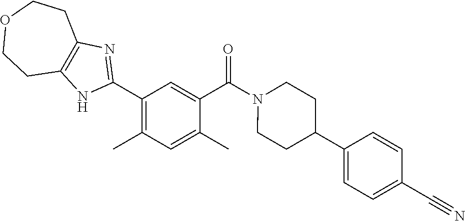 [SMILES: Cc1cc(C)c(-c2nc3c([nH]2)CCOCC3)cc1C(=O)N1CCC(c2ccc(C#N)cc2)CC1] | 0.470 |
| 001-435 | 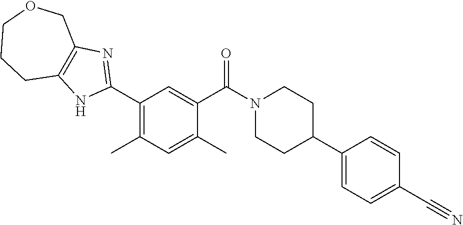 [SMILES: Cc1cc(C)c(-c2nc3c([nH]2)CCCOC3)cc1C(=O)N1CCC(c2ccc(C#N)cc2)CC1] | 0.365 |
| 001-436 | 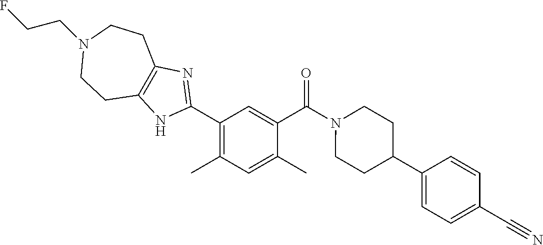 [SMILES: Cc1cc(C)c(-c2nc3c([nH]2)CCN(CCF)CC3)cc1C(=O)N1CCC(c2ccc(C#N)cc2)CC1] | 0.040 |
| 001-437 | 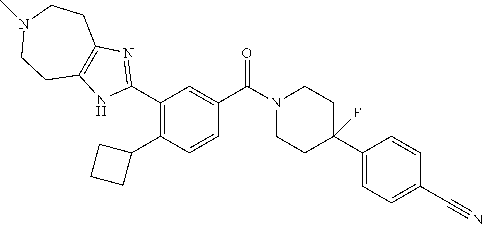 [SMILES: CN1CCc2nc(-c3cc(C(=O)N4CCC(F)(c5ccc(C#N)cc5)CC4)ccc3C3CCC3)[nH]c2CC1] | 0.050 |
| 001-189 | 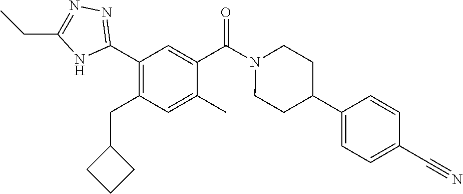 [SMILES: CCc1nnc(-c2cc(C(=O)N3CCC(c4ccc(C#N)cc4)CC3)c(C)cc2CC2CCC2)[nH]1] | 0.270 |
| 001-190 | 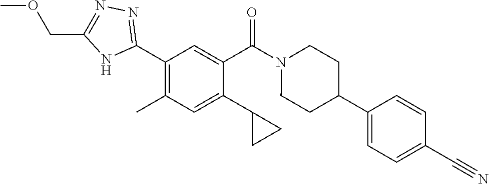 [SMILES: COCc1nnc(-c2cc(C(=O)N3CCC(c4ccc(C#N)cc4)CC3)c(C3CC3)cc2C)[nH]1] | 5.060 |
| 001-191 | 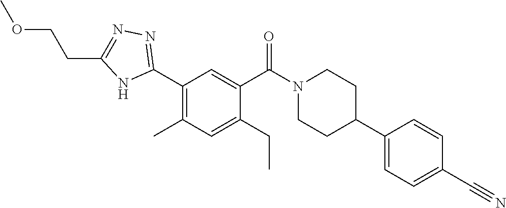 [SMILES: CCc1cc(C)c(-c2nnc(CCOC)[nH]2)cc1C(=O)N1CCC(c2ccc(C#N)cc2)CC1] | 0.200 |
| 001-192 | 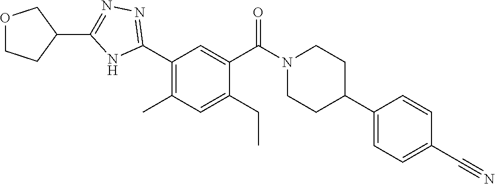 [SMILES: CCc1cc(C)c(-c2nnc(C3CCOC3)[nH]2)cc1C(=O)N1CCC(c2ccc(C#N)cc2)CC1] | 0.300 |
| 001-193 | 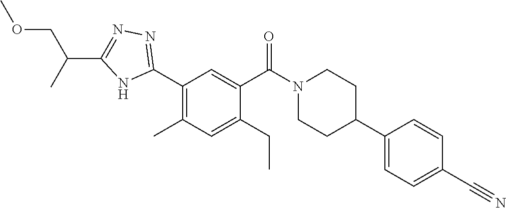 [SMILES: CCc1cc(C)c(-c2nnc(C(C)COC)[nH]2)cc1C(=O)N1CCC(c2ccc(C#N)cc2)CC1] | 0.440 |
| 001-194 | 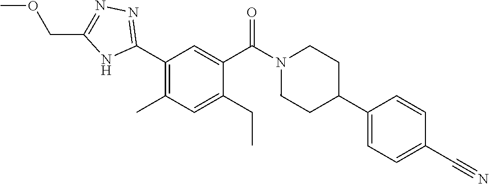 [SMILES: CCc1cc(C)c(-c2nnc(COC)[nH]2)cc1C(=O)N1CCC(c2ccc(C#N)cc2)CC1] | 0.400 |
| 001-438 | 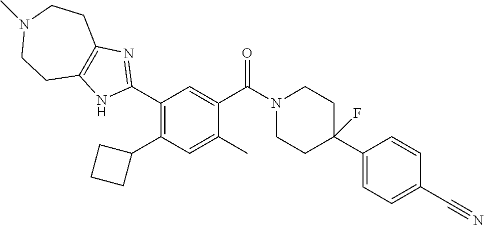 [SMILES: Cc1cc(C2CCC2)c(-c2nc3c([nH]2)CCN(C)CC3)cc1C(=O)N1CCC(F)(c2ccc(C#N)cc2)CC1] | 0.060 |
| 001-439 | 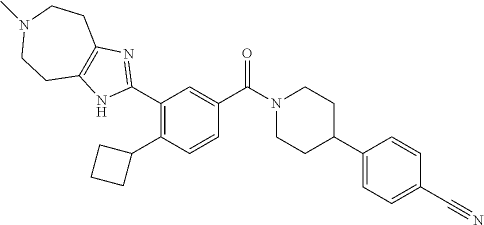 [SMILES: CN1CCc2nc(-c3cc(C(=O)N4CCC(c5ccc(C#N)cc5)CC4)ccc3C3CCC3)[nH]c2CC1] | 0.110 |
| 001-440 | 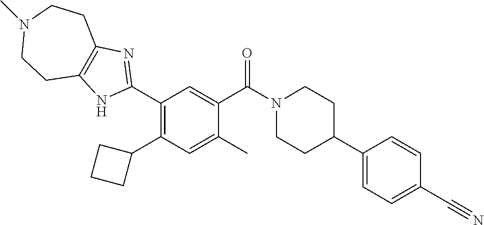 [SMILES: Cc1cc(C2CCC2)c(-c2nc3c([nH]2)CCN(C)CC3)cc1C(=O)N1CCC(c2ccc(C#N)cc2)CC1] | 0.050 |
| 001-441 | 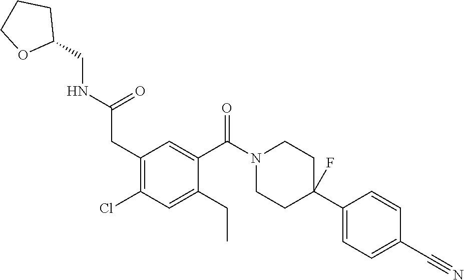 [SMILES: CCc1cc(Cl)c(CC(=O)NC[C@H]2CCCO2)cc1C(=O)N1CCC(F)(c2ccc(C#N)cc2)CC1] | 0.140 |
| 001-442 | 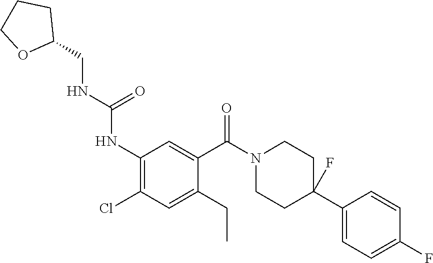 [SMILES: CCc1cc(Cl)c(NC(=O)NC[C@H]2CCCO2)cc1C(=O)N1CCC(F)(c2ccc(F)cc2)CC1] | 12.480 |
| 001-443 | 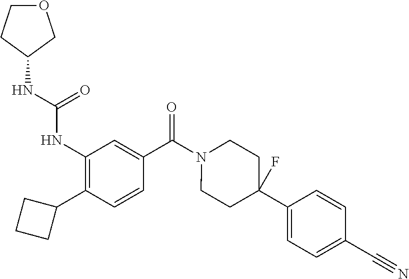 [SMILES: N#Cc1ccc(C2(F)CCN(C(=O)c3ccc(C4CCC4)c(NC(=O)N[C@@H]4CCOC4)c3)CC2)cc1] | 0.060 |
| 001-195 | 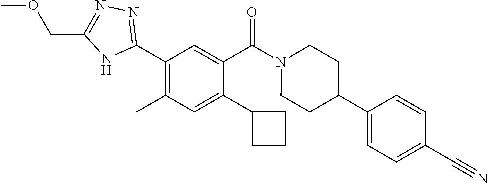 [SMILES: COCc1nnc(-c2cc(C(=O)N3CCC(c4ccc(C#N)cc4)CC3)c(C3CCC3)cc2C)[nH]1] | >50 |
| 001-196 | 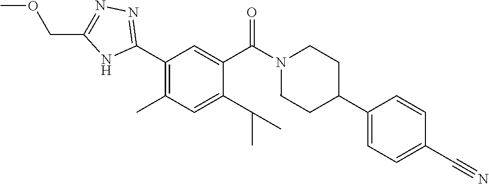 [SMILES: COCc1nnc(-c2cc(C(=O)N3CCC(c4ccc(C#N)cc4)CC3)c(C(C)C)cc2C)[nH]1] | >50 |
| 001-197 | 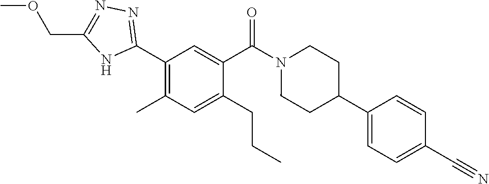 [SMILES: CCCc1cc(C)c(-c2nnc(COC)[nH]2)cc1C(=O)N1CCC(c2ccc(C#N)cc2)CC1] | 11.105 |
| 001-198 | 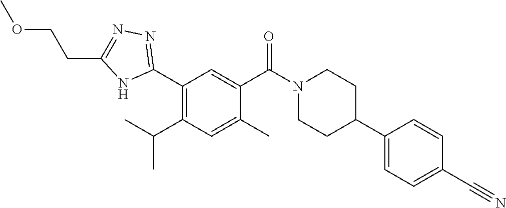 [SMILES: COCCc1nnc(-c2cc(C(=O)N3CCC(c4ccc(C#N)cc4)CC3)c(C)cc2C(C)C)[nH]1] | 0.705 |
| 001-199 | 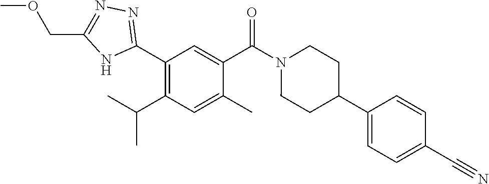 [SMILES: COCc1nnc(-c2cc(C(=O)N3CCC(c4ccc(C#N)cc4)CC3)c(C)cc2C(C)C)[nH]1] | 1.060 |
| 001-200 | 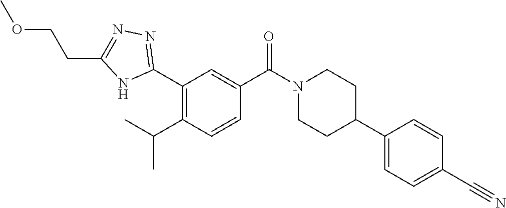 [SMILES: COCCc1nnc(-c2cc(C(=O)N3CCC(c4ccc(C#N)cc4)CC3)ccc2C(C)C)[nH]1] | 13.520 |
| 001-201 | 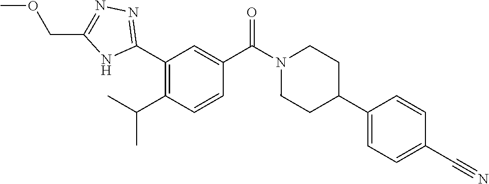 [SMILES: COCc1nnc(-c2cc(C(=O)N3CCC(c4ccc(C#N)cc4)CC3)ccc2C(C)C)[nH]1] | 23.140 |
| 001-202 | 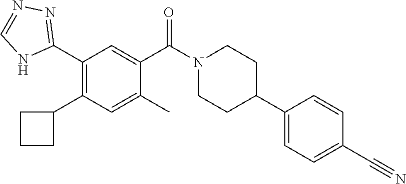 [SMILES: Cc1cc(C2CCC2)c(-c2nnc[nH]2)cc1C(=O)N1CCC(c2ccc(C#N)cc2)CC1] | 0.055 |
| 001-444 | 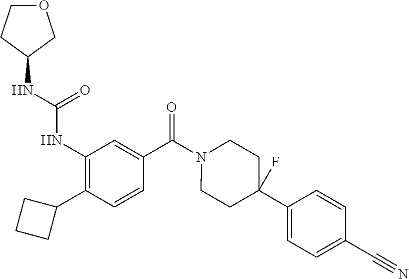 [SMILES: N#Cc1ccc(C2(F)CCN(C(=O)c3ccc(C4CCC4)c(NC(=O)N[C@H]4CCOC4)c3)CC2)cc1] | 0.040 |
| 001-445 | 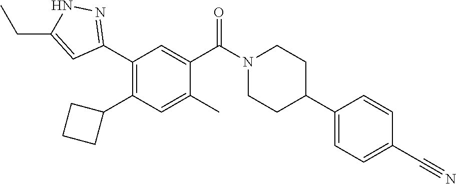 [SMILES: CCc1cc(-c2cc(C(=O)N3CCC(c4ccc(C#N)cc4)CC3)c(C)cc2C2CCC2)n[nH]1] | 0.490 |
| 001-446 | 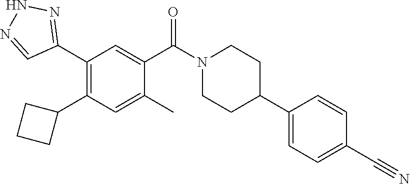 [SMILES: Cc1cc(C2CCC2)c(-c2cn[nH]n2)cc1C(=O)N1CCC(c2ccc(C#N)cc2)CC1] | 0.390 |
| 001-447 | 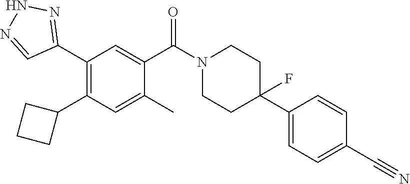 [SMILES: Cc1cc(C2CCC2)c(-c2cn[nH]n2)cc1C(=O)N1CCC(F)(c2ccc(C#N)cc2)CC1] | |
| 001-448 | 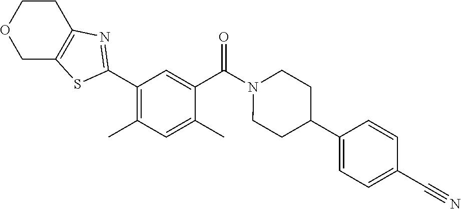 [SMILES: Cc1cc(C)c(-c2nc3c(s2)COCC3)cc1C(=O)N1CCC(c2ccc(C#N)cc2)CC1] | 10.610 |
| 001-449 | 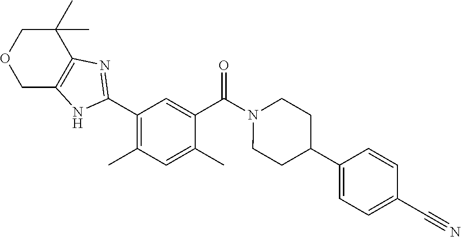 [SMILES: Cc1cc(C)c(-c2nc3c([nH]2)COCC3(C)C)cc1C(=O)N1CCC(c2ccc(C#N)cc2)CC1] | 0.135 |
| 001-450 | 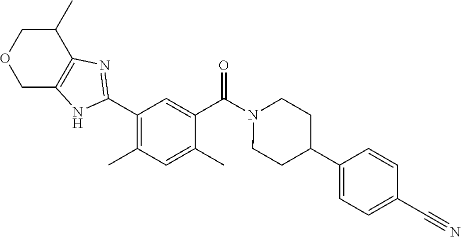 [SMILES: Cc1cc(C)c(-c2nc3c([nH]2)COCC3C)cc1C(=O)N1CCC(c2ccc(C#N)cc2)CC1] | 0.105 |
| 001-451 | 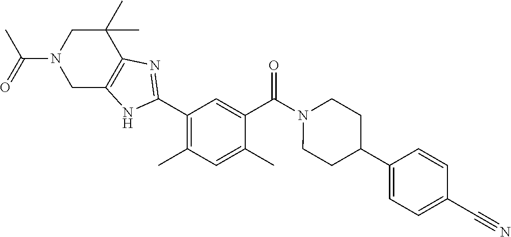 [SMILES: CC(=O)N1Cc2[nH]c(-c3cc(C(=O)N4CCC(c5ccc(C#N)cc5)CC4)c(C)cc3C)nc2C(C)(C)C1] | 0.070 |
| 001-203 | 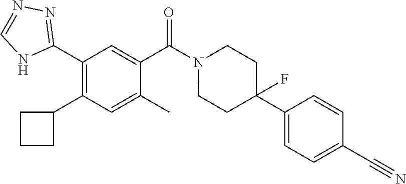 [SMILES: Cc1cc(C2CCC2)c(-c2nnc[nH]2)cc1C(=O)N1CCC(F)(c2ccc(C#N)cc2)CC1] | 0.045 |
| 001-204 | 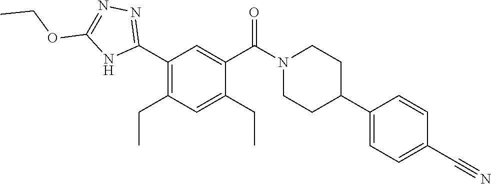 [SMILES: CCOc1nnc(-c2cc(C(=O)N3CCC(c4ccc(C#N)cc4)CC3)c(CC)cc2CC)[nH]1] | 0.195 |
| 001-205 | 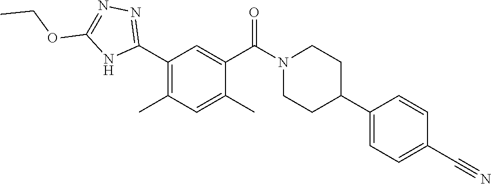 [SMILES: CCOc1nnc(-c2cc(C(=O)N3CCC(c4ccc(C#N)cc4)CC3)c(C)cc2C)[nH]1] | 0.220 |
| 001-206 | 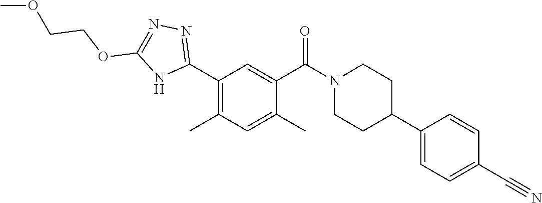 [SMILES: COCCOc1nnc(-c2cc(C(=O)N3CCC(c4ccc(C#N)cc4)CC3)c(C)cc2C)[nH]1] | 0.320 |
| 001-207 | 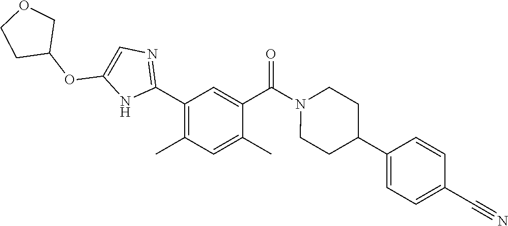 [SMILES: Cc1cc(C)c(-c2ncc(OC3CCOC3)[nH]2)cc1C(=O)N1CCC(c2ccc(C#N)cc2)CC1] | 0.300 |
| 001-208 | 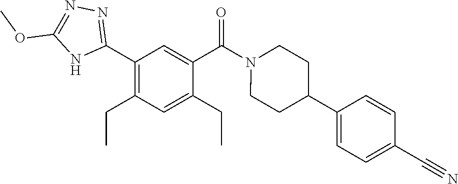 [SMILES: CCc1cc(CC)c(-c2nnc(OC)[nH]2)cc1C(=O)N1CCC(c2ccc(C#N)cc2)CC1] | 0.100 |
| 001-209 | 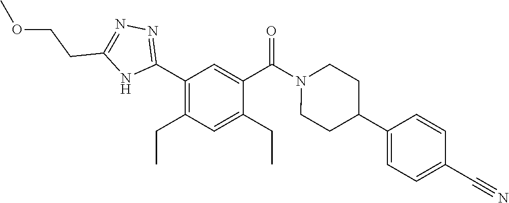 [SMILES: CCc1cc(CC)c(-c2nnc(CCOC)[nH]2)cc1C(=O)N1CCC(c2ccc(C#N)cc2)CC1] | 0.105 |
| 001-210 | 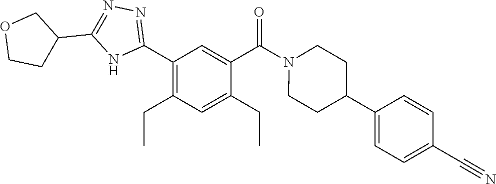 [SMILES: CCc1cc(CC)c(-c2nnc(C3CCOC3)[nH]2)cc1C(=O)N1CCC(c2ccc(C#N)cc2)CC1] | 0.310 |
| 001-211 | 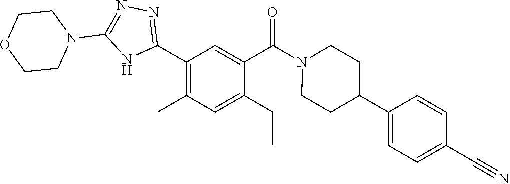 [SMILES: CCc1cc(C)c(-c2nnc(N3CCOCC3)[nH]2)cc1C(=O)N1CCC(c2ccc(C#N)cc2)CC1] | 0.830 |
| 001-452 | 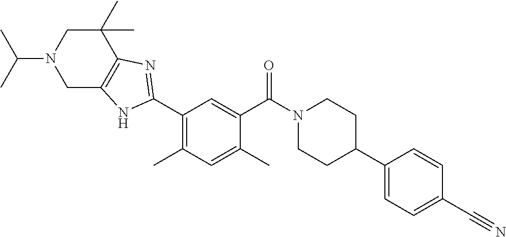 [SMILES: Cc1cc(C)c(-c2nc3c([nH]2)CN(C(C)C)CC3(C)C)cc1C(=O)N1CCC(c2ccc(C#N)cc2)CC1] | 0.130 |
| 001-453 | 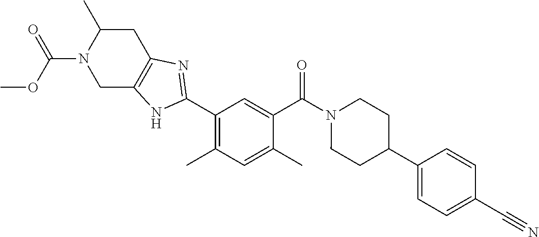 [SMILES: COC(=O)N1Cc2[nH]c(-c3cc(C(=O)N4CCC(c5ccc(C#N)cc5)CC4)c(C)cc3C)nc2CC1C] | 0.070 |
| 001-454 | 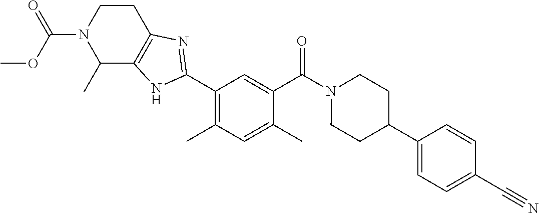 [SMILES: COC(=O)N1CCc2nc(-c3cc(C(=O)N4CCC(c5ccc(C#N)cc5)CC4)c(C)cc3C)[nH]c2C1C] | 0.077 |
| 001-455 | 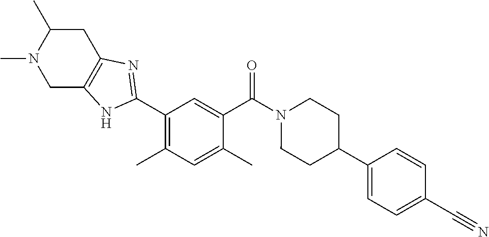 [SMILES: Cc1cc(C)c(-c2nc3c([nH]2)CN(C)C(C)C3)cc1C(=O)N1CCC(c2ccc(C#N)cc2)CC1] | 0.130 |
| 001-456 | 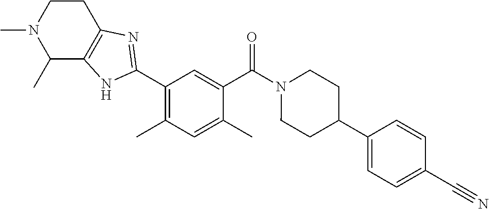 [SMILES: Cc1cc(C)c(-c2nc3c([nH]2)C(C)N(C)CC3)cc1C(=O)N1CCC(c2ccc(C#N)cc2)CC1] | 0.135 |
| 001-457 | 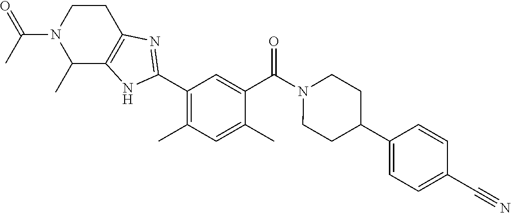 [SMILES: CC(=O)N1CCc2nc(-c3cc(C(=O)N4CCC(c5ccc(C#N)cc5)CC4)c(C)cc3C)[nH]c2C1C] | 0.080 |
| 001-458 | 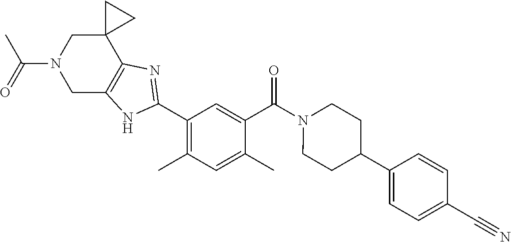 [SMILES: CC(=O)N1Cc2[nH]c(-c3cc(C(=O)N4CCC(c5ccc(C#N)cc5)CC4)c(C)cc3C)nc2C2(CC2)C1] | 0.130 |
| 001-459 | 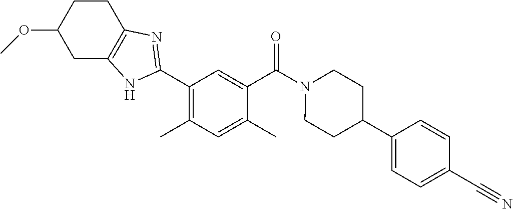 [SMILES: COC1CCc2nc(-c3cc(C(=O)N4CCC(c5ccc(C#N)cc5)CC4)c(C)cc3C)[nH]c2C1] | 0.310 |
| 001-460 | 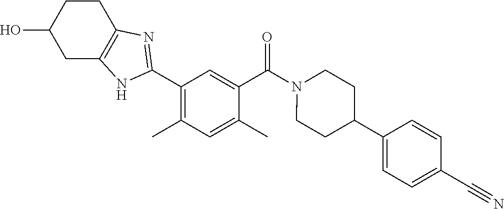 [SMILES: Cc1cc(C)c(-c2nc3c([nH]2)CC(O)CC3)cc1C(=O)N1CCC(c2ccc(C#N)cc2)CC1] | 0.090 |
| 001-212 | 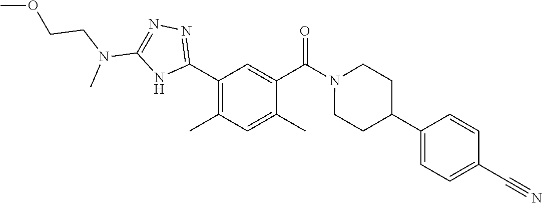 [SMILES: COCCN(C)c1nnc(-c2cc(C(=O)N3CCC(c4ccc(C#N)cc4)CC3)c(C)cc2C)[nH]1] | 7.190 |
| 001-213 | 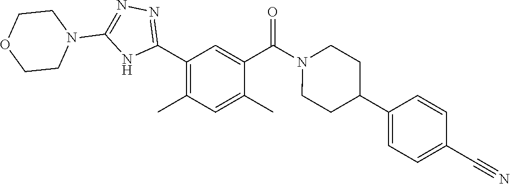 [SMILES: Cc1cc(C)c(-c2nnc(N3CCOCC3)[nH]2)cc1C(=O)N1CCC(c2ccc(C#N)cc2)CC1] | 0.790 |
| 001-214 | 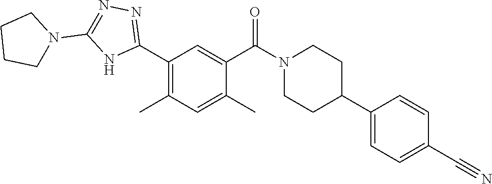 [SMILES: Cc1cc(C)c(-c2nnc(N3CCCC3)[nH]2)cc1C(=O)N1CCC(c2ccc(C#N)cc2)CC1] | 28.760 |
| 001-215 | 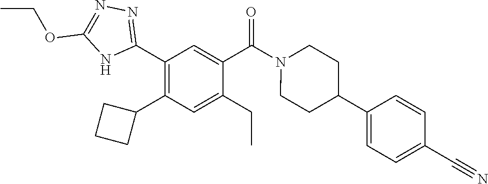 [SMILES: CCOc1nnc(-c2cc(C(=O)N3CCC(c4ccc(C#N)cc4)CC3)c(CC)cc2C2CCC2)[nH]1] | 0.080 |
| 001-216 | 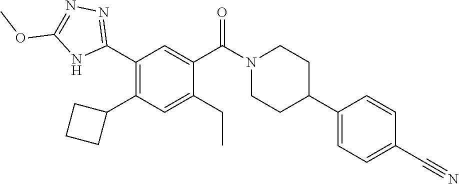 [SMILES: CCc1cc(C2CCC2)c(-c2nnc(OC)[nH]2)cc1C(=O)N1CCC(c2ccc(C#N)cc2)CC1] | 0.090 |
| 001-217 | 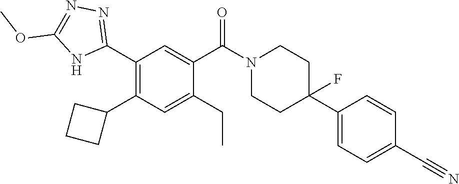 [SMILES: CCc1cc(C2CCC2)c(-c2nnc(OC)[nH]2)cc1C(=O)N1CCC(F)(c2ccc(C#N)cc2)CC1] | 0.100 |
| 001-218 | 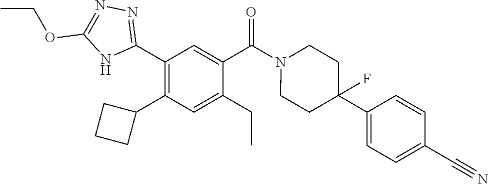 [SMILES: CCOc1nnc(-c2cc(C(=O)N3CCC(F)(c4ccc(C#N)cc4)CC3)c(CC)cc2C2CCC2)[nH]1] | 0.170 |
| 001-219 | 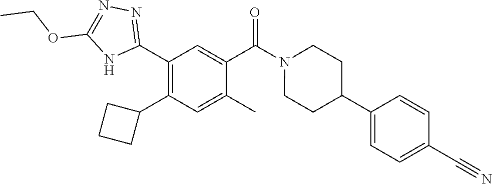 [SMILES: CCOc1nnc(-c2cc(C(=O)N3CCC(c4ccc(C#N)cc4)CC3)c(C)cc2C2CCC2)[nH]1] | 0.085 |
| 001-461 | 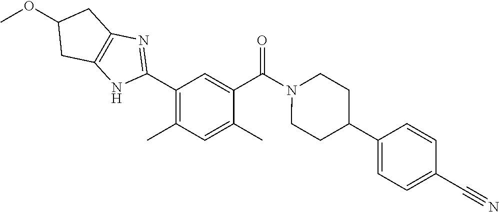 [SMILES: COC1Cc2nc(-c3cc(C(=O)N4CCC(c5ccc(C#N)cc5)CC4)c(C)cc3C)[nH]c2C1] | 0.105 |
| 001-462 | 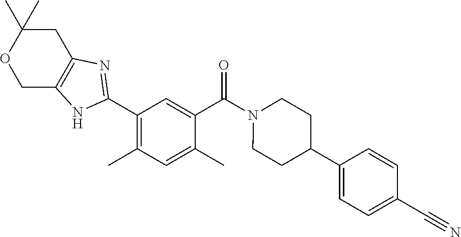 [SMILES: Cc1cc(C)c(-c2nc3c([nH]2)COC(C)(C)C3)cc1C(=O)N1CCC(c2ccc(C#N)cc2)CC1] | 0.780 |
| 001-463 | 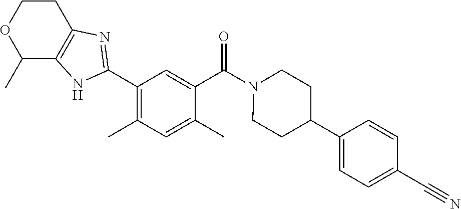 [SMILES: Cc1cc(C)c(-c2nc3c([nH]2)C(C)OCC3)cc1C(=O)N1CCC(c2ccc(C#N)cc2)CC1] | 0.440 |
| 001-464 | 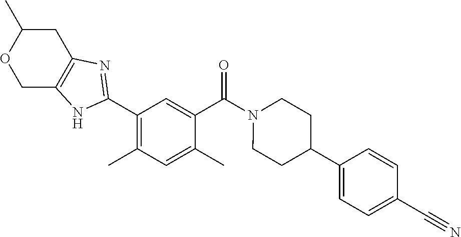 [SMILES: Cc1cc(C)c(-c2nc3c([nH]2)COC(C)C3)cc1C(=O)N1CCC(c2ccc(C#N)cc2)CC1] | 0.065 |
| 001-465 | 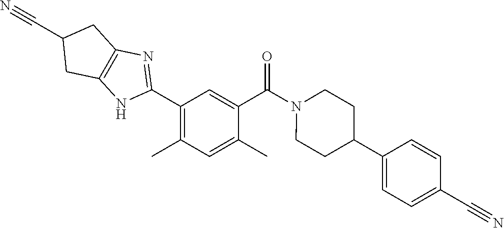 [SMILES: Cc1cc(C)c(-c2nc3c([nH]2)CC(C#N)C3)cc1C(=O)N1CCC(c2ccc(C#N)cc2)CC1] | 0.050 |
| 001-466 | 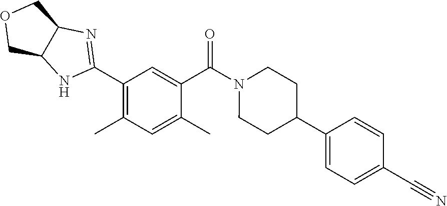 [SMILES: Cc1cc(C)c(C2=N[C@@H]3COC[C@@H]3N2)cc1C(=O)N1CCC(c2ccc(C#N)cc2)CC1] | 34.670 |
| 001-467 | 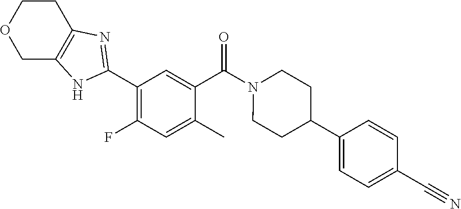 [SMILES: Cc1cc(F)c(-c2nc3c([nH]2)COCC3)cc1C(=O)N1CCC(c2ccc(C#N)cc2)CC1] | 1.810 |
| 001-468 | 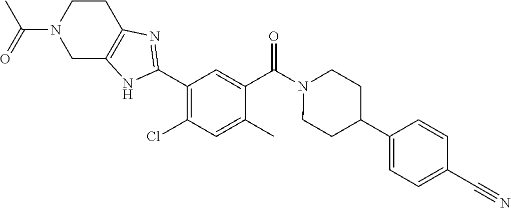 [SMILES: CC(=O)N1CCc2nc(-c3cc(C(=O)N4CCC(c5ccc(C#N)cc5)CC4)c(C)cc3Cl)[nH]c2C1] | 0.170 |
| 001-220 | 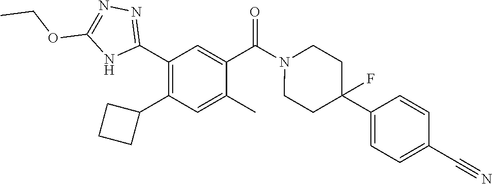 [SMILES: CCOc1nnc(-c2cc(C(=O)N3CCC(F)(c4ccc(C#N)cc4)CC3)c(C)cc2C2CCC2)[nH]1] | 0.055 |
| 001-221 | 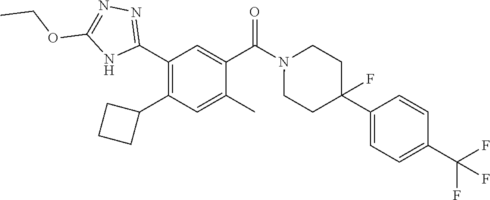 [SMILES: CCOc1nnc(-c2cc(C(=O)N3CCC(F)(c4ccc(C(F)(F)F)cc4)CC3)c(C)cc2C2CCC2)[nH]1] | 0.250 |
| 001-222 | 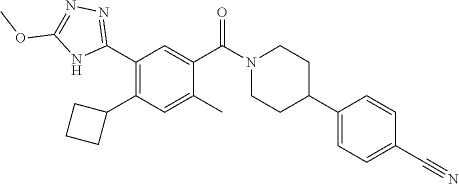 [SMILES: COc1nnc(-c2cc(C(=O)N3CCC(c4ccc(C#N)cc4)CC3)c(C)cc2C2CCC2)[nH]1] | 0.065 |
| 001-223 | 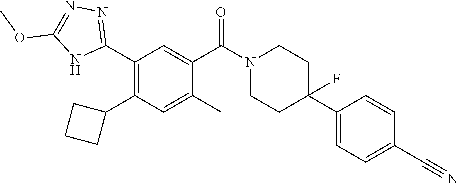 [SMILES: COc1nnc(-c2cc(C(=O)N3CCC(F)(c4ccc(C#N)cc4)CC3)c(C)cc2C2CCC2)[nH]1] | 0.030 |
| 001-224 | 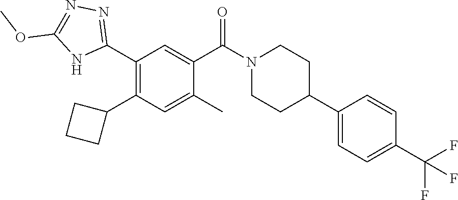 [SMILES: COc1nnc(-c2cc(C(=O)N3CCC(c4ccc(C(F)(F)F)cc4)CC3)c(C)cc2C2CCC2)[nH]1] | 0.160 |
| 001-225 | 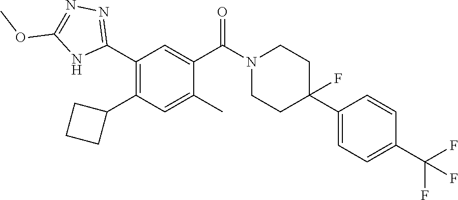 [SMILES: COc1nnc(-c2cc(C(=O)N3CCC(F)(c4ccc(C(F)(F)F)cc4)CC3)c(C)cc2C2CCC2)[nH]1] | 0.140 |
| 001-226 | 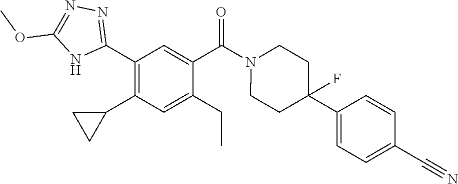 [SMILES: CCc1cc(C2CC2)c(-c2nnc(OC)[nH]2)cc1C(=O)N1CCC(F)(c2ccc(C#N)cc2)CC1] | 0.055 |
| 001-227 | 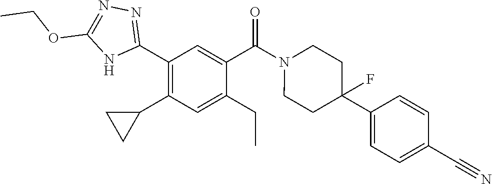 [SMILES: CCOc1nnc(-c2cc(C(=O)N3CCC(F)(c4ccc(C#N)cc4)CC3)c(CC)cc2C2CC2)[nH]1] | 0.070 |
| 001-469 | 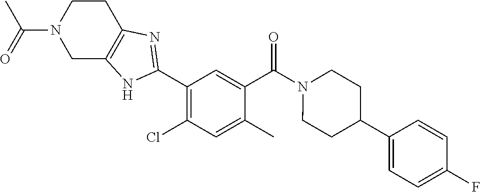 [SMILES: CC(=O)N1CCc2nc(-c3cc(C(=O)N4CCC(c5ccc(F)cc5)CC4)c(C)cc3Cl)[nH]c2C1] | 2.100 |
| 001-470 | 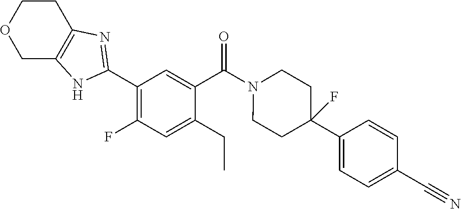 [SMILES: CCc1cc(F)c(-c2nc3c([nH]2)COCC3)cc1C(=O)N1CCC(F)(c2ccc(C#N)cc2)CC1] | 1.130 |
| 001-471 | 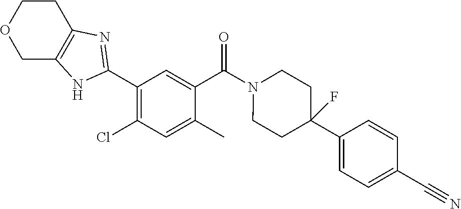 [SMILES: Cc1cc(Cl)c(-c2nc3c([nH]2)COCC3)cc1C(=O)N1CCC(F)(c2ccc(C#N)cc2)CC1] | 0.450 |
| 001-472 | 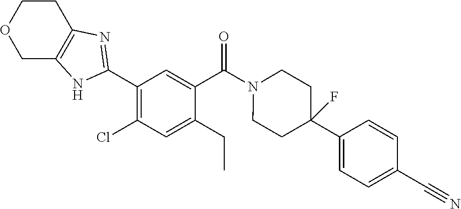 [SMILES: CCc1cc(Cl)c(-c2nc3c([nH]2)COCC3)cc1C(=O)N1CCC(F)(c2ccc(C#N)cc2)CC1] | 0.230 |
| 001-473 | 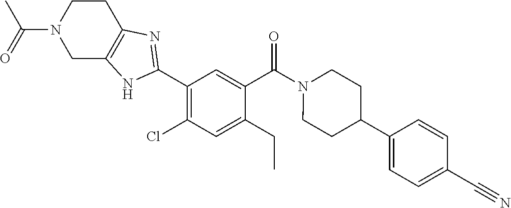 [SMILES: CCc1cc(Cl)c(-c2nc3c([nH]2)CN(C(C)=O)CC3)cc1C(=O)N1CCC(c2ccc(C#N)cc2)CC1] | 0.110 |
| 001-474 | 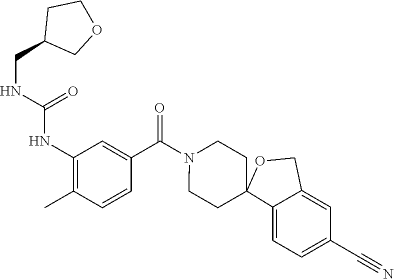 [SMILES: Cc1ccc(C(=O)N2CCC3(CC2)OCc2cc(C#N)ccc23)cc1NC(=O)NC[C@H]1CCOC1] | 0.180 |
| 001-475 | 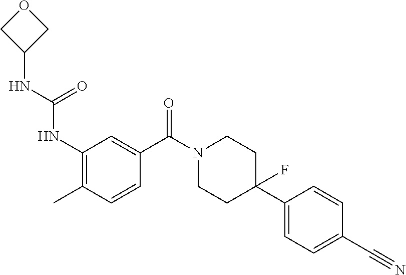 [SMILES: Cc1ccc(C(=O)N2CCC(F)(c3ccc(C#N)cc3)CC2)cc1NC(=O)NC1COC1] | 0.050 |
| 001-476 | 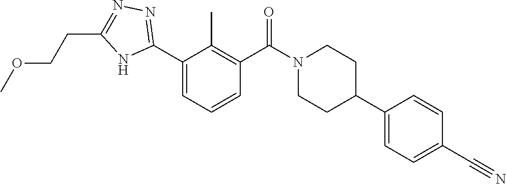 [SMILES: COCCc1nnc(-c2cccc(C(=O)N3CCC(c4ccc(C#N)cc4)CC3)c2C)[nH]1] | >50 |
| 001-228 | 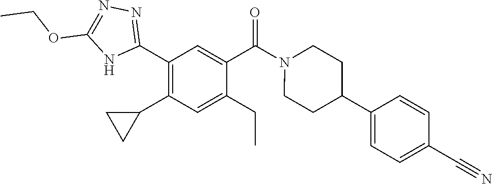 [SMILES: CCOc1nnc(-c2cc(C(=O)N3CCC(c4ccc(C#N)cc4)CC3)c(CC)cc2C2CC2)[nH]1] | 0.110 |
| 001-229 | 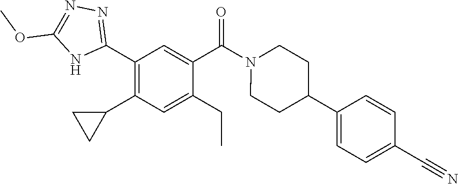 [SMILES: CCc1cc(C2CC2)c(-c2nnc(OC)[nH]2)cc1C(=O)N1CCC(c2ccc(C#N)cc2)CC1] | 0.090 |
| 001-230 | 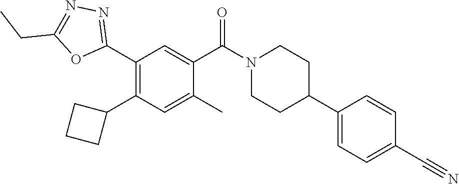 [SMILES: CCc1nnc(-c2cc(C(=O)N3CCC(c4ccc(C#N)cc4)CC3)c(C)cc2C2CCC2)o1] | 30.990 |
| 001-231 | 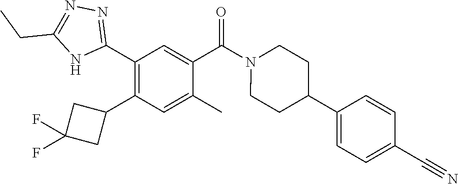 [SMILES: CCc1nnc(-c2cc(C(=O)N3CCC(c4ccc(C#N)cc4)CC3)c(C)cc2C2CC(F)(F)C2)[nH]1] | 0.300 |
| 001-232 | 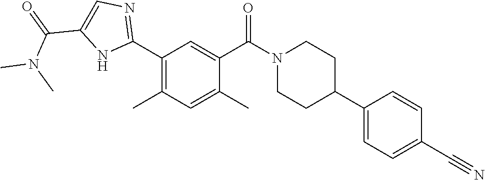 [SMILES: Cc1cc(C)c(-c2ncc(C(=O)N(C)C)[nH]2)cc1C(=O)N1CCC(c2ccc(C#N)cc2)CC1] | 0.650 |
| 001-233 | 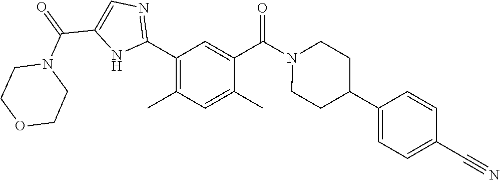 [SMILES: Cc1cc(C)c(-c2ncc(C(=O)N3CCOCC3)[nH]2)cc1C(=O)N1CCC(c2ccc(C#N)cc2)CC1] | 1.630 |
| 001-234 | 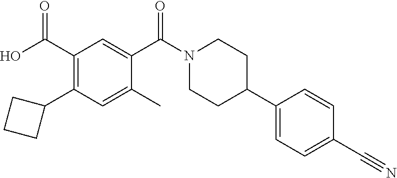 [SMILES: Cc1cc(C2CCC2)c(C(=O)O)cc1C(=O)N1CCC(c2ccc(C#N)cc2)CC1] | >50 |
| 001-235 | 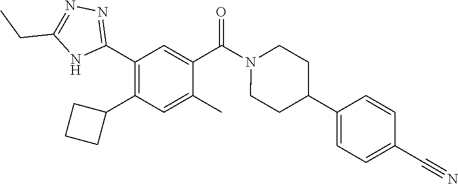 [SMILES: CCc1nnc(-c2cc(C(=O)N3CCC(c4ccc(C#N)cc4)CC3)c(C)cc2C2CCC2)[nH]1] | 0.030 |
| 001-236 | 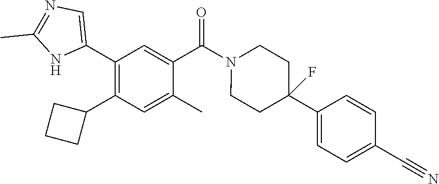 [SMILES: Cc1ncc(-c2cc(C(=O)N3CCC(F)(c4ccc(C#N)cc4)CC3)c(C)cc2C2CCC2)[nH]1] | 0.060 |
| 001-477 | 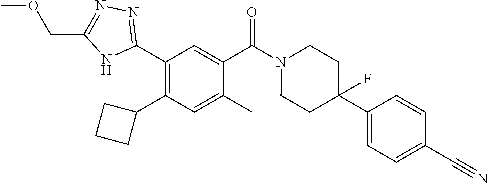 [SMILES: COCc1nnc(-c2cc(C(=O)N3CCC(F)(c4ccc(C#N)cc4)CC3)c(C)cc2C2CCC2)[nH]1] | 0.130 |
| 001-478 | 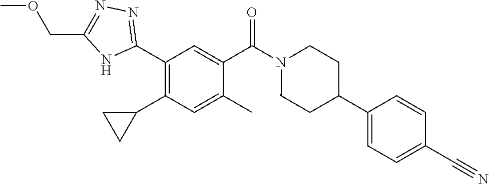 [SMILES: COCc1nnc(-c2cc(C(=O)N3CCC(c4ccc(C#N)cc4)CC3)c(C)cc2C2CC2)[nH]1] | 0.420 |
| 001-479 | 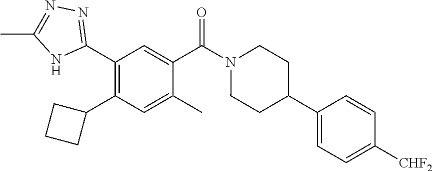 [SMILES: Cc1nnc(-c2cc(C(=O)N3CCC(c4ccc(C(F)F)cc4)CC3)c(C)cc2C2CCC2)[nH]1] | 0.110 |
| 001-480 | 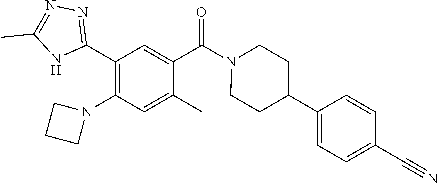 [SMILES: Cc1nnc(-c2cc(C(=O)N3CCC(c4ccc(C#N)cc4)CC3)c(C)cc2N2CCC2)[nH]1] | 0.180 |
| 001-481 | 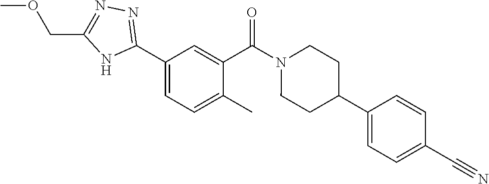 [SMILES: COCc1nnc(-c2ccc(C)c(C(=O)N3CCC(c4ccc(C#N)cc4)CC3)c2)[nH]1] | 21.240 |
| 001-482 | 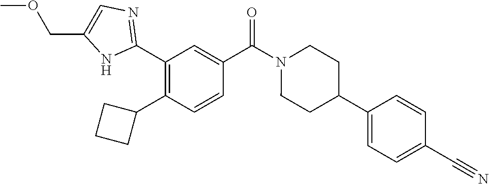 [SMILES: COCc1cnc(-c2cc(C(=O)N3CCC(c4ccc(C#N)cc4)CC3)ccc2C2CCC2)[nH]1] | 0.240 |
| 001-483 | 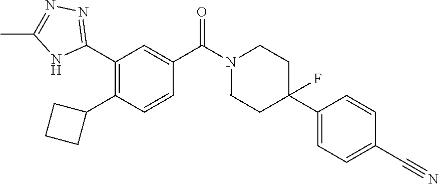 [SMILES: Cc1nnc(-c2cc(C(=O)N3CCC(F)(c4ccc(C#N)cc4)CC3)ccc2C2CCC2)[nH]1] | 0.100 |
| 001-484 | 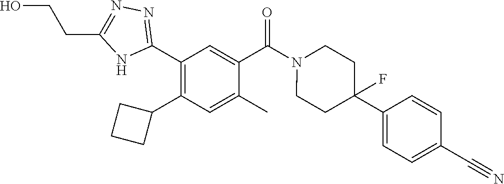 [SMILES: Cc1cc(C2CCC2)c(-c2nnc(CCO)[nH]2)cc1C(=O)N1CCC(F)(c2ccc(C#N)cc2)CC1] | 0.070 |
| 001-485 | 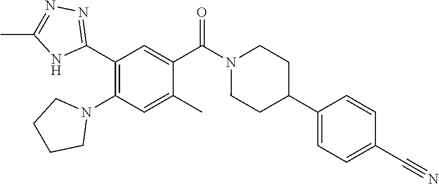 [SMILES: Cc1nnc(-c2cc(C(=O)N3CCC(c4ccc(C#N)cc4)CC3)c(C)cc2N2CCCC2)[nH]1] | 0.130 |
| 001-237 | 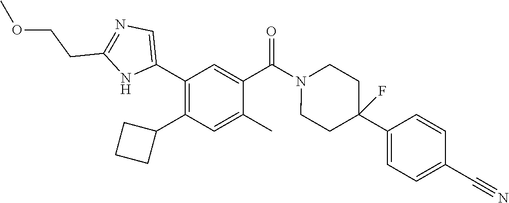 [SMILES: COCCc1ncc(-c2cc(C(=O)N3CCC(F)(c4ccc(C#N)cc4)CC3)c(C)cc2C2CCC2)[nH]1] | 0.080 |
| 001-238 | 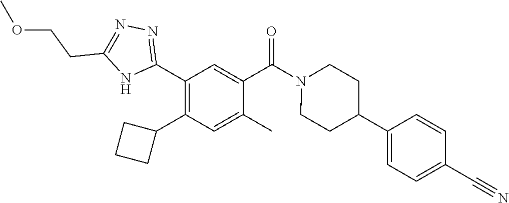 [SMILES: COCCc1nnc(-c2cc(C(=O)N3CCC(c4ccc(C#N)cc4)CC3)c(C)cc2C2CCC2)[nH]1] | 0.100 |
| 001-239 | 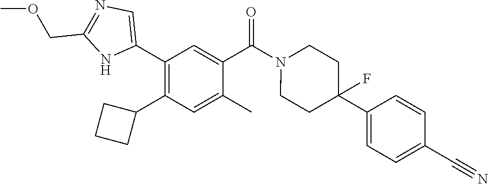 [SMILES: COCc1ncc(-c2cc(C(=O)N3CCC(F)(c4ccc(C#N)cc4)CC3)c(C)cc2C2CCC2)[nH]1] | 0.050 |
| 001-240 | 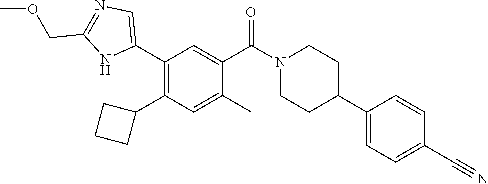 [SMILES: COCc1ncc(-c2cc(C(=O)N3CCC(c4ccc(C#N)cc4)CC3)c(C)cc2C2CCC2)[nH]1] | 0.050 |
| 001-241 | 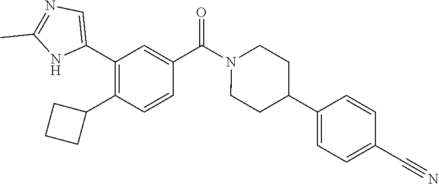 [SMILES: Cc1ncc(-c2cc(C(=O)N3CCC(c4ccc(C#N)cc4)CC3)ccc2C2CCC2)[nH]1] | 0.120 |
| 001-242 | 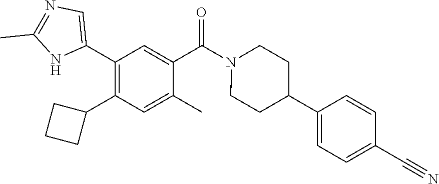 [SMILES: Cc1ncc(-c2cc(C(=O)N3CCC(c4ccc(C#N)cc4)CC3)c(C)cc2C2CCC2)[nH]1] | 0.050 |
| 001-243 | 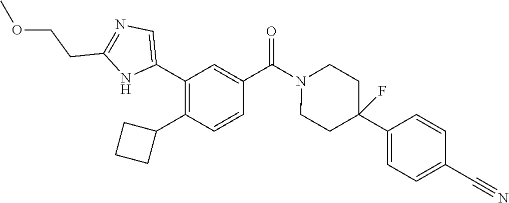 [SMILES: COCCc1ncc(-c2cc(C(=O)N3CCC(F)(c4ccc(C#N)cc4)CC3)ccc2C2CCC2)[nH]1] | 0.120 |
| 001-486 | 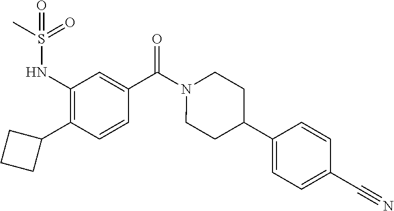 [SMILES: CS(=O)(=O)Nc1cc(C(=O)N2CCC(c3ccc(C#N)cc3)CC2)ccc1C1CCC1] | 0.540 |
| 001-487 | 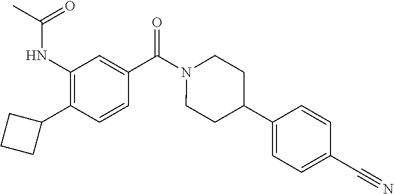 [SMILES: CC(=O)Nc1cc(C(=O)N2CCC(c3ccc(C#N)cc3)CC2)ccc1C1CCC1] | 0.380 |
| 001-488 | 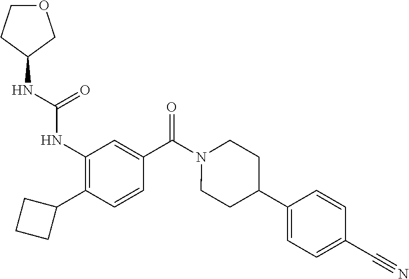 [SMILES: N#Cc1ccc(C2CCN(C(=O)c3ccc(C4CCC4)c(NC(=O)N[C@H]4CCOC4)c3)CC2)cc1] | 0.030 |
| 001-489 | 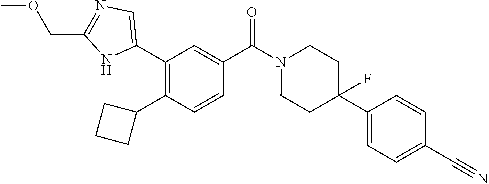 [SMILES: COCc1ncc(-c2cc(C(=O)N3CCC(F)(c4ccc(C#N)cc4)CC3)ccc2C2CCC2)[nH]1] | 0.180 |
| 001-490 | 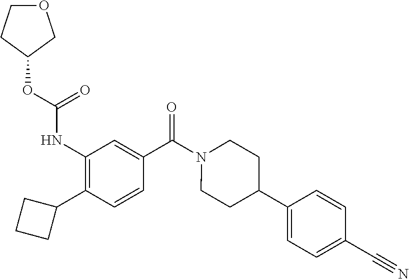 [SMILES: N#Cc1ccc(C2CCN(C(=O)c3ccc(C4CCC4)c(NC(=O)O[C@@H]4CCOC4)c3)CC2)cc1] | 0.090 |
| 001-491 | 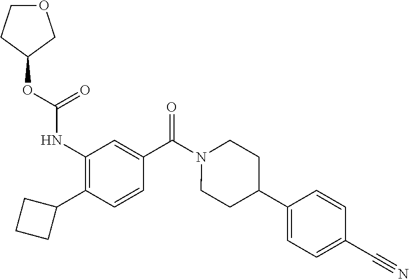 [SMILES: N#Cc1ccc(C2CCN(C(=O)c3ccc(C4CCC4)c(NC(=O)O[C@H]4CCOC4)c3)CC2)cc1] | 0.070 |
| 001-492 | 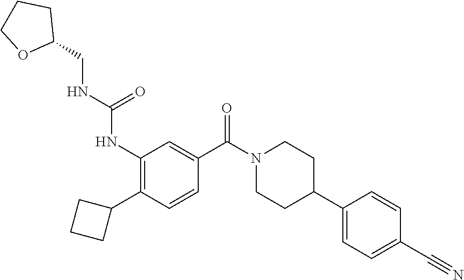 [SMILES: N#Cc1ccc(C2CCN(C(=O)c3ccc(C4CCC4)c(NC(=O)NC[C@H]4CCCO4)c3)CC2)cc1] | 0.030 |
| 001-244 | 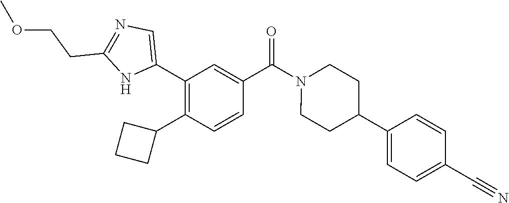 [SMILES: COCCc1ncc(-c2cc(C(=O)N3CCC(c4ccc(C#N)cc4)CC3)ccc2C2CCC2)[nH]1] | 0.220 |
| 001-245 | 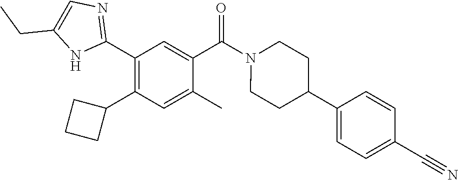 [SMILES: CCc1cnc(-c2cc(C(=O)N3CCC(c4ccc(C#N)cc4)CC3)c(C)cc2C2CCC2)[nH]1] | 0.020 |
| 001-246 | 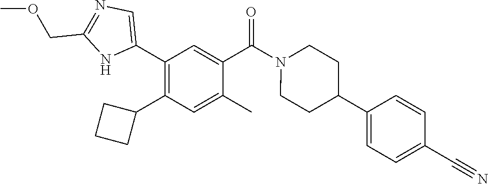 [SMILES: COCc1ncc(-c2cc(C(=O)N3CCC(c4ccc(C#N)cc4)CC3)c(C)cc2C2CCC2)[nH]1] | 0.050 |
| 001-247 | 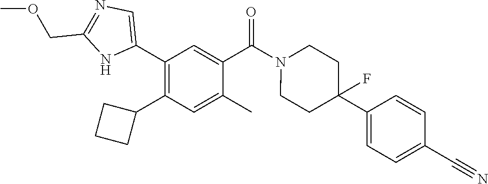 [SMILES: COCc1ncc(-c2cc(C(=O)N3CCC(F)(c4ccc(C#N)cc4)CC3)c(C)cc2C2CCC2)[nH]1] | 0.020 |
| 001-248 | 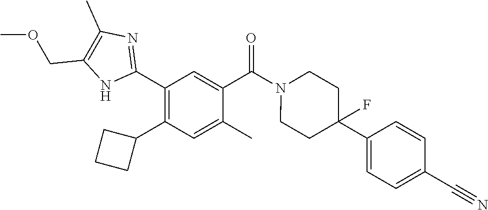 [SMILES: COCc1[nH]c(-c2cc(C(=O)N3CCC(F)(c4ccc(C#N)cc4)CC3)c(C)cc2C2CCC2)nc1C] | 0.060 |
| 001-249 | 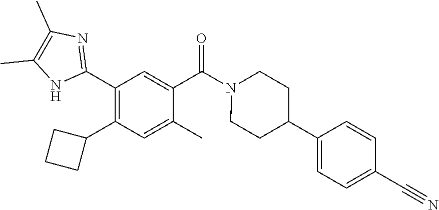 [SMILES: Cc1cc(C2CCC2)c(-c2nc(C)c(C)[nH]2)cc1C(=O)N1CCC(c2ccc(C#N)cc2)CC1] | 0.070 |
| 001-250 | 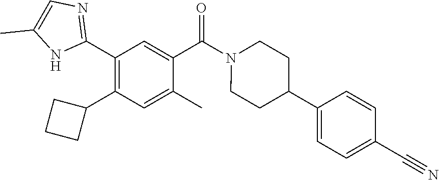 [SMILES: Cc1cnc(-c2cc(C(=O)N3CCC(c4ccc(C#N)cc4)CC3)c(C)cc2C2CCC2)[nH]1] | 0.030 |
| 001-493 | 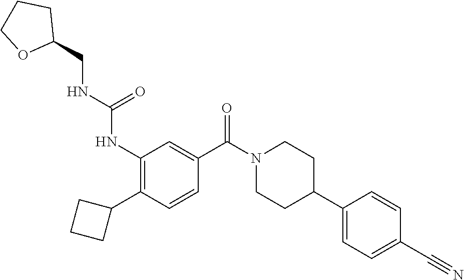 [SMILES: N#Cc1ccc(C2CCN(C(=O)c3ccc(C4CCC4)c(NC(=O)NC[C@@H]4CCCO4)c3)CC2)cc1] | 0.090 |
| 001-494 | 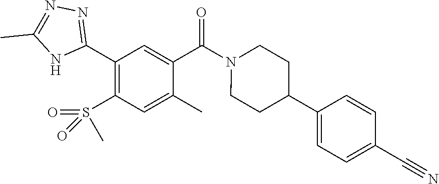 [SMILES: Cc1nnc(-c2cc(C(=O)N3CCC(c4ccc(C#N)cc4)CC3)c(C)cc2S(C)(=O)=O)[nH]1] | >50 |
| 001-495 | 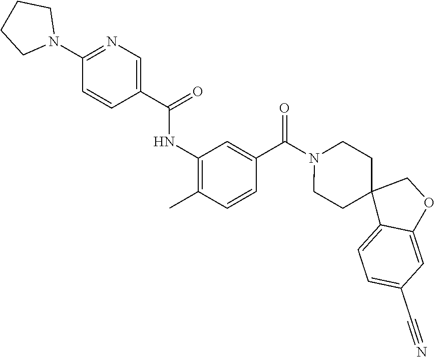 [SMILES: Cc1ccc(C(=O)N2CCC3(CC2)COc2cc(C#N)ccc23)cc1NC(=O)c1ccc(N2CCCC2)nc1] | |
| 001-496 | 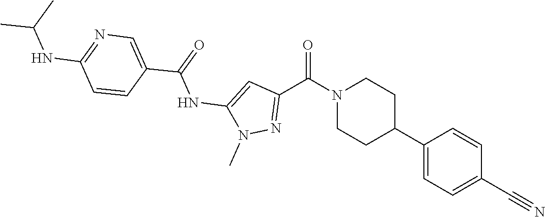 [SMILES: CC(C)Nc1ccc(C(=O)Nc2cc(C(=O)N3CCC(c4ccc(C#N)cc4)CC3)nn2C)cn1] | |
| 001-497 | 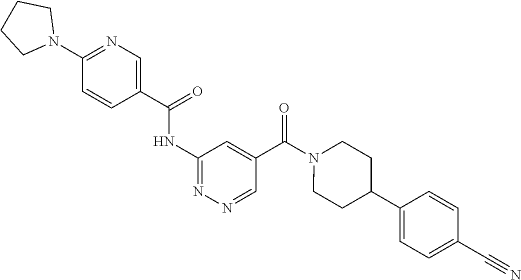 [SMILES: N#Cc1ccc(C2CCN(C(=O)c3cnnc(NC(=O)c4ccc(N5CCCC5)nc4)c3)CC2)cc1] | |
| 001-498 | 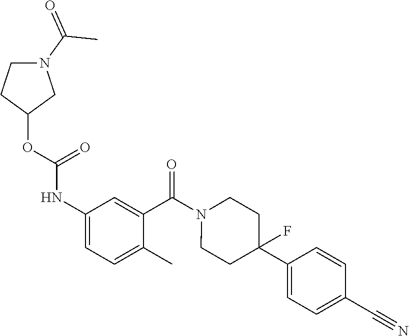 [SMILES: CC(=O)N1CCC(OC(=O)Nc2ccc(C)c(C(=O)N3CCC(F)(c4ccc(C#N)cc4)CC3)c2)C1] | |
| 001-498 | 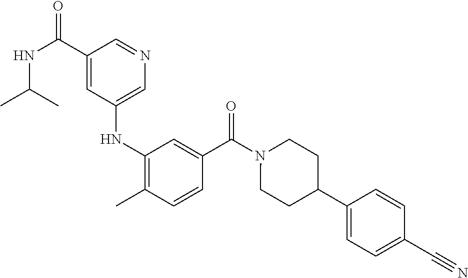 [SMILES: Cc1ccc(C(=O)N2CCC(c3ccc(C#N)cc3)CC2)cc1Nc1cncc(C(=O)NC(C)C)c1] | |
| 001-499 | 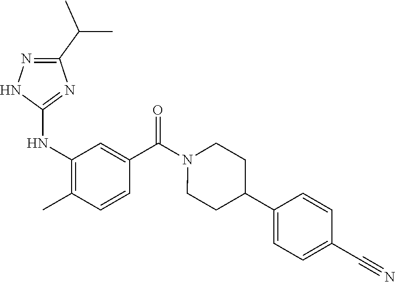 [SMILES: Cc1ccc(C(=O)N2CCC(c3ccc(C#N)cc3)CC2)cc1Nc1nc(C(C)C)n[nH]1] | |
| 001-500 | 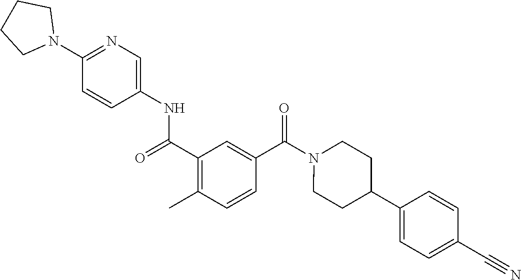 [SMILES: Cc1ccc(C(=O)N2CCC(c3ccc(C#N)cc3)CC2)cc1C(=O)Nc1ccc(N2CCCC2)nc1] | |

TABLE C-2
Fatty Acid Synthase Inbititors

| | | Hu |
|---|---|---|
| Cmpd | | FASN |
| No. | Structure | IC₅₀ (μM) |
| 002-1 | 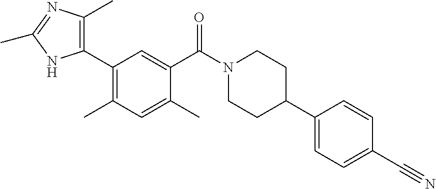 [SMILES: Cc1nc(C)c(-c2cc(C(=O)N3CCC(c4ccc(C#N)cc4)CC3)c(C)cc2C)[nH]1] | 0.024 |
| 002-2 | 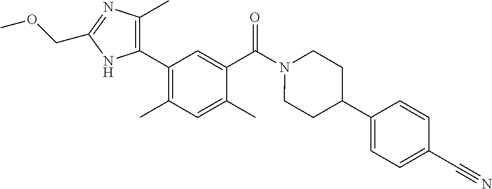 [SMILES: COCc1nc(C)c(-c2cc(C(=O)N3CCC(c4ccc(C#N)cc4)CC3)c(C)cc2C)[nH]1] | 0.041 |
| 002-3 | 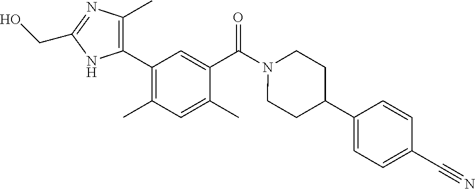 [SMILES: Cc1cc(C)c(-c2[nH]c(CO)nc2C)cc1C(=O)N1CCC(c2ccc(C#N)cc2)CC1] | 0.032 |
| 002-4 | 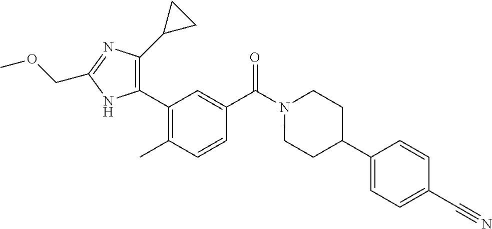 [SMILES: COCc1nc(C2CC2)c(-c2cc(C(=O)N3CCC(c4ccc(C#N)cc4)CC3)ccc2C)[nH]1] | 0.053 |
| 002-5 | 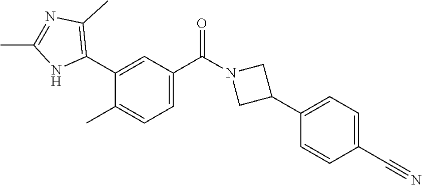 [SMILES: Cc1nc(C)c(-c2cc(C(=O)N3CC(c4ccc(C#N)cc4)C3)ccc2C)[nH]1] | 0.010 |
| 002-6 | 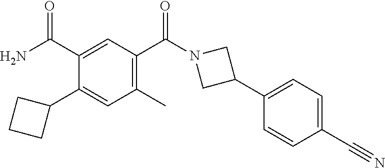 [SMILES: Cc1cc(C2CCC2)c(C(N)=O)cc1C(=O)N1CC(c2ccc(C#N)cc2)C1] | 0.105 |
| 002-7 | 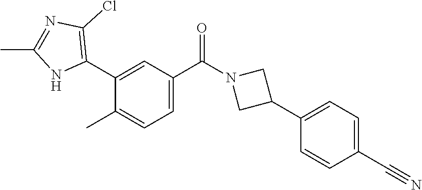 [SMILES: Cc1nc(Cl)c(-c2cc(C(=O)N3CC(c4ccc(C#N)cc4)C3)ccc2C)[nH]1] | 0.006 |
| 002-8 | 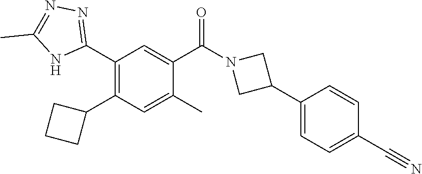 [SMILES: Cc1nnc(-c2cc(C(=O)N3CC(c4ccc(C#N)cc4)C3)c(C)cc2C2CCC2)[nH]1] | 0.040 |
| 002-9 | 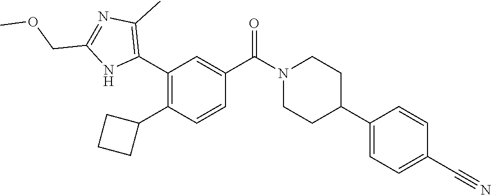 [SMILES: COCc1nc(C)c(-c2cc(C(=O)N3CCC(c4ccc(C#N)cc4)CC3)ccc2C2CCC2)[nH]1] | 0.025 |
| 002-10 | 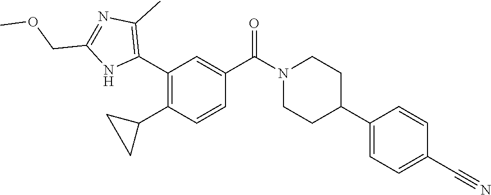 [SMILES: COCc1nc(C)c(-c2cc(C(=O)N3CCC(c4ccc(C#N)cc4)CC3)ccc2C2CC2)[nH]1] | 0.027 |
| 002-11 | 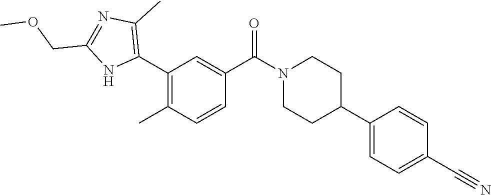 [SMILES: COCc1nc(C)c(-c2cc(C(=O)N3CCC(c4ccc(C#N)cc4)CC3)ccc2C)[nH]1] | 0.036 |
| 002-12 | 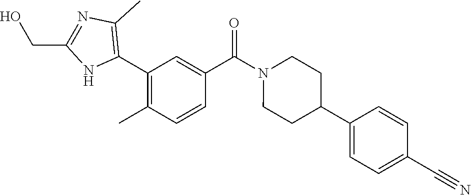 [SMILES: Cc1ccc(C(=O)N2CCC(c3ccc(C#N)cc3)CC2)cc1-c1[nH]c(CO)nc1C] | 0.036 |
| 002-13 | 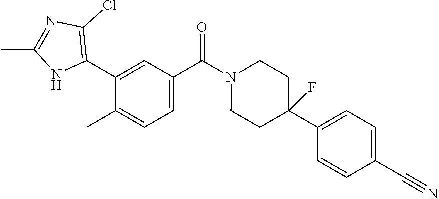 [SMILES: Cc1nc(Cl)c(-c2cc(C(=O)N3CCC(F)(c4ccc(C#N)cc4)CC3)ccc2C)[nH]1] | 0.006 |
| 002-14 | 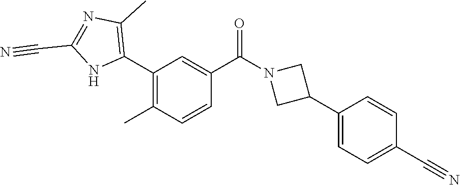 [SMILES: Cc1ccc(C(=O)N2CC(c3ccc(C#N)cc3)C2)cc1-c1[nH]c(C#N)nc1C] | 0.027 |
| 002-15 | 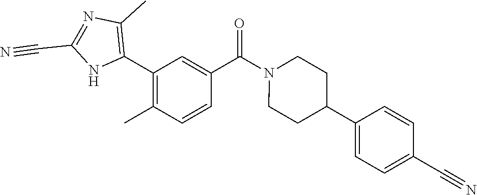 [SMILES: Cc1ccc(C(=O)N2CCC(c3ccc(C#N)cc3)CC2)cc1-c1[nH]c(C#N)nc1C] | 0.026 |
| 002-16 | 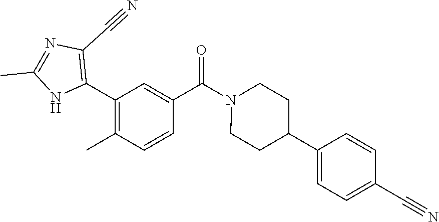 [SMILES: Cc1nc(C#N)c(-c2cc(C(=O)N3CCC(c4ccc(C#N)cc4)CC3)ccc2C)[nH]1] | 0.018 |
| 002-17 | 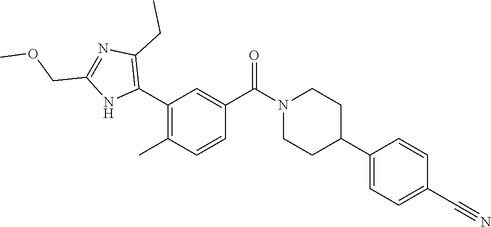 [SMILES: CCc1nc(COC)[nH]c1-c1cc(C(=O)N2CCC(c3ccc(C#N)cc3)CC2)ccc1C] | 0.045 |
| 002-18 | 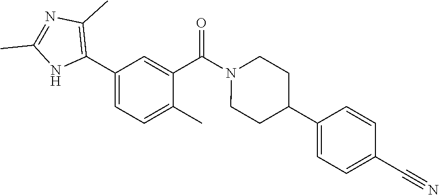 [SMILES: Cc1nc(C)c(-c2ccc(C)c(C(=O)N3CCC(c4ccc(C#N)cc4)CC3)c2)[nH]1] | 0.540 |
| 002-19 | 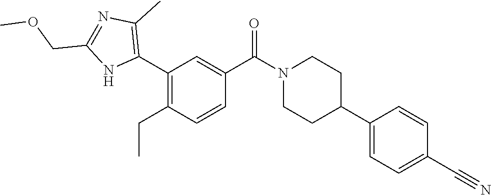 [SMILES: CCc1ccc(C(=O)N2CCC(c3ccc(C#N)cc3)CC2)cc1-c1[nH]c(COC)nc1C] | 0.030 |
| 002-20 | 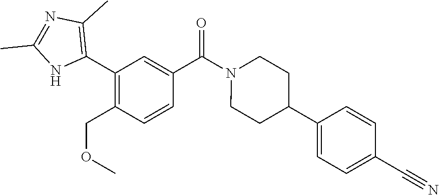 [SMILES: COCc1ccc(C(=O)N2CCC(c3ccc(C#N)cc3)CC2)cc1-c1[nH]c(C)nc1C] | 0.165 |
| 002-21 | 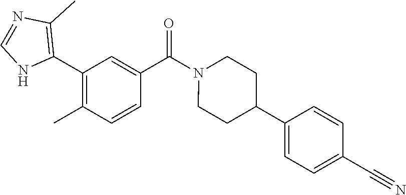 [SMILES: Cc1ccc(C(=O)N2CCC(c3ccc(C#N)cc3)CC2)cc1-c1[nH]cnc1C] | 0.207 |
| 002-22 | 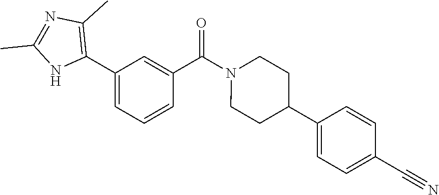 [SMILES: Cc1nc(C)c(-c2cccc(C(=O)N3CCC(c4ccc(C#N)cc4)CC3)c2)[nH]1] | 1.690 |
| 002-23 | 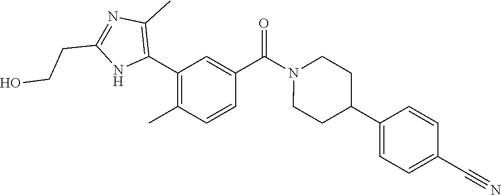 [SMILES: Cc1ccc(C(=O)N2CCC(c3ccc(C#N)cc3)CC2)cc1-c1[nH]c(CCO)nc1C] | 0.029 |
| 002-24 | 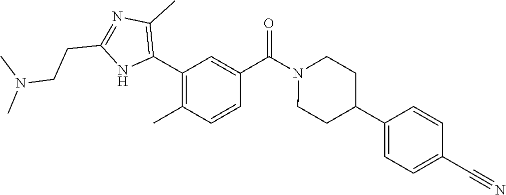 [SMILES: Cc1ccc(C(=O)N2CCC(c3ccc(C#N)cc3)CC2)cc1-c1[nH]c(CCN(C)C)nc1C] | 0.041 |
| 002-25 | 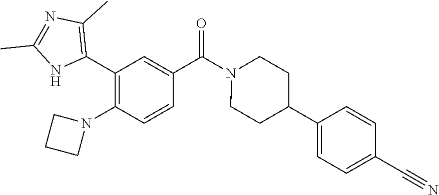 [SMILES: Cc1nc(C)c(-c2cc(C(=O)N3CCC(c4ccc(C#N)cc4)CC3)ccc2N2CCC2)[nH]1] | |
| 002-26 | 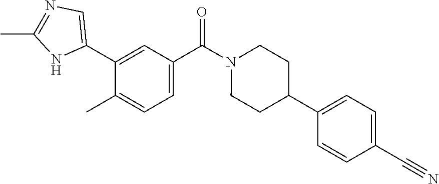 [SMILES: Cc1ncc(-c2cc(C(=O)N3CCC(c4ccc(C#N)cc4)CC3)ccc2C)[nH]1] | 0.245 |
| 002-27 | 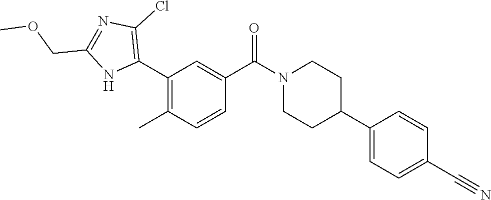 [SMILES: COCc1nc(Cl)c(-c2cc(C(=O)N3CCC(c4ccc(C#N)cc4)CC3)ccc2C)[nH]1] | 0.009 |
| 002-28 | 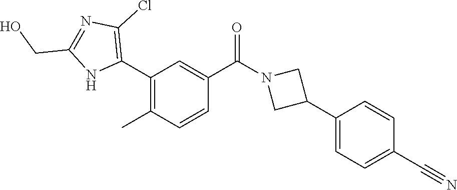 [SMILES: Cc1ccc(C(=O)N2CC(c3ccc(C#N)cc3)C2)cc1-c1[nH]c(CO)nc1Cl] | 0.048 |
| 002-29 | 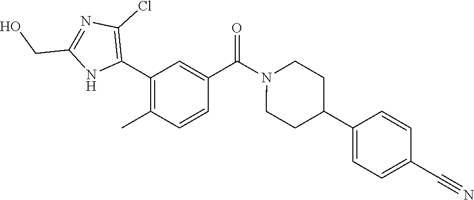 [SMILES: Cc1ccc(C(=O)N2CCC(c3ccc(C#N)cc3)CC2)cc1-c1[nH]c(CO)nc1Cl] | 0.024 |
| 002-30 | 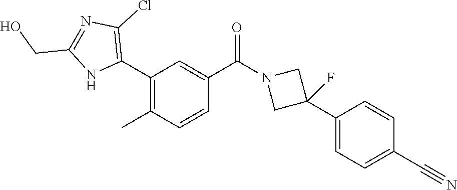 [SMILES: Cc1ccc(C(=O)N2CC(F)(c3ccc(C#N)cc3)C2)cc1-c1[nH]c(CO)nc1Cl] | 0.016 |
| 002-31 | 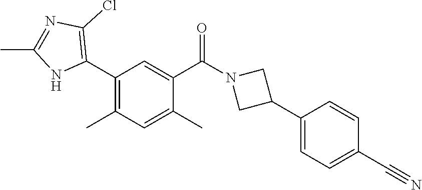 [SMILES: Cc1nc(Cl)c(-c2cc(C(=O)N3CC(c4ccc(C#N)cc4)C3)c(C)cc2C)[nH]1] | 0.011 |
| 002-32 | 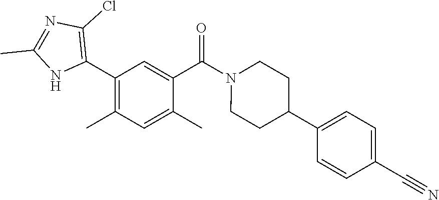 [SMILES: Cc1nc(Cl)c(-c2cc(C(=O)N3CCC(c4ccc(C#N)cc4)CC3)c(C)cc2C)[nH]1] | 0.012 |
| 002-33 | 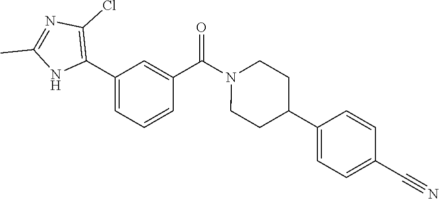 [SMILES: Cc1nc(Cl)c(-c2cccc(C(=O)N3CCC(c4ccc(C#N)cc4)CC3)c2)[nH]1] | 0.080 |
| 002-34 | 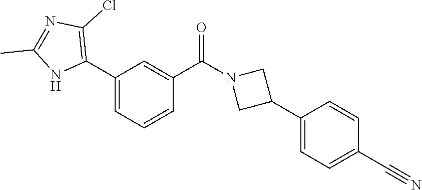 [SMILES: Cc1nc(Cl)c(-c2cccc(C(=O)N3CC(c4ccc(C#N)cc4)C3)c2)[nH]1] | 0.048 |
| 002-35 | 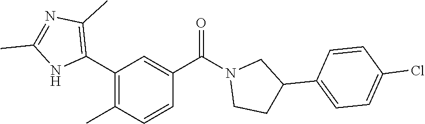 [SMILES: Cc1nc(C)c(-c2cc(C(=O)N3CCC(c4ccc(Cl)cc4)C3)ccc2C)[nH]1] | 2.870 |
| 002-36 | 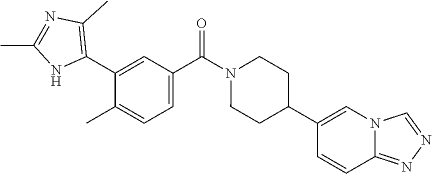 [SMILES: Cc1nc(C)c(-c2cc(C(=O)N3CCC(c4ccc5nncn5c4)CC3)ccc2C)[nH]1] | 9.190 |
| 002-37 | 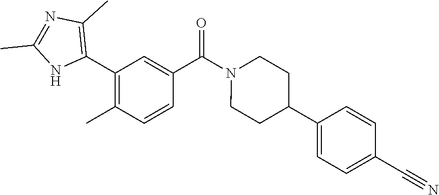 [SMILES: Cc1nc(C)c(-c2cc(C(=O)N3CCC(c4ccc(C#N)cc4)CC3)ccc2C)[nH]1] | 0.032 |
| 002-38 | 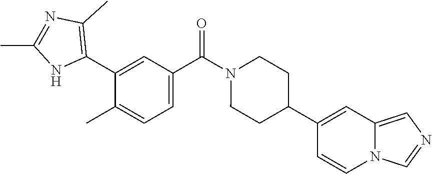 [SMILES: Cc1nc(C)c(-c2cc(C(=O)N3CCC(c4ccn5cncc5c4)CC3)ccc2C)[nH]1] | 0.104 |
| 002-39 | 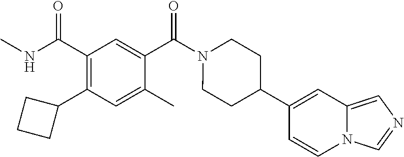 [SMILES: CNC(=O)c1cc(C(=O)N2CCC(c3ccn4cncc4c3)CC2)c(C)cc1C1CCC1] | 0.420 |
| 002-40 | 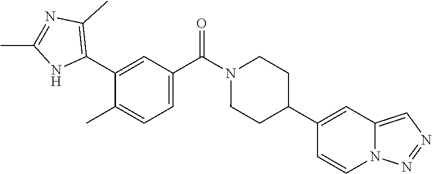 [SMILES: Cc1nc(C)c(-c2cc(C(=O)N3CCC(c4ccn5nncc5c4)CC3)ccc2C)[nH]1] | 0.620 |
| 002-41 | 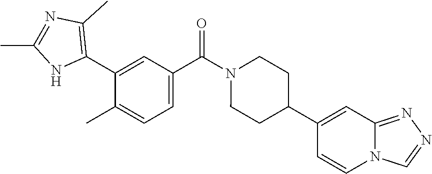 [SMILES: Cc1nc(C)c(-c2cc(C(=O)N3CCC(c4ccn5cnnc5c4)CC3)ccc2C)[nH]1] | 50.000 |
| 002-42 | 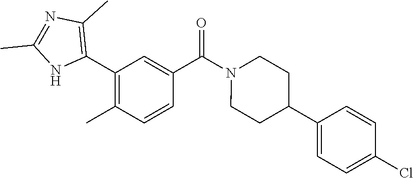 [SMILES: Cc1nc(C)c(-c2cc(C(=O)N3CCC(c4ccc(Cl)cc4)CC3)ccc2C)[nH]1] | 0.032 |
| 002-43 | 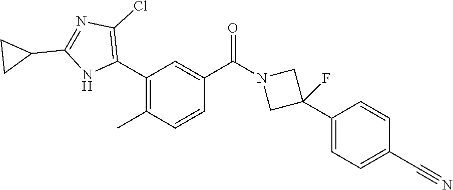 [SMILES: Cc1ccc(C(=O)N2CC(F)(c3ccc(C#N)cc3)C2)cc1-c1[nH]c(C2CC2)nc1Cl] | 0.012 |
| 002-44 | 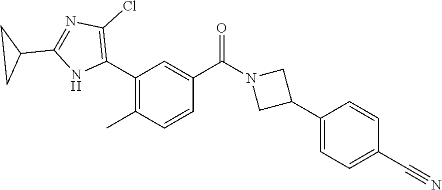 [SMILES: Cc1ccc(C(=O)N2CC(c3ccc(C#N)cc3)C2)cc1-c1[nH]c(C2CC2)nc1Cl] | 0.007 |
| 002-45 | 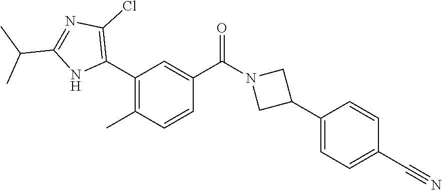 [SMILES: Cc1ccc(C(=O)N2CC(c3ccc(C#N)cc3)C2)cc1-c1[nH]c(C(C)C)nc1Cl] | 0.009 |
| 002-46 | 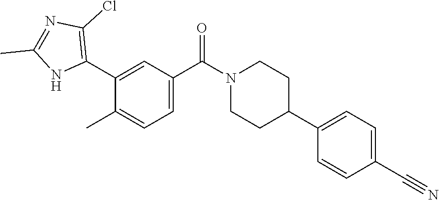 [SMILES: Cc1nc(Cl)c(-c2cc(C(=O)N3CCC(c4ccc(C#N)cc4)CC3)ccc2C)[nH]1] | 0.005 |
| 002-47 | 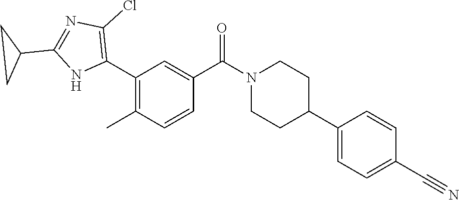 [SMILES: Cc1ccc(C(=O)N2CCC(c3ccc(C#N)cc3)CC2)cc1-c1[nH]c(C2CC2)nc1Cl] | 0.007 |
| 002-48 | 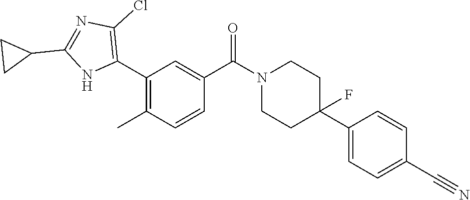 [SMILES: Cc1ccc(C(=O)N2CCC(F)(c3ccc(C#N)cc3)CC2)cc1-c1[nH]c(C2CC2)nc1Cl] | 0.009 |
| 002-49 | 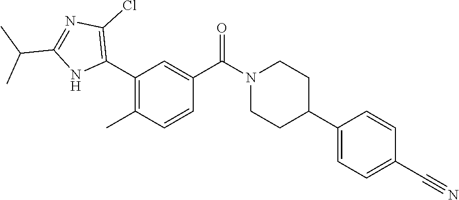 [SMILES: Cc1ccc(C(=O)N2CCC(c3ccc(C#N)cc3)CC2)cc1-c1[nH]c(C(C)C)nc1Cl] | 0.009 |
| 002-50 | 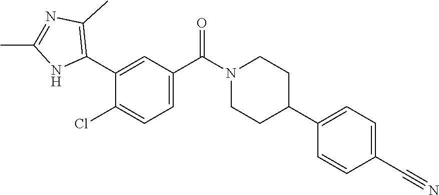 [SMILES: Cc1nc(C)c(-c2cc(C(=O)N3CCC(c4ccc(C#N)cc4)CC3)ccc2Cl)[nH]1] | 0.145 |
| 002-51 | 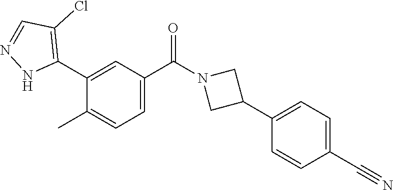 [SMILES: Cc1ccc(C(=O)N2CC(c3ccc(C#N)cc3)C2)cc1-c1[nH]ncc1Cl] | 0.019 |
| 002-52 | 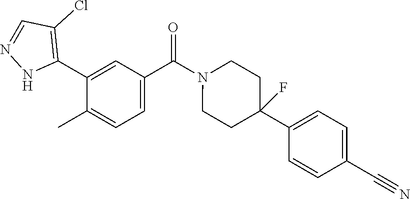 [SMILES: Cc1ccc(C(=O)N2CCC(F)(c3ccc(C#N)cc3)CC2)cc1-c1[nH]ncc1Cl] | 0.046 |
| 002-53 | 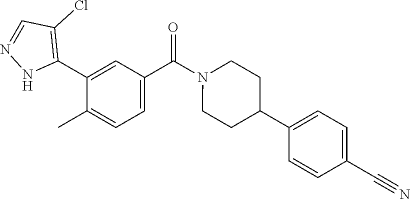 [SMILES: Cc1ccc(C(=O)N2CCC(c3ccc(C#N)cc3)CC2)cc1-c1[nH]ncc1Cl] | 0.033 |
| 002-54 | 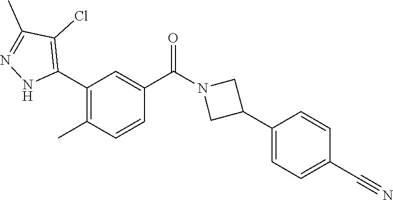 [SMILES: Cc1ccc(C(=O)N2CC(c3ccc(C#N)cc3)C2)cc1-c1[nH]nc(C)c1Cl] | 0.020 |
| 002-55 | 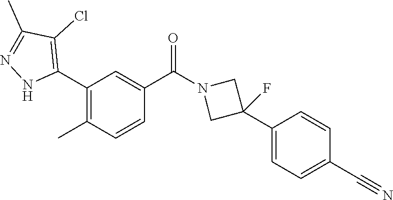 [SMILES: Cc1ccc(C(=O)N2CC(F)(c3ccc(C#N)cc3)C2)cc1-c1[nH]nc(C)c1Cl] | 0.086 |
| 002-56 | 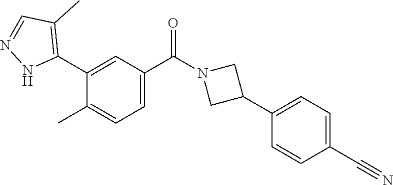 [SMILES: Cc1ccc(C(=O)N2CC(c3ccc(C#N)cc3)C2)cc1-c1[nH]ncc1C] | 0.021 |
| 002-57 | 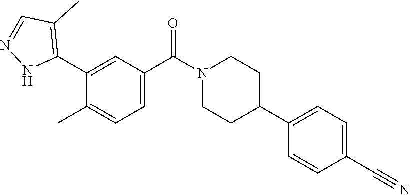 [SMILES: Cc1ccc(C(=O)N2CCC(c3ccc(C#N)cc3)CC2)cc1-c1[nH]ncc1C] | 0.097 |
| 002-58 | 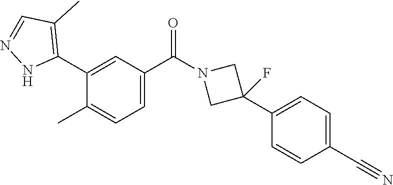 [SMILES: Cc1ccc(C(=O)N2CC(F)(c3ccc(C#N)cc3)C2)cc1-c1[nH]ncc1C] | 0.102 |
| 002-59 | 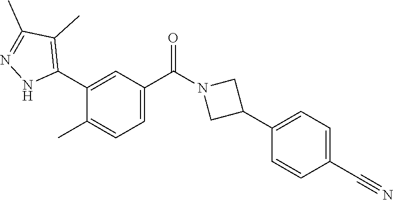 [SMILES: Cc1ccc(C(=O)N2CC(c3ccc(C#N)cc3)C2)cc1-c1[nH]nc(C)c1C] | 0.033 |
| 002-60 | 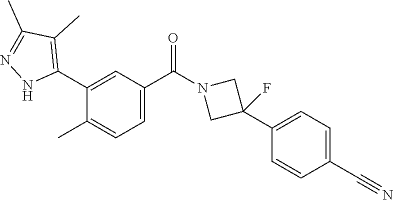 [SMILES: Cc1ccc(C(=O)N2CC(F)(c3ccc(C#N)cc3)C2)cc1-c1[nH]nc(C)c1C] | 0.110 |
| 002-61 | 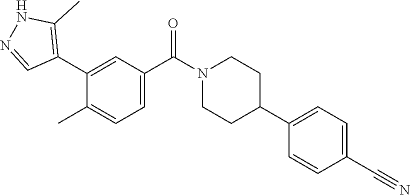 [SMILES: Cc1ccc(C(=O)N2CCC(c3ccc(C#N)cc3)CC2)cc1-c1cn[nH]c1C] | 0.310 |
| 002-62 | 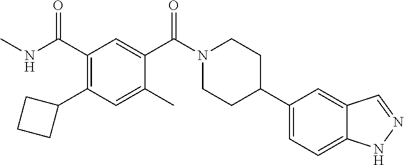 [SMILES: CNC(=O)c1cc(C(=O)N2CCC(c3ccc4[nH]ncc4c3)CC2)c(C)cc1C1CCC1] | 0.650 |
| 002-63 | 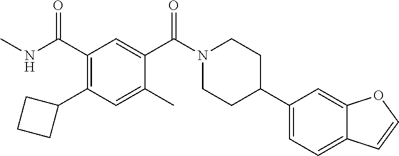 [SMILES: CNC(=O)c1cc(C(=O)N2CCC(c3ccc4ccoc4c3)CC2)c(C)cc1C1CCC1] | 1.180 |
| 002-64 | 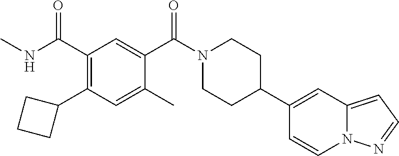 [SMILES: CNC(=O)c1cc(C(=O)N2CCC(c3ccn4nccc4c3)CC2)c(C)cc1C1CCC1] | 1.550 |
| 002-65 | 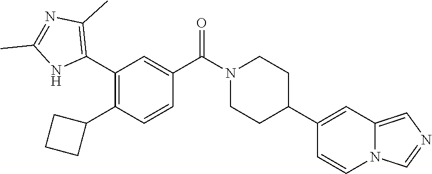 [SMILES: Cc1nc(C)c(-c2cc(C(=O)N3CCC(c4ccn5cncc5c4)CC3)ccc2C2CCC2)[nH]1] | 0.050 |
| 002-66 | 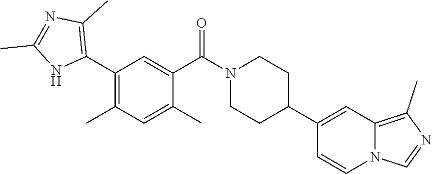 [SMILES: Cc1nc(C)c(-c2cc(C(=O)N3CCC(c4ccn5cnc(C)c5c4)CC3)c(C)cc2C)[nH]1] | 2.370 |
| 002-67 | 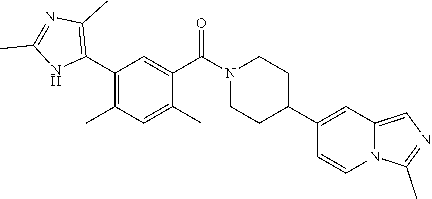 [SMILES: Cc1nc(C)c(-c2cc(C(=O)N3CCC(c4ccn5c(C)ncc5c4)CC3)c(C)cc2C)[nH]1] | 2.490 |
| 002-68 | 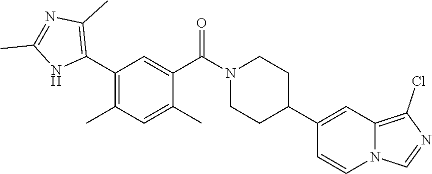 [SMILES: Cc1nc(C)c(-c2cc(C(=O)N3CCC(c4ccn5cnc(Cl)c5c4)CC3)c(C)cc2C)[nH]1] | 0.150 |
| 002-69 | 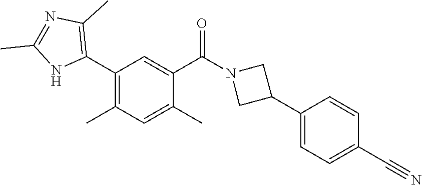 [SMILES: Cc1nc(C)c(-c2cc(C(=O)N3CC(c4ccc(C#N)cc4)C3)c(C)cc2C)[nH]1] | 0.018 |
| 002-70 | 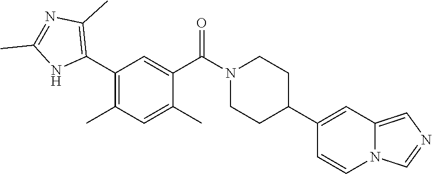 [SMILES: Cc1nc(C)c(-c2cc(C(=O)N3CCC(c4ccn5cncc5c4)CC3)c(C)cc2C)[nH]1] | 0.205 |
| 002-71 | 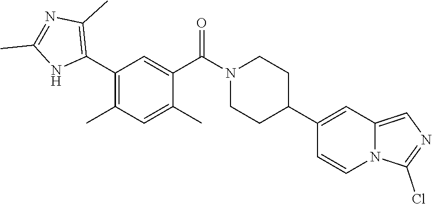 [SMILES: Cc1nc(C)c(-c2cc(C(=O)N3CCC(c4ccn5c(Cl)ncc5c4)CC3)c(C)cc2C)[nH]1] | 0.300 |
| 002-72 | 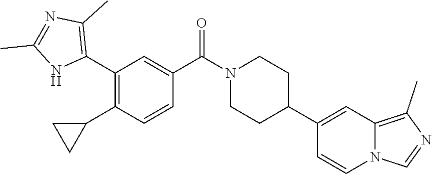 [SMILES: Cc1nc(C)c(-c2cc(C(=O)N3CCC(c4ccn5cnc(C)c5c4)CC3)ccc2C2CC2)[nH]1] | 0.420 |
| 002-73 | 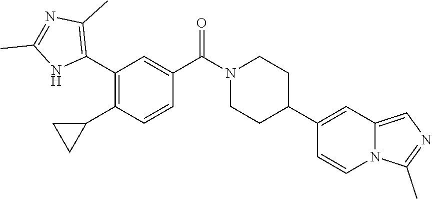 [SMILES: Cc1nc(C)c(-c2cc(C(=O)N3CCC(c4ccn5c(C)ncc5c4)CC3)ccc2C2CC2)[nH]1] | 1.000 |
| 002-74 | 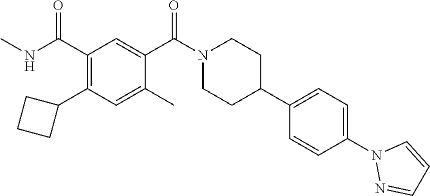 [SMILES: CNC(=O)c1cc(C(=O)N2CCC(c3ccc(-n4cccn4)cc3)CC2)c(C)cc1C1CCC1] | 0.460 |
| 002-75 | 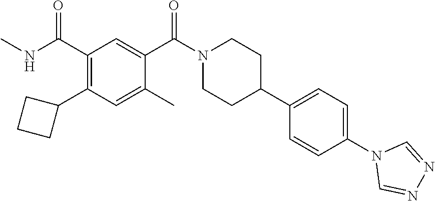 [SMILES: CNC(=O)c1cc(C(=O)N2CCC(c3ccc(-n4cnnc4)cc3)CC2)c(C)cc1C1CCC1] | 0.073 |
| 002-76 | 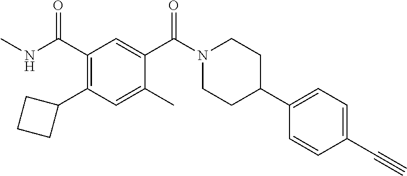 [SMILES: C#Cc1ccc(C2CCN(C(=O)c3cc(C(=O)NC)c(C4CCC4)cc3C)CC2)cc1] | 0.069 |
| 002-77 | 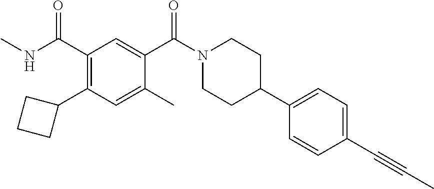 [SMILES: CC#Cc1ccc(C2CCN(C(=O)c3cc(C(=O)NC)c(C4CCC4)cc3C)CC2)cc1] | 0.125 |
| 002-78 | 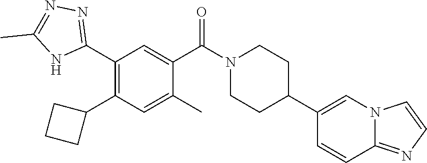 [SMILES: Cc1nnc(-c2cc(C(=O)N3CCC(c4ccc5nccn5c4)CC3)c(C)cc2C2CCC2)[nH]1] | 3.670 |
| 002-79 | 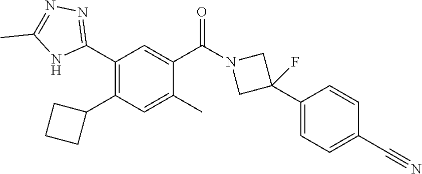 [SMILES: Cc1nnc(-c2cc(C(=O)N3CC(F)(c4ccc(C#N)cc4)C3)c(C)cc2C2CCC2)[nH]1] | 0.059 |
| 002-80 | 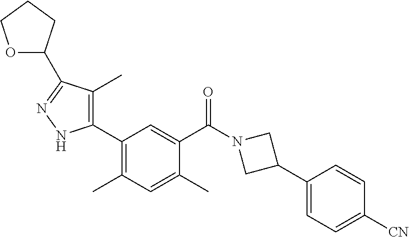 [SMILES: Cc1cc(C)c(-c2[nH]nc(C3CCCO3)c2C)cc1C(=O)N1CC(c2ccc(C#N)cc2)C1] | 0.057 |
| 002-81 | 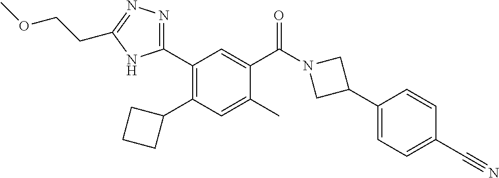 [SMILES: COCCc1nnc(-c2cc(C(=O)N3CC(c4ccc(C#N)cc4)C3)c(C)cc2C2CCC2)[nH]1] | 0.054 |
| 002-82 | 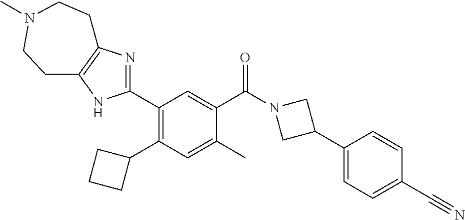 [SMILES: Cc1cc(C2CCC2)c(-c2nc3c([nH]2)CCN(C)CC3)cc1C(=O)N1CC(c2ccc(C#N)cc2)C1] | 0.020 |
| 002-83 | 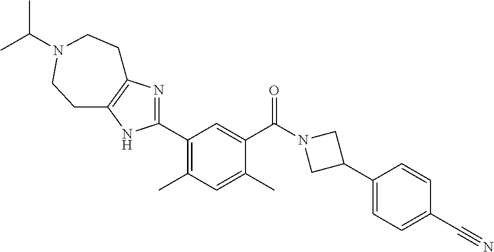 [SMILES: Cc1cc(C)c(-c2nc3c([nH]2)CCN(C(C)C)CC3)cc1C(=O)N1CC(c2ccc(C#N)cc2)C1] | 0.062 |
| 002-84 | 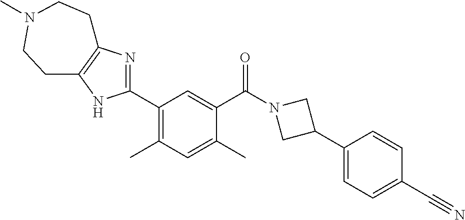 [SMILES: Cc1cc(C)c(-c2nc3c([nH]2)CCN(C)CC3)cc1C(=O)N1CC(c2ccc(C#N)cc2)C1] | 0.112 |
| 002-85 | 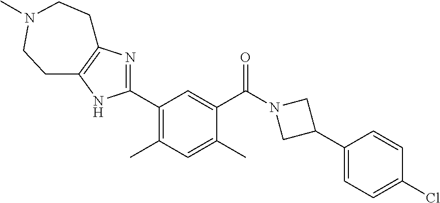 [SMILES: Cc1cc(C)c(-c2nc3c([nH]2)CCN(C)CC3)cc1C(=O)N1CC(c2ccc(Cl)cc2)C1] | 0.057 |
| 002-86 | 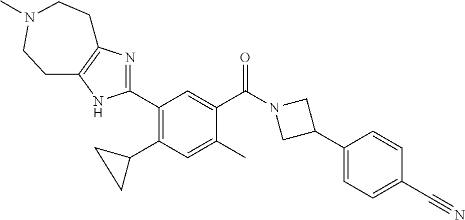 [SMILES: Cc1cc(C2CC2)c(-c2nc3c([nH]2)CCN(C)CC3)cc1C(=O)N1CC(c2ccc(C#N)cc2)C1] | 0.086 |
| 002-87 | 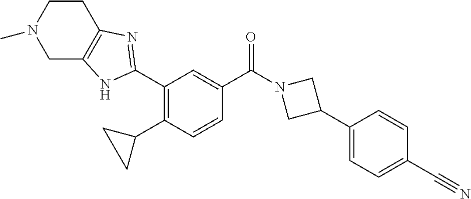 [SMILES: CN1CCc2nc(-c3cc(C(=O)N4CC(c5ccc(C#N)cc5)C4)ccc3C3CC3)[nH]c2C1] | 0.040 |
| 002-88 | 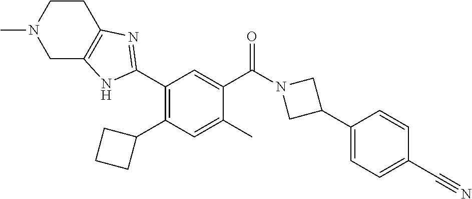 [SMILES: Cc1cc(C2CCC2)c(-c2nc3c([nH]2)CN(C)CC3)cc1C(=O)N1CC(c2ccc(C#N)cc2)C1] | 0.021 |
| 002-89 | 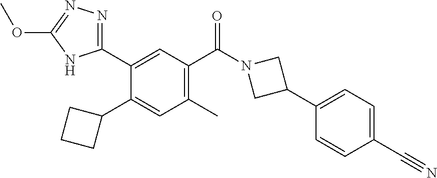 [SMILES: COc1nnc(-c2cc(C(=O)N3CC(c4ccc(C#N)cc4)C3)c(C)cc2C2CCC2)[nH]1] | 0.049 |
| 002-90 | 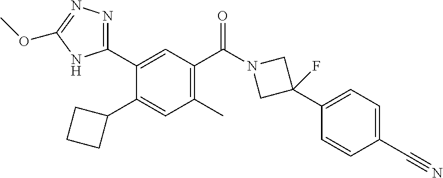 [SMILES: COc1nnc(-c2cc(C(=O)N3CC(F)(c4ccc(C#N)cc4)C3)c(C)cc2C2CCC2)[nH]1] | 0.071 |
| 002-91 | 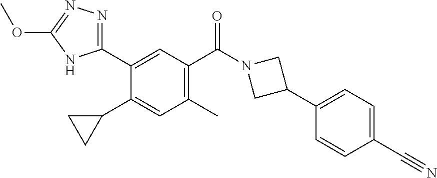 [SMILES: COc1nnc(-c2cc(C(=O)N3CC(c4ccc(C#N)cc4)C3)c(C)cc2C2CC2)[nH]1] | 0.140 |
| 002-92 | 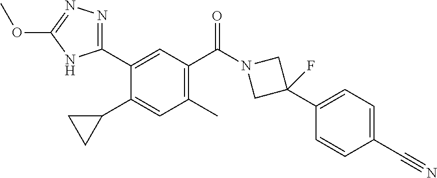 [SMILES: COc1nnc(-c2cc(C(=O)N3CC(F)(c4ccc(C#N)cc4)C3)c(C)cc2C2CC2)[nH]1] | 0.140 |
| 002-93 | 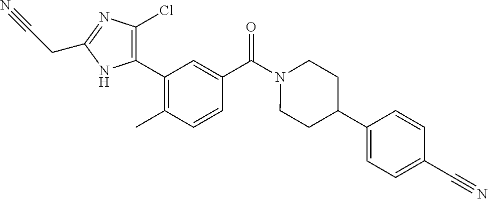 [SMILES: Cc1ccc(C(=O)N2CCC(c3ccc(C#N)cc3)CC2)cc1-c1[nH]c(CC#N)nc1Cl] | 0.034 |
| 002-94 | 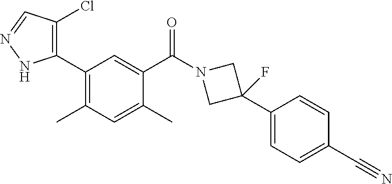 [SMILES: Cc1cc(C)c(-c2[nH]ncc2Cl)cc1C(=O)N1CC(F)(c2ccc(C#N)cc2)C1] | 0.029 |
| 002-95 | 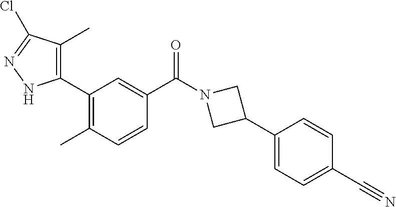 [SMILES: Cc1ccc(C(=O)N2CC(c3ccc(C#N)cc3)C2)cc1-c1[nH]nc(Cl)c1C] | 0.030 |
| 002-96 | 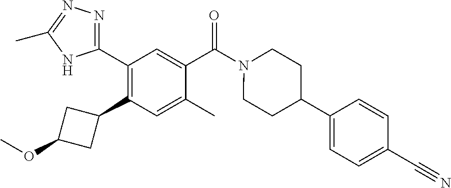 [SMILES: CO[C@H]1C[C@@H](c2cc(C)c(C(=O)N3CCC(c4ccc(C#N)cc4)CC3)cc2-c2nnc(C)[nH]2)C1] | 0.270 |
| 002-97 | 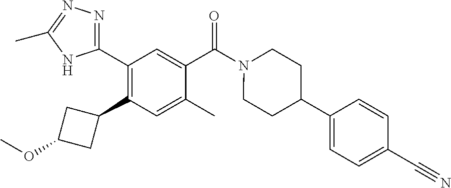 [SMILES: CO[C@H]1C[C@H](c2cc(C)c(C(=O)N3CCC(c4ccc(C#N)cc4)CC3)cc2-c2nnc(C)[nH]2)C1] | 0.190 |
| 002-98 | 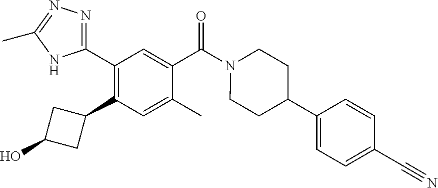 [SMILES: Cc1nnc(-c2cc(C(=O)N3CCC(c4ccc(C#N)cc4)CC3)c(C)cc2[C@H]2C[C@@H](O)C2)[nH]1] | 0.240 |
| 002-99 | 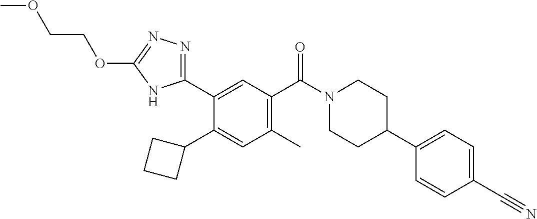 [SMILES: COCCOc1nnc(-c2cc(C(=O)N3CCC(c4ccc(C#N)cc4)CC3)c(C)cc2C2CCC2)[nH]1] | 0.045 |
| 002-100 | 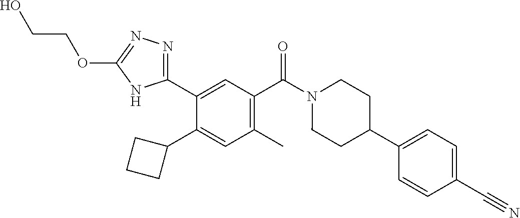 [SMILES: Cc1cc(C2CCC2)c(-c2nnc(OCCO)[nH]2)cc1C(=O)N1CCC(c2ccc(C#N)cc2)CC1] | 0.055 |
| 002-101 | 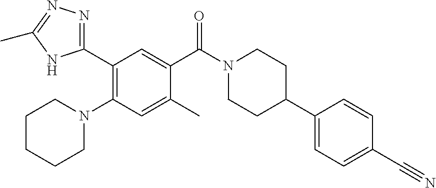 [SMILES: Cc1nnc(-c2cc(C(=O)N3CCC(c4ccc(C#N)cc4)CC3)c(C)cc2N2CCCCC2)[nH]1] | 0.530 |
| 002-102 | 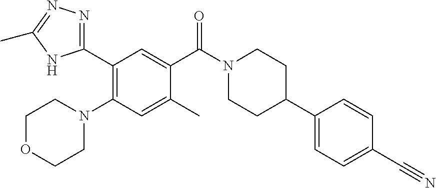 [SMILES: Cc1nnc(-c2cc(C(=O)N3CCC(c4ccc(C#N)cc4)CC3)c(C)cc2N2CCOCC2)[nH]1] | 0.890 |
| 002-103 | 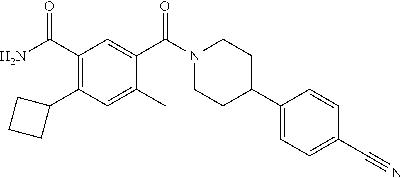 [SMILES: Cc1cc(C2CCC2)c(C(N)=O)cc1C(=O)N1CCC(c2ccc(C#N)cc2)CC1] | 0.135 |
| 002-104 | 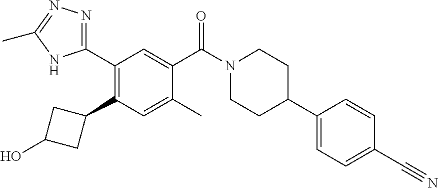 | 0.070 |
| 002-105 | 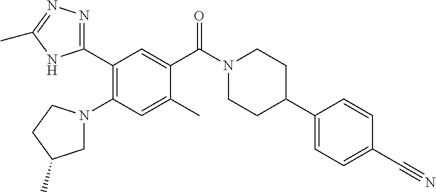 [SMILES: Cc1nnc(-c2cc(C(=O)N3CCC(c4ccc(C#N)cc4)CC3)c(C)cc2N2CC[C@@H](C)C2)[nH]1] | 0.070 |
| 002-106 | 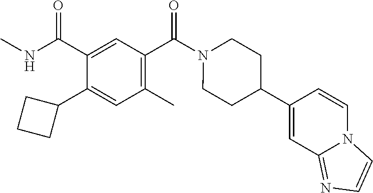 [SMILES: CNC(=O)c1cc(C(=O)N2CCC(c3ccn4ccnc4c3)CC2)c(C)cc1C1CCC1] | 50.000 |
| 002-107 | 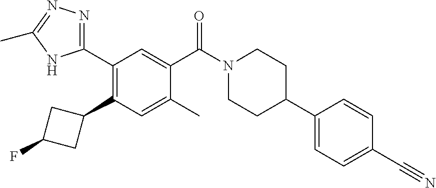 [SMILES: Cc1nnc(-c2cc(C(=O)N3CCC(c4ccc(C#N)cc4)CC3)c(C)cc2[C@H]2C[C@@H](F)C2)[nH]1] | 5.580 |
| 002-108 | 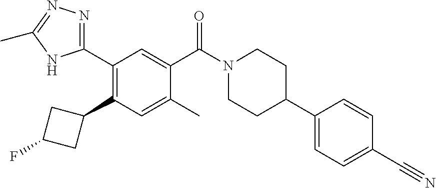 [SMILES: Cc1nnc(-c2cc(C(=O)N3CCC(c4ccc(C#N)cc4)CC3)c(C)cc2[C@H]2C[C@H](F)C2)[nH]1] | 2.250 |
| 002-109 | 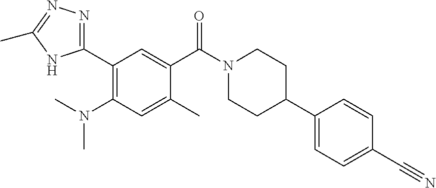 [SMILES: Cc1nnc(-c2cc(C(=O)N3CCC(c4ccc(C#N)cc4)CC3)c(C)cc2N(C)C)[nH]1] | 0.280 |
| 002-110 | 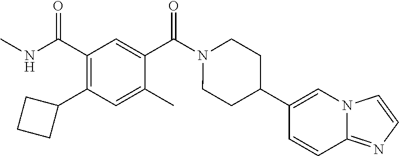 [SMILES: CNC(=O)c1cc(C(=O)N2CCC(c3ccc4nccn4c3)CC2)c(C)cc1C1CCC1] | 11.020 |
| 002-111 | 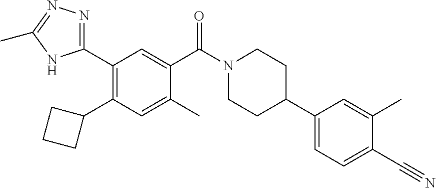 [SMILES: Cc1nnc(-c2cc(C(=O)N3CCC(c4ccc(C#N)c(C)c4)CC3)c(C)cc2C2CCC2)[nH]1] | 0.230 |
| 002-112 | 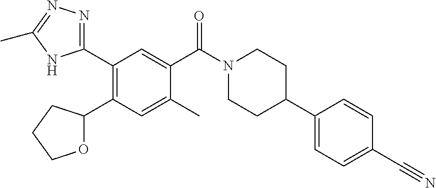 [SMILES: Cc1nnc(-c2cc(C(=O)N3CCC(c4ccc(C#N)cc4)CC3)c(C)cc2C2CCCO2)[nH]1] | 0.840 |
| 002-113 | 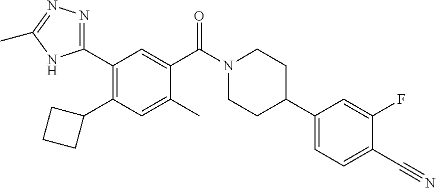 [SMILES: Cc1nnc(-c2cc(C(=O)N3CCC(c4ccc(C#N)c(F)c4)CC3)c(C)cc2C2CCC2)[nH]1] | 0.145 |
| 002-114 | 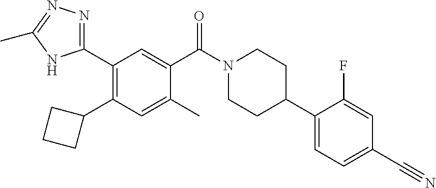 [SMILES: Cc1nnc(-c2cc(C(=O)N3CCC(c4ccc(C#N)cc4F)CC3)c(C)cc2C2CCC2)[nH]1] | 0.200 |
| 002-115 | 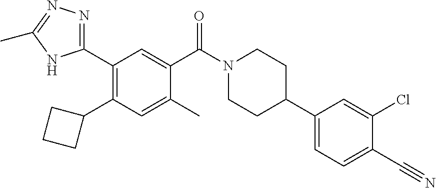 [SMILES: Cc1nnc(-c2cc(C(=O)N3CCC(c4ccc(C#N)c(Cl)c4)CC3)c(C)cc2C2CCC2)[nH]1] | 0.320 |
| 002-116 | 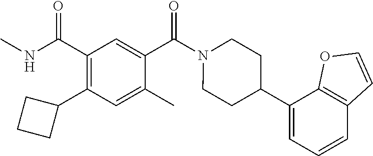 [SMILES: CNC(=O)c1cc(C(=O)N2CCC(c3cccc4ccoc34)CC2)c(C)cc1C1CCC1] | 50.000 |
| 002-117 | 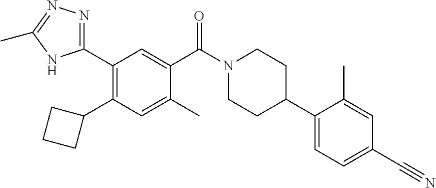 [SMILES: Cc1nnc(-c2cc(C(=O)N3CCC(c4ccc(C#N)cc4C)CC3)c(C)cc2C2CCC2)[nH]1] | 0.980 |
| 002-118 | 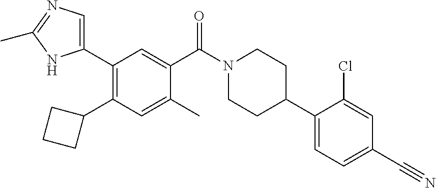 [SMILES: Cc1ncc(-c2cc(C(=O)N3CCC(c4ccc(C#N)cc4Cl)CC3)c(C)cc2C2CCC2)[nH]1] | 0.680 |
| 002-119 | 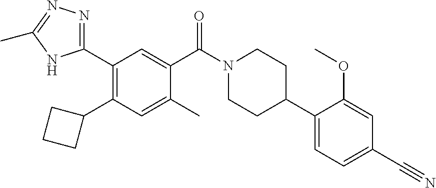 [SMILES: COc1cc(C#N)ccc1C1CCN(C(=O)c2cc(-c3nnc(C)[nH]3)c(C3CCC3)cc2C)CC1] | 4.480 |
| 002-120 | 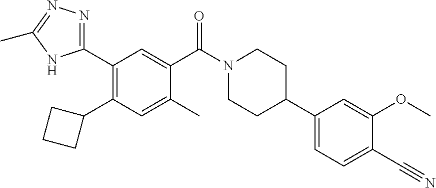 | 1.300 |
| 002-121 | 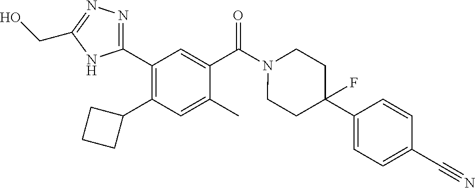 [SMILES: Cc1cc(C2CCC2)c(-c2nnc(CO)[nH]2)cc1C(=O)N1CCC(F)(c2ccc(C#N)cc2)CC1] | 0.030 |
| 002-122 | 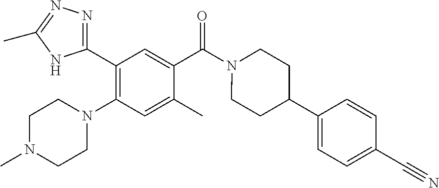 [SMILES: Cc1nnc(-c2cc(C(=O)N3CCC(c4ccc(C#N)cc4)CC3)c(C)cc2N2CCN(C)CC2)[nH]1] | 3.440 |
| 002-123 | 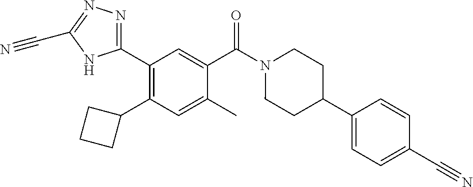 [SMILES: Cc1cc(C2CCC2)c(-c2nnc(C#N)[nH]2)cc1C(=O)N1CCC(c2ccc(C#N)cc2)CC1] | 3.710 |
| 002-124 | 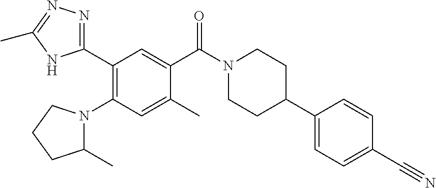 [SMILES: Cc1nnc(-c2cc(C(=O)N3CCC(c4ccc(C#N)cc4)CC3)c(C)cc2N2CCCC2C)[nH]1] | 0.140 |
| 002-125 | 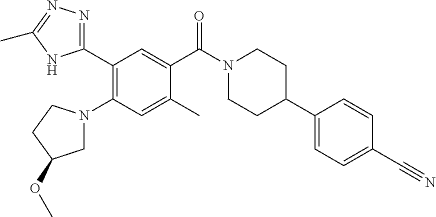 [SMILES: CO[C@H]1CCN(c2cc(C)c(C(=O)N3CCC(c4ccc(C#N)cc4)CC3)cc2-c2nnc(C)[nH]2)C1] | 0.110 |
| 002-126 | 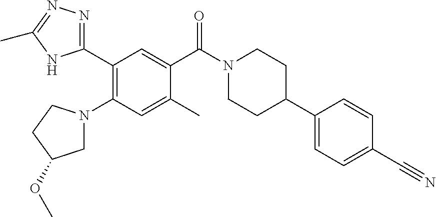 [SMILES: CO[C@@H]1CCN(c2cc(C)c(C(=O)N3CCC(c4ccc(C#N)cc4)CC3)cc2-c2nnc(C)[nH]2)C1] | 1.130 |
| 002-127 | 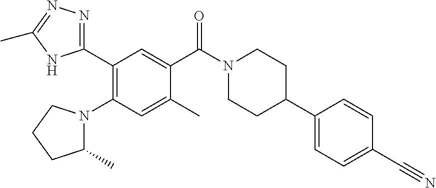 [SMILES: Cc1nnc(-c2cc(C(=O)N3CCC(c4ccc(C#N)cc4)CC3)c(C)cc2N2CCC[C@H]2C)[nH]1] | 3.270 |
| 002-128 | 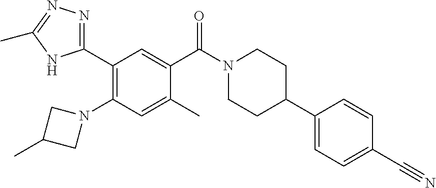 [SMILES: Cc1nnc(-c2cc(C(=O)N3CCC(c4ccc(C#N)cc4)CC3)c(C)cc2N2CC(C)C2)[nH]1] | 0.098 |
| 002-129 | 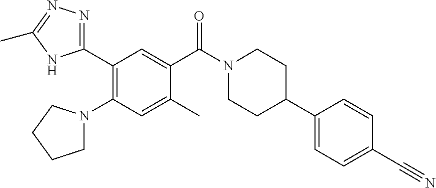 [SMILES: Cc1nnc(-c2cc(C(=O)N3CCC(c4ccc(C#N)cc4)CC3)c(C)cc2N2CCCC2)[nH]1] | 1.210 |
| 002-130 | 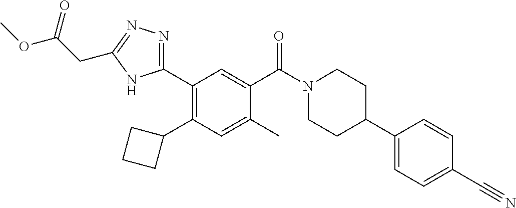 [SMILES: COC(=O)Cc1nnc(-c2cc(C(=O)N3CCC(c4ccc(C#N)cc4)CC3)c(C)cc2C2CCC2)[nH]1] | 0.082 |
| 002-131 | 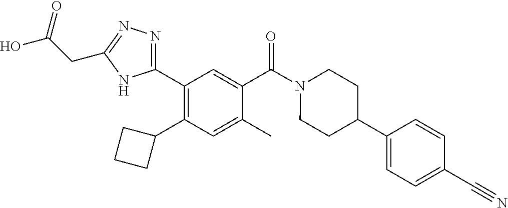 [SMILES: Cc1cc(C2CCC2)c(-c2nnc(CC(=O)O)[nH]2)cc1C(=O)N1CCC(c2ccc(C#N)cc2)CC1] | 0.185 |
| 002-132 | 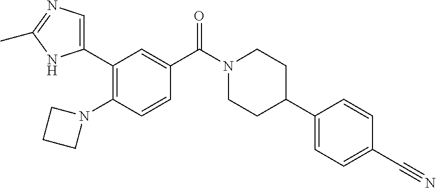 [SMILES: Cc1ncc(-c2cc(C(=O)N3CCC(c4ccc(C#N)cc4)CC3)ccc2N2CCC2)[nH]1] | 0.210 |
| 002-133 | 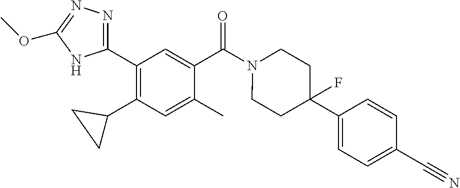 [SMILES: COc1nnc(-c2cc(C(=O)N3CCC(F)(c4ccc(C#N)cc4)CC3)c(C)cc2C2CC2)[nH]1] | 0.065 |
| 002-134 | 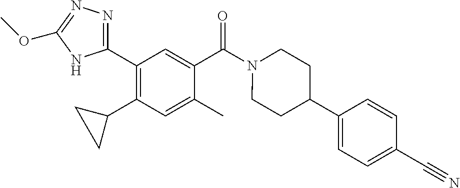 [SMILES: COc1nnc(-c2cc(C(=O)N3CCC(c4ccc(C#N)cc4)CC3)c(C)cc2C2CC2)[nH]1] | 0.099 |
| 002-135 | 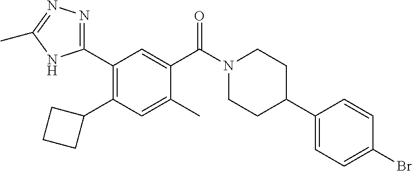 [SMILES: Cc1nnc(-c2cc(C(=O)N3CCC(c4ccc(Br)cc4)CC3)c(C)cc2C2CCC2)[nH]1] | |
| 002-136 | 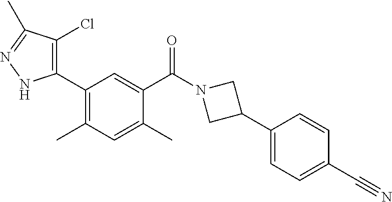 [SMILES: Cc1cc(C)c(-c2[nH]nc(C)c2Cl)cc1C(=O)N1CC(c2ccc(C#N)cc2)C1] | 0.026 |
| 002-137 | 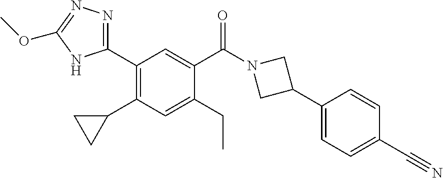 [SMILES: CCc1cc(C2CC2)c(-c2nnc(OC)[nH]2)cc1C(=O)N1CC(c2ccc(C#N)cc2)C1] | 0.105 |
| 002-138 | 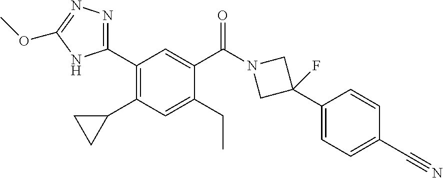 [SMILES: CCc1cc(C2CC2)c(-c2nnc(OC)[nH]2)cc1C(=O)N1CC(F)(c2ccc(C#N)cc2)C1] | 0.120 |
| 002-139 | 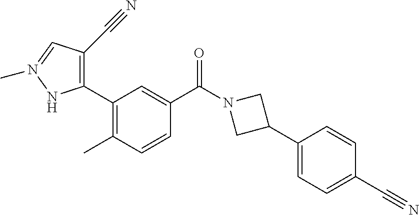 | 0.017 |
| 002-140 | 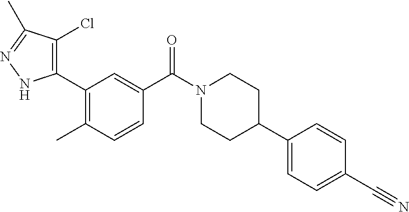 [SMILES: Cc1ccc(C(=O)N2CCC(c3ccc(C#N)cc3)CC2)cc1-c1[nH]nc(C)c1Cl] | 0.080 |
| 002-141 | 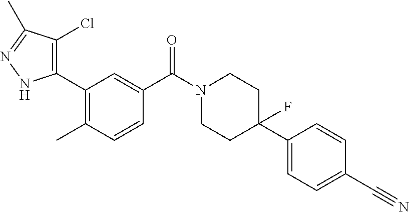 [SMILES: Cc1ccc(C(=O)N2CCC(F)(c3ccc(C#N)cc3)CC2)cc1-c1[nH]nc(C)c1Cl] | 0.099 |
| 002-142 | 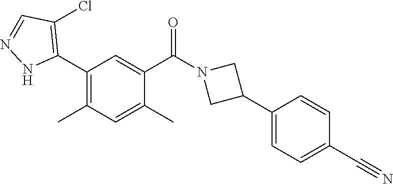 [SMILES: Cc1cc(C)c(-c2[nH]ncc2Cl)cc1C(=O)N1CC(c2ccc(C#N)cc2)C1] | 0.024 |
| 002-143 | 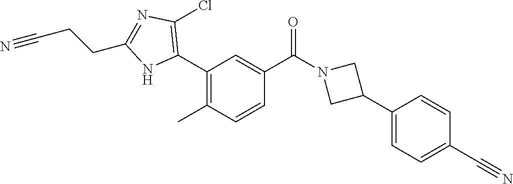 [SMILES: Cc1ccc(C(=O)N2CC(c3ccc(C#N)cc3)C2)cc1-c1[nH]c(CCC#N)nc1Cl] | 0.018 |
| 002-144 | 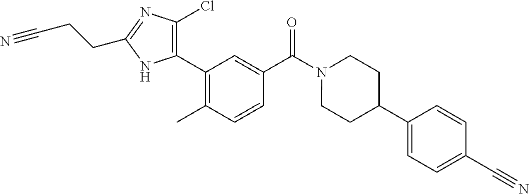 [SMILES: Cc1ccc(C(=O)N2CCC(c3ccc(C#N)cc3)CC2)cc1-c1[nH]c(CCC#N)nc1Cl] | 0.020 |
| 002-145 | 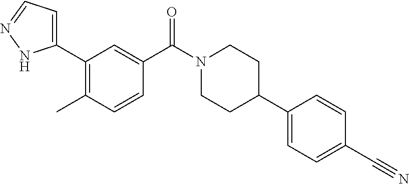 [SMILES: Cc1ccc(C(=O)N2CCC(c3ccc(C#N)cc3)CC2)cc1-c1ccn[nH]1] | 2.170 |
| 002-146 | 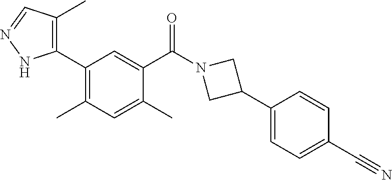 [SMILES: Cc1cc(C)c(-c2[nH]ncc2C)cc1C(=O)N1CC(c2ccc(C#N)cc2)C1] | 0.062 |
| 002-147 | 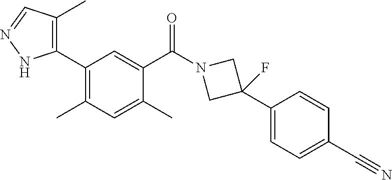 [SMILES: Cc1cc(C)c(-c2[nH]ncc2C)cc1C(=O)N1CC(F)(c2ccc(C#N)cc2)C1] | 0.120 |
| 002-148 | 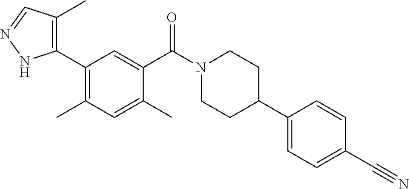 [SMILES: Cc1cc(C)c(-c2[nH]ncc2C)cc1C(=O)N1CCC(c2ccc(C#N)cc2)CC1] | 0.084 |
| 002-149 | 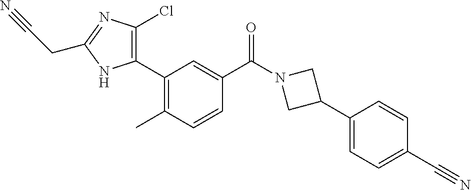 [SMILES: Cc1ccc(C(=O)N2CC(c3ccc(C#N)cc3)C2)cc1-c1[nH]c(CC#N)nc1Cl] | 0.021 |
| 002-150 | 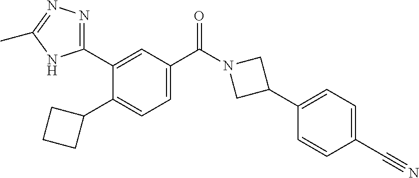 [SMILES: Cc1nnc(-c2cc(C(=O)N3CC(c4ccc(C#N)cc4)C3)ccc2C2CCC2)[nH]1] | 0.097 |
| 002-151 | 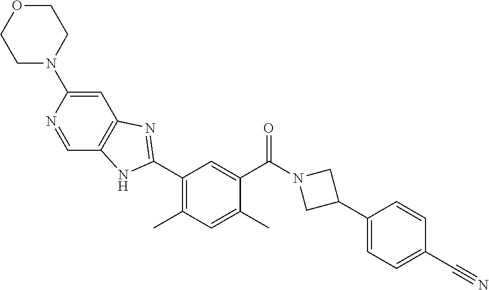 [SMILES: Cc1cc(C)c(-c2nc3cc(N4CCOCC4)ncc3[nH]2)cc1C(=O)N1CC(c2ccc(C#N)cc2)C1] | 0.057 |
| 002-152 | 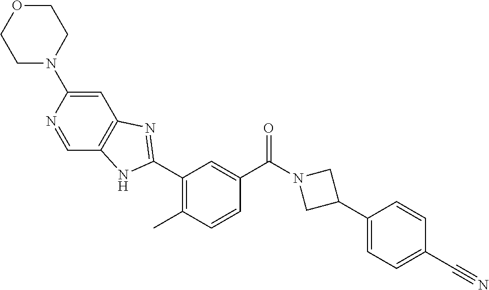 [SMILES: Cc1ccc(C(=O)N2CC(c3ccc(C#N)cc3)C2)cc1-c1nc2cc(N3CCOCC3)ncc2[nH]1] | 0.290 |
| 002-153 | 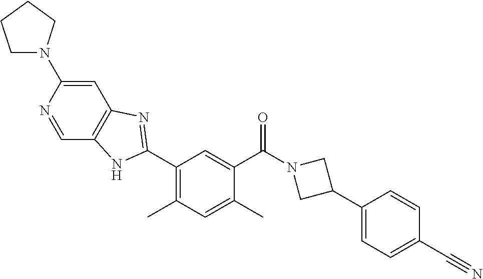 [SMILES: Cc1cc(C)c(-c2nc3cc(N4CCCC4)ncc3[nH]2)cc1C(=O)N1CC(c2ccc(C#N)cc2)C1] | 0.036 |
| 002-154 | 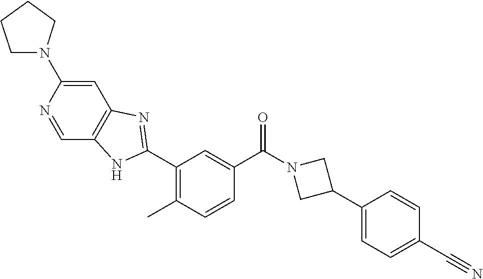 [SMILES: Cc1ccc(C(=O)N2CC(c3ccc(C#N)cc3)C2)cc1-c1nc2cc(N3CCCC3)ncc2[nH]1] | 0.094 |
| 002-155 | 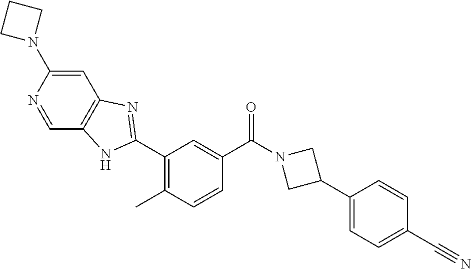 [SMILES: Cc1ccc(C(=O)N2CC(c3ccc(C#N)cc3)C2)cc1-c1nc2cc(N3CCC3)ncc2[nH]1] | 0.590 |
| 002-156 | 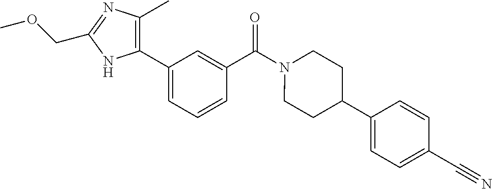 [SMILES: COCc1nc(C)c(-c2cccc(C(=O)N3CCC(c4ccc(C#N)cc4)CC3)c2)[nH]1] | 1.495 |
| 002-157 | 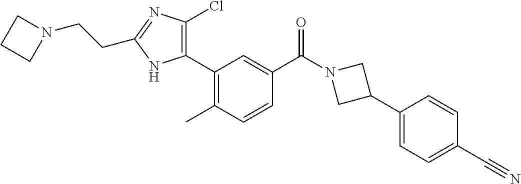 [SMILES: Cc1ccc(C(=O)N2CC(c3ccc(C#N)cc3)C2)cc1-c1[nH]c(CCN2CCC2)nc1Cl] | 0.210 |
| 002-158 | 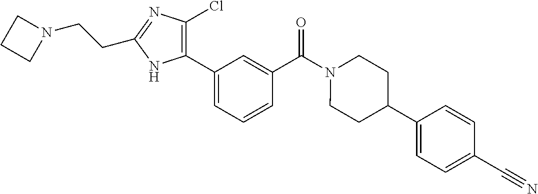 [SMILES: N#Cc1ccc(C2CCN(C(=O)c3cccc(-c4[nH]c(CCN5CCC5)nc4Cl)c3)CC2)cc1] | |
| 002-159 | 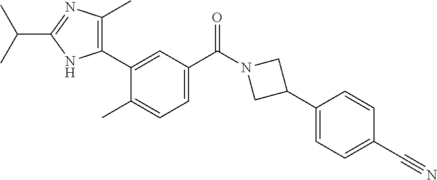 [SMILES: Cc1ccc(C(=O)N2CC(c3ccc(C#N)cc3)C2)cc1-c1[nH]c(C(C)C)nc1C] | 0.021 |
| 002-160 | 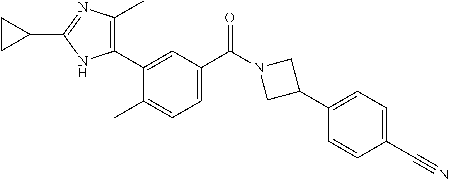 [SMILES: Cc1ccc(C(=O)N2CC(c3ccc(C#N)cc3)C2)cc1-c1[nH]c(C2CC2)nc1C] | 0.017 |
| 002-161 | 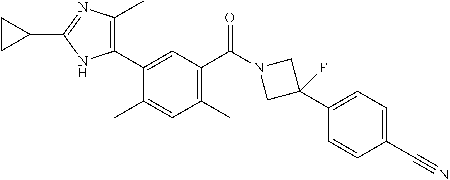 [SMILES: Cc1cc(C)c(-c2[nH]c(C3CC3)nc2C)cc1C(=O)N1CC(F)(c2ccc(C#N)cc2)C1] | 0.026 |
| 002-162 | 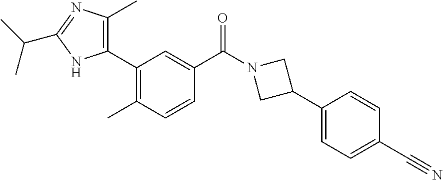 [SMILES: Cc1ccc(C(=O)N2CC(c3ccc(C#N)cc3)C2)cc1-c1[nH]c(C(C)C)nc1C] | 0.019 |
| 002-163 | 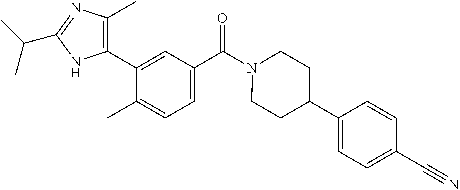 [SMILES: Cc1ccc(C(=O)N2CCC(c3ccc(C#N)cc3)CC2)cc1-c1[nH]c(C(C)C)nc1C] | 0.037 |
| 002-164 | 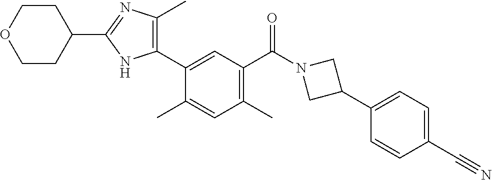 [SMILES: Cc1cc(C)c(-c2[nH]c(C3CCOCC3)nc2C)cc1C(=O)N1CC(c2ccc(C#N)cc2)C1] | 0.019 |
| 002-165 | 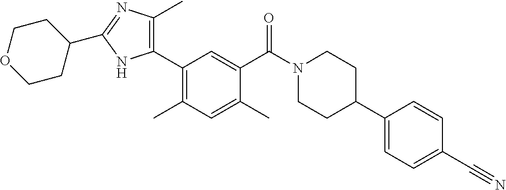 [SMILES: Cc1cc(C)c(-c2[nH]c(C3CCOCC3)nc2C)cc1C(=O)N1CCC(c2ccc(C#N)cc2)CC1] | 0.038 |
| 002-166 | 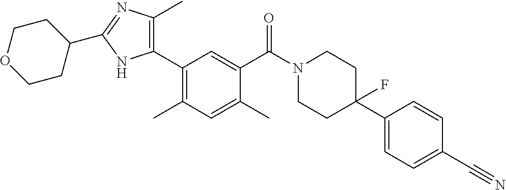 [SMILES: Cc1cc(C)c(-c2[nH]c(C3CCOCC3)nc2C)cc1C(=O)N1CCC(F)(c2ccc(C#N)cc2)CC1] | 0.040 |
| 002-167 | 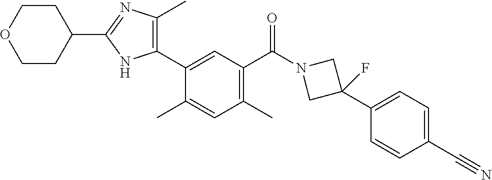 [SMILES: Cc1cc(C)c(-c2[nH]c(C3CCOCC3)nc2C)cc1C(=O)N1CC(F)(c2ccc(C#N)cc2)C1] | 0.061 |
| 002-168 | 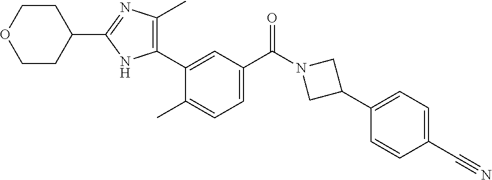 [SMILES: Cc1ccc(C(=O)N2CC(c3ccc(C#N)cc3)C2)cc1-c1[nH]c(C2CCOCC2)nc1C] | 0.011 |
| 002-169 | 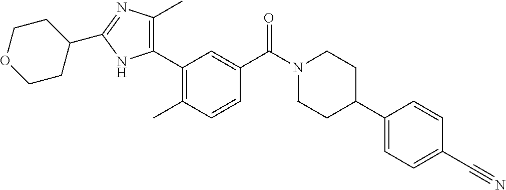 [SMILES: Cc1ccc(C(=O)N2CCC(c3ccc(C#N)cc3)CC2)cc1-c1[nH]c(C2CCOCC2)nc1C] | 0.035 |
| 002-170 | 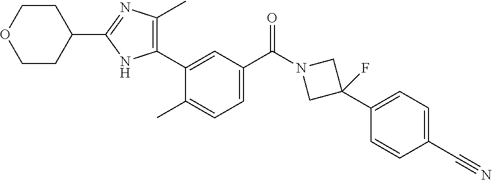 [SMILES: Cc1ccc(C(=O)N2CC(F)(c3ccc(C#N)cc3)C2)cc1-c1[nH]c(C2CCOCC2)nc1C] | 0.040 |
| 002-171 | 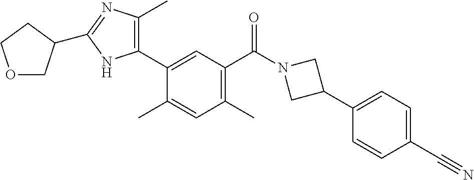 [SMILES: Cc1cc(C)c(-c2[nH]c(C3CCOC3)nc2C)cc1C(=O)N1CC(c2ccc(C#N)cc2)C1] | 0.021 |
| 002-172 | 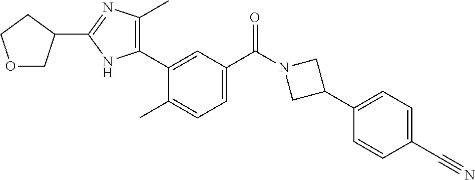 [SMILES: Cc1ccc(C(=O)N2CC(c3ccc(C#N)cc3)C2)cc1-c1[nH]c(C2CCOC2)nc1C] | 0.011 |
| 002-173 | 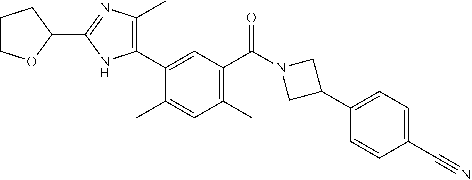 [SMILES: Cc1cc(C)c(-c2[nH]c(C3CCCO3)nc2C)cc1C(=O)N1CC(c2ccc(C#N)cc2)C1] | 0.035 |
| 002-174 | 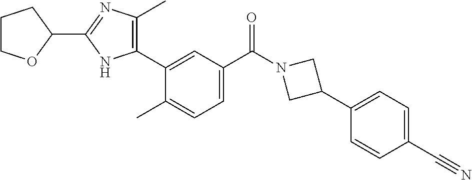 [SMILES: Cc1ccc(C(=O)N2CC(c3ccc(C#N)cc3)C2)cc1-c1[nH]c(C2CCCO2)nc1C] | 0.037 |
| 002-175 | 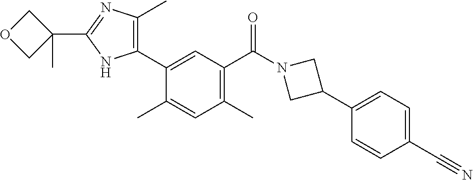 [SMILES: Cc1cc(C)c(-c2[nH]c(C3(C)COC3)nc2C)cc1C(=O)N1CC(c2ccc(C#N)cc2)C1] | 0.020 |
| 002-176 | 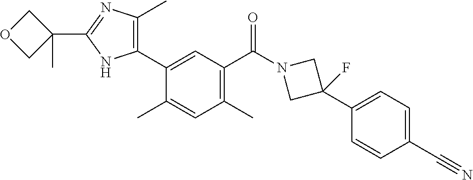 [SMILES: Cc1cc(C)c(-c2[nH]c(C3(C)COC3)nc2C)cc1C(=O)N1CC(F)(c2ccc(C#N)cc2)C1] | 0.024 |
| 002-177 | 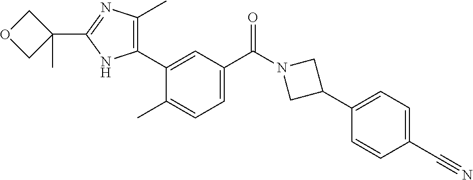 [SMILES: Cc1ccc(C(=O)N2CC(c3ccc(C#N)cc3)C2)cc1-c1[nH]c(C2(C)COC2)nc1C] | 0.011 |
| 002-178 | 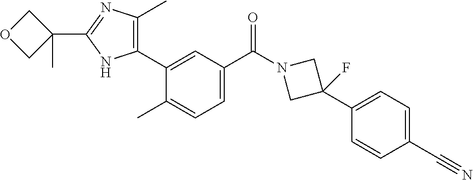 [SMILES: Cc1ccc(C(=O)N2CC(F)(c3ccc(C#N)cc3)C2)cc1-c1[nH]c(C2(C)COC2)nc1C] | 0.027 |
| 002-179 | 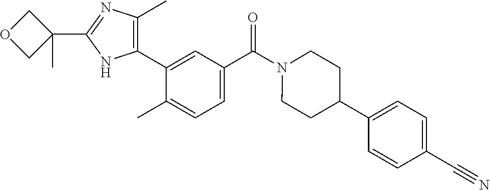 [SMILES: Cc1ccc(C(=O)N2CCC(c3ccc(C#N)cc3)CC2)cc1-c1[nH]c(C2(C)COC2)nc1C] | 0.025 |
| 002-180 | 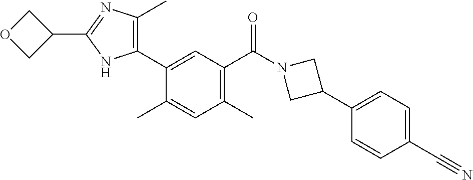 [SMILES: Cc1cc(C)c(-c2[nH]c(C3COC3)nc2C)cc1C(=O)N1CC(c2ccc(C#N)cc2)C1] | 0.034 |
| 002-181 | 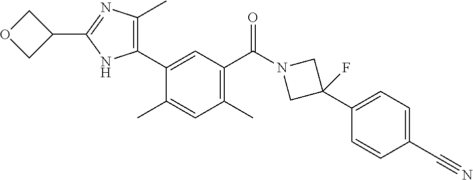 [SMILES: Cc1cc(C)c(-c2[nH]c(C3COC3)nc2C)cc1C(=O)N1CC(F)(c2ccc(C#N)cc2)C1] | 0.068 |
| 002-182 | 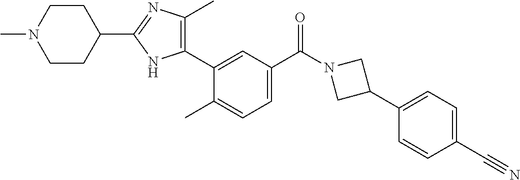 [SMILES: Cc1ccc(C(=O)N2CC(c3ccc(C#N)cc3)C2)cc1-c1[nH]c(C2CCN(C)CC2)nc1C] | 0.038 |
| 002-183 | 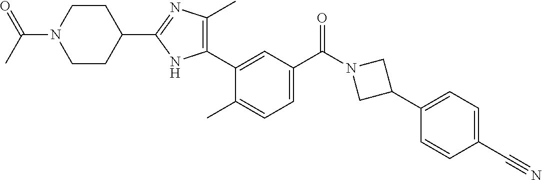 [SMILES: CC(=O)N1CCC(c2nc(C)c(-c3cc(C(=O)N4CC(c5ccc(C#N)cc5)C4)ccc3C)[nH]2)CC1] | 0.029 |
| 002-184 | 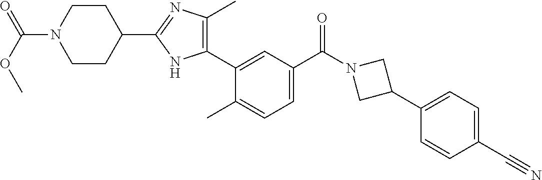 [SMILES: COC(=O)N1CCC(c2nc(C)c(-c3cc(C(=O)N4CC(c5ccc(C#N)cc5)C4)ccc3C)[nH]2)CC1] | 0.009 |
| 002-185 | 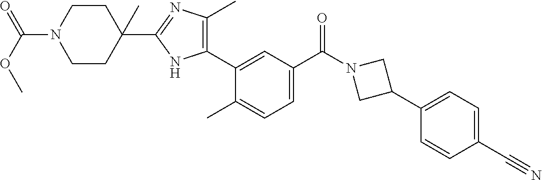 [SMILES: COC(=O)N1CCC(C)(c2nc(C)c(-c3cc(C(=O)N4CC(c5ccc(C#N)cc5)C4)ccc3C)[nH]2)CC1] | 0.033 |
| 002-186 | 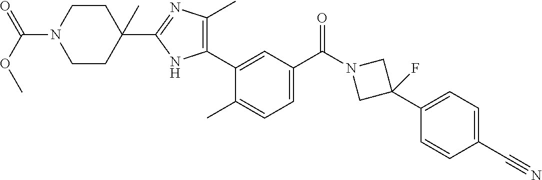 [SMILES: COC(=O)N1CCC(C)(c2nc(C)c(-c3cc(C(=O)N4CC(F)(c5ccc(C#N)cc5)C4)ccc3C)[nH]2)CC1] | 0.066 |
| 002-187 | 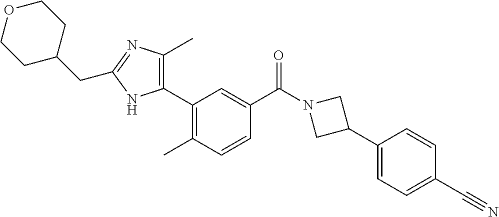 [SMILES: Cc1ccc(C(=O)N2CC(c3ccc(C#N)cc3)C2)cc1-c1[nH]c(CC2CCOCC2)nc1C] | 0.063 |
| 002-188 | 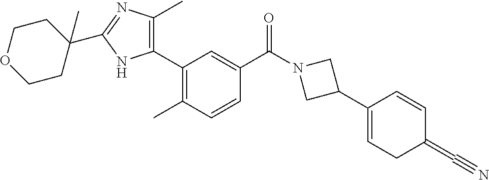 | 0.052 |
| 002-189 | 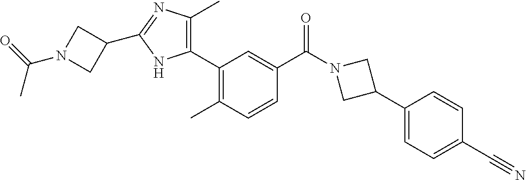 [SMILES: CC(=O)N1CC(c2nc(C)c(-c3cc(C(=O)N4CC(c5ccc(C#N)cc5)C4)ccc3C)[nH]2)C1] | 0.020 |
| 002-190 | 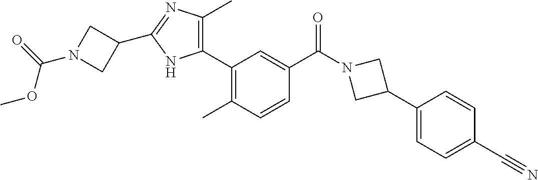 [SMILES: COC(=O)N1CC(c2nc(C)c(-c3cc(C(=O)N4CC(c5ccc(C#N)cc5)C4)ccc3C)[nH]2)C1] | 0.008 |
| 002-191 | 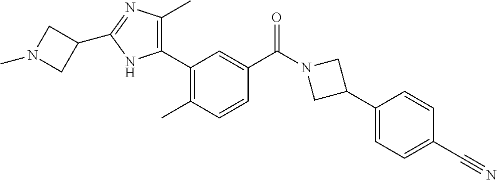 [SMILES: Cc1ccc(C(=O)N2CC(c3ccc(C#N)cc3)C2)cc1-c1[nH]c(C2CN(C)C2)nc1C] | 0.065 |
| 002-192 | 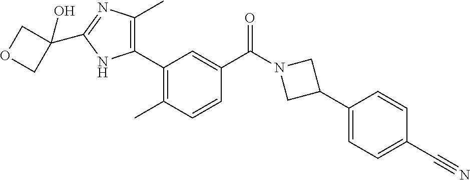 [SMILES: Cc1ccc(C(=O)N2CC(c3ccc(C#N)cc3)C2)cc1-c1[nH]c(C2(O)COC2)nc1C] | 0.024 |
| 002-193 | 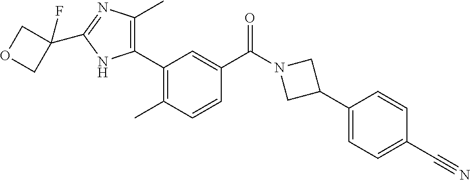 [SMILES: Cc1ccc(C(=O)N2CC(c3ccc(C#N)cc3)C2)cc1-c1[nH]c(C2(F)COC2)nc1C] | 0.065 |
| 002-194 | 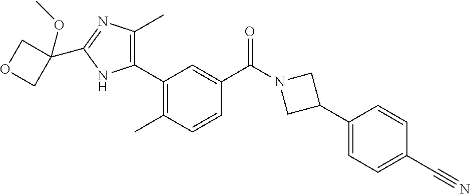 [SMILES: COC1(c2nc(C)c(-c3cc(C(=O)N4CC(c5ccc(C#N)cc5)C4)ccc3C)[nH]2)COC1] | 0.025 |
| 002-195 | 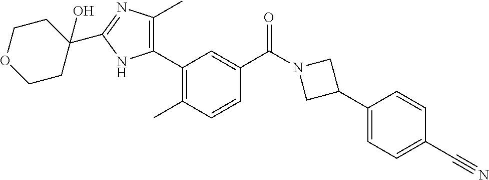 [SMILES: Cc1ccc(C(=O)N2CC(c3ccc(C#N)cc3)C2)cc1-c1[nH]c(C2(O)CCOCC2)nc1C] | 0.013 |
| 002-196 | 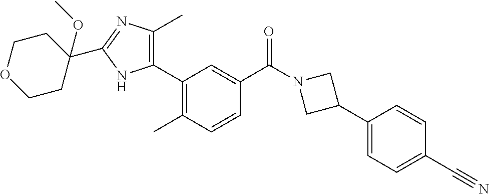 [SMILES: COC1(c2nc(C)c(-c3cc(C(=O)N4CC(c5ccc(C#N)cc5)C4)ccc3C)[nH]2)CCOCC1] | 0.015 |
| 002-197 | 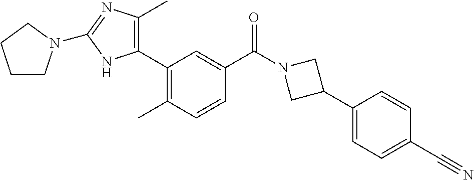 [SMILES: Cc1ccc(C(=O)N2CC(c3ccc(C#N)cc3)C2)cc1-c1[nH]c(N2CCCC2)nc1C] | 0.870 |
| 002-198 | 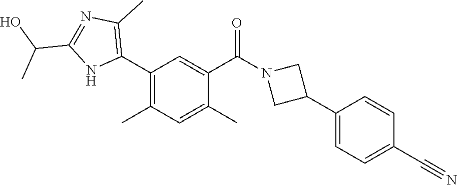 [SMILES: Cc1cc(C)c(-c2[nH]c(C(C)O)nc2C)cc1C(=O)N1CC(c2ccc(C#N)cc2)C1] | 0.054 |
| 002-199 | 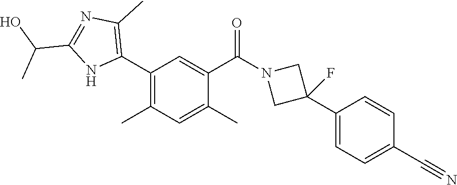 [SMILES: Cc1cc(C)c(-c2[nH]c(C(C)O)nc2C)cc1C(=O)N1CC(F)(c2ccc(C#N)cc2)C1] | 0.110 |
| 002-200 | 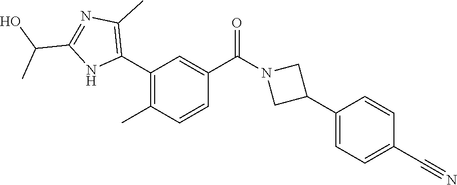 [SMILES: Cc1ccc(C(=O)N2CC(c3ccc(C#N)cc3)C2)cc1-c1[nH]c(C(C)O)nc1C] | 0.027 |
| 002-201 | 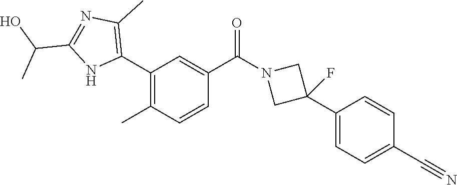 [SMILES: Cc1ccc(C(=O)N2CC(F)(c3ccc(C#N)cc3)C2)cc1-c1[nH]c(C(C)O)nc1C] | 0.130 |
| 002-202 | 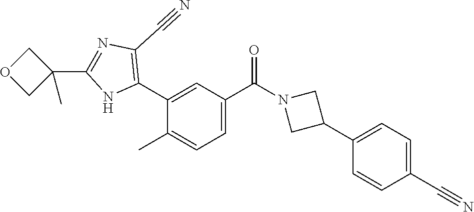 [SMILES: Cc1ccc(C(=O)N2CC(c3ccc(C#N)cc3)C2)cc1-c1[nH]c(C2(C)COC2)nc1C#N] | 0.020 |
| 002-203 | 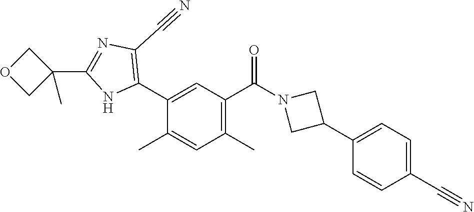 [SMILES: Cc1cc(C)c(-c2[nH]c(C3(C)COC3)nc2C#N)cc1C(=O)N1CC(c2ccc(C#N)cc2)C1] | 0.035 |
| 002-204 | 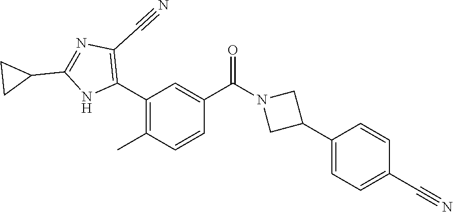 [SMILES: Cc1ccc(C(=O)N2CC(c3ccc(C#N)cc3)C2)cc1-c1[nH]c(C2CC2)nc1C#N] | 0.010 |
| 002-205 | 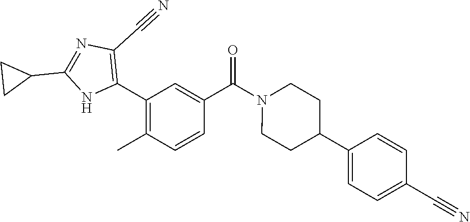 [SMILES: Cc1ccc(C(=O)N2CCC(c3ccc(C#N)cc3)CC2)cc1-c1[nH]c(C2CC2)nc1C#N] | 0.016 |
| 002-206 | 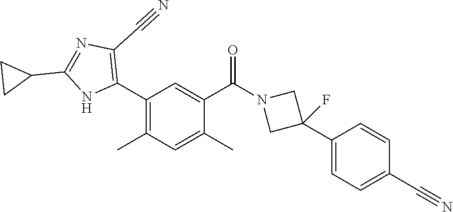 [SMILES: Cc1cc(C)c(-c2[nH]c(C3CC3)nc2C#N)cc1C(=O)N1CC(F)(c2ccc(C#N)cc2)C1] | 0.024 |
| 002-207 | 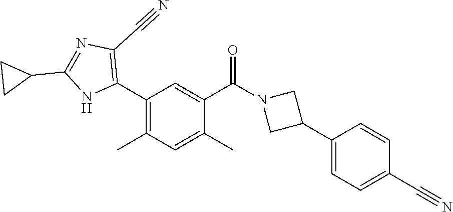 [SMILES: Cc1cc(C)c(-c2[nH]c(C3CC3)nc2C#N)cc1C(=O)N1CC(c2ccc(C#N)cc2)C1] | 0.035 |
| 002-208 | 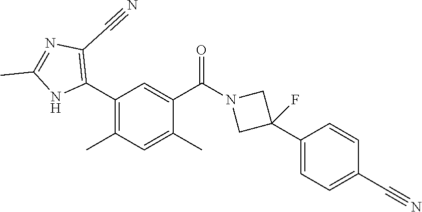 [SMILES: Cc1nc(C#N)c(-c2cc(C(=O)N3CC(F)(c4ccc(C#N)cc4)C3)c(C)cc2C)[nH]1] | 0.036 |
| 002-209 | 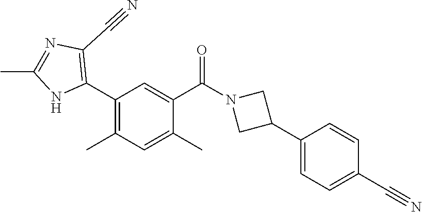 [SMILES: Cc1nc(C#N)c(-c2cc(C(=O)N3CC(c4ccc(C#N)cc4)C3)c(C)cc2C)[nH]1] | 0.024 |
| 002-210 | 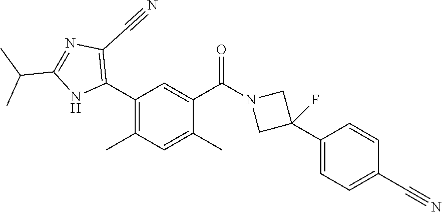 [SMILES: Cc1cc(C)c(-c2[nH]c(C(C)C)nc2C#N)cc1C(=O)N1CC(F)(c2ccc(C#N)cc2)C1] | 0.020 |
| 002-211 | 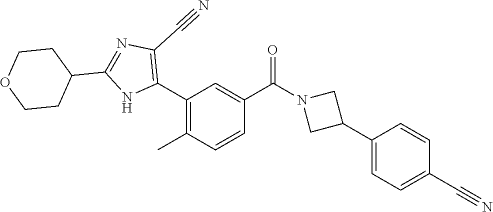 [SMILES: Cc1ccc(C(=O)N2CC(c3ccc(C#N)cc3)C2)cc1-c1[nH]c(C2CCOCC2)nc1C#N] | 0.011 |
| 002-212 | 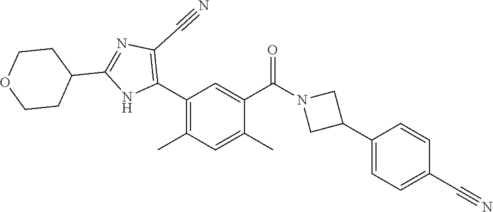 [SMILES: Cc1cc(C)c(-c2[nH]c(C3CCOCC3)nc2C#N)cc1C(=O)N1CC(c2ccc(C#N)cc2)C1] | 0.018 |
| 002-213 | 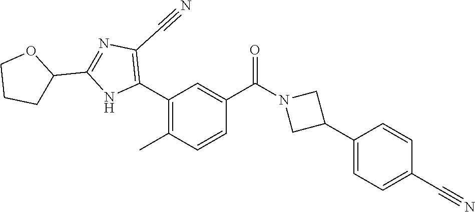 [SMILES: Cc1ccc(C(=O)N2CC(c3ccc(C#N)cc3)C2)cc1-c1[nH]c(C2CCCO2)nc1C#N] | 0.065 |
| 002-214 | 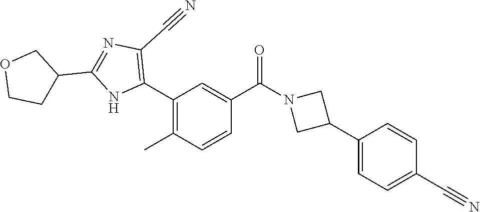 [SMILES: Cc1ccc(C(=O)N2CC(c3ccc(C#N)cc3)C2)cc1-c1[nH]c(C2CCOC2)nc1C#N] | 0.014 |
| 002-215 | 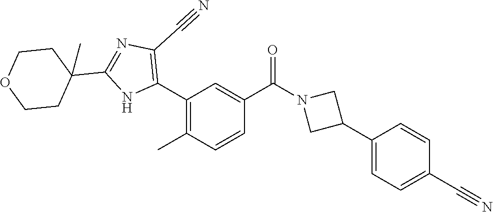 [SMILES: Cc1ccc(C(=O)N2CC(c3ccc(C#N)cc3)C2)cc1-c1[nH]c(C2(C)CCOCC2)nc1C#N] | 0.048 |
| 002-216 | 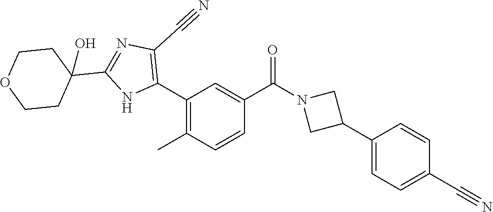 [SMILES: Cc1ccc(C(=O)N2CC(c3ccc(C#N)cc3)C2)cc1-c1[nH]c(C2(O)CCOCC2)nc1C#N] | 0.079 |
| 002-217 | 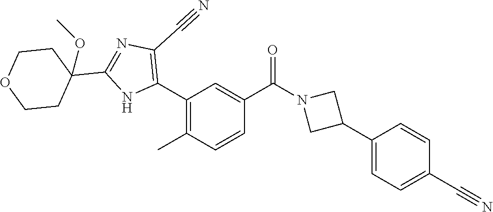 [SMILES: COC1(c2nc(C#N)c(-c3cc(C(=O)N4CC(c5ccc(C#N)cc5)C4)ccc3C)[nH]2)CCOCC1] | 0.052 |
| 002-218 | 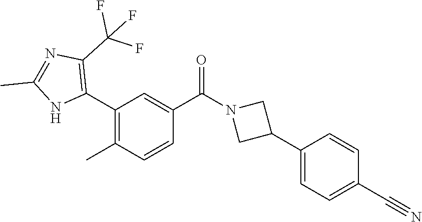 [SMILES: Cc1nc(C(F)(F)F)c(-c2cc(C(=O)N3CC(c4ccc(C#N)cc4)C3)ccc2C)[nH]1] | 0.015 |
| 002-219 | 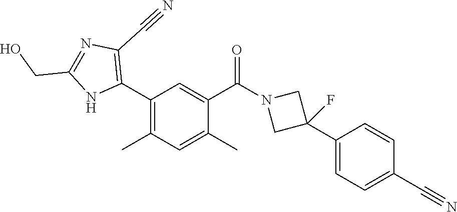 [SMILES: Cc1cc(C)c(-c2[nH]c(CO)nc2C#N)cc1C(=O)N1CC(F)(c2ccc(C#N)cc2)C1] | 0.059 |
| 002-220 | 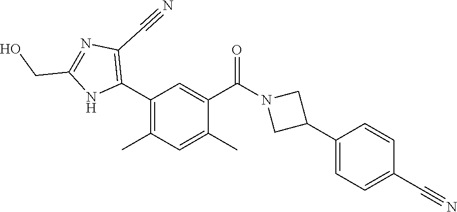 [SMILES: Cc1cc(C)c(-c2[nH]c(CO)nc2C#N)cc1C(=O)N1CC(c2ccc(C#N)cc2)C1] | 0.050 |
| 002-221 | 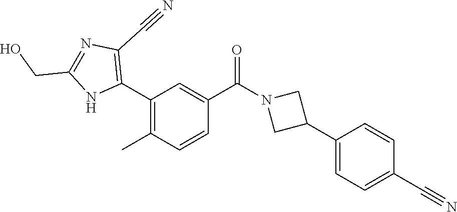 [SMILES: Cc1ccc(C(=O)N2CC(c3ccc(C#N)cc3)C2)cc1-c1[nH]c(CO)nc1C#N] | 0.043 |
| 002-222 | 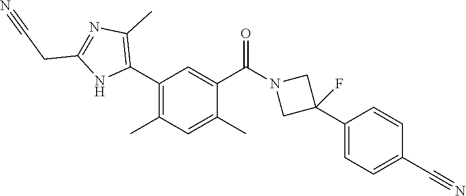 [SMILES: Cc1cc(C)c(-c2[nH]c(CC#N)nc2C)cc1C(=O)N1CC(F)(c2ccc(C#N)cc2)C1] | 0.053 |
| 002-223 | 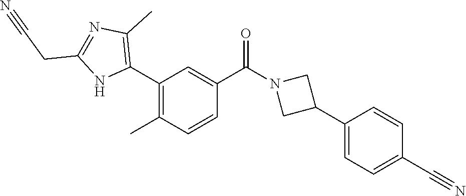 [SMILES: Cc1ccc(C(=O)N2CC(c3ccc(C#N)cc3)C2)cc1-c1[nH]c(CC#N)nc1C] | 0.023 |
| 002-224 | 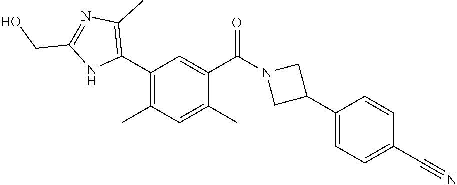 [SMILES: Cc1cc(C)c(-c2[nH]c(CO)nc2C)cc1C(=O)N1CC(c2ccc(C#N)cc2)C1] | 0.031 |
| 002-225 | 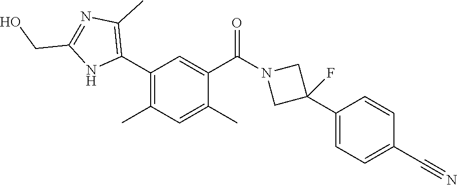 [SMILES: Cc1cc(C)c(-c2[nH]c(CO)nc2C)cc1C(=O)N1CC(F)(c2ccc(C#N)cc2)C1] | 0.059 |
| 002-226 | 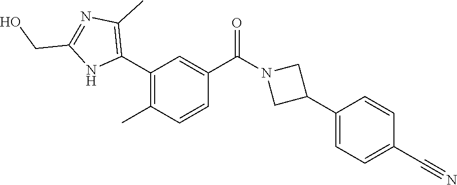 [SMILES: Cc1ccc(C(=O)N2CC(c3ccc(C#N)cc3)C2)cc1-c1[nH]c(CO)nc1C] | 0.088 |
| 002-220 | 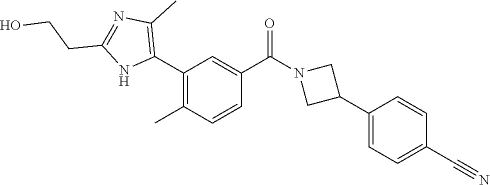 [SMILES: Cc1ccc(C(=O)N2CC(c3ccc(C#N)cc3)C2)cc1-c1[nH]c(CCO)nc1C] | 0.012 |
| 002-228 | 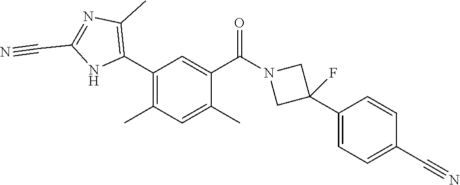 [SMILES: Cc1cc(C)c(-c2[nH]c(C#N)nc2C)cc1C(=O)N1CC(F)(c2ccc(C#N)cc2)C1] | 0.027 |
| 002-229 | 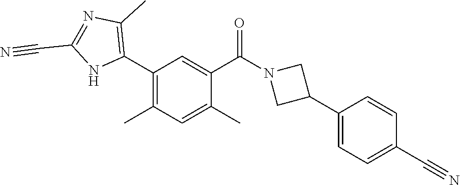 [SMILES: Cc1cc(C)c(-c2[nH]c(C#N)nc2C)cc1C(=O)N1CC(c2ccc(C#N)cc2)C1] | 0.042 |
| 002-230 | 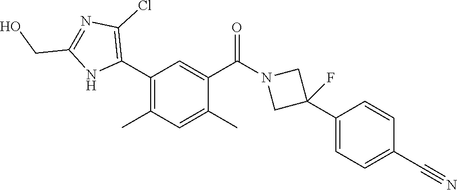 [SMILES: Cc1cc(C)c(-c2[nH]c(CO)nc2Cl)cc1C(=O)N1CC(F)(c2ccc(C#N)cc2)C1] | 0.017 |
| 002-230 | 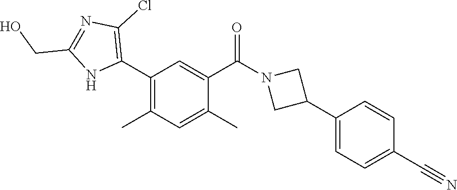 [SMILES: Cc1cc(C)c(-c2[nH]c(CO)nc2Cl)cc1C(=O)N1CC(c2ccc(C#N)cc2)C1] | 0.019 |
| 002-232 | 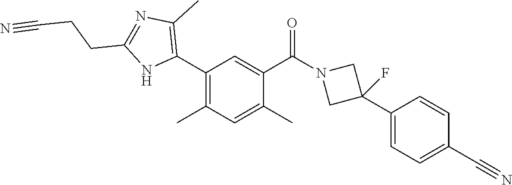 [SMILES: Cc1cc(C)c(-c2[nH]c(CCC#N)nc2C)cc1C(=O)N1CC(F)(c2ccc(C#N)cc2)C1] | 0.020 |
| 002-233 | 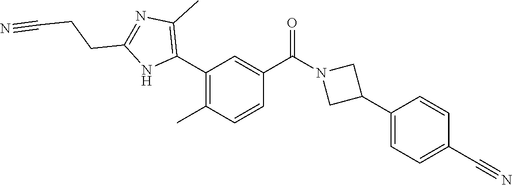 [SMILES: Cc1ccc(C(=O)N2CC(c3ccc(C#N)cc3)C2)cc1-c1[nH]c(CCC#N)nc1C] | 0.011 |
| 002-234 | 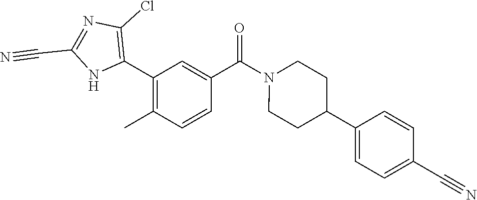 [SMILES: Cc1ccc(C(=O)N2CCC(c3ccc(C#N)cc3)CC2)cc1-c1[nH]c(C#N)nc1Cl] | 0.053 |
| 002-235 | 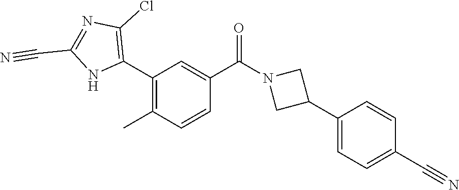 [SMILES: Cc1ccc(C(=O)N2CC(c3ccc(C#N)cc3)C2)cc1-c1[nH]c(C#N)nc1Cl] | 0.039 |
| 002-236 | 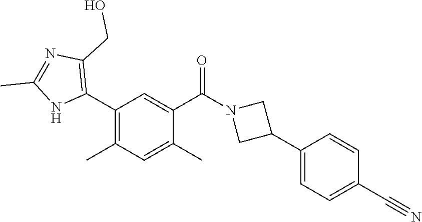 [SMILES: Cc1nc(CO)c(-c2cc(C(=O)N3CC(c4ccc(C#N)cc4)C3)c(C)cc2C)[nH]1] | 0.019 |
| 002-237 | 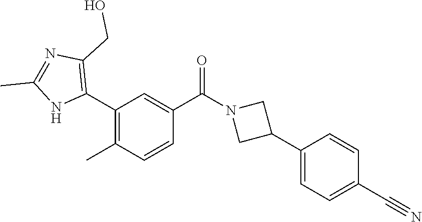 [SMILES: Cc1nc(CO)c(-c2cc(C(=O)N3CC(c4ccc(C#N)cc4)C3)ccc2C)[nH]1] | 0.016 |
| 002-238 | 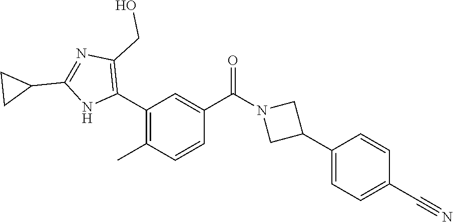 [SMILES: Cc1ccc(C(=O)N2CC(c3ccc(C#N)cc3)C2)cc1-c1[nH]c(C2CC2)nc1CO] | 0.0001 |
| 002-239 | 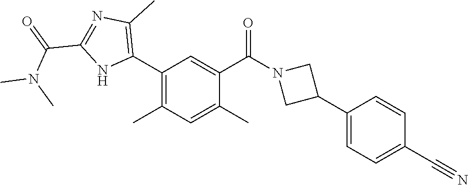 [SMILES: Cc1cc(C)c(-c2[nH]c(C(=O)N(C)C)nc2C)cc1C(=O)N1CC(c2ccc(C#N)cc2)C1] | 0.440 |
| 002-240 | 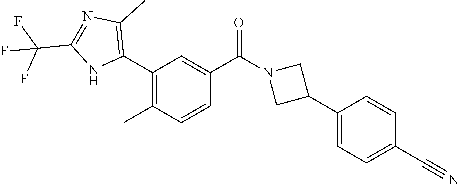 [SMILES: Cc1ccc(C(=O)N2CC(c3ccc(C#N)cc3)C2)cc1-c1[nH]c(C(F)(F)F)nc1C] | 0.053 |
| 002-241 | 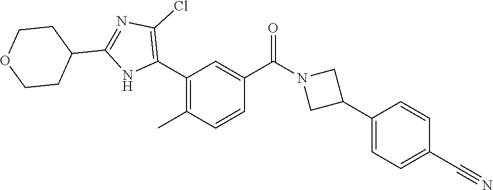 [SMILES: Cc1ccc(C(=O)N2CC(c3ccc(C#N)cc3)C2)cc1-c1[nH]c(C2CCOCC2)nc1Cl] | 0.009 |
| 002-242 | 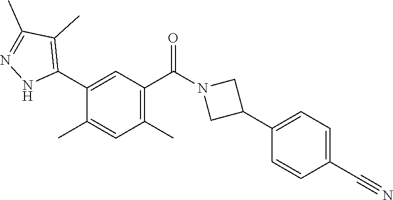 [SMILES: Cc1cc(C)c(-c2[nH]nc(C)c2C)cc1C(=O)N1CC(c2ccc(C#N)cc2)C1] | 0.060 |
| 002-243 | 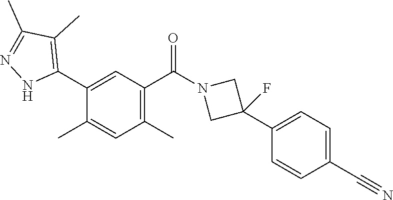 [SMILES: Cc1cc(C)c(-c2[nH]nc(C)c2C)cc1C(=O)N1CC(F)(c2ccc(C#N)cc2)C1] | 0.170 |
| 002-244 | 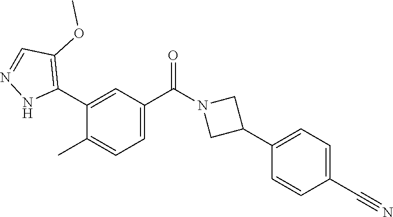 [SMILES: COc1cn[nH]c1-c1cc(C(=O)N2CC(c3ccc(C#N)cc3)C2)ccc1C] | 0.150 |
| 002-245 | 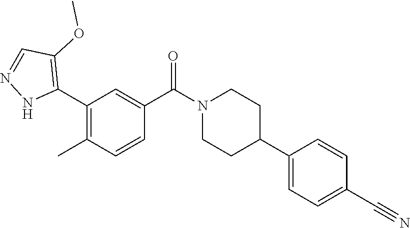 [SMILES: COc1cn[nH]c1-c1cc(C(=O)N2CCC(c3ccc(C#N)cc3)CC2)ccc1C] | 0.109 |
| 002-246 | 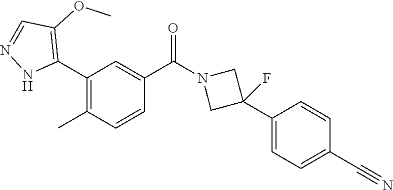 [SMILES: COc1cn[nH]c1-c1cc(C(=O)N2CC(F)(c3ccc(C#N)cc3)C2)ccc1C] | 0.340 |
| 002-247 | 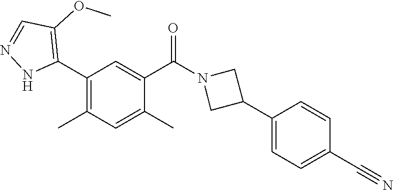 [SMILES: COc1cn[nH]c1-c1cc(C(=O)N2CC(c3ccc(C#N)cc3)C2)c(C)cc1C] | 0.110 |
| 002-248 | 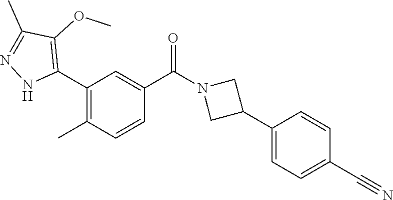 [SMILES: COc1c(C)n[nH]c1-c1cc(C(=O)N2CC(c3ccc(C#N)cc3)C2)ccc1C] | 0.075 |
| 002-249 | 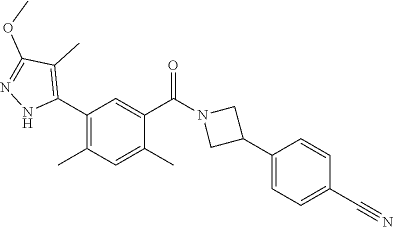 [SMILES: COc1n[nH]c(-c2cc(C(=O)N3CC(c4ccc(C#N)cc4)C3)c(C)cc2C)c1C] | 0.043 |
| 002-250 | 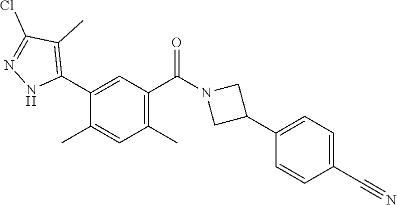 [SMILES: Cc1cc(C)c(-c2[nH]nc(Cl)c2C)cc1C(=O)N1CC(c2ccc(C#N)cc2)C1] | 0.051 |
| 002-251 | 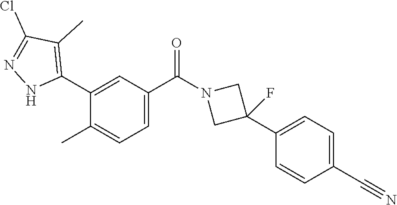 [SMILES: Cc1ccc(C(=O)N2CC(F)(c3ccc(C#N)cc3)C2)cc1-c1[nH]nc(Cl)c1C] | 0.178 |
| 002-252 | 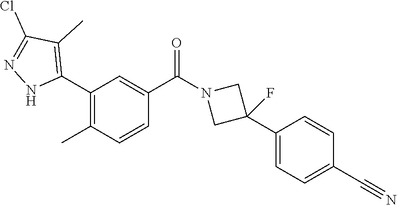 [SMILES: Cc1ccc(C(=O)N2CC(F)(c3ccc(C#N)cc3)C2)cc1-c1[nH]nc(Cl)c1C] | 0.095 |
| 002-253 | 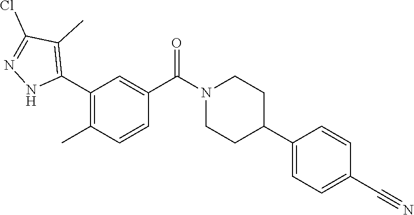 [SMILES: Cc1ccc(C(=O)N2CCC(c3ccc(C#N)cc3)CC2)cc1-c1[nH]nc(Cl)c1C] | 0.080 |
| 002-255 | 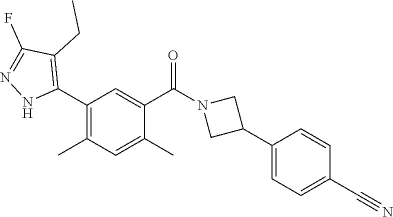 [SMILES: CCc1c(F)n[nH]c1-c1cc(C(=O)N2CC(c3ccc(C#N)cc3)C2)c(C)cc1C] | 0.063 |
| 002-256 | 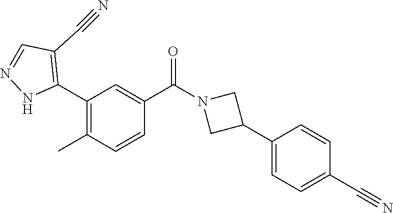 [SMILES: Cc1ccc(C(=O)N2CC(c3ccc(C#N)cc3)C2)cc1-c1[nH]ncc1C#N] | 0.210 |
| 002-257 | 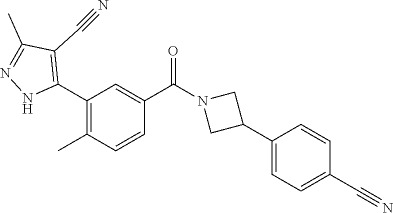 [SMILES: Cc1ccc(C(=O)N2CC(c3ccc(C#N)cc3)C2)cc1-c1[nH]nc(C)c1C#N] | 0.440 |
| 002-258 | 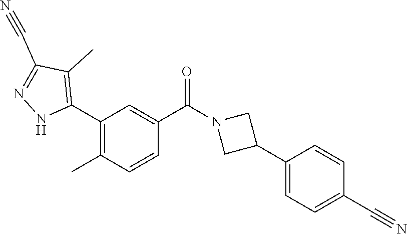 [SMILES: Cc1ccc(C(=O)N2CC(c3ccc(C#N)cc3)C2)cc1-c1[nH]nc(C#N)c1C] | 0.058 |
| 002-259 | 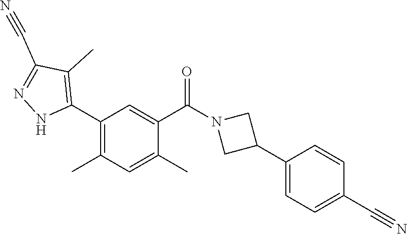 [SMILES: Cc1cc(C)c(-c2[nH]nc(C#N)c2C)cc1C(=O)N1CC(c2ccc(C#N)cc2)C1] | 0.140 |
| 002-260 | 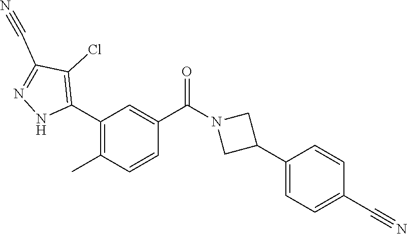 [SMILES: Cc1ccc(C(=O)N2CC(c3ccc(C#N)cc3)C2)cc1-c1[nH]nc(C#N)c1Cl] | 0.067 |
| 002-261 | 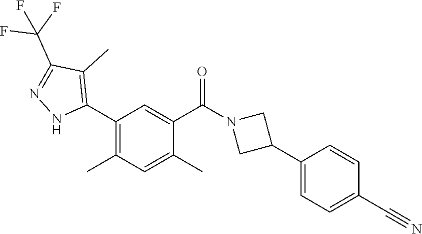 [SMILES: Cc1cc(C)c(-c2[nH]nc(C(F)(F)F)c2C)cc1C(=O)N1CC(c2ccc(C#N)cc2)C1] | 0.190 |
| 002-262 | 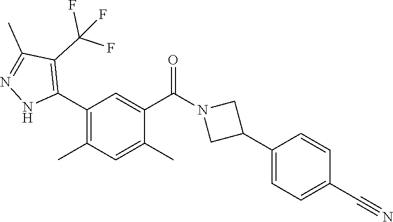 [SMILES: Cc1cc(C)c(-c2[nH]nc(C)c2C(F)(F)F)cc1C(=O)N1CC(c2ccc(C#N)cc2)C1] | 0.140 |
| 002-263 | 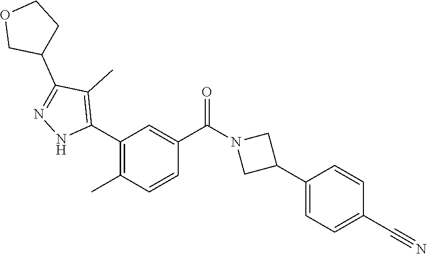 [SMILES: Cc1ccc(C(=O)N2CC(c3ccc(C#N)cc3)C2)cc1-c1[nH]nc(C2CCOC2)c1C] | 0.094 |
| 002-264 | 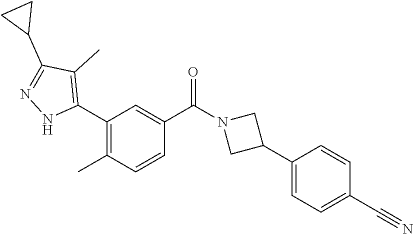 [SMILES: Cc1ccc(C(=O)N2CC(c3ccc(C#N)cc3)C2)cc1-c1[nH]nc(C2CC2)c1C] | 0.056 |
| 002-265 | 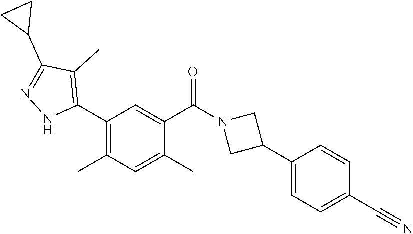 [SMILES: Cc1cc(C)c(-c2[nH]nc(C3CC3)c2C)cc1C(=O)N1CC(c2ccc(C#N)cc2)C1] | 0.110 |
| 002-266 | 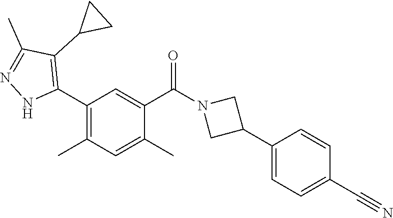 [SMILES: Cc1cc(C)c(-c2[nH]nc(C)c2C2CC2)cc1C(=O)N1CC(c2ccc(C#N)cc2)C1] | 0.200 |
| 002-267 | 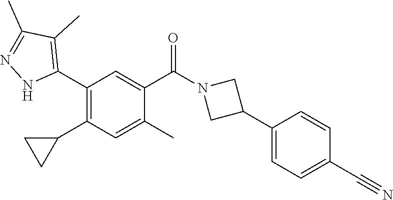 [SMILES: Cc1cc(C2CC2)c(-c2[nH]nc(C)c2C)cc1C(=O)N1CC(c2ccc(C#N)cc2)C1] | 0.100 |
| 002-268 | 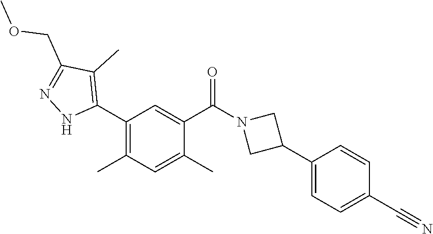 [SMILES: COCc1n[nH]c(-c2cc(C(=O)N3CC(c4ccc(C#N)cc4)C3)c(C)cc2C)c1C] | 0.059 |
| 002-269 | 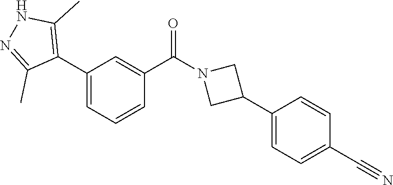 [SMILES: Cc1n[nH]c(C)c1-c1cccc(C(=O)N2CC(c3ccc(C#N)cc3)C2)c1] | 50.000 |
| 002-270 | 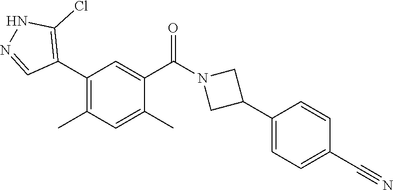 [SMILES: Cc1cc(C)c(-c2cn[nH]c2Cl)cc1C(=O)N1CC(c2ccc(C#N)cc2)C1] | |
| 002-271 | 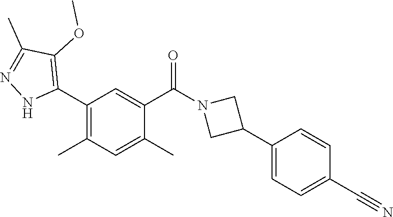 [SMILES: COc1c(C)n[nH]c1-c1cc(C(=O)N2CC(c3ccc(C#N)cc3)C2)c(C)cc1C] | |
| 002-272 | 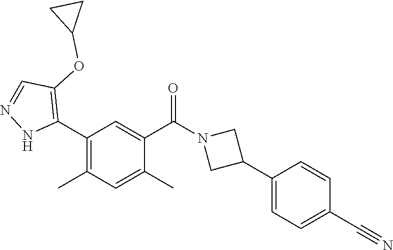 [SMILES: Cc1cc(C)c(-c2[nH]ncc2OC2CC2)cc1C(=O)N1CC(c2ccc(C#N)cc2)C1] | |
| 002-273 | 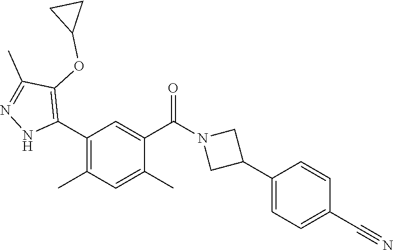 [SMILES: Cc1cc(C)c(-c2[nH]nc(C)c2OC2CC2)cc1C(=O)N1CC(c2ccc(C#N)cc2)C1] | |
| 002-274 | 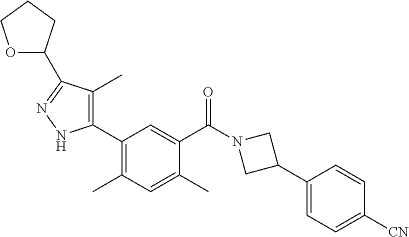 [SMILES: Cc1cc(C)c(-c2[nH]nc(C3CCCO3)c2C)cc1C(=O)N1CC(c2ccc(C#N)cc2)C1] | 0.051 |
| 002-275 | 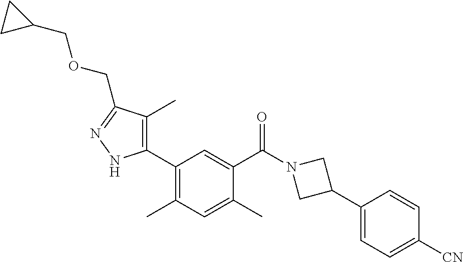 [SMILES: Cc1cc(C)c(-c2[nH]nc(COCC3CC3)c2C)cc1C(=O)N1CC(c2ccc(C#N)cc2)C1] | 0.057 |
| 002-276 | 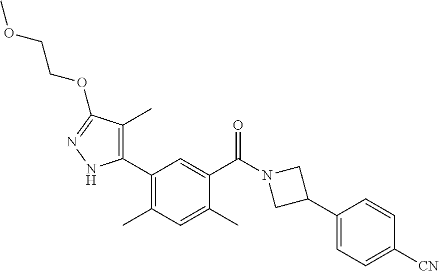 [SMILES: COCCOc1n[nH]c(-c2cc(C(=O)N3CC(c4ccc(C#N)cc4)C3)c(C)cc2C)c1C] | 50 |
| 002-277 | 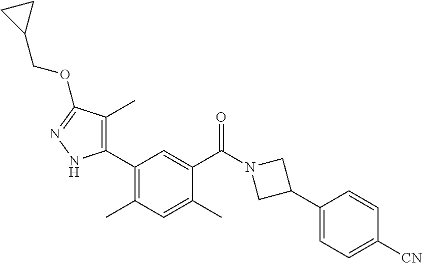 [SMILES: Cc1cc(C)c(-c2[nH]nc(OCC3CC3)c2C)cc1C(=O)N1CC(c2ccc(C#N)cc2)C1] | 50 |
| 002-278 | 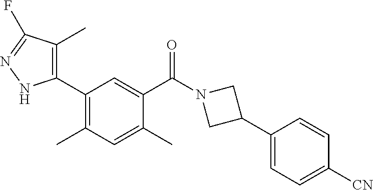 [SMILES: Cc1cc(C)c(-c2[nH]nc(F)c2C)cc1C(=O)N1CC(c2ccc(C#N)cc2)C1] | |
| 002-279 | 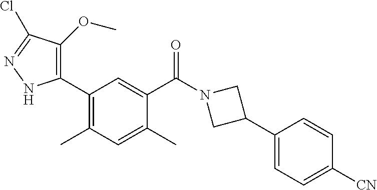 [SMILES: COc1c(Cl)n[nH]c1-c1cc(C(=O)N2CC(c3ccc(C#N)cc3)C2)c(C)cc1C] | 0.11 |
| 002-280 | 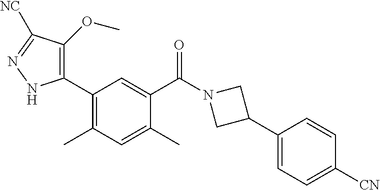 [SMILES: COc1c(C#N)n[nH]c1-c1cc(C(=O)N2CC(c3ccc(C#N)cc3)C2)c(C)cc1C] | 0.089 |
| 002-281 | 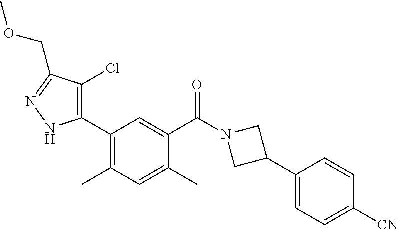 [SMILES: COCc1n[nH]c(-c2cc(C(=O)N3CC(c4ccc(C#N)cc4)C3)c(C)cc2C)c1Cl] | 0.072 |
| 002-282 | 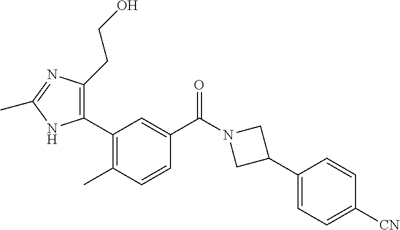 [SMILES: Cc1nc(CCO)c(-c2cc(C(=O)N3CC(c4ccc(C#N)cc4)C3)ccc2C)[nH]1] | 0.091 |
| 002-283 | 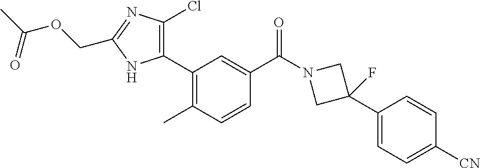 [SMILES: CC(=O)OCc1nc(Cl)c(-c2cc(C(=O)N3CC(F)(c4ccc(C#N)cc4)C3)ccc2C)[nH]1] | 0.064 |
| 002-284 | 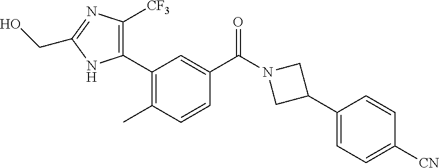 [SMILES: Cc1ccc(C(=O)N2CC(c3ccc(C#N)cc3)C2)cc1-c1[nH]c(CO)nc1C(F)(F)F] | 0.044 |
| 002-285 | 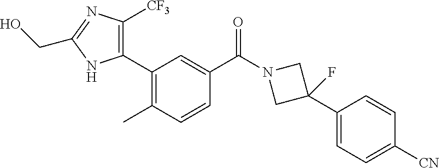 [SMILES: Cc1ccc(C(=O)N2CC(F)(c3ccc(C#N)cc3)C2)cc1-c1[nH]c(CO)nc1C(F)(F)F] | 0.048 |
| 002-286 | 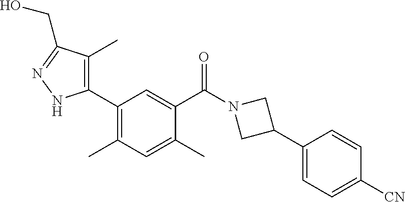 [SMILES: Cc1cc(C)c(-c2[nH]nc(CO)c2C)cc1C(=O)N1CC(c2ccc(C#N)cc2)C1] | 0.061 |
| 002-287 | 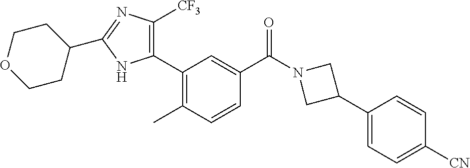 [SMILES: Cc1ccc(C(=O)N2CC(c3ccc(C#N)cc3)C2)cc1-c1[nH]c(C2CCOCC2)nc1C(F)(F)F] | 0.029 |
| 002-288 | 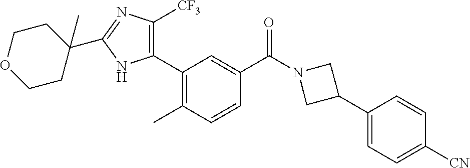 [SMILES: Cc1ccc(C(=O)N2CC(c3ccc(C#N)cc3)C2)cc1-c1[nH]c(C2(C)CCOCC2)nc1C(F)(F)F] | 0.026 |
| 002-289 | 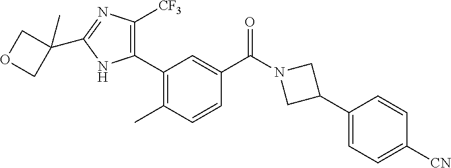 [SMILES: Cc1ccc(C(=O)N2CC(c3ccc(C#N)cc3)C2)cc1-c1[nH]c(C2(C)COC2)nc1C(F)(F)F] | 0.018 |
| 002-290 | 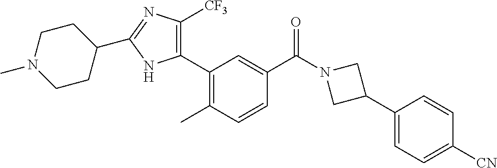 [SMILES: Cc1ccc(C(=O)N2CC(c3ccc(C#N)cc3)C2)cc1-c1[nH]c(C2CCN(C)CC2)nc1C(F)(F)F] | 0.021 |
| 002-291 | 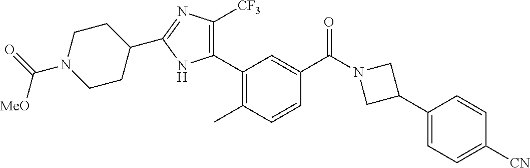 [SMILES: COC(=O)N1CCC(c2nc(C(F)(F)F)c(-c3cc(C(=O)N4CC(c5ccc(C#N)cc5)C4)ccc3C)[nH]2)CC1] | 0.02 |
| 002-292 | 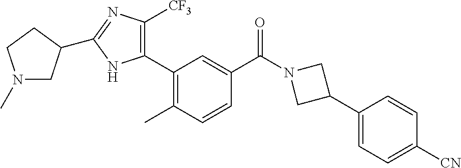 [SMILES: Cc1ccc(C(=O)N2CC(c3ccc(C#N)cc3)C2)cc1-c1[nH]c(C2CCN(C)C2)nc1C(F)(F)F] | 0.044 |
| 002-293 | 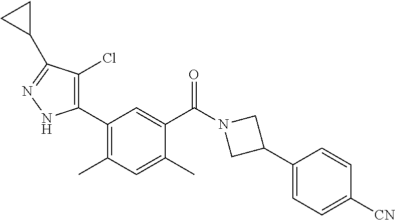 [SMILES: Cc1cc(C)c(-c2[nH]nc(C3CC3)c2Cl)cc1C(=O)N1CC(c2ccc(C#N)cc2)C1] | 0.064 |
| 002-294 | 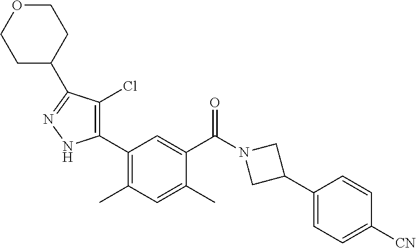 [SMILES: Cc1cc(C)c(-c2[nH]nc(C3CCOCC3)c2Cl)cc1C(=O)N1CC(c2ccc(C#N)cc2)C1] | 0.049 |
| 002-295 | 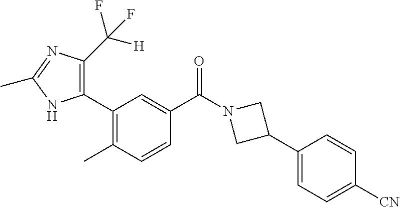 [SMILES: Cc1nc(C(F)F)c(-c2cc(C(=O)N3CC(c4ccc(C#N)cc4)C3)ccc2C)[nH]1] | 0.031 |
| 002-296 | 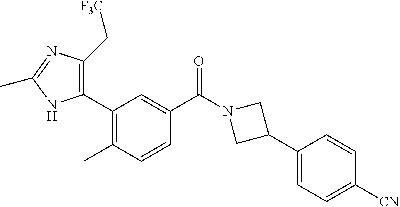 [SMILES: Cc1nc(CC(F)(F)F)c(-c2cc(C(=O)N3CC(c4ccc(C#N)cc4)C3)ccc2C)[nH]1] | 0.024 |
| 002-297 | 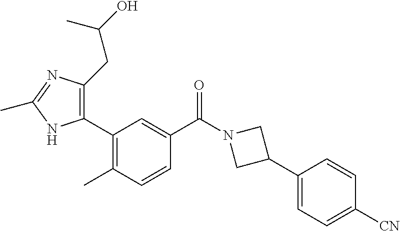 [SMILES: Cc1nc(CC(C)O)c(-c2cc(C(=O)N3CC(c4ccc(C#N)cc4)C3)ccc2C)[nH]1] | |
| 002-298 | 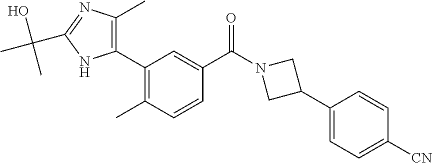 [SMILES: Cc1ccc(C(=O)N2CC(c3ccc(C#N)cc3)C2)cc1-c1[nH]c(C(C)(C)O)nc1C] | |
| 002-299 | 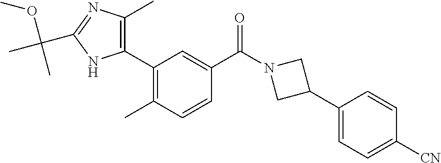 [SMILES: COC(C)(C)c1nc(C)c(-c2cc(C(=O)N3CC(c4ccc(C#N)cc4)C3)ccc2C)[nH]1] | |
| 002-300A | 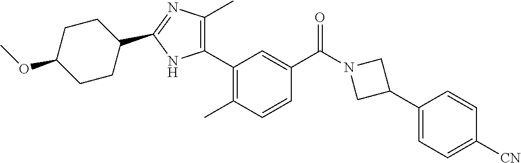 | 0.026 |
| 002-300B | 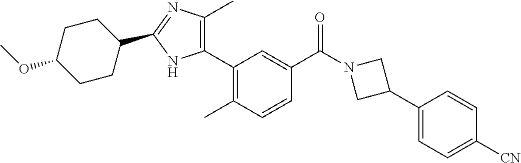 | 0.024 |
| 002-301 | 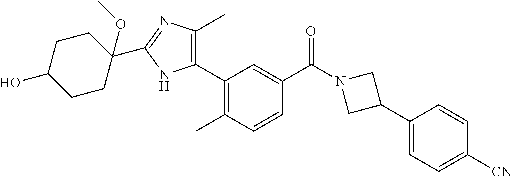 [SMILES: COC1(c2nc(C)c(-c3cc(C(=O)N4CC(c5ccc(C#N)cc5)C4)ccc3C)[nH]2)CCC(O)CC1] | |
| 002-302 | 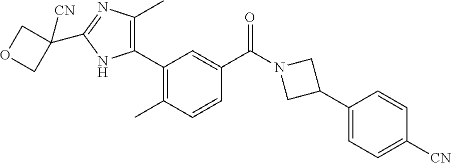 [SMILES: Cc1ccc(C(=O)N2CC(c3ccc(C#N)cc3)C2)cc1-c1[nH]c(C2(C#N)COC2)nc1C] | |
| 002-303 | 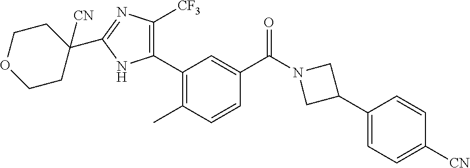 [SMILES: Cc1ccc(C(=O)N2CC(c3ccc(C#N)cc3)C2)cc1-c1[nH]c(C2(C#N)CCOCC2)nc1C(F)(F)F] | |
| 002-304 | 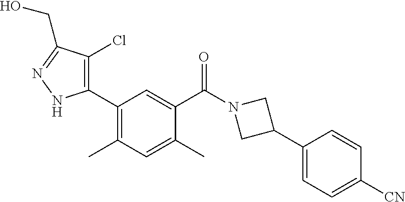 [SMILES: Cc1cc(C)c(-c2[nH]nc(CO)c2Cl)cc1C(=O)N1CC(c2ccc(C#N)cc2)C1] | 0.021 |
| 002-305 | 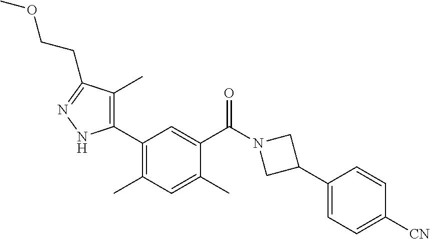 [SMILES: COCCc1n[nH]c(-c2cc(C(=O)N3CC(c4ccc(C#N)cc4)C3)c(C)cc2C)c1C] | 0.078 |
| 002-306 | 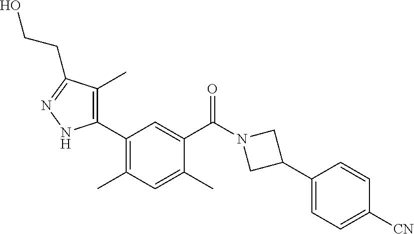 [SMILES: Cc1cc(C)c(-c2[nH]nc(CCO)c2C)cc1C(=O)N1CC(c2ccc(C#N)cc2)C1] | |
| 002-307 | 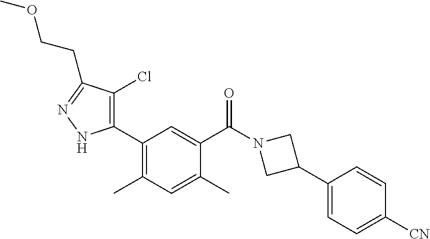 [SMILES: COCCc1n[nH]c(-c2cc(C(=O)N3CC(c4ccc(C#N)cc4)C3)c(C)cc2C)c1Cl] | 0.056 |
| 002-308 | 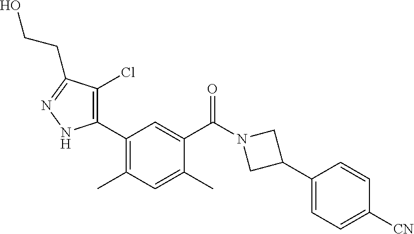 [SMILES: Cc1cc(C)c(-c2[nH]nc(CCO)c2Cl)cc1C(=O)N1CC(c2ccc(C#N)cc2)C1] | |
| 002-309 | 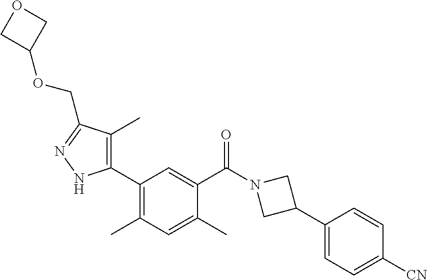 [SMILES: Cc1cc(C)c(-c2[nH]nc(COC3COC3)c2C)cc1C(=O)N1CC(c2ccc(C#N)cc2)C1] | 0.037 |
| 002-310 | 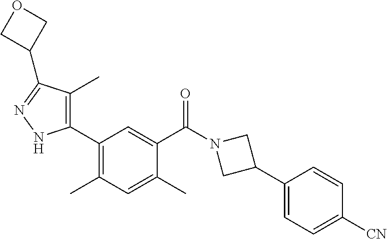 [SMILES: Cc1cc(C)c(-c2[nH]nc(C3COC3)c2C)cc1C(=O)N1CC(c2ccc(C#N)cc2)C1] | |
| 002-311 | 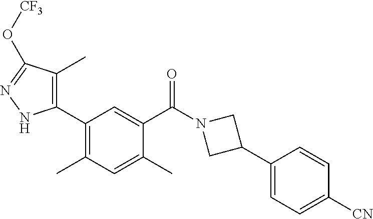 [SMILES: Cc1cc(C)c(-c2[nH]nc(OC(F)(F)F)c2C)cc1C(=O)N1CC(c2ccc(C#N)cc2)C1] | |
| 002-312 | 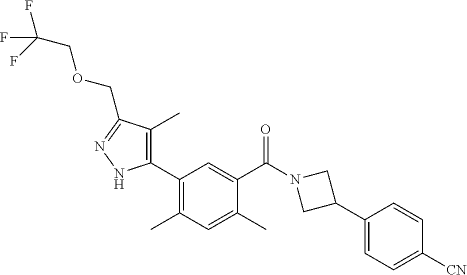 [SMILES: Cc1cc(C)c(-c2[nH]nc(COCC(F)(F)F)c2C)cc1C(=O)N1CC(c2ccc(C#N)cc2)C1] | 0.037 |
| 002-313 | 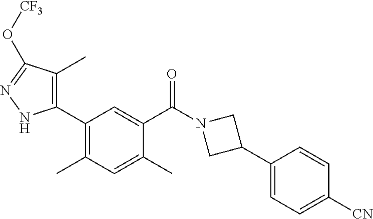 [SMILES: Cc1cc(C)c(-c2[nH]nc(OC(F)(F)F)c2C)cc1C(=O)N1CC(c2ccc(C#N)cc2)C1] | 0.1 |
| 002-314 | 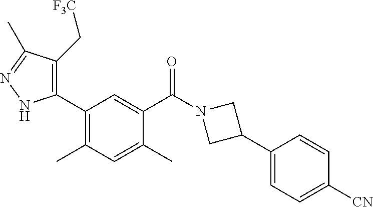 [SMILES: Cc1cc(C)c(-c2[nH]nc(C)c2CC(F)(F)F)cc1C(=O)N1CC(c2ccc(C#N)cc2)C1] | 0.46 |
| 002-315 | 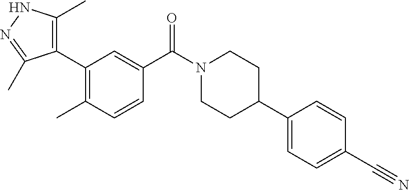 [SMILES: Cc1ccc(C(=O)N2CCC(c3ccc(C#N)cc3)CC2)cc1-c1c(C)n[nH]c1C] | |
| 002-316 | 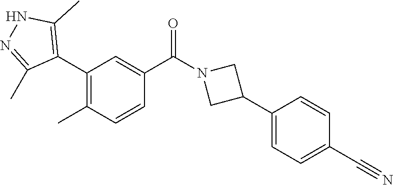 [SMILES: Cc1ccc(C(=O)N2CC(c3ccc(C#N)cc3)C2)cc1-c1c(C)n[nH]c1C] | |
| 002-317 | 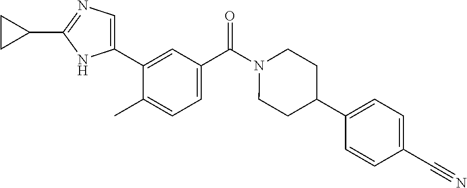 [SMILES: Cc1ccc(C(=O)N2CCC(c3ccc(C#N)cc3)CC2)cc1-c1cnc(C2CC2)[nH]1] | |
| 002-318 | 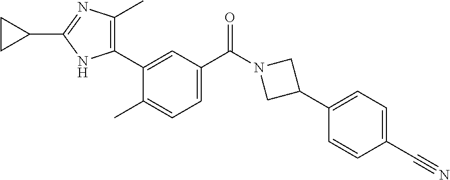 [SMILES: Cc1ccc(C(=O)N2CC(c3ccc(C#N)cc3)C2)cc1-c1[nH]c(C2CC2)nc1C] | |
| 002-320 | 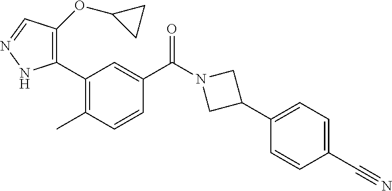 [SMILES: Cc1ccc(C(=O)N2CC(c3ccc(C#N)cc3)C2)cc1-c1[nH]ncc1OC1CC1] | |
| 002-321 | 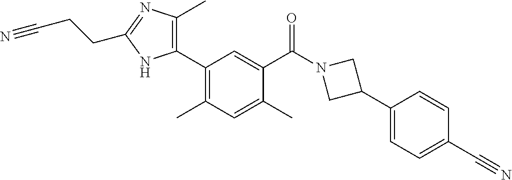 [SMILES: Cc1cc(C)c(-c2[nH]c(CCC#N)nc2C)cc1C(=O)N1CC(c2ccc(C#N)cc2)C1] | |
| 002-322 | 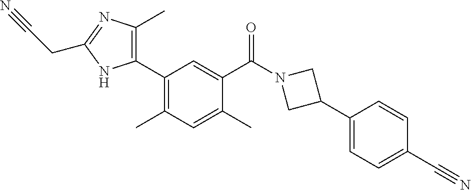 [SMILES: Cc1cc(C)c(-c2[nH]c(CC#N)nc2C)cc1C(=O)N1CC(c2ccc(C#N)cc2)C1] | |
| 002-323 | 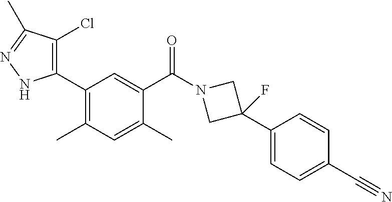 [SMILES: Cc1cc(C)c(-c2[nH]nc(C)c2Cl)cc1C(=O)N1CC(F)(c2ccc(C#N)cc2)C1] | |
| 002-324 | 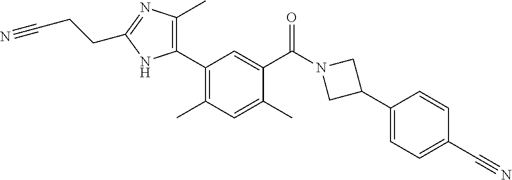 [SMILES: Cc1cc(C)c(-c2[nH]c(CCC#N)nc2C)cc1C(=O)N1CC(c2ccc(C#N)cc2)C1] | |
| 002-325 | 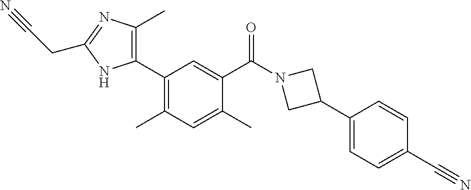 [SMILES: Cc1cc(C)c(-c2[nH]c(CC#N)nc2C)cc1C(=O)N1CC(c2ccc(C#N)cc2)C1] | |
| 002-326 | 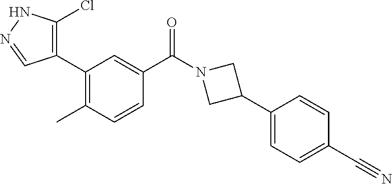 [SMILES: Cc1ccc(C(=O)N2CC(c3ccc(C#N)cc3)C2)cc1-c1cn[nH]c1Cl] | |
| 002-327 | 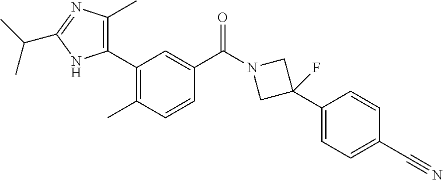 [SMILES: Cc1ccc(C(=O)N2CC(F)(c3ccc(C#N)cc3)C2)cc1-c1[nH]c(C(C)C)nc1C] | |
| 002-328 | 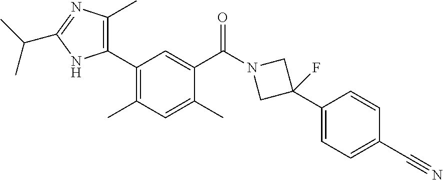 [SMILES: Cc1cc(C)c(-c2[nH]c(C(C)C)nc2C)cc1C(=O)N1CC(F)(c2ccc(C#N)cc2)C1] | |
| 002-329 | 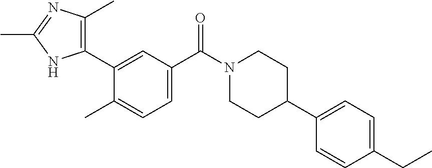 [SMILES: CCc1ccc(C2CCN(C(=O)c3ccc(C)c(-c4[nH]c(C)nc4C)c3)CC2)cc1] | 0.089 |
| 002-330 | 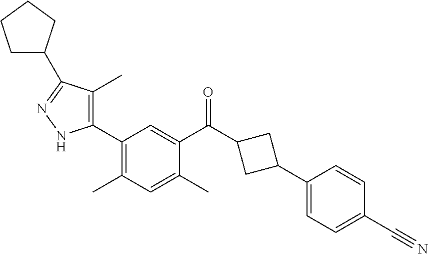 [SMILES: Cc1cc(C)c(-c2[nH]nc(C3CCCC3)c2C)cc1C(=O)C1CC(c2ccc(C#N)cc2)C1] | 0.1 |
| 002-331 | 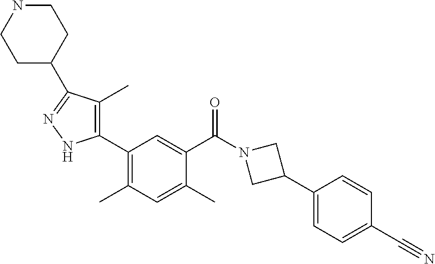 [SMILES: Cc1cc(C)c(-c2[nH]nc(C3CCNCC3)c2C)cc1C(=O)N1CC(c2ccc(C#N)cc2)C1] | 0.11 |
| 002-332 | 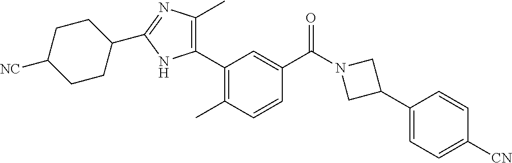 [SMILES: Cc1ccc(C(=O)N2CC(c3ccc(C#N)cc3)C2)cc1-c1[nH]c(C2CCC(C#N)CC2)nc1C] | 0.01 |
| 002-333 | 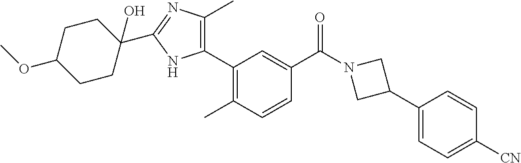 [SMILES: COC1CCC(O)(c2nc(C)c(-c3cc(C(=O)N4CC(c5ccc(C#N)cc5)C4)ccc3C)[nH]2)CC1] | |
| 002-334 | 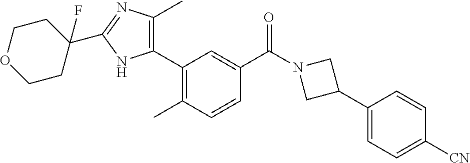 [SMILES: Cc1ccc(C(=O)N2CC(c3ccc(C#N)cc3)C2)cc1-c1[nH]c(C2(F)CCOCC2)nc1C] | |
| 002-335 | 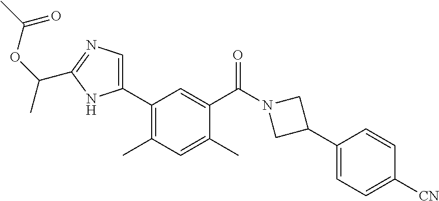 [SMILES: CC(=O)OC(C)c1ncc(-c2cc(C(=O)N3CC(c4ccc(C#N)cc4)C3)c(C)cc2C)[nH]1] | |
| 002-336 | 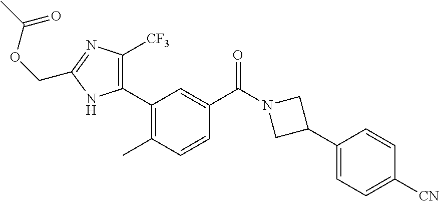 [SMILES: CC(=O)OCc1nc(C(F)(F)F)c(-c2cc(C(=O)N3CC(c4ccc(C#N)cc4)C3)ccc2C)[nH]1] | |
| 002-337 | 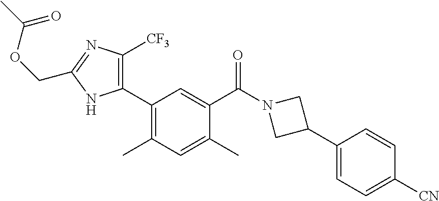 [SMILES: CC(=O)OCc1nc(C(F)(F)F)c(-c2cc(C(=O)N3CC(c4ccc(C#N)cc4)C3)c(C)cc2C)[nH]1] | |
| 002-338 | 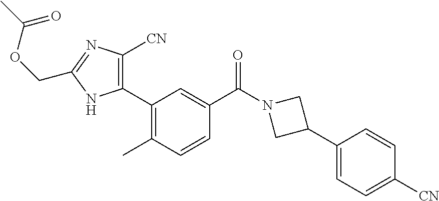 [SMILES: CC(=O)OCc1nc(C#N)c(-c2cc(C(=O)N3CC(c4ccc(C#N)cc4)C3)ccc2C)[nH]1] | |
| 002-339 | 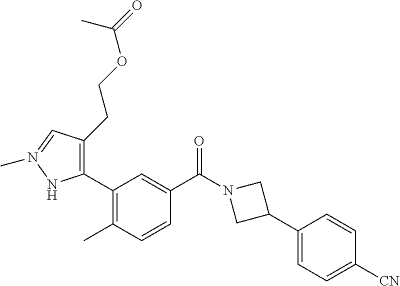 | |
| 002-340 | 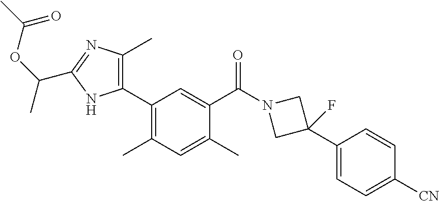 [SMILES: CC(=O)OC(C)c1nc(C)c(-c2cc(C(=O)N3CC(F)(c4ccc(C#N)cc4)C3)c(C)cc2C)[nH]1] | |
| 002-341 | 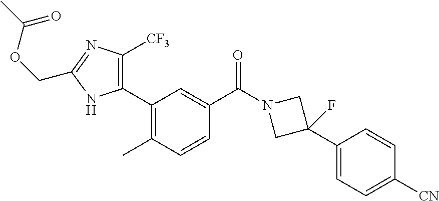 [SMILES: CC(=O)OCc1nc(C(F)(F)F)c(-c2cc(C(=O)N3CC(F)(c4ccc(C#N)cc4)C3)ccc2C)[nH]1] | |
| 002-342 | 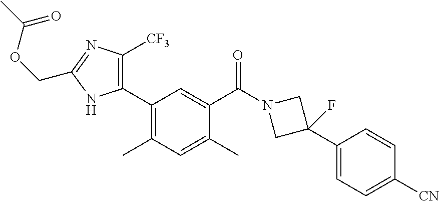 [SMILES: CC(=O)OCc1nc(C(F)(F)F)c(-c2cc(C(=O)N3CC(F)(c4ccc(C#N)cc4)C3)c(C)cc2C)[nH]1] | |
| 002-343 | 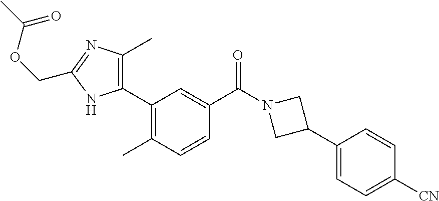 [SMILES: CC(=O)OCc1nc(C)c(-c2cc(C(=O)N3CC(c4ccc(C#N)cc4)C3)ccc2C)[nH]1] | |
| 002-344 | 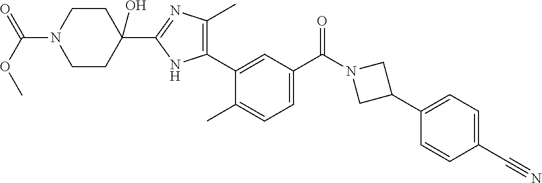 [SMILES: COC(=O)N1CCC(O)(c2nc(C)c(-c3cc(C(=O)N4CC(c5ccc(C#N)cc5)C4)ccc3C)[nH]2)CC1] | 0.024 |
| 002-345 | 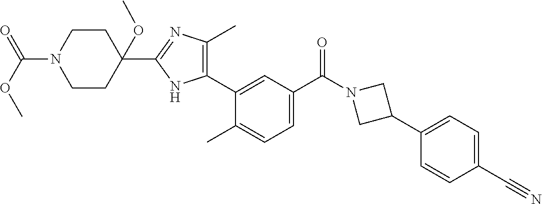 [SMILES: COC(=O)N1CCC(OC)(c2nc(C)c(-c3cc(C(=O)N4CC(c5ccc(C#N)cc5)C4)ccc3C)[nH]2)CC1] | 0.035 |
| 002-346 | 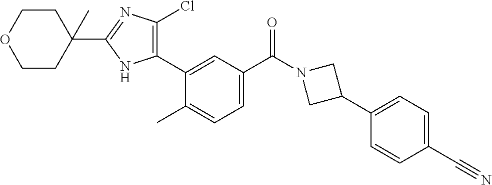 [SMILES: Cc1ccc(C(=O)N2CC(c3ccc(C#N)cc3)C2)cc1-c1[nH]c(C2(C)CCOCC2)nc1Cl] | 0.018 |
| 002-347 | 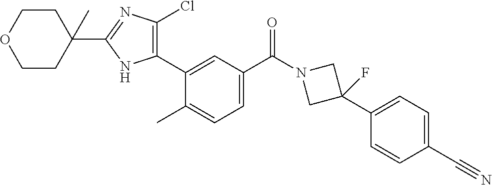 [SMILES: Cc1ccc(C(=O)N2CC(F)(c3ccc(C#N)cc3)C2)cc1-c1[nH]c(C2(C)CCOCC2)nc1Cl] | 0.016 |
| 002-348 | 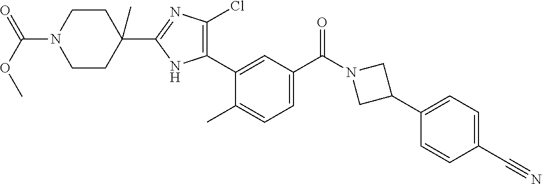 [SMILES: COC(=O)N1CCC(C)(c2nc(Cl)c(-c3cc(C(=O)N4CC(c5ccc(C#N)cc5)C4)ccc3C)[nH]2)CC1] | 0.014 |
| 002-349 | 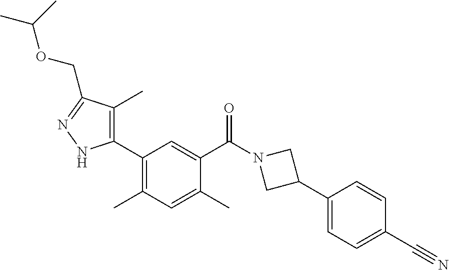 [SMILES: Cc1cc(C)c(-c2[nH]nc(COC(C)C)c2C)cc1C(=O)N1CC(c2ccc(C#N)cc2)C1] | 0.019 |
| 002-350 | 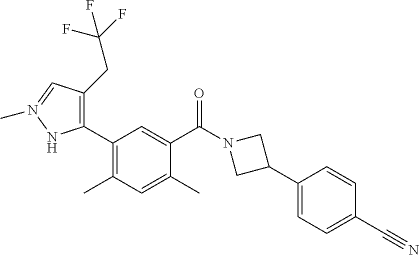 | 0.055 |
| 002-351 | 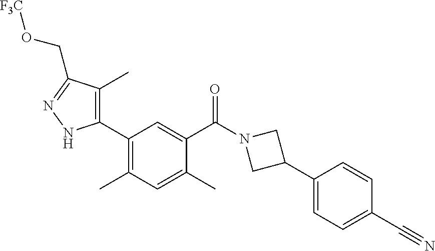 [SMILES: Cc1cc(C)c(-c2[nH]nc(COC(F)(F)F)c2C)cc1C(=O)N1CC(c2ccc(C#N)cc2)C1] | |
| 002-352 | 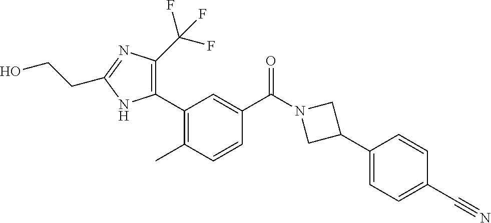 [SMILES: Cc1ccc(C(=O)N2CC(c3ccc(C#N)cc3)C2)cc1-c1[nH]c(CCO)nc1C(F)(F)F] | 0.011 |
| 002-353 | 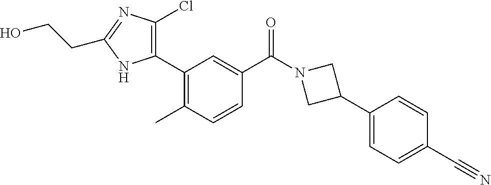 [SMILES: Cc1ccc(C(=O)N2CC(c3ccc(C#N)cc3)C2)cc1-c1[nH]c(CCO)nc1Cl] | 0.009 |
| 002-354 | 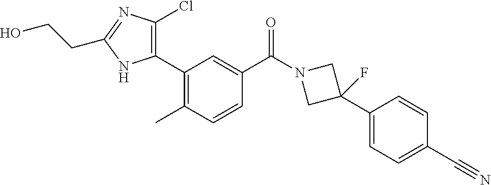 [SMILES: Cc1ccc(C(=O)N2CC(F)(c3ccc(C#N)cc3)C2)cc1-c1[nH]c(CCO)nc1Cl] | 0.018 |
| 002-355 | 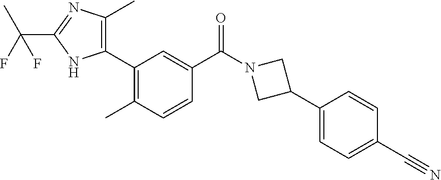 [SMILES: Cc1ccc(C(=O)N2CC(c3ccc(C#N)cc3)C2)cc1-c1[nH]c(C(C)(F)F)nc1C] | 0.082 |
| 002-356 | 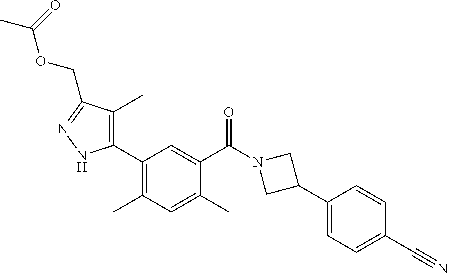 [SMILES: CC(=O)OCc1n[nH]c(-c2cc(C(=O)N3CC(c4ccc(C#N)cc4)C3)c(C)cc2C)c1C] | 0.851 |
| 002-357 | 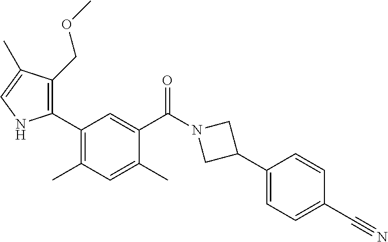 [SMILES: COCc1c(C)c[nH]c1-c1cc(C(=O)N2CC(c3ccc(C#N)cc3)C2)c(C)cc1C] | 0.087 |
| 002-358 | 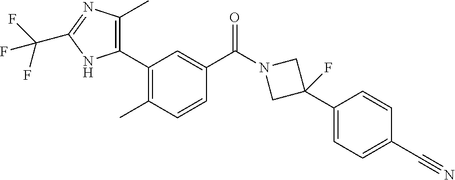 [SMILES: Cc1ccc(C(=O)N2CC(F)(c3ccc(C#N)cc3)C2)cc1-c1[nH]c(C(F)(F)F)nc1C] | 0.1001 |
| 002-359 | 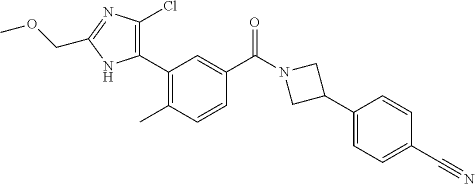 [SMILES: COCc1nc(Cl)c(-c2cc(C(=O)N3CC(c4ccc(C#N)cc4)C3)ccc2C)[nH]1] | 0.014 |
| 002-360 | 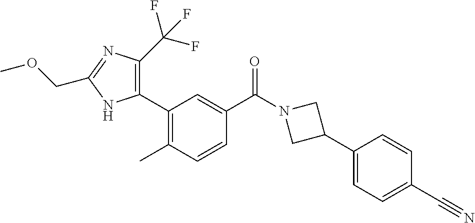 [SMILES: COCc1nc(C(F)(F)F)c(-c2cc(C(=O)N3CC(c4ccc(C#N)cc4)C3)ccc2C)[nH]1] | 0.012 |
| 002-36 | 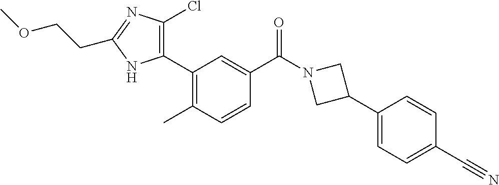 [SMILES: COCCc1nc(Cl)c(-c2cc(C(=O)N3CC(c4ccc(C#N)cc4)C3)ccc2C)[nH]1] | 0.013 |
| 002-362 | 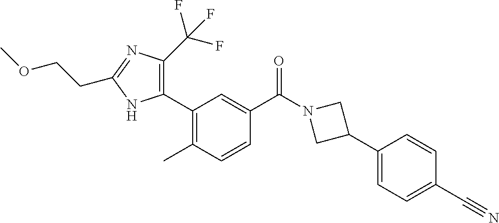 [SMILES: COCCc1nc(C(F)(F)F)c(-c2cc(C(=O)N3CC(c4ccc(C#N)cc4)C3)ccc2C)[nH]1] | 0.011 |
| 002-363 | 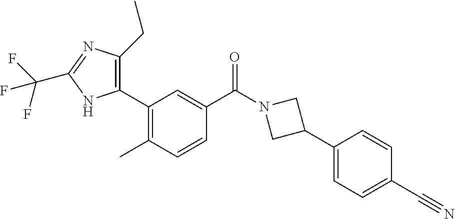 [SMILES: CCc1nc(C(F)(F)F)[nH]c1-c1cc(C(=O)N2CC(c3ccc(C#N)cc3)C2)ccc1C] | 0.021 |
| 002-364 | 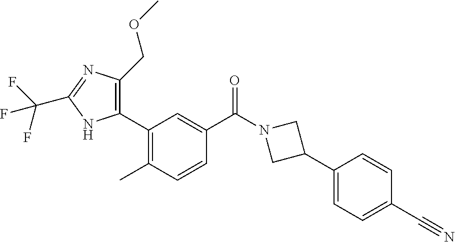 [SMILES: COCc1nc(C(F)(F)F)[nH]c1-c1cc(C(=O)N2CC(c3ccc(C#N)cc3)C2)ccc1C] | 0.040 |
| 002-365 | 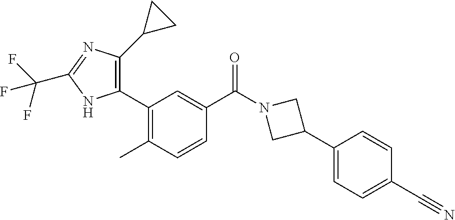 [SMILES: Cc1ccc(C(=O)N2CC(c3ccc(C#N)cc3)C2)cc1-c1[nH]c(C(F)(F)F)nc1C1CC1] | 0.023 |
| 002-366 | 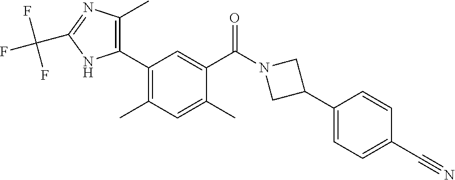 [SMILES: Cc1cc(C)c(-c2[nH]c(C(F)(F)F)nc2C)cc1C(=O)N1CC(c2ccc(C#N)cc2)C1] | 0.022 |
| 002-367 | 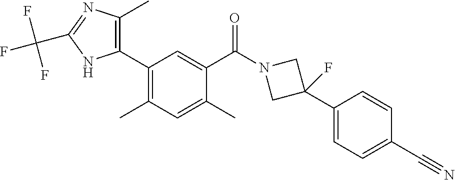 [SMILES: Cc1cc(C)c(-c2[nH]c(C(F)(F)F)nc2C)cc1C(=O)N1CC(F)(c2ccc(C#N)cc2)C1] | 0.040 |
| 002-368 | 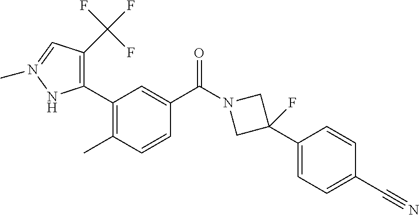 | 0.012 |
| 002-369 | 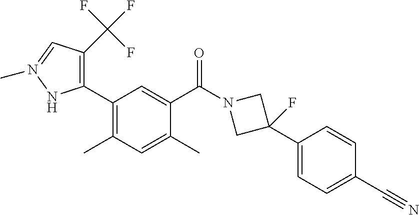 | 0.014 |
| 002-370 | 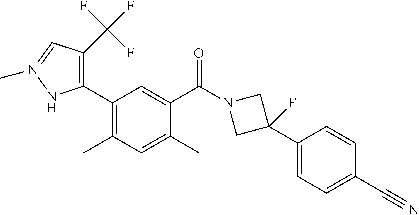 | 0.012 |
| 002-371 | 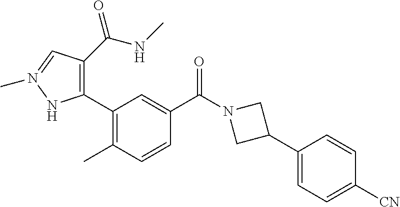 | 0.82 |
| 002-374 | 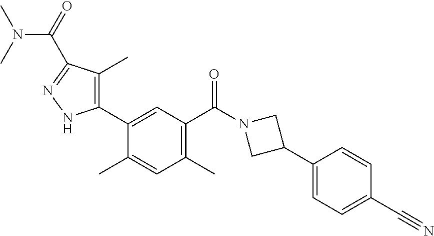 [SMILES: Cc1cc(C)c(-c2[nH]nc(C(=O)N(C)C)c2C)cc1C(=O)N1CC(c2ccc(C#N)cc2)C1] | 0.024 |
| 002-375 | 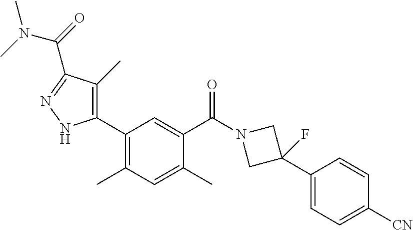 [SMILES: Cc1cc(C)c(-c2[nH]nc(C(=O)N(C)C)c2C)cc1C(=O)N1CC(F)(c2ccc(C#N)cc2)C1] | |
| 002-376 | 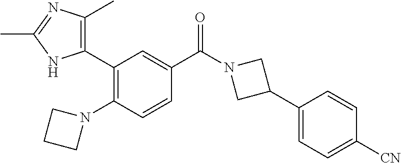 [SMILES: Cc1nc(C)c(-c2cc(C(=O)N3CC(c4ccc(C#N)cc4)C3)ccc2N2CCC2)[nH]1] | 0.029 |
| 002-377 | 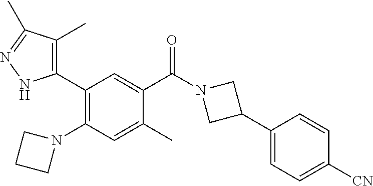 [SMILES: Cc1cc(N2CCC2)c(-c2[nH]nc(C)c2C)cc1C(=O)N1CC(c2ccc(C#N)cc2)C1] | 0.029 |
| 002-378 | 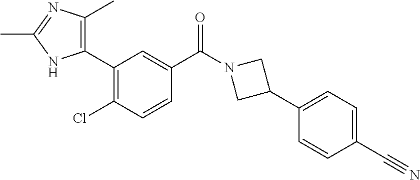 [SMILES: Cc1nc(C)c(-c2cc(C(=O)N3CC(c4ccc(C#N)cc4)C3)ccc2Cl)[nH]1] | 0.072 |
| 002-379 | 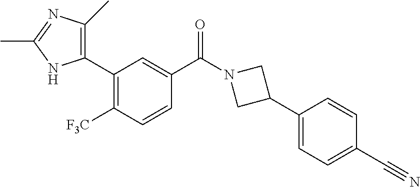 [SMILES: Cc1nc(C)c(-c2cc(C(=O)N3CC(c4ccc(C#N)cc4)C3)ccc2C(F)(F)F)[nH]1] | 0.059 |
| 002-380 | 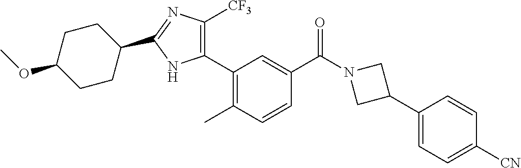 [SMILES: CO[C@H]1CC[C@@H](c2nc(C(F)(F)F)c(-c3cc(C(=O)N4CC(c5ccc(C#N)cc5)C4)ccc3C)[nH]2)CC1] | 0.013 |
| 002-381 | 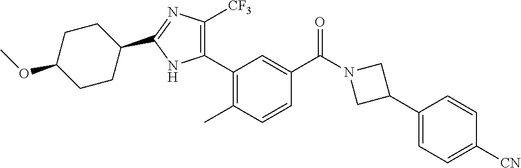 [SMILES: CO[C@H]1CC[C@@H](c2nc(C(F)(F)F)c(-c3cc(C(=O)N4CC(c5ccc(C#N)cc5)C4)ccc3C)[nH]2)CC1] | 0.009 |
| 002-382 | 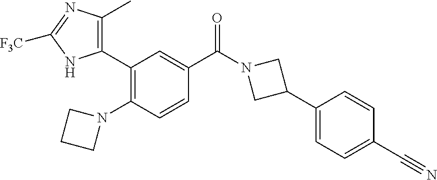 [SMILES: Cc1nc(C(F)(F)F)[nH]c1-c1cc(C(=O)N2CC(c3ccc(C#N)cc3)C2)ccc1N1CCC1] | 0.018 |
| 002-383 | 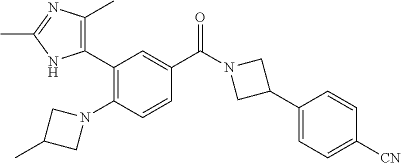 [SMILES: Cc1nc(C)c(-c2cc(C(=O)N3CC(c4ccc(C#N)cc4)C3)ccc2N2CC(C)C2)[nH]1] | 0.024 |
| 002-384 | 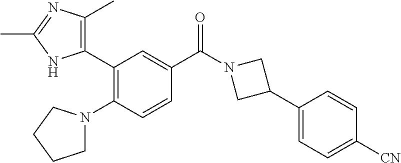 [SMILES: Cc1nc(C)c(-c2cc(C(=O)N3CC(c4ccc(C#N)cc4)C3)ccc2N2CCCC2)[nH]1] | 0.012 |
| 002-385 | 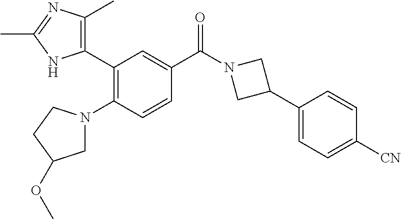 [SMILES: COC1CCN(c2ccc(C(=O)N3CC(c4ccc(C#N)cc4)C3)cc2-c2[nH]c(C)nc2C)C1] | 0.04 |
| 002-386 | 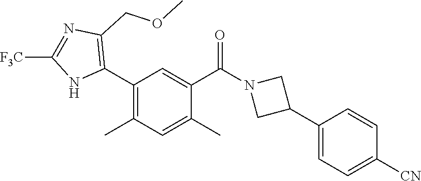 [SMILES: COCc1nc(C(F)(F)F)[nH]c1-c1cc(C(=O)N2CC(c3ccc(C#N)cc3)C2)c(C)cc1C] | |

TABLE C-3
Fatty Acid Synthase Inhibitors

| | | Hu | | | FASN |
|---|---|---|---|---|---|
| | | FASN | | | (human) |
| Cmpd | | IC50 | Cmpd | | IC50 |
| No. | Structure | (μM) | No. | Structure | (μM) |
| 005-1 | 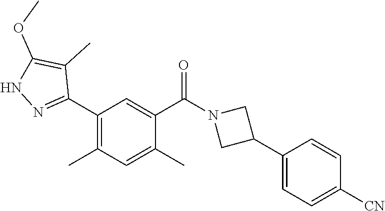 [SMILES: COc1[nH]nc(-c2cc(C(=O)N3CC(c4ccc(C#N)cc4)C3)c(C)cc2C)c1C] | 0.039 | 005-12 | 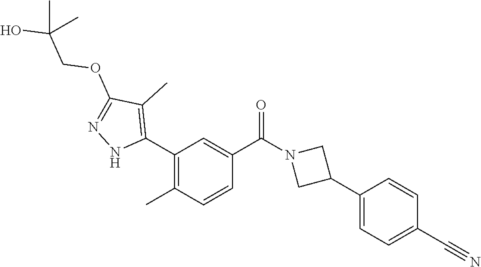 [SMILES: Cc1ccc(C(=O)N2CC(c3ccc(C#N)cc3)C2)cc1-c1[nH]nc(OCC(C)(C)O)c1C] | 0.01 |
| 005-2 | 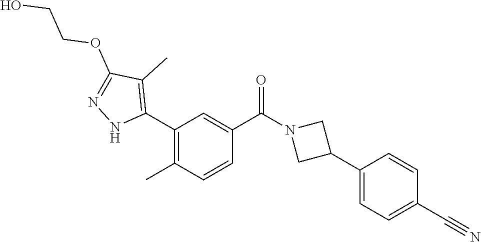 [SMILES: Cc1ccc(C(=O)N2CC(c3ccc(C#N)cc3)C2)cc1-c1[nH]nc(OCCO)c1C] | 0.023 | 005-13 | 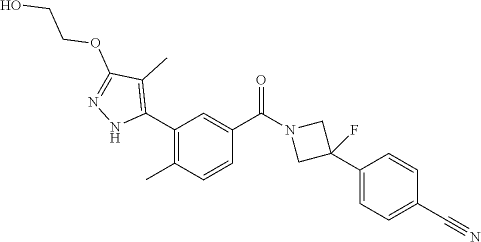 [SMILES: Cc1ccc(C(=O)N2CC(F)(c3ccc(C#N)cc3)C2)cc1-c1[nH]nc(OCCO)c1C] | 0.19 |
| 005-3 | 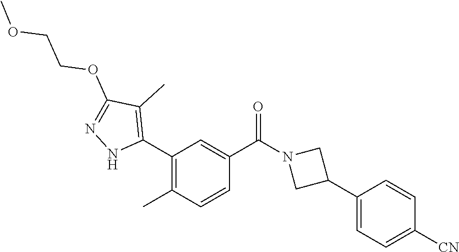 [SMILES: COCCOc1n[nH]c(-c2cc(C(=O)N3CC(c4ccc(C#N)cc4)C3)ccc2C)c1C] | 0.015 | 005-14 | 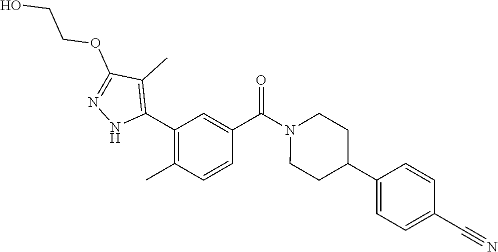 [SMILES: Cc1ccc(C(=O)N2CCC(c3ccc(C#N)cc3)CC2)cc1-c1[nH]nc(OCCO)c1C] | 0.084 |
| 005-4 | 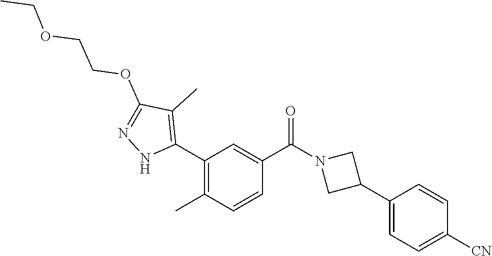 [SMILES: CCOCCOc1n[nH]c(-c2cc(C(=O)N3CC(c4ccc(C#N)cc4)C3)ccc2C)c1C] | 0.02 | 005-15 | 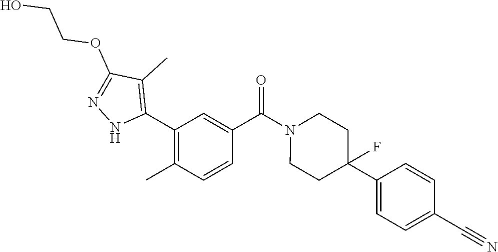 [SMILES: Cc1ccc(C(=O)N2CCC(F)(c3ccc(C#N)cc3)CC2)cc1-c1[nH]nc(OCCO)c1C] | 0.23 |
| 005-5 | 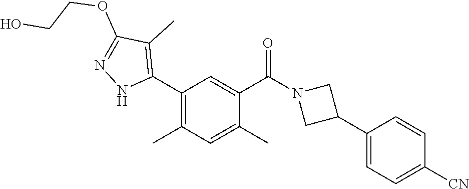 [SMILES: Cc1cc(C)c(-c2[nH]nc(OCCO)c2C)cc1C(=O)N1CC(c2ccc(C#N)cc2)C1] | 0.019 | 005-16 | 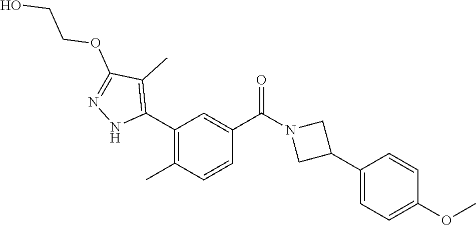 [SMILES: COc1ccc(C2CN(C(=O)c3ccc(C)c(-c4[nH]nc(OCCO)c4C)c3)C2)cc1] | 0.039 |
| 005-6 | 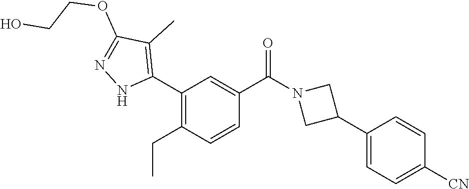 [SMILES: CCc1ccc(C(=O)N2CC(c3ccc(C#N)cc3)C2)cc1-c1[nH]nc(OCCO)c1C] | 0.016 | 005-17 | 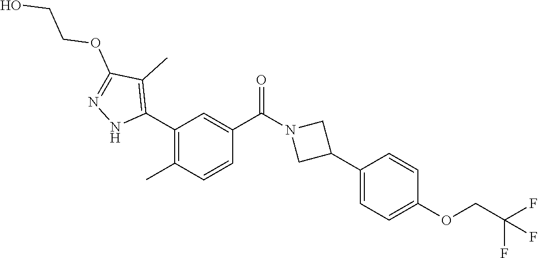 [SMILES: Cc1ccc(C(=O)N2CC(c3ccc(OCC(F)(F)F)cc3)C2)cc1-c1[nH]nc(OCCO)c1C] | 0.039 |
| 005-7 | 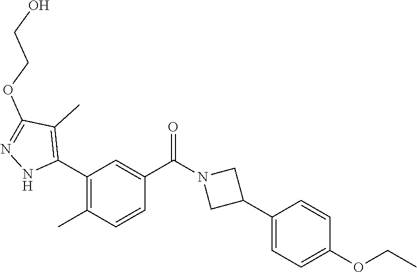 [SMILES: CCOc1ccc(C2CN(C(=O)c3ccc(C)c(-c4[nH]nc(OCCO)c4C)c3)C2)cc1] | 0.022 | 005-18 | 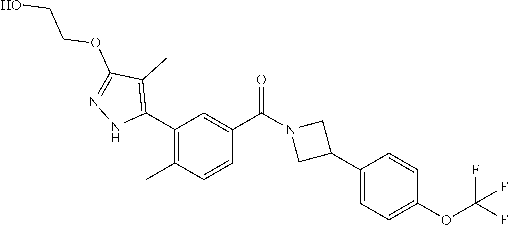 [SMILES: Cc1ccc(C(=O)N2CC(c3ccc(OC(F)(F)F)cc3)C2)cc1-c1[nH]nc(OCCO)c1C] | 0.037 |
| 005-8 | 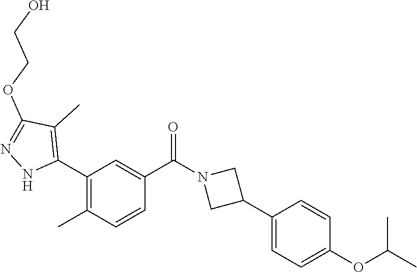 [SMILES: Cc1ccc(C(=O)N2CC(c3ccc(OC(C)C)cc3)C2)cc1-c1[nH]nc(OCCO)c1C] | 0.035 | 005-19 | 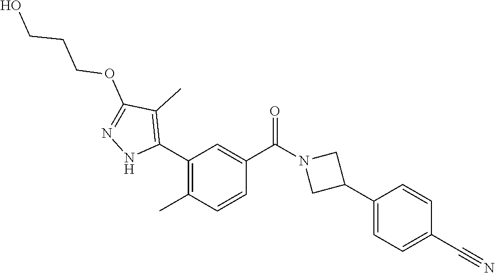 [SMILES: Cc1ccc(C(=O)N2CC(c3ccc(C#N)cc3)C2)cc1-c1[nH]nc(OCCCO)c1C] | 0.066 |
| 005-9 | 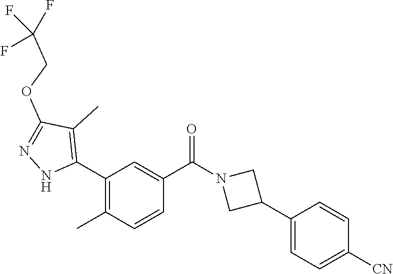 [SMILES: Cc1ccc(C(=O)N2CC(c3ccc(C#N)cc3)C2)cc1-c1[nH]nc(OCC(F)(F)F)c1C] | | 005-20 | 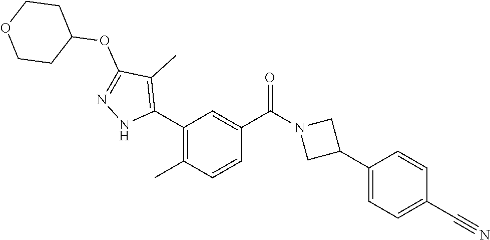 [SMILES: Cc1ccc(C(=O)N2CC(c3ccc(C#N)cc3)C2)cc1-c1[nH]nc(OC2CCOCC2)c1C] | 0.058 |
| 005-10 | 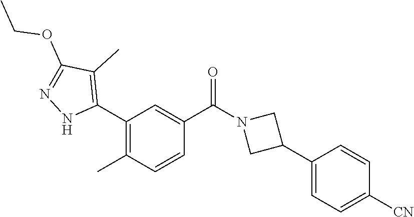 [SMILES: CCOc1n[nH]c(-c2cc(C(=O)N3CC(c4ccc(C#N)cc4)C3)ccc2C)c1C] | | 005-11 | 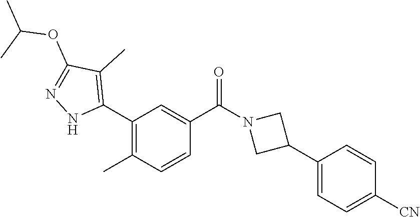 [SMILES: Cc1ccc(C(=O)N2CC(c3ccc(C#N)cc3)C2)cc1-c1[nH]nc(OC(C)C)c1C] | 005-11 |

What is claimed:

1.-6. (canceled)

7. A method of treating a subject having or suspected of having non-alcoholic steatohepatitis (NASH) or non-alcoholic fatty liver disease (NAFLD) with a fatty acid synthase (FASN) inhibitor, comprising:
receiving, by processing circuitry, input data associated with the subject, the input data including biochemical data characterizing a state of the subject;
applying, by the processing circuitry, a machine learning model to the input data to generate an output value predicting a percent change in liver fat following administration of the FASN inhibitor;
comparing, by the processing circuitry, the output value of the machine learning model to a treatment threshold value; and
administering an effective amount of the FASN inhibitor to the subject if the comparing indicates the predicted percent change in liver fat exceeds the treatment threshold value.

8. The method of claim 7, wherein the biochemical data characterizing the state of the subject includes at least one bile acid, at least one secondary bile acid, at least one bile acid derivative, at least one amino acid, at least one amino acid derivative, and/or at least one glycerophospholipids.

9. The method of claim 7, wherein the biochemical data characterizing the state of the subject include ursodeoxycholic acid, hyodeoxycholic acid, DL-2-aminocaprylic acid, D(−)-2-aminobutyric acid, sarcosine, glycoursodeoxycholic acid, and/or PC(O-18:0/22:4).

10. The method of claim 7, wherein the machine learning model is a random decision forest.

11. The method of claim 10, wherein the biochemical data included in the random decision forest is based on mean decrease in impurity.

12. The method of claim 7, wherein the machine learning model is a support-vector machine.

13. The method of claim 12, wherein the biochemical data included in the support-vector machine is based on a permutation importance algorithm.

14.-28. (canceled)

29. A method for administration of a fatty acid synthase (FASN) inhibitor to a subject with non-alcoholic steatohepatitis (NASH) or non-alcoholic fatty liver disease (NAFLD), comprising:
determining a predicted responsiveness of the subject with NASH or NAFLD to the FASN inhibitor by calculating a prognostic value based on results of a biomarker assay performed on a biological sample obtained from the subject with NASH or NAFLD; and
administering the FASN inhibitor to the subject with NASH or NAFLD when the calculated prognostic value indicates a high predicted responsiveness of the subject with NASH or NAFLD to the FASN inhibitor.

30. The method of claim 29, wherein the biological sample obtained from the subject with NASH includes, as biomarkers, ursodeoxycholic acid, hyodeoxycholic acid, DL-2-aminocaprylic acid, glycoursodeoxycholic acid, sarcosine, and/or PC(O-18:0/22:4).

31. The method of claim 29, wherein the prognostic value is obtained by applying a machine learning model to the results of the biomarker assay, the machine learning model being trained on a database of training data, the database including biomarker data characterizing a state of a reference subject before administration of the FASN inhibitor and a corresponding percent change in liver fat following administration of the FASN inhibitor.

32.-33. (canceled)

34. The method of claim 7, wherein the FASN inhibitor is a compound of Formula (IX-1), and wherein R¹ is H, —CN, halogen, or C₁-C₄ straight or branched alkyl; each R² is independently H; R³ is H or halogen; R²¹ is C₁-C₄ straight or branched alkyl or C₃-C₅ cycloalkyl; R²² is H or C₁-C₂ alkyl; and R²⁴ is H or C₁-C₄ straight or branched alkyl.

35. The method of claim 7, wherein the FASN inhibitor is a compound of Formula (IX-1), and wherein R¹ is —CN; each R² is independently H; R³ is H; R²¹ is C₃-C₅ cycloalkyl; R²² is H or C₁-C₂ alkyl; and R²⁴ is C₁-C₄ straight or branched alkyl.

36.-39. (canceled)

40. The method of claim 7, wherein the FASN inhibitor is a compound of Formula (XX-1), and wherein L-Ar is 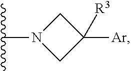
Ar is 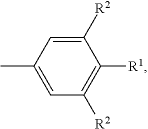
R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl), —O—(C₁-C₄ alkyl)-O—(C₁-C₄ alkyl), or —O-(4- to 6-membered heterocycle), wherein R²⁴ is optionally substituted with one or more hydroxyl or halogen; and R²⁵ is H, halogen, or C₁-C₄ alkyl.

41. The method of claim 7, wherein the FASN inhibitor is a compound of Formula (XX-1), and wherein L-Ar is 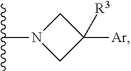
Ar is 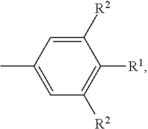
R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl) substituted with one or more hydroxyl or halogen; and R²⁵ is C₁-C₄ alkyl.

42. The method of claim 7, wherein the FASN inhibitor is a compound selected from: 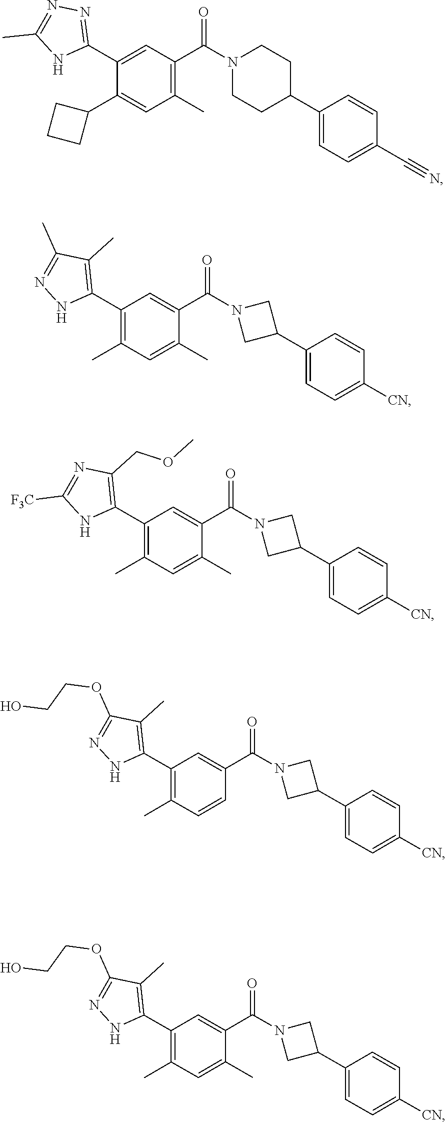 [SMILES: Cc1nnc(-c2cc(C(=O)N3CCC(c4ccc(C#N)cc4)CC3)c(C)cc2C2CCC2)[nH]1 ; Cc1cc(C)c(-c2[nH]nc(C)c2C)cc1C(=O)N1CC(c2ccc(C#N)cc2)C1 ; COCc1nc(C(F)(F)F)[nH]c1-c1cc(C(=O)N2CC(c3ccc(C#N)cc3)C2)c(C)cc1C ; Cc1ccc(C(=O)N2CC(c3ccc(C#N)cc3)C2)cc1-c1[nH]nc(OCCO)c1C ; Cc1cc(C)c(-c2[nH]nc(OCCO)c2C)cc1C(=O)N1CC(c2ccc(C#N)cc2)C1]
and pharmaceutically acceptable salts thereof.

43.-44. (canceled)

45. The method of claim 29, wherein the FASN inhibitor is a compound of Formula (IX-1), and wherein R¹ is H, —CN, halogen, or C₁-C₄ straight or branched alkyl; each R² is independently H; R³ is H or halogen; R²¹ is C₁-C₄ straight or branched alkyl or C₃-C₅ cycloalkyl; R²² is H or C₁-C₂ alkyl; and R²⁴ is H or C₁-C₄ straight or branched alkyl.

46. The method of claim 29, wherein the FASN inhibitor is a compound of Formula (IX-1), and wherein R¹ is —CN; each R² is independently H; R³ is H; R²¹ is C₃-C₅ cycloalkyl; R²² is H or C₁-C₂ alkyl; and R²⁴ is C₁-C₄ straight or branched alkyl.

47. The method of claim 29, wherein the FASN inhibitor is a compound of Formula (XX-1), and wherein L-Ar is 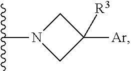
Ar is 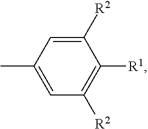
R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl), —O—(C₁-C₄ alkyl)-O—(C₁-C₄ alkyl), or —O-(4- to 6-membered heterocycle), wherein R²⁴ is optionally substituted with one or more hydroxyl or halogen; and R²⁵ is H, halogen, or C₁-C₄ alkyl.

48. The method of claim 29, wherein the FASN inhibitor is a compound of Formula (XX-1), and wherein L-Ar is 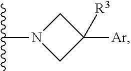
Ar is 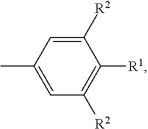
R¹ is —CN or —O—(C₁-C₄ alkyl) optionally substituted with one or more halogen; each R² is independently H; R³ is H or F; R²¹ is H or C₁-C₄ alkyl; R²² is H or C₁-C₂ alkyl; R²⁴ is —O—(C₁-C₄ alkyl) substituted with one or more hydroxyl or halogen; and R²⁵ is C₁-C₄ alkyl.

49. The method of claim 29, wherein the FASN inhibitor is a compound selected from: 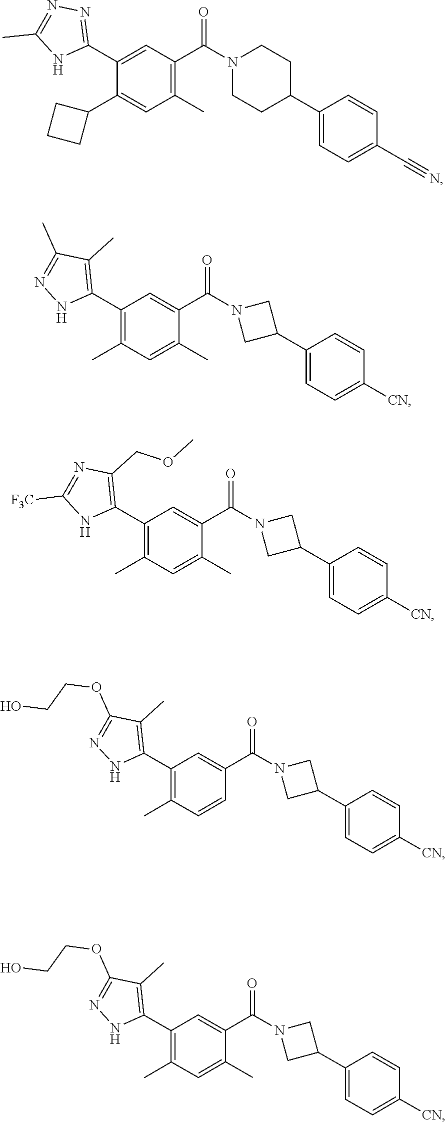 [SMILES: Cc1nnc(-c2cc(C(=O)N3CCC(c4ccc(C#N)cc4)CC3)c(C)cc2C2CCC2)[nH]1 ; Cc1cc(C)c(-c2[nH]nc(C)c2C)cc1C(=O)N1CC(c2ccc(C#N)cc2)C1 ; COCc1nc(C(F)(F)F)[nH]c1-c1cc(C(=O)N2CC(c3ccc(C#N)cc3)C2)c(C)cc1C ; Cc1ccc(C(=O)N2CC(c3ccc(C#N)cc3)C2)cc1-c1[nH]nc(OCCO)c1C ; Cc1cc(C)c(-c2[nH]nc(OCCO)c2C)cc1C(=O)N1CC(c2ccc(C#N)cc2)C1]
and pharmaceutically acceptable salts thereof.
